(19) United States
(12) Patent Application Publication
(10) Pub. No.: US 2026/0335371 A1
(43) Pub. Date: Oct. 29, 2026

(54) DIAGNOSTIC AND THERAPEUTIC USES OF COMPOSITIONS COMPRISING PURIFIED, ENRICHED POTENT EXOSOMES CONTAINING DISEASE-BASED AND THERAPY BASED SIGNATURE CARGO

(71) Applicant: Spiritus Therapeutics, Inc., New York, NY (US)

(72) Inventors: Jodi Gurney, New York, NY (US); Robin Smith, New York, NY (US)

(73) Assignee: Spiritus Therapeutics, Inc., (omitted)

(21) Appl. No.: 19/727,328

(22) Filed: Jul. 1, 2026

Related U.S. Application Data
(63) Continuation-in-part of application No. 17/700,283, filed on Mar. 21, 2022, now abandoned.
(60) Provisional application No. 63/164,430, filed on Mar. 22, 2021.
(60) Provisional application No. 63/219,277, filed on Jul. 7, 2021.

(51) Int. Cl. A61K 35/28; A61K 31/7105; A61K 41/00; C12N 5/0775; C12N 15/11

(52) U.S. Cl. CPC A61K 35/28; A61K 31/7105; A61K 41/0052; C12N 5/0668; C12N 15/111; C12N 2310/141

(57) ABSTRACT

The present disclosure provides a composition comprising a pharmaceutical carrier and a purified population of potent exosomes isolated from extracellular vesicles (EVs) that originate from mesenchymal stem cells (MSCs). The purified population of potent exosomes comprises an identity signature, total protein of about 1 mg; and a cargo comprising one or more miRNAs, wherein the miRNA cargo is tuned by a timed exposure first to heat and then to light, wherein lot to lot and batch to batch variability relative to prior lots of the composition is below a significance level of ≤0.05. It further provides a method for preparing the tuned population of exosomes that originate from MSCs and methods for treating various conditions comprising administering the composition comprising the population of exosomes comprising the tuned miRNA cargo.

### CROSS-REFERENCE TO RELATED APPLICATIONS

This application is a continuation-in-part of U.S. patent application Ser. No. 17/700,283, filed Mar. 21, 2022, which claims priority to and the benefit of U.S. Provisional Patent Application No. 63/219,277, filed Jul. 7, 2021, and U.S. Provisional Patent Application No. 63/164,430, filed Mar. 22, 2021. The disclosures of each of these applications are incorporated herein by reference in their entirety.

### FIELD OF INVENTION

The described invention generally relates to mesenchymal stem cell-derived extracellular vesicles, their cargo, including a disease-based signature of their cargo, and therapy-based signatures of compositions comprising extracellular vesicles and their application for diagnostic and therapeutic uses.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 30, 2026, is named 2746-00303USCIP-SEQLIST-ST26.xml and is 7,303 bytes in size. The sequence listing contained in this .XML file is part of the specification and is hereby incorporated by reference herein in its entirety.

### BACKGROUND OF THE INVENTION

The therapeutic efficacy of mesenchymal stem cells (MSCs) that was predicated on the differentiation potential of MSCs now is increasingly attributed to the paracrine effects of these cells through the release of extracellular vesicles (EVs), particularly exosomes [Tan, S S H et al. Tissue Engineering, Part B. (2020) DOI: 10.1089/ten.teb.2019.0326].

miRNA Expression in MSCs and Microvesicles

It is now established that MSCs secrete microvesicles (MVs) (Id., citing Bruno S, et al. J. Am. Soc. Nephrol. (2009) 20: 1053-67; Collino, F. et al. PLoS One (2010) 5: e11803). An miRNA profile on MVs from MSCs and human liver stem cells (HLSCs) showed that MVs contained a pattern of miRNAs shared with their cells of origin (Collino F, et al. PLoS One (2010) 5: e11803).

Accumulating data suggest that MVs serve as a means of cell-to-cell communication through which genetic information or gene products are transferred and cell activities are regulated (Id., citing Carmussi, et al. Kidney Int. (2010) 78: 838-848; Lotvall, J., Valadi, H. Cell Adh. Mgr. (2007) 1: 156-58). MSC EVs have been shown to harbor a variety of mRNAs and miRNAs (Carmussi, et al. Kidney Int. (2010) 78: 838-848; Lotvall, J., Valadi, H. Cell Adh. Mgr. (2007) 1: 156-58; Chen, T S et al. Nucleic Acids Res. (2010) 38: 215-224). Differential miRNA expression profiles in MSCs and MVs derived from MSCs have been observed (Carmussi, et al. Kidney Int. (2010) 78: 838-848; Chen, T S et al. Nucleic Acids Res. (2010) 38: 215-224). Microarray analysis for the presence of miRNAs revealed that the secreted RNA contained many miRNAs that were essentially a subset of those in MSCs. 9 of the 13 members in one of the most highly conserved and developmentally important human let-7 family were expressed in MSCs (citing Jerome T, et al. Curr. Genomics. 2007; 8: 229-233; Roush S, Slack F J. Trends Cell Biol. 2008; 18: 505-516.). They were: hsa-let-7a, hsa-let-7b, hsa-let-7c, hsa-let-7d, hsa-let-7e, hsa-let-7f, hsa-let-7g, hsa-let-7i, and hsa-miR-98. Of these, only hsa-let-7a, -7b, -7c, and -7d were detected in the conditioned medium (CM) (Chen, T S et al. Nucleic Acids Res. (2010) 38: 215-224). The passenger miRNA sequences of -7b and -7d also were detected in the CM and not detectable in MSCs (Id.). These differences suggested that secretion of miRNAs including passenger miRNA sequences is a selective, and not a random process, by MSCs (Id.). Microarray analysis also revealed the presence of miRNA-923, a degradative product of ribosomal RNA (Id.). Therefore, while the secretion did not contain intact rRNA, it contained degraded ribosomal RNA and possibly degraded mRNA (Id.). MSCs were found to preferentially secrete miRNA in the precursor instead of the mature form; these pre-miRNAs were enriched in MVs, which were readily taken up by neighbor cells, suggesting a potential mechanism in regulation of activities of other cells (Id.).

### MSC EVs in Treatment of Organ Fibrosis

Beneficial effects have been described from MSC-exosomes in several disease models, e.g., in promoting repair and regeneration of osteochondral defects and alleviating osteoarthritis degeneration [Tan, S S H et al., Tissue Engineering: Part B (2020) doi: 10.1089/ten.teb.2019.0326]; in promoting functional recovery and neurovascular plasticity following traumatic brain injury [Willis, G R et al. Front. Cardiovasc. Med. (2017) 4: article 63, citing Zhang, Y et al. J. Neurosurg. (2015) 11: 856-67]; in reducing myocardial infarction size [Id., citing Lai, R C, et al. Stem Cell Res. (2010) 4: 214-22; Arslan, F. et al. Stem Cell Res. (2013) 10: 301-312]; ameliorating hypoxia-induced pulmonary hypertension [Id., citing Lee, C. et al. Circulation (2012) 126: 2601-11], aiding repair of kidney injury [Id., citing Dorronosoro, A., Robbins, PD. Stem Cell Res. Ther. (2013) 4: 39; Zhang, G. Exp. Ther. Med. (2016) 1: 1519-25], and orchestrating neurological protection by the transfer of miRNA [Id., citing Xin, H. et al. Stem Cells (2012) 30: 1556-64; Kalani, A., Tyagi, N. Neural Regen. Res. (2015) 10: 1565-7].

MSC-derived EVs also have shown protective effects in several models of organ injury and fibrosis. For example, the therapeutic effects of EVs produced by human BM MSCs (MEx) were tested in a murine bleomycin-induced pulmonary fibrosis model [Mansouri, N. et al., JCI Insight (2019) 4 (21). doi: 19.1172/jci.insight. 128060]. Exosomes isolated from fraction 9 of concentrated cell culture supernatants after flotation on an iodixanol cushion were about 34-150 mm in diameter and positive for established exosome markers (CD63, ALEX, Flotillin-1 and CD9). Adult C57BL/6 mice were challenged with endotracheal instillation of bleomycin and treated with 8.6±1×10¹⁰ MEx concurrently, or for reversal models, at day 7 or 21. Experimental groups were assessed at day 7, 14, or 28. Bleomycin-challenged mice presented with severe septal thickening and prominent fibrosis, which was effectively prevented or reversed by MEx treatment. MEx treatment modulated alveolar macrophage and monocyte populations in the lung, shifting the proportions of lung proinflammatory/classical and nonclassical monocytes and alveolar macrophages toward the monocyte/macrophage profiles of control mice. Bleomycin-induced elevation in the gene expression levels of proinflammatory cytokines was reduced by Mex administration. MEx delivered i.v. concomitantly with bleomycin shifted the bone marrow nonclassical monocyte profile toward that of their untreated counterparts. Transplantation of MEx-preconditioned BM-derived monocytes alleviated core features of pulmonary fibrosis and lung inflammation. Proteomic analysis revealed that MEx therapy reprogrammed bone marrow derived monocytes to a nonclassical anti-inflammatory phenotype. A bolus dose of purified MSC-exosomes significantly improved lung morphology and pulmonary development, decreased lung fibrosis, and ameliorated pulmonary vascular remodeling in a neonatal hyperoxia-induced murine model of bronchopulmonary dysplasia (BPD). While term mouse lungs in this model present a development stage resembling that of a human preterm neonate between 14 and 28 weeks gestation, these lungs, albeit in the saccular stage, are competent for proper gas exchange, whereas human preterm neonates often require supplemental oxygen and surfactant administration [Id.]. It was suggested that MSC-exosomes can modulate proinflammatory signaling and immune responses in the hyperoxic lung via modulation of lung macrophage phenotype. (Willis, G R, et al. Am. J. Respir. Crit. Care Med. (2018) 197 (1): 104-116).

In murine models of kidney injury, MSC-derived EVs protected against renal injury by reducing levels of creatinine, uric acid, lymphocyte response and fibrosis through shuttling miR-let7c to induce renal tubular cell proliferation (Kusuma G D, et al. Front Pharmacol. 2018; 9: 1199, citing Wang B, et al. Mol Ther. 2016 August; 24(7): 1290-301). In a murine model of carbon tetrachloride-induced hepatic injury, concurrent treatments of MSC-EVs attenuated the injury by increasing the proliferation, survival and prevented the apoptosis of hepatocytes (Id., citing Tan C Y, et al. Stem Cell Res Ther. 2014; 5(3): 76). In animal models of lung injury, MSC- and human aortic endothelial cell (hAEC)-EVs have been shown to reduce pulmonary inflammation, improved lung tissue recovery and supported the proliferation of alveolar type II and bronchioalveolar stem cells (Id., citing Rubenfeld G D, et al. N Engl J Med. 2005 Oct. 20; 353(16): 1685-93; Cruz F F, et al. Stem Cells Transl Med. 2015 November; 4(11): 1302-16; Monsel A, et al. Am J Respir Crit Care Med. 2015 Aug. 1; 192(3): 324-36; Tan J L, et al. Stem Cells Transl Med. 2018 February; 7(2): 180-196). In models of stroke, MSC-EVs delivery of miR-133b directly to neurite cells reportedly enhanced the outgrowth of neurites resulting in increased proliferation of neuroblasts and endothelial cells (Id., citing Xin H, et al. Stem Cells. 2013 December; 31(12): 2737-46). Additionally, Anderson et al. showed through a comprehensive proteomic analysis that MSC-derived EVs mediated angiogenesis via NF-κB signaling (Anderson J D, et al. Stem Cells. 2016 March; 34(3): 601-13), while Zhang et al. (Stem Cells Transl Med. 2015 May; 4(5): 513-22) showed that UC MSC-EVs mediated angiogenesis via the Wnt4/β-catenin pathway.

Cocksackievirus B (CVB), a member of the Picornaviridae family, is a common enterovirus that can cause various human systemic inflammatory disease, such as myocarditis, meningitis, and pancreatitis; the six CVB serotypes are each responsible for different diseases and symptoms [Li, X et al. Cell Death & Disease (2015) 10: 691, citing Sin, J. et al, J. Viol. (2017) 91: e0134-17; Wang, Y. et al. PLoS Pathog. (2018) 14: e10068872; An, B. et al. J. Cardiovasc. Pharmacol. (2017) 69: 305-313]. Viral myocarditis is a common cause of dilated cardiomyopathy and sudden cardiac death. Several preclinical stem cell therapies have made some progress in reducing inflammation and improving myocardial function, but they still have limitations [Id., citing Werner, L. et al. J. Mol. Cell Cardio. (2005) 39: 691-97; Miteva, K. et al. Stem Cells Trans. Med. (2017) 6: 1249-61; Van Linthout, S. et al. Eur. Heart J. (2011) 32: 2168-78].

Cardiac progenitor cells CPCs are a group of heterogeneous cells distributed throughout the heart and able to differentiate into several cell types, including cardiomyocytes, vascular smooth muscle cells, and endothelial cells. Direct transdifferentiating into cardiac tissue is considered unlikely. The mechanism of adult stem cell therapy has been tested to be mediated through paracrine release of EVs containing growth factors and cytokines to exert anti-apoptosis effects, suppress immunity, and promote angiogenesis [Id., citing Le, T., Chang, J. Cell Death Discov. (2016) 2: 16052; Chimenti, I. et al. Circulation Res. (2010) 106: 971-80]. As a cell-free method, exosomes could avoid many of the limitations of cell therapy.

The role of exosomes isolated from expanded rat CPCs isolated and cultured from rat heart tissue was determined. Exosomes were isolated and purified using ExoQuick-TC™ Exosome Isolation Reagent (System Biosciences, USA). The presence and size of exosomes was determined using transmission electron microscopy. H9C2 cells also were used as an in vitro model; H9C2 myoblasts are a cell model used as an alternative for cardiomyocytes. To assess in vitro uptake of the CPC exosomes by H9C2 cells, purified exosomes were labeled with green fluorescent labeling (DiO perchlorate, Dio). The fluorescent green labeled CPC-exosomes were incubated with H9C2 cells for 12 h at 37 C and visualized by fluorescence microscopy. Cells in CVB3 groups were infected at 100 TCID50 with CVB3 or with FMEM containing 2% exosome free FBS as a negative control. TCID50 (median tissue culture infectious dose) signifies the concentration at which 50% of the cells are infected when a test tube or well plate upon which cells have been cultured is inoculated with a diluted solution of viral fluid.

For in vivo experiments, rats were injected with CVB3 containing 10⁴ TCID50 through the tail vein to establish the viral myocarditis model. Rats infected with CVB3 Nancy strain were injected with CPC-Ex i.v. at 24 h post-infection (p.i). All rats were sacrificed by cervical dislocation at 7 days p.i. and heart tissue acquired. Tissue structure was observed using H & E staining and TEM. Apoptosis of myocardial cells was detected by TdT-mediated dUTP nick-end labeling (TUNEL) and immunofluorescence. Apoptosis was determined by FACS analysis of cells stained with Annexin-V FITC and propidium iodide by flow cytometry. CVB3-induced apoptosis was inhibited by CPC-Ex at 12, 24 and 48 h. p.i.

After injection of CPC-Exosomes, the apoptotic rate of rat cardiomyocytes was significantly lower than that of the control group, and CPC-Exosomes improve myocardial function. The data indicate that the CPC-exosomes reduced the CVB3-induced apoptosis by inhibiting viral replication. Protein expression levels of Akt, mTor, p70S6K and 4EBP1 did not change much after injection of CPC-exosomes. Phosphorylation of Akt, mTor, and p70S6K was enhanced, and that of 4EBP1 was inhibited compared with the control groups.

Previous studies had shown that the Akt/mTOR signaling pathway plays an important role in anti-apoptosis and regulation of cellular transcription by activating p70S6K and 4EBP1. p70S6K is a serine/threonine-protein kinase that acts downstream of mTOR signaling in response to growth factors and nutrients to promote cell proliferation, cell growth and cell cycle progression. Eukaryotic translation initiation factor 4E (eIF4E)-binding protein 1 (4EBP1) is a member of a family of translation repressor proteins, and a well-known substrate of mechanistic target of rapamycin (mTOR) signaling pathway [Qin, X et al. Cell Cycle (2016) 15(6): 781-86]. The expression levels of related proteins and their phosphorylation levels was measured to further determine whether the mTOR signaling pathway is involved in this anti-apoptotic effect. When CPC-exosomes were added to cells in vitro and in vivo after CVB3 infection there was an increase in the expression of 4EBP1 and a decrease of its phosphorylation, together with reduced VP1 expression. It was hypothesized that since CVB3 replication can be suppressed by non-phosphorylated 4EBP1, which can lead to suppression of translation initiation of 5′TOP mRNA [Li, X et al. Cell Death & Disease (2019) 10: 691, citing Zhang, H M et al. Biochem. J. (2016) 473: 473-485], CPC-exosomes could inhibit CVB3 replication, thereby reducing the CVB3-induced apoptosis by inhibiting phosphorylation of 4EBP1. TOP mRNAs are vertebrate transcripts which contain a 5′ terminal oligopyrimidine tract (5′TOP), encode for ribosomal proteins and elongation factors 1alpha and 2, and are candidates for growth-dependent translational control mediated through their 5′TOP. (Avni, D. et al. Nucleic Acid Res. (1997) 25(5): 995-1001). In Akt2, p70S6K and 4EBP1 overexpression groups, CPC-exosomes promoted CVB3-induced apoptosis, viral capsid protein VP1 expression and cleavage of caspase 3.

### Extracellular Vesicles

Extracellular vesicles (EVs) are nano-sized (typically 50-200 nm in diameter) vesicles of different sizes, cargo, and surface markers that are secreted into the extracellular environment through a variety of mechanisms. They carry various components of the cytoplasm and cell membrane that are selectively loaded into these vesicles. They are secreted by all forms of living cells and play essential roles in different physiological functions and pathological processes. They also have been utilized as diagnostic markers and therapeutic tools in several conditions.

Three types of EVs are biologically distinguishable from one another via the distinct processes through which they are released by the cell. However, the experimental classification of these vesicles is less clear as there is no consensus on what criteria to use for their differentiation. Microvesicles (MVs), or ectosomes (200-2000 nm) arise by direct budding through the cell membrane to the outside of the cell [Shao, H. et al. Chem Rev. (2018) 118 (4): 1917-50]. Microvesicles are enriched in integrins, selectins, and CD40. Exosomes are produced through the inward invagination of the endosomal membrane pathway [Id., citing Thery, C. et al. Nat. Rev. Immunol. (2002) 2: 569-79; Kowal, J. et al. Curr. Opin. Cell Biol. (2014) 29: 116-25]. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-15; van Neil, G. et al. Nat. Rev. Mol. Cell Biol. (2018) 19: 213-28; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, NP, Llorente, A. Cell Mol. Life Sci. (2018) 75: 193-208). Small vesicles can be formed by further inward budding of the limiting membrane inside an endosome, leading to the formation of a multivesicular body (MVB), characterized by the presence of intraluminal vesicles [Shao, H. et al. Chem Rev. (2018) 118 (4): 1917-50, citing Piper, RC, Katzmann, DJ. Annu. Rev. Cell Dev. Biol. (2007) 23: 519-47]. During this process, cytosolic contents, transmembrane and peripheral proteins are incorporated into the invaginating membrane [Id., citing Hurley, JH, Hanson, PI. Nat. Rev. Mol. Cell Biol. (2010) 11: 556-66]. ESEs can mature into late-sorting endosomes (LSEs) and eventually generate MVBs, which are also called multivesicular endosomes. MVBs form by inward invagination of the endosomal limiting membrane (that is, double invagination of the plasma membrane). MVBs may then fuse with the lysosome, leading to the degradation of vesicular contents [Id., citing Vlassov, A V et al. Biochim. Biophys. Acta (2012) 1820: 940-48]. Alternatively, MVBs may fuse with the plasma membrane of the cell, releasing vesicles in an exocytotic fashion to the extracellular space. [Id., citing Colombo, M. et al. Annu. Rev. Cell Dev. Biol. (2014) 30: 255-89, Thery, C. F1000 Biol. Rep. (2011) 3: 15] The released exosomes are small membrane-bound lipid vesicles that have a diameter ranging from 30-200 nm. Because of the double invagination processes, protein topology in exosomes is in the same orientation as in the plasma membrane of cells. Apoptotic bodies (APBs) (800-5000 nm diameter) arise from the cell during the process of apoptosis, and emerge either by separation of the membrane blebs or from apoptopodia that arise during the process of apoptosis [Xu X., et al. Biosci. Rep. (2019) 39 (1): BSR20180992].

Collectively, EVs contain an abundance of cellular cargos [Id., citing Kalra, H. et al. PLoS Biol. (2012) 10: e100450; Keerthikumar, S. et al. J. Mol. Biol. (2016) 428: 688-92; Choi, D S et al. Mass Spectrom. Rev. (2015) 34: 474-90]. Consistent with their biogenesis, the membrane composition of microvesicles reflects most closely the plasma membrane of the parent cells [Id., citing Yanez-Mo, M. et al. J. Extracell. Vesicles (2015) 4: 27066]. Consistent with their endosomal origin, the lipid membrane of exosomes is rich in cholesterol, sphingomyelin, and ceramide that are typical of lipid rafts [Tan, S S H et al. Tissue Engineering: Part B (2020) doi: 10.1089/ten.teb.2019.0326].

In contrast, a specific subset of endosomal proteins has been identified in exosomes, suggesting a sorting mechanism during exosomal development. The endosomal sorting complex required for transport (ESCRT) has been extensively characterized for regulating and channeling specific molecules into the intraluminal vesicles of the MVBs [Shao, H. et al. Chem Rev. (2018) 118 (4): 1917-50, citing Hurley, JH, Hanson, PI. Nat. Rev. Mol. Cell Biol. (2010) 11: 556-66; Henne, W M et al. Dev. Cell (2011) 21: 77-91]. The ESCRT, with its four main complexes (ESCRT 0, I, II, and III) is responsible for delivering ubiquitinated proteins for lysosomal degradation and protein recycling [Id., citing Wollert, T., Hurley, JH. Nature (2010) 464: 864-9]. Studies have shown that the depletion of specific ESCRT-family proteins can alter the protein content of exosomes and the rate of exosome release from cells [Id., citing Colombo, M. et al., .J. Cell Sci. (2013) 126: 5553-65]. Components of the ESCRT system, such as TSG101 and Alix [Id., citing Kowal, J. et al. Curr. Opin. Cell Biol. (2014) 29: 116-25] are found enriched in exosomes and thus are used as markers for exosome identification [Id., citing Lotvall, J. et al. J. Extracell. Vesicles (2014) 3: 26913].

Other ESCRT-independent processes also seem to participate, possibly in an intertwined manner, in exosome formation and release. As such, exosomes are also enriched with molecules involved in ESCRT-independent mechanisms. For example, the tetraspanin proteins such as CD9, CD63 and CD81 have been shown to participate in endosomal vesicle trafficking [Id., citing van Neil, G. et al. Dev. Cell. (2011) 21: 708-21; Verweij, F J et al., EMBO J. (2011) 30: 2115-29] The involvement of the Rab family of small GTPases in vesicle trafficking and fusion with the plasma membrane also suggests a role of these proteins in exosome release [Id., citing Vanlandingham, PA, Ceresa, BP. J. Biol. Chem. (2009) 284: 12110-24; Ostrowski, M. et al. Nat. Cell Biol. (2010) 12: 19-30; Zeigerer, A. et al. Nature (2012) 485: 465-70]. In addition, sphingomyelinase has been demonstrated to be involved in vesicle release, as supported by elevated levels of ceramide in exosomes and a reduction in exosome release upon inhibition of sphingomyelinase [Id., citing Trajkovic, K. et al. Science (2008) 319: 1244-7].

### Proteins Enriched in EVs

EV proteins derive mainly from cellular plasma membrane, cytosol, but not from other intracellular organelles (e.g., Golgi apparatus, endoplasmic reticulum, and nucleus) [Id., citing Simpson, R J et al. Expert Rev. Proteomics (2009) 6: 267-83; Raimondo, F. et al. Proteomics (2011) 11: 709-20; Choi, D S et al. Mass Spectrum Rev. (2015) 34: 474-90]. This protein constitution of EV is indicative of vesicle biogenesis and cargo sorting [Id., citing Kowal, J. et al. Proc. Natl Acad. Sci. USA (2016) 113: E968-77]

### Membrane Proteins

In mammalian vesicles, both transmembrane and lipid-bound extracellular proteins (e.g., lactadherin) are found associated with microvesicles and exosomes [Id., citing Lotvall, J. et al. J. Extracell Vesicles. (2014) 3: 26913] Within the group of transmembrane proteins, exosomes are enriched with tetraspanins (e.g., CD9, CD63, CD81), a superfamily of proteins with four transmembrane domains [Id., citing van Niel, G. et al. Dev. Cell (2011) 21: 708-21; Velrweij, F J et al. EMBOJ. (2011) 30: 2115-29]. Tetraspanins are involved in membrane trafficking and biosynthetic maturation, [Id., citing Perez-Hernandez, D. et al. J. Biol. Chem. (2013) 288: 11649-61; Andreu, Z., Yanez-Mo, M. Front. Immunol (2014) 5: 442] and are highly expressed in exosomes. Tetraspanins, however, are not uniquely expressed in exosomes alone. [Id., citing Lotvall, J. et al. J. Extracell. Vesicles (2014) 3: 26913]. Reflecting their derivation from the plasma membrane of cells, EVs are enriched with specific transmembrane protein receptors (e.g., epidermal growth factor receptors/EGFRs6 [Id., citing Al-Nedawi, K. et al. Proc. Natl Acad. Sci. USA (2009) 106: 3794-9] and adhesion proteins (e.g., epithelial cell adhesion molecule/EpCAM [Id., citing Im, H. et al. Nat. Biotechnol. (2014) 32: 490-5; Tauro, B J et al. Mol. Cell Proteomics (2013) 12: 587-98].

### Intravesicular Proteins

EV-associated intravesicular proteins have diverse functions. They include cytosolic proteins that have membrane- or receptor binding capacity, such as TSG101, ALIX, annexins and Rabs, which are involved in vesicle trafficking. EVs are also enriched with cytoskeletal proteins (e.g., actins, myosins, tubulins), molecular chaperones (e.g., heat-shock proteins/HSPs), metabolic enzymes (e.g., enolases, glyceraldehyde 3-phosphate dehydrogenase/GAPDH) and ribosomal proteins [Id, citing Lotvall, J. et al. J. Extracell. Vesicles (2014) 3: 26913; Choi, D S et al. Mass Spectrom. Rev. (2015) 34: 474-90]. It has been reported that EV protein cargoes can be effectively transported to and received by recipient cells to elicit potent cellular responses in vitro and in vivo [Lai, C P et al. Nat. Communic. (2015) 6: 7029; Mittelbrunn, M., Sanchez-Madrid, F. Nat. Rev. Mol. Cell Biol. (2012) 13: 328-35].

### Nucleic Acids

Both exosomes and microvesicles also contain nucleic acids include miRNAs, mRNAs [Id., citing Valadi, H. et al. Nat. Cell Biol. (2007) 9: 654-9; Skog, J. et al. Nat. Cell Biol. (2008) 10: 1470-6], DNA [Id., citing Balaj, L. et al. Nat. Commun. (2011) 2: 180; Thakur, B K et al. Cell Res. (2014) 24: 766-9] and other non-coding RNAs [Id., citing Wei, Z. et al. Nat. Commun. (2017) 8: 1145] RNA types are summarized in Table 1.

TABLE 1

| RNA | Functions | Coding | Typical Size | |
|---|---|---|---|---|
| mRNA | Protein translation | Yes | 400-12,000 nt, | |
| | | | average ≈2100 nt | |
| microRNA | Post-transcriptional | No | 17-24 | nt |
| (miRNA) | gene silencing | | | |
| Y RNA | Component of Ro60 | No | ≈100 | nt |
| | ribonucleoprotein | | | |
| | particle; initiation | | | |
| | factor for DNA | | | |
| | replication | | | |
| Signal Recognition | Component of SRP | No | ≈280 | nt |
| particle RNA | ribonucleoprotein | | | |
| (SRP RNA) | complex that directs | | | |
| | protein trafficking | | | |
| Transfer RNA | Adapter for matching | No | 76-90 | nt |
| (tRNA) | amino acid to mRNA | | | |
| Ribosomal RNA | RNA component of | No | 185 (1.9 kb) | |
| (rRNA) | ribosomes | | 28S (5.0 kb) | |
| Small nuclear | RNA processing | No | ≈150 | nt |
| RNA (snRNA) | such as mRNA | | | |
| | splicing | | | |
| Small nucleolar | Guiding chemical | No | 20-24 | nt |
| RNA (snoRNA) | modifications of | | | |
| | other RNAs | | | |
| Long noncoding | Many, including in- | No | >100 | nt |
| RNA (lncRNA) | transcription and | | | |
| | post-transcription | | | |
| | regulation | | | |

mRNA

mRNAs are a large family of coding RNA molecules that specify protein sequence information. Studies have reported that EVs contain a substantial proportion of their parent cells' mRNA pool, many of which are cell type-specific mRNA. [Shao, H. et al. Chem Rev. (2018) 118 (4): 1917-50, citing Wei, Z. et al. Nat. Commun. (2017) 8: 1145; Batagov, A O, Kurochkin, IF. Biol. Direct (2013) 8: 12] These mRNA molecules, often in fragmented form, reside within EVs and are protected from RNase degradation. Furthermore, the fraction of polyadenylated mRNA molecules in EVs suggest that some of them (<2 kb) are capable of encoding polypeptides in support of protein synthesis (i.e., functionality in protein translation). This has been confirmed in multiple studies through different translation assays in recipient cells [Id., citing Valadi, H. et al. Nat. Cell Biol. (2007) 9: 654-9; Skog, J. et al. Nat. Cell Biol. (2008) 10: 1470-6; Lai, C P et al. Nat. Commun. (2015) 6: 7029].

miRNA

miRNAs are a class of small, noncoding RNAs (typically 17-24 nucleotides) which mediate post-transcriptional gene silencing usually by targeting the 3′ untranslated region of mRNAs. By suppressing protein translation, EV miRNAs are powerful regulators for a wide range of biological processes [Id., citing Mittelbrunn, M. et al. Nat. Commun. (2011) 2: 282; Redzic, J S et al. Semin. Cancer Biol. (2014) 28: 14-23]. miRNAs can also exist in multiple stable forms when circulating in bodily fluids. For example, in addition to being packaged into EVs, circulating miRNAs can also be loaded onto high-density lipoprotein [Id., citing Vickers, K C et al. Nat. Cell Biol. (2011) 13: 423-33; Wagner, J. et al. Arterioscler. Thromb. Vasc. Biol. (2013) 33: 1392-400] or bound to AGO2 protein outside the vesicles [Id., citing Arroyo, et al. Proc. Natl Acad. Sci. USA (2011) 108: 5003-8; Turchinovich, A. et al. Methods Mol. Biol. (2013) 1024: 97-107]. The distribution of miRNAs within EVs remains unclear [Id., citing Min, PK & Chan, SY. Eur. J. Clin. Invest. (2015) 45: 860-74; Turchinovich, A. et al. Methods Mol. Biol. (2013) 1024: 97-107; Chevillet, J R et al. Proc. Natl. Acad. Sci. USA (2014) 111: 14888-93]. As in the case of mRNA, miRNA profiles in EVs reflect their cell of origin but differs somewhat from their parental cells. Some miRNAs have been found preferentially sorted into EVs and remaining functional in recipient cells to regulate protein translation. [Id., citing Villarroya-Beltri, C. et al. nat. Commun. (2013) 4: 2980; Koppers-Lalic, D. et al. Cell Rep. (2014) 8: 1649-58; Santangelo, L. et al. Cell Rep. (2016) 17: 799-808; Teng, Y. et al. Nat. Commu. (2017) 8: 14448]

### Other RNA Types

In addition to mRNA and miRNA, many noncoding RNA types have been identified in EVs through next generation sequencing [Id., citing Huang, X. et al. BMC Genomics (2013) 14: 319; Conley, A.et al. RNA Biol. (2017) 14: 305-16]. These RNAs include transfer RNA (tRNA), ribosomal RNA (rRNA), small nuclear RNA (snRNA), small nucleolar RNA (snoRNA), as well as long noncoding RNA (lncRNA) [Id., citing Wei, Z. et al. Nat. Commun. (2017) 8: 1145; Huang, X. et al. BMC Genomics (2013) 14: 319; Crescitelli, R. et al. J. Extracell. Vesicles (2013) 2: 20677].

Small (about 20-30 nucleotide (nt)) noncoding RNAs regulate eukaryotic genes and genomes (Carthew, R W and Sontheimer, EJ. Cell (2009) 136: 642-55). This regulation can occur at multiple levels of genome function, including chromatin structure, chromosome segregation, transcription, RNA processing, RNA stability, and translation (Id.). The effects of small RNAs on gene expression and control are generally inhibitory, and the corresponding regulatory mechanisms are therefore collectively subsumed under the heading of RNA silencing (Id.). The central theme that runs throughout is that the small RNAs serve as specificity factors that direct bound effector proteins to target nucleic acid molecules via base-pairing interactions (Id.). Invariably, the core component of the effector machinery is a member of the Argonaute protein superfamily (Id.).

There are three main categories of small RNAs: short interfering RNAs (siRNAs), microRNAs (miRNAs), and piwi-interacting RNAs (piRNAs) (Id.). siRNAs and miRNAs are the most broadly distributed in both phylogenetic and physiological terms and are characterized by the double-stranded nature of their precursors (Id.). In contrast, piRNAs are primarily found in animals, exert their functions most clearly in the germline, and derive from precursors that are poorly understood, but appear to be single stranded (Id.). Where siRNAs and miRNAs bind to members of the Ago clade of Argonaute proteins, piRNAs bind to members of the Piwi clade (Id.).

The signature components of RNA silencing are Dicers, Agos, and ~21-23 nt duplex-derived RNAs (Id.). Both siRNA and miRNA small RNAs depend on Dicer enzymes to excise them from their precursors, and Ago proteins to support their silencing effector functions (Id.).

RNase III enzymes, which are dsRNA-specific nucleases, are the source of miRNA/siRNA biogenesis (Id.). One class of large RNase III enzymes has several domains in a specific order from the amino to carboxy terminus: a DEXD/H ATPase domain, a DUF283 domain, a PAZ domain, two tandem RNase III domains, and a dsRNA-binding domain (Id.). Some members of this family differ slightly from this arrangement (Id.).

The PAZ and RNase III domains play central roles in excising siRNAs preferentially from ends of dsRNA molecules. PAZ domains are shared with Argonaute proteins and are specialized to bind RNA ends, especially duplex ends with short (~2 nt) 3′ overhangs. An end engages the Dicer PAZ domain, and the substrate dsRNA then extends approximately two helical turns along the surface of the protein before it reaches a single processing center that resides in a cleft of an intramolecular dimer involving the RNase III domains. Each of the two RNase IIII active sites cleaves one of the two strands, leading to staggered duplex scission to generate new ends with ~2-3′ nt overhangs. The reaction leaves a 5′ monophosphate on the product ends, consistent with a requirement for this group during later stages of silencing. This general model pertains equally to pre-miRNA stem-loop substrates and to long, perfectly base-paired dsRNAs. In some species, different functional categories of small RNAs exhibit slightly different lengths; this appears to be dictated by the distance between the PAZ domain and the processing center in the relevant Dicer enzyme (Id.).

The roles of the ATPase domain probably vary among different forms of Dicer (Id.). ATP promotes dsRNA processing by Drosophila Dicer 2 and C. elegans Dcr-1, and mutations predicted to cripple ATPase activity in Drosophila Dicer-2 specifically abolish dsRNA processing. In contrast, ATP is dispensable for dsRNA processing by human Der (hDcr), and an ATPase defective mutant exhibits no processing defect (Id.).

Dicers isolated from their natural sources generally are found in a heterodimeric complex with a protein that contains two or three double stranded Ras binding domains (dsRBDs); the Ras-binding domain (RBD) is an independent domain of about 75 residues, which is sufficient for GTP-dependent binding of Ras and other G alpha GTPases. Both hDcr and Drosophila Dcr-2 process dsRNAs effectively in the absence of the heterodimeric partner (TRBP and R2D2, respectively). In at least some cases, the role of Dicer in silencing extends beyond dsRNA processing and into the pathway of RISC assembly; this activity is much more dependent on the dsRBD partner protein (Id.).

### Argonautes

The Argonaute superfamily can be divided into three separate subgroups: the Piwi clade that binds piRNAs, the Ago clade that associates with miRNAs and siRNAs, and a third clade described in nematodes. All gene regulatory phenomena involving ~20-30 nt RNAs are thought to require one or more Argonaute proteins, which are the central, defining components of an RNA-induced silencing complex (RISC). The double-stranded products of Dicer enter into a RISC assembly pathway that involves duplex unwinding, culminating in the stable association of only one of the two strands with the Ago effector protein. This guide strand directs target recognition by Watson-Crick base pairing; the other strand of the original small RNA duplex (the passenger strand) is discarded (Id.).

Argonaute proteins are defined by the presence of four domains: the PAZ domain (shared with Dicer enzymes), the PIWI domain that is unique to the Argonaute superfamily, and the N and Mid domains. The overall protein structure is bi-lobed, with one lobe consisting of the PAZ domain and the other lobe consisting of the PIWI domain flanked by N-terminal (N) and middle (Mid) domains. The Argonaute PAZ domain has RNA 3′ terminus binding activity, and the co-crystal structures reveal that this function is used in guide strand binding. The other end of the guide strand engages a 5′ phosphate binding pocket in the Mid domain, and the remainder of the guide tracks along a positively charged surface to which each of the domains contributes. The protein-DNA contacts are dominated by sugar-phosphate backbone interactions. Guide strand nucleotides 2-6, which are especially important for target recognition, are stacked with their Watson-Crick faces exposed and available for base pairing (Id.).

The PIWI domain adopts an RNase H-like fold that in some cases can catalyze guide strand-dependent endonucleolytic cleavage of a base pair target. This initial cut represents the critical first step in a subset of small RNA silencing events that proceed through RNA destabilization. Not all Argonaute proteins have endonucleolytic activity, and those that lack it usually also lack critical active-site residues that coordinate a presumptive catalytic metal ion (Id.).

In humans, four of the eight Argonaute proteins are from the Ago clade and associate with both siRNAs and miRNAs (Id.).

### MicroRNA Biogenesis

MicroRNAs are found in plant and animal branches of Eukaryotes and are encoded by a bewildering array of genes. Transcription of miRNAs is typically performed by RNA polymerase II, and transcripts are capped and polyadenylated. Although some animal miRNAs are individually produced from separate transcription units, many more are produced from transcription units that make more than one product. A transcript may encode clusters of distinct miRNAs, or it may encode miRNA and protein. The latter type of transcript is organized such that the miRNA sequence is located within an intron. Many new animal miRNAs are thought to arise from accumulation of nucleotide sequence changes and not from gene duplication (Carthew, R W and Sontheimer, EJ. Cell (2009) 136: 642-55).

The resulting primary or pri-miRNA transcript extends both 5′ and 3′ from the miRNA sequence, and two sequential processing reactions trim the transcript into the mature miRNA. Processing depends on the miRNA sequence folding into a step-loop structure. A typical animal pri-miRNA consists of an imperfectly paired stem of ~33 bp, with a terminal loop and flanking segments. The first processing step, which occurs in the nucleus, excises the stem-loop from the remainder of the transcript to create a pre-miRNA product. For most pri-miRNAs, a nuclear member of the RNase III family (Drosha in animals) carries out this cleavage reaction. Although Drosha catalyzes pri-miRNA processing, it depends on a protein cofactor, which contains two dsRBD domains and stably associates with the ribonuclease to form the microprocessor complex (Id.).

An alternative pathway uses splicing of pri-miRNA transcripts to liberate introns that precisely mimic the structural features of pre-miRNAs. These introns then enter the miRNA processing pathway without the aid of the Microprocessor (Id.).

The second processing step excises the terminal loop from the pre-miRNA stem to create a mature miRNA duplex of approximately 22 bp length. In animals, the pre-miRNA is exported from the nucleus, and the canonical Dicer enzyme carries out the cleavage reaction in the cytoplasm (Id.).

MicroRNAs behave like traditional polymeric products of gene activity, such that most species of a miRNA have highly exact ends, although there is a little variation. This feature of miRNAs may allow them to interact with greater specificity on substrate mRNAs without a need for stringent complementarity or large overlap (Id.).

Consequently, the processing machinery is constructed to produce miRNA duplexes with highly exact ends. The first cut, carried out by Drosha with the aid of its dsRBD domain binding partner protein (called DGCR8), is most critical. DGCR8 directly interacts with the pri-miRNA stem and flanking single-stranded segments. The cleavage site is determined by the distance from the stem-flank junction, which is precisely one turn of a dsRNA helix (11 bp) and is the minimal processing length for an RNase III enzyme. Although Drosha carries out the cleavage reaction, it relies upon DGCR8 to serve as a molecular anchor that properly positions Drosha's catalytic site the correct distance from the stem-flank junction. Thus, the endpoint of the stem is a critical determinant for one end of the mature miRNA (Id.).

The second cut performed by Dicer defines the other end of the mature miRNA. Dicer will cleave anywhere along a dsRNA molecule but has a strong preference for the terminus. The PAZ domain of Dicer interacts with the 3′ overhang at the terminus and determines the cleavage site in a ruler-like fashion. The RNase III catalytic sites are positioned two helical turns or 22 bp away from the terminus/PAZ portion of the Dicer-RNA complex (Id.).

While regulation of miRNA biogenesis has not been extensively studied, a surprising number of miRNA genes are formed under the control of the very targets that they regulate. A rationale behind these double-negative regulatory relationships is that tight regulation of miRNA biogenesis is crucial. Mis-expression of miRNAs frequently mimics loss of function phenotypes for their targets. This would be prevented if biogenesis of a miRNA is strictly controlled by its targets. The restriction would also explain how off-targeting effects by wayward miRNAs are carefully limited (Id.).

### MicroRNA Associations

The mature miRNA duplex is a short-lived entity; it is rapidly unwound when it associates with an Ago protein. Unwinding occurs so rapidly after duplex formation, because the two processes are physically coupled due to Ago2's presence in a complex with Dicer and TRBP, the double-stranded RNA binding protein that loads siRNA into the RISC (Id.).

miRNA unwinding is accompanied by differential strand retention, i.e., one strand is retained while the other strand is lost. Strand retention is based on the relative thermodynamic stability of the duplex's ends. Although the rule is that the 5′ terminus of the retained strand is at the less stably base-paired end of the duplex, this rule is not absolute. The other strand is appreciably detected in Ago complexes, lending ambiguity to the notion of strand asymmetry. Although either strand can become stably associated with Ago proteins, the more commonly associate strand is termed the miRNA strand; the other strand is called the miRNA* strand. miRNA unwinding is not accompanied by cleavage of the ejected strand by the associated Ago (Id.).

The mammalian Dicer/Ag/miRNA complex is associated with other proteins, e.g., Gemin3, Gemin4, Mov10, and Imp8, as well as the mammalian protein GW182, associate with Ago2. GW182 is both necessary and sufficient for miRNA-bound Ago to silence gene expression. Thus miRNA-bound Ago in association with GW182 can be thought of as the miRISC complex (Id.).

Post-Transcriptional Repression by miRNAs

An miRNA acts as an adaptor for miRISC to specifically recognize and regulate particular mRNAs. If miRISC is tethered to a heterologous RNA recognition factor, the factor enables miRISC to recognize and repress mRNAs that lack miRNA-binding sites. With few exceptions, miRNA-binding sites in animal mRNAs lie in the 3′ untranslated region (UTR) and are usually present in multiple copies. Most animal miRNAs bind with mismatches and bulges, although a key feature of recognition involves Watson-Crick base pairing of miRNA nucleotides 2-8, representing the seed region (Id.).

While it was thought that perfect complementarity allows Ago-catalyzed cleavage of the mRNA strand, whereas central mismatches exclude cleavage and promote repression of mRNA translation, it appears that translational repression is the default mechanism by which miRNAs repress gene expression, both in animals and plants. Perfectly complementary miRNAs may additionally engage in mRNA cleavage such that their effects are the result of both mechanisms (Id.).

The mechanisms by which miRISC regulates translation have been subject to ongoing debate. The fundamental issue of whether repression occurs at translation initiation or post-initiation has not yet been resolved. There are three competing models for how miRISC represses initiation. One proposes that there is competition between miRISC and elF4E for binding to the mRNA 5′ cap structure. A second model has proposed that miRISC stimulates de-adenylation of the mRNA tail; translation is repressed because the cap and PABP1-free tail of the deadenylated mRNA are unable to circularize. A third model has proposed that miRISC blocks association of the 60S ribosomal subunit with the 40S preinitiation complex, i.e., the recruitment of eIF6 by miRISC may repress translation by preventing the assembly of translationally competent ribosomes at the start codon (Id.).

It is unclear why some targets are degraded and others are not (Id.).

Without being limited by any particular theory, it appears that the mode of regulation of any miRNA (repression vs. activation) in the context of the whole cell and the myriad activities that affect posttranscriptional gene regulation may be context dependent (Id.).

The cell's position in the cell cycle is one such context. For example, miRNA let-7 and an artificial miRNA (CXCR-4) repress translation in proliferating human cells, but change into translational activators when the cell cycle is arrested at the G1 checkpoint by serum starvation. Aphidicollin-induced arrest at G1 also generates translational activation, whereas nocodazole-induced arrest at G2/M generates translational repression. Lymphocyte growth arrest induces TNFα expression that is required for macrophage maturation; miR-369-3p switches from a repressor to an activator of TNFα translation when cells in culture are growth arrested (Id., citing Vasudevan, S.et al. Science (2007) 318: 1931-34).

Binding site position is another context. Interaction of miR-10a with the 5′UTR of certain ribosomal subunit mRNAs leads to their activated translation, whereas interaction with the 3′UTR leads to repression (Id., citing Orom, U A et al. (2008) Mol. Cell 30: 460-71).

Another context is how small RNA regulation is organized and modulated within the cell. Ago proteins are frequently associated with membrane trafficking compartments, such as the Golgi and ER (Id., citing Cikaluk, D. E. et al. Mol. Biol. Cell (1999) 10: 3357-72). It has been hypothesized that miRISC factors might become anchored in certain subcellular compartments, e.g., P bodies or GW bodies, two separate pools of sequestered non-translating RNAs (Patel, P H, et al. PLos One (2016) 11(3): e015029). Subunits of miRISC (miRNAs, Ago and GW1821) and their repressed targets also are enriched in GW bodies. While GW bodies are not essential for miRNA repression, GW body formation requires an intact miRNA pathway (Carthew, R W and Sontheimer, EJ. Cell (2009) 136: 642-55).

miRNA Expression

MicroRNAs regulate gene expression at the post-transcriptional level. The exact functional outcome of an miRNA may be determined by multiple features, including the cell type affected, the inducing signal, and the transcriptomic profile of the cell, which ultimately affect the availability and ability to engage different target mRNAs and bring about its unique responses. Indeed, data suggest that miRNAs may play different roles in diverse biological contexts [Lee, H-M et al. BMB Rep. (2016) 49 (6): 311-18].

miR-29: levels of miR-29, including miR-29a, miR-29b, and miR-29c, are significantly lower in fibrotic livers as shown in human liver cirrhosis, as well as in two different fibrotic animal molecules (carbon tetrachloride and bile duct ligation, while their down regulation affects hepatic stellate cell (HSC) activation [Huang, Y-H et al. Intl J. Molec. Sci. (2018) 19: 1889, citing Mann, J. et al. Gastroenterology (2010) 138: 705-14; Roderburg, C. et al. Hepatology (2011) 53: 209-18; Sekiya, Y. et al. Biochem. Biophys. Res. Commun. (2011) 412: 74-9]. It has been reported that TGF-β1 was capable of mediating the downregulation of miR-29 in HSCs [Id., citing Roderburg, C. et al. Hepatology (2011) 53: 209-18]; the same was reported in the study of Bandyopadhyay et al. who found this effect to be specific to HSC [Id., citing Bandyopadhyay, S. et al. J. Infect. Dis. (2011) 203: 1753-62]. The overexpression of miR-29 in murine HSC results in the down regulation of collagen expression, including collagen-1a1 and collagen-4α1 [Id., citing Roderburg, C. et al. Hepatology (2011) 53: 209-18; Bandyopadhyay, S. et al. J. Infect. Dis. (2011) 203: 1753-62; Huang, J. et al. Int. J. Mol. Sci. (2014) 15: 9360-71] by directly targeting the mRNA expression of these extracellular matrix genes.

miR-29 family clusters also have emerged as a major anti-fibrotic player in kidney fibrosis associated with Smad-dependent and Smad-independent pathways (Srivastava, S P et al. Front. Pharmacol. (2019) 10: 904, citing Chung A C and Lan H Y. Front. Physiol. (2015) 6: 50). The expression level of members of the miR-29 family is significantly suppressed in both renal fibrosis (Id., citing Lan, HY. Clin. Exp. Pharmacol. Physiol. (2012) 39: 731-38; Meng, X M et al. Clin Sci. (London) (2013) 124: 243-54; Srivastava, S P et al Fibrogenesis Tissue Repair (2014) 7: 12) and diabetic (Id., citing Srivastava, S P et al. Sci Rep. (2016) 6: 29884) and hypertensive nephropathy (Id., citing Wei, Q. et al. IUBMB Life (2013) 65: 602-14). miR-29 is downstream of Smad3 and can suppress upstream TGFβ-Smad3 signaling by miR-29b-mediated negative feedback (Id., citing He, Y. et al. Biochimie (2013) 95: 1355-59). miR-29b binds to the coding region of TGFβ1 mRNA at exon 3, which blocks the translation of TGFβ1, resulting in the suppression of Smad3-dependent fibrosis (Id., citing Zhang, Y. et al. Mol. Ther. (2014) 22: 974-85). miR-29 binds to the promoter region of smad3 and exerts anti-fibrotic properties. In vitro, overexpression of miR-29 inhibited, but knockdown of miR-29 enhanced, TGFβ1-induced expression of collagens I and III in cultured proximal tubular epithelial cells (TECs) (Id., citing Qin, W. et al. J. Am. Soc. Nephrol. (2011) 22: 1462-74; Wang, B. et al. J. Am. Soc. Nephrol. (2012) 23: 252-65; Qi, R and Yang, C. Cell Death Dis. (2018) 9: 1126). However, ultrasound-mediated gene delivery of miR-29 blocked progressive renal fibrosis in obstructive nephropathy (UUO) (Id., citing Qin, W. et al. J. Am. Soc. Nephrol. (2011) 22: 1462-74; Qi, R and Yang, C. Cell Death Dis. (2018) 9: 1126)). Data from various studies have shown that members of the miR-29 family target different isoforms of collagen and have an anti-fibrotic role (Id., citing Wang, B. et al. J. Am. Soc. Nephrol. (2012) 23: 252-65; Qi, R and Yang, C. Cell Death Dis. (2018) 9: 1126). TGFβ1 inhibits the beneficial role of miR-29 family by down-regulating the expression in TECs (Id., citing Du, B. et al. FEBS Lett. (2010) 584: 811-16; Wang, B. et al. J. Am. Soc. Nephrol. (2012) 23: 252-65), mesangial cells (Id., citing Wang, B. et al. J. Am. Soc. Nephrol. (2012) 23: 252-65), and podocytes (Id., citing Wang, B. et al. J. Am. Soc. Nephrol. (2012) 23: 252-65). miR-29b suppression contributes to progressive renal injury in several mouse models of chronic kidney disease (CKD) (Id., citing Qin, W. et al. J. Am. Soc. Nephrol. (2011) 22: 1462-74; Wang, B. et al. J. Am. Soc. Nephrol. (2012) 23: 252-65; Ramdas, V.et al. Am. J. Pathol. (2013) 183: 1885-96); however, overexpression of miR-29b provides a therapeutic benefit in unilateral ureter obstruction (UUO) and db/db obese mice (Id., citing Qin, W. et al. J. Am. Soc. Nephrol. (2011) 22: 1462-74; Chen, H Y et al. Mol. Ther. (2014) 22: 842-53). In db/db mice, miR-29a has been shown to be elevated in the liver and to regulate gluconeogenesis Id., citing Pandey, A K et al. Mol. Cell Endocrinol. (2011) 332: 125-33). Treatment of rats with losartan caused a remarkable increase in the level of miR-29b expression, which was linked with lower expression of collagen, fibronectin, and laminin, and provided protection from kidney fibrosis (Id., citing Wang, B. et al. J. Am. Soc. Nephrol. (2012) 23: 252-65). miR-29 family clusters also inhibit elevated dipeptidyl dipeptidase-4 (DPP-4) protein levels by targeting the 3′UTR of its mRNA (Id., citing Kanasaki, K. et al. Diabetes (2014) 63: 2120-31; Shi, S. et al. Kidney Int. (2015) 88: 479-89). TGFβ2-mediated induction of DPP-4 and down-regulation of miR-29 are associated with endothelial to mesenchymal transition (EndMT) (Id., citing Kanasaki, K. et al. Diabetes (2014) 63: 2120-31; Shi, S. et al. Kidney Int. (2015) 88: 479-89). miR-29 and TGFβ signaling exhibit a negative feedback loop and regulate each other, as induction of TGFβ signaling suppresses downstream miR-29 (Id., citing Kanasaki, K. et al. Diabetes (2014) 63: 2120-31) and miR-29 suppresses upstream TGFβ signaling (Id., citing Zhang, Y. et al. Mol. Ther. (2014) 22: 974-85), This relationship supports an anti-fibrotic role of miR-29 in kidney fibrosis.

miR 10a: Differential expression of miR-10a has been implicated in regulating a pro-inflammatory endothelial phenotype. Kumar, S. et al. Vascul. Pharmacol. (2019) 114: 76-92, citing Fang, Y. et al. Proc. Natl Acad. Sci. USA (2010) 107 (30) 13450-55]. Stable blood flow upregulates the expression of miR-10a in the endothelium. Loss of miR-10a results in activation of NfκB via MAP3K7 and βTRC, both of which promote IκB degradation and p65 translocation, resulting in endothelial inflammation in a porcine model of atherosclerosis.

In a bleomycin-induced pulmonary fibrosis model in mouse, miRNAs deregulated in the late period (days 14 and 21) after bleomycin injury were demonstrated to target key components in the TGF-β signaling pathway. These miRNAs include miR-196b, miR-704, miR-717, miR-16, miR-195, miR-10a, miR-211, miR-34a, miR-367, miR-21, and let-7f, which target TGF-β family members such as TGF-β2 and 3, TGF-β receptors such as TGF-β receptor I and II, Smad family members including Smad 3, 6, and 7, and procollagen type 1 alpha 2 [Xie, T. et al. Physiol. Genomics (2011) 43 (9): 479-87].

Another study reported that in mouse, the hepatic fibrosis tissue transforming growth factor (TGF)-β1/Smads signal transduction pathway correlated with the progression of hepatic fibrosis. It is known that transforming growth factor (TGF)-β1 induces hepatic fibrosis [Zhou, G. et al. Exp. Ther. Med. (2016) 12 (30: 1719-22), citing Tomita, K. et al. Gut (2006) 55: 415-24], and that Smad protein, a key active substrate of TGF-β1 family receptor kinase [Id., citing Heldin, C H et al. Nature (1997) 390: 465-71] constitutes the negative feedback loop in TGF-β signal transduction and exerts an anti-fibrosis effect [Id., citing Kavsak, P. et al. Mol Cell 1 (2000) 6: 1365-75; Padda, R S et al. Am. J. Physiol. Gastroint. Liver Physiol. (2015) 308: G251-61]. Therefore, together, Smad and TGF-β1 cause HSC activation, and initiate collagen gene expression, resulting in the genesis of hepatic fibrosis. Forty healthy female 8-week-old C57BL6/J mice were randomly divided into a control group (intraperitoneal injection of 5 μl/g normal saline, twice per week for 8 weeks) and a hepatic fibrosis group (intraperitoneal injection of 5 μl/g 10% CCI4 olive oil, twice per week for 8 weeks), with 20 mice per group. RT-PCR was used to test miR-10a expression in cells in both groups. Cell culture and transfection of miR-10a mimics were conducted in the two groups and a Cell Counting Kit-8 was used to test the expression of TGF-β1 and Smad7 in hepatic fibroblasts. It was found that miR-10a expression was significantly increased in the hepatic fibrosis group compared with the control group (P<0.05), and that the expression level of miR-10a was significantly increased in the group transfected with miR-10a mimics compared with the control group (P<0.05). A high expression of miR-10a significantly increased TGF-β1 expression and reduced Smad7 expression in the hepatic fibrosis group (P<0.05) thus exerting a hepatic fibrosis-promoting effect by regulating the TGF-β1/Smads signal transduction pathway [Zhou, G. et al. Exp. Ther. Med. (2016) 12 (30): 1719-22].

Another study indicated that down-regulation of miR-10a may inhibit collagen formation, reduce atrial structure remodeling, and decrease proliferation of cardiac fibroblasts, eventually suppressing cardiac fibrosis in a rat model of atrial fibrillation via inhibition of the TGF-β1/Smads signaling pathway. Overexpressed miR-10a significantly prolonged the duration of AF, further elevated the collagen volume fraction (CVF), and increased the viability of CFs in AF rats; these findings were in contrast with the findings for rats with inhibition of miR-10a (all P<0.05). Moreover, miR-10a overexpression could promote miR-10a, collagen-I, collagen III, α-SMA, and TGF-β1 protein expression and increase the levels of hydroxyproline but reduced Smad7 protein expression in atrial tissues and CFs in AF rats. Not surprisingly, inhibiting miR-10a led to completely contrasting results (all P<0.05). Moreover, TGF-β1 treatment could reverse the inhibitory effect of miR-10a down-regulation on cardiac fibrosis in CFs. Bioinformatics analysis and luciferase reporter assay results demonstrated that miR-10a bound directly to the 3′-UTR of BCL6, which is involved in cell growth and proliferation [Li, P F et al. Biosci. Rep. (2019) 39 (20: BSR20181931).

miR34a: MiR34a inhibits sirtuin-1 and shows increased expression in peripheral lungs and epithelial cells of COPD patients and is correlated with increased expression of senescence markers in lung cells [Barnes P J et al. Am. J. Respir. Crit. Care Med. (2019) 200 (5): 556-64, citing Baker, J. et al. Sci. Rep. (2016) 6: 358710]. It also regulates sitruin-6, but not other sirtuins. MiR-34a is increased by oxidative stress through activation of PI3K-mTOR signaling and leading to a parallel reduction in Sirt1 and Sirt6, whereas other sirtuins are unchanged, as in COPD lungs. An antagomir of miR-34 restores sirt1 and sirt6 in senescent small airway epithelial cells from COPD patients, reduces markers of cellular senescence (p16, p21, p53), reduces the SASP response (TNFα, IL-1β, IL-6, CCL2, CXCL8, MMP9), and increases proliferation of senescent epithelial cells by reversing cell cycle arrest [Id., citing Baker, J. et al. Sci. Rep. (2016) 6: 358710]. miR-34a also is increased in COPD macrophages and may be associated with impaired phagocytosis and uptake of apoptotic cells (efferocytosis) observed in this disease [Id., citing McCubbrey, Al et al. Immunol. (2016) 196: 1366-75].

One study provided evidence that several miRNAs (e.g., miR-21, miR-34a, let-7e, miR-99b, miR-125a, and miR-342 were coordinately upregulated during monocyte-derived dendritic cell (MDDC) differentiation, and validated two genes (WNT1 and JAG1) targeted by 3 of these miRNAs (miR-21, miR-34a, and let-7e) as being involved in MDDC differentiation. Antagonizing the differential expression of miR-21 and miR-34a by either transfection of miRNA inhibitors or by exogenous addition of Wnt-1 and Jagged-1 resulted in stalling MDDC differentiation, suggesting that this regulatory pathway is necessary for MDCC differentiation [Hashimi, S T et al. Blood (2009) 114 (2): 404-14].

miR-34a (as well as miR-27a, miR-28a are highly expressed in the myocardium during congestive heart failure [Climent, M. et al. Intl J. Mol. Sci. (2020) 21: 4370, citing Tian, C. et al. Am. J. Physiol. Hear. Cir. Physiol. (2018) 314: H928-H939].

In the lung, miR-34a has been correlated to the antioxidant function of flaxseed in radiotherapy, and more generally, the miR-34 family members have been suggested to be involved in COPD [Id, citing Mizuno, S. et al. Chest (2012) 142: 663-73; Christofidou-Solomidou, M. et al. Cancer Biol. Ther. (2014) 15: 930-37; Zhang, L. et al. J. Exp. Clin. Cancer Res. (2019) 38: 53]. In the heart, an increase of miRNA has been observed in pre-diabetic and diabetic patients [Id., citing Kong, L. et al. Acta Diabetol. (2011) 48: 61-69], where high glucose levels are known to induce an accumulation of ROS. MiR-34a was found upregulated in diabetic mouse hearts and to regulate redox signaling pathways [Id. Citing Costantino, S. et al. Eur. Heart J. (2016) 37: 572-6]. Moreover, it was reported that miR-34a upregulation in diabetic mice led to dysregulation of endothelial cells by targeting Sirt1 [Id., citing Li, Q. et al. Arterioscler. Thromb. Vasc. Biol. (20016) 36: 2394-2403]. In an independent study, in vitro experiments using cardiomyocytes in high glucose conditions confirmed the induction of high levels of miR-34a and targeting of Sirt1 [Id., citing Zhu, Y. et al. Artif. Cells Nanomed. Biotechnol. (2019) 47: 4172-81], ultimately leading to oxidative stress.

miR125: miR-125a-3p can inhibit antimicrobial responses and host defenses against mycobacterial infection by targeting the gene encoding autophagy UV radiation-resistance-associated protein [Lee, H-M et al. BMB Rep. (2016) 49 (6): 311-18, citing Kim, J K et al. J. Immunol. (2015) 194: 5355-65]. In addition, miR-125a-5p can promote IL-4-induced expression of the alternative M2 phenotype by targeting KLF13, a transcriptional factor that is active during T lymphocyte activation and inflammation [Id., citing Banerjee, S. et al. J. Biol. Chem. (2013) 288: 35428-36]. miR-125a-5p plays an important role in inhibiting the classical M1-type activation induced by LPS stimulation and also can suppress the phagocytic and bactericidal activities associated with macrophage M1 functionality [Id., citing Banerjee, S. et al. J. Biol. Chem. (2013) 288: 35428-36]. Together, these data suggest that miR-125a may inhibit innate macrophage responses by regulating macrophage differentiation, inflammation, and autophagy. The expression of miR-125b-5p (which has the same core sequence as miR-125a-5p) is modulated by NF-κB signaling. miR-125b-5p targets the 3′UTR region of TNF-α gene to negatively regulate the inflammatory response Id., citing Tili, E. et al. J. Immunol. (2007) 179: 5082-89].

miR 181: The evolutionary conserved miR-181 family consists of 6 members, genetically clustered into pairs, miR-181a/b-1 (on chromosome 1 in mice and humans), miR-181a/b-2 (on chromosome 2 in mice and 9 in humans), and miR-181c/d (on chromosome 8 in mice and 19 in humans) [Grewers, Z. and Krueger, A. Intl J. Mol. Sci. (2020) 21 (17): 6200]. miR-181a is dynamically expressed in T cells over the lifetime of an organism, with its levels progressively decreasing with increasing age [Id., citing Palin, A C et al. J. Immunol. (2013) 190: 2682-91; Li, g. et al. Nat. Med. (2012) 18: 1518-24]. An ectopic increase of miR-181a expression in mature T cells resulted in increased sensitivity to peptide antigens as well as increased basal phosphorylation levels of Lck and Erk. Likewise, the inhibition of miR-181a via antagomirs reduced TCR sensitivity. Consistent with its role in modulation of TCR signaling, multiple phosphatases involved in T cell receptor signaling such as SHP-2, PTPN22, DUSP5, or DUSP6, were identified as targets of miR-181a [Id., citing Li, Q.-J et al. Cell (2007) 129: 147-61]. Although SHP-1 was not directly repressed, ectopic expression of miR-181a interfered with SHP-1 forming a physical interaction with Lck, suggesting that miR-181a might also indirectly modulate TCR signaling via SHP-1. Accumulating evidence indicates that miR-181a/b-1 predominantly acts as a rheostat of TCR signaling in both thymocytes and peripheral T cells, most likely by interfering with TCR signal strength via a co-targeting network of negative regulatory phosphatases. Consistent with its dynamic expression profile, the consequences of loss of miR-181a/b-1 are generally more severe during intrathymic T cell development, in particular of agonist-selected T cell populations.

MiR-181c has been found to be deleterious in the cardiac setting, but protective in the pulmonary system. Das et al. delivered miR-181c into rats by using nanoparticles and found that it targeted the mitochondrial cytochrome c oxidase subunit 1 (mt-COX1). COX is the last enzyme of the mitochondrial respiratory chain and the major oxygen consumer enzyme in the cells [Climent, M. et al. Intl J. Mol. Sci. (2020) 21: 4370, citing Bourens, M. et al. Antioxid. Redox Signal. (2013) 19: 19940-52; Dennerlein, D. and Rehling, P. J. Cell Sci. (2015) 128: 833-37]. Indeed, by delivering miR-181c, the authors observed a significantly aberrant consumption of oxygen, ROS production, and mitochondrial membrane potential in cardiac mitochondria isolated from miR-181c-nanoparticle-treated animals, suggesting that miR-181c targets mitochondrial genes, therefore causing cardiac dysfunction [Id., citing Das, S. et al. PLoS One (2014) 9: e96820].

In the lung, miR-181c expression levels were found to be low in the tissue of COPD patients and overexpression of this miRNA was shown to inhibit cigarette smoke-induced COPD in mice. MiR-181c was found to target CNN1 (Cysr61) and its overexpression to decrease the inflammatory response, neutrophil infiltration, and inflammatory cytokines induced by cigarette smoking, as well as the reactive oxygen species (ROS) generation [Id., citing Du, Y. et al. Respir. Res. (2017) 18: 155]. However, the exact mechanism by which miR-181c regulates ROS in COPD has not yet been elucidated. Another member of the miR-181 family was also found to cause a reduction of the levels of ROS in the pulmonary system. Jiang et al. discovered that the expression of miR-181a was downregulated in lungs of LPS-challenged mice and that the Toll-Like Receptor 4 (TLR4) was a target of miR-181a. When miR-181a was overexpressed through a mimic transfection, the LPS-induced inflammatory response was alleviated. The authors found that overexpression of miR-181a reduced the LPS-induced intracellular ROS accumulation, similarly to what happened by siTLR4 transfection. Finally, this study suggested that miR-181a could reduce LPS-induced inflammation by targeting TLR4 and subsequently reduce ROS accumulation [Id., citing Jiang, K. et al. Front. Pharmacol. (2018) 9: 142].

miR Let7: miR-let-7 family clusters demonstrate an anti-fibrotic role in lung fibrosis [Srivastava, S P et al., Front. Pharmacol. (2019) 10: 904, citing Pandit, K. V et al., Am. J. Respir. Crit. Care Med. (2010) 182: 220-29; Rajasekaran, S. et al. Front. Pharmacol. (2015) 6: 254), cardiac fibrosis (Id., citing Wang, X. et al. Hypertension (2015) 66: 776-785), and renal fibrosis (Id., citing Brennan, E P et al. J. Am. Soc. Nephrol. (2013) 24: 627-37; Srivastava, S P et al. Fibrogenesis Tissue Repair (2014) 7: 12; Srivastava, S P et al. Sci. Rep. (2016) 6: 29884). It was shown that TGFβ1 reinforces its signaling by mitigating miR-let-7b production, which targets the 3′UTR of TGFβR1 mRNA in rat tubule epithelial cells (TECs) (Id., citing Wang, B. et al. Kidney Int. (2014) 85: 352-61). Down-regulated miR-let-7b expression was found in mouse models of diabetic (Id., citing Nagai, T. et al. (2014) Biomed. Res. Int. (2014) 696475) and non-diabetic renal fibrosis (Id., citing Brennan et al., 2013). Similarly, miR-let-7c targets TGFβR1, collagen type 1 alpha 1 (COL1A1), collagen type 1 alpha 2 (COL1A2), and thrombospondin in human TECs (Id., citing Brennan, E P et al. J. Am. Soc. Nephrol. (2013) 24: 627-37). Lipoxins, which are endogenously produced lipid mediators, decrease renal fibrosis in a UUO model in the rats by elevating miR-let-7c expression (Id., citing Brennan, E P et al. J. Am. Soc. Nephrol. (2013) 24: 627-37), promote the resolution of inflammation, and inhibit fibrosis in cultured human proximal tubular epithelial (HK-2) cells (Id., citing Brennan, E P et al. J. Am. Soc. Nephrol. (2013) 24: 627-37). Lipoxin A4 (LXA4) has been shown to decrease TGFβ1-induced expression of mesenchymal markers, i.e., fibronectin, N-cadherin, thrombospondin, and the notch ligand jagged-1 in HK-2 cells through a mechanism by inducing of miR-let-7c (Id., citing Brennan, E P et al. J. Am. Soc. Nephrol. (2013) 24: 627-37)). In the UUO model of renal fibrosis, the expression level of miR-let-7c was up-regulated by treatment with LXA4 analog. LXA4 treatment caused up-regulation of miR-let-7c and inhibited TGFβR 1 and its associated signaling. Therefore, LXA4-associated up-regulation of miR-let-7c expression suppresses TGFβ1-induced fibrosis, which is a key pathway that is dysregulated in human renal fibrosis.

miR146a, an NF-κB-associated gene [Lee, H-M et al. BMB Rep. (2016) 49 (6): 311-18, citing Taganov, K D et al. Proc. Natl Acad. Sci. USA (2006) 103: 12481-86] plays a role in negative regulation of the production of proinflammatory cytokines, thus modulating the severity of the inflammatory response [Id., citing Perry, M M et al. J. Immunol. (2008) 180: 5689-98]. MiR-146a plays a critical role in regulating the proliferation of immune cells and inhibiting inflammatory responses [Id., citing Boldin, M P et al. J. Exp. Med. (2011) 208: 1189-1201; Zhao, J L et al. Proc. Natl Acad. Sci. USA (2011) 108: 9184-9]. An miR-146a deficiency in mice is associated with chronic dysregulation of NF-κB signaling, yielding a phenotype with characteristics of myeloid malignancy [Id., citing Zhao, J L et al. Proc. Natl Acad. Sci. USA (2011) 108: 9184-9]. Both miR-146a and miR-146b can regulate inflammatory responses by targeting mRNAs encoding IRAK-1 and TRAF6 [Id., citing Taganov, K D et al. Proc. Natl. Acad. Sci. USA (2006) 103: 12481-6; Saba, R. et al. Front. Immunol. (2014) 5: 578; Cui, J G et al. J. Biol. Chem. (2010) 285: 38951-60; Park, H. et al. J. Biol. Chem. (2015) 290: 2831-41]. miR-146a plays an important role in the expression of tight junction proteins claudin-1 and JAM-A, suggesting that miR-146a is essential to the maintenance of tight junction barrier and innate immune defense [Id., citing Miyata, R. et al. Eur. J. Pharmacol. (2015) 761: 375-82]. In primary human keratinocytes, miR-146a can inhibit the development of NF-κB-dependent inflammatory responses by directly targeting recruitment (by the upstream nuclear factor kappa B) of the following three signal transducers: caspase domain-containing protein 10, IL-1 receptor-associated kinase 1, and chemokine (C-C motif) ligand (CCL) 5 [Id., citing Rebane, A. et al. J. Allergy Clin. Immunol. (2014) 134 (3811): 836-47]. Moreover, TLR2 stimulation can trigger sustained expression of miR-146a, which in turn will suppress the synthesis of IL-8, CCL20, and TNF-α in primary human keratinocytes [Id., citing Meisgen, F. et al. J. Invest. Dermatol. (2014) 134: 1931-40]. In addition, activation of TLR4 signaling can upregulate miR-146b expression in human monocytes via the action of IL-10-mediated STAT3-dependent pathway [Id., citing Curtale, G. et al. Proc. Nat. Acad. Sci. USA (2013) 110: 11499-504]. In turn, miR-146b can negatively regulate LPS-mediated production of many proinflammatory cytokines and chemokines. MiR-146b fulfills these roles by targeting many components of signaling pathways, including TLR4, MyD88, IRAK-1, and TRAF6 [Id., citing Curtale, G. et al. Proc. Nat. Acad. Sci. USA (2013) 110: 11499-504].

miR 199: The regulatory effects of miR-199a are diverse. A large number of studies have indicated that the two mature types of miR-199a regulate the activities of normal cells to participate in corresponding physiological or pathological processes. For example, in the lung, expression of miR-199a-5p is upregulated by caveolin-1, which promotes lung fibroblast proliferation and differentiation; the high expression of miR0199s-5p promotes the formation of pulmonary fibrosis through the activation of the TGF-β signaling pathway by Caveolin-1 [Wang, Q. et al., Cancer Management & Res. (2019) 11: 10327-35, citing Lino, Cardenas, C L et al. PLoS Genet. (2013) 9: e1003291]. Past studies have shown that miR-199a can induce apoptosis, either by upregulating the level of pro-apoptotic protein or decreasing the expression of anti-apoptotic protein in most situations. miR-199a-3p has been reported to cause more pronounced apoptosis than miR-199a-5p in cancer cells; in A549 cells, the apoptosis pathway induced by miR-199a-5p is caspase-dependent, whereas that induced by miR-199a-3p is caspase independent [Id., citing Kim, S. et al. J. Biol. Chem. (2008) 283: 18158-66]. However, in some cases, miR-199a is involved in the anti-apoptosis effect; one study reported that miR-199a-5p is down-regulated and apoptosis is increased on a decline in oxygen tension of cardiac myocytes. Dual-luciferase reporting system assay revealed that HIF-1α is targeted gene of miR-144-5p. The results also showed that Sirt1 is a direct target of miR-199a-5p and is responsible for down-regulating prolyl hydroxylase 2, which is required for stabilization of HIF-1α. These results indicate that miR-199a can inhibit cardiomyocyte apoptosis under hypoxic conditions [Id., citing Rane, S. et al. Cir. Res. (2009) 104: 879-86]. Other results demonstrate that overexpression of miR-199a-3p suppresses the p53/miR-3p/suppressor of cytokine signaling 7 (SOCS7) pathway, which suppresses SOCS7 signaling for STAT3 activation and renal fibrosis [Id., citing Yang, R. et al. Sci. Rep. UK (2017) 7: 43409], Overexpression of miR-199a-5p also can impair autophagy and activate the mTOR/GSK3β signaling pathway, inhibit the activity of proteins, such as Atg5, Atg12, BECN1, and LCB3; and induce cardiac hypertrophy in mice [Id., citing Id., citing Li, Z. et al. Cell Death Differ. (2017) 14: 1205-13].

In one study, miR34a and miR-199a-5p were overexpressed in the lungs of 55 COPD patients compared to histologically healthy lungs. In vitro studies and analysis of COPD lung tissues showed that miR-199a-5p was associated with hypoxia-inducible factor-1α (HIF-1α) expression. The authors further investigated the relationship between oxidative stress/miR-34a/miR-199a-5p in COPD and suggested that oxidative stress induces miR-34 upregulation through the upregulation of p53. MiR-34 inhibited the activation and phosphorylation of AKT, which conversely caused miR-199a-5p upregulation. Finally, miR-199a-5p reduced the expression of HIF-1α, which can impair the VEGF expression that together with AKT inaction leads to cell apoptosis and emphysema. One caveat of the study was that more than 40% of the patients analyzed had lung cancer as well [Climent, M. et al. Intl J. Mol. Sci. (2020) 21: 4370, citing Mizuno, S. et al. Chest (2012) 142: 64: 151-60].

miR 145: MiR-145, an miRNA known to regulate cancer and avascular smooth muscle cell phenotype [Climent, M. et al. Intl J. Mol. Sci. (2020) 21: 4370, citing Hu, H. et al. Lung Cancer (2016) 97: 87-94; Climent, M. et al. Cir. Res. (2015) 116: 1753-64] significantly lowered intracellular calcium and suppressed H2O2-mediated calcium overload in rat ventricular cardiomyocytes [Id., citing Cha, M. et al. Biochem. Biophys. Res. Commun. (2013) 435: 720-26]. MiR-145 also targeted Bcl2/adenovirus E1B 19 kDa-interacting protein 3 (Bnip3), which plays a critical function in the mitochondria, i.e., mediating apoptosis and sensing oxidative stress in the cytoplasm. Downregulation of Bnip3 by miR-145 was reported to cause a reduction in ROS production, showing a miR-145 protective role in cardiomyocytes undergoing oxidative stress as well as in the heart of mice subjected to I/R [Id., citing Li, R. et al. PLoS ONE (2012) 7: e44907].

It has been reported that miR-145 expression is upregulated in TGF-β1-treated lung fibroblasts in vitro and that miR-145 expression is also increased in the lungs of patients with idiopathic pulmonary fibrosis as compared to in normal human lungs. Overexpression of miR-145 in lung fibroblasts increased SMA-α expression, enhanced contractility, and promoted formation of focal and fibrillar adhesions. In contrast, miR-145 deficiency diminished TGF-β1 induced SMA-α expression. miR-145 did not affect the activity of TGF-β1 but promoted the activation of latent TGF-β1. miR-145 targets KLF4, a known negative regulator of SMA-α expression. miR-145−/−mice are protected from bleomycin-induced pulmonary fibrosis [Yang, S. et al. FASEB J. (2013) 27 (6): 2382-91].

miR-21: MiR-21 is a highly expressed miRNA in mammalian cells and is associated with different types of cancer. Several studies have reported a major contribution of miR-21 to apoptosis in both heart and lung tissues in oxidative stress.

In pulmonary vascular smooth muscle cells (VSMCs) undergoing oxidative stress, miR-21 has been reported to target PDCD4, exerting a protective role as it does in cardiac myocytes [Climent, M. et al. Int. J. Mol Sci. (2020) 21: 4370, citing Cheng, Y. et al. J. Mol. Cell Cardio. (2009) 47: 5-14] and human aortic endothelial cells (HAECs) [Id., citing Rippe, C. et al. Exp. Gerontol. (2012) 47: 45-51]. In the lung, chronic hypoxia causes a massive ROS production leading to pulmonary oxidative stress, which results in pulmonary vascular remodeling [Id., citing Araneda, O F and Tuesta, M. Oxid. Med. Cell Longev. (2012) 2012; Jiang, C. et al. Allergy Asthma Clin. Immunol. (2019) 15: 33]. Sarkar et al. found that hypoxia could induce the proliferation of pulmonary arterial smooth muscle cells (PASMCs) through the upregulation of miR-21 [Id., citing Sarkar, J. et al. Am. J. Physiol. Lung Cell Mol. Physio. (2010) 299: 861-71]. Therefore, miR-21 was upregulated and actively participated in ROS response during pulmonary remodeling [Id., citing Jiang, C. et al. Allergy Asthma Clin. Immunol. (2019) 15: 33].

High miR-21 levels are a marker of immune cell activation in multiple contexts, although whether or not this reflects a cause or consequence of activation remains to be determined. (Sheedy, FJ. Front. Immunology (2015) 6: article 19). miR-21 expression is RNA polymerase II-dependent and derived from a primary transcript that is both capped and polyadenylated (Id., citing Cai, X. RNA (2004) 10: 1957-66). Similar to regular coding mRNAs, miR-21 expression is dynamically regulated by complex signaling pathways and can be enhanced by extracellular signals during immune cell development. Monocyte activation with phorbol 12-myristate 13-acetate (PMA, also known as 12-O-tetradecanoylphorbol-13-acetate or TPA) (Id., citing Kassashima, K. et al. Biochem. Biophys. Res. Comun. (2004) 322: 403-10) all trans retinoic acid to generate neutrophils (Id., citing Lu, J et al. Nature (2005) 435: 834-8), GM-CSF/IL-4 treatment to generate immature DCs (Id., citing Cekiaite, L. et al. Front. Biosci (Elite Ed.) (2010) 2: 818-28; Hashimi, S T et al. Blood (2009) 114: 404-14) treatment with LPS to generate activated macrophages (Id., citing Sheedy, F J et al. Nat. Immunol. (2010) 11: 141-7; Lu, T X et al. J. Immunol. (2010) 11: 141-7), and LPS-mediated B-cell activation (Id., citing 3), all revealed significant upregulation of miR-21. MiR-21 exhibits diversity in the signals, transcription factors and proposed binding sites that regulate its expression in diverse contexts. The complexity of the predicted promoter region of primary miRNA transcript (pri-miR-21) (Id., citing Fujita, S. et al. J. Mol. Biol. (2008) 378: 492-504; Loffler, D. et al. Blood (2007) 110: 1330-3) and the occurrence of alternative transcription start sites (Id., citing Ribas, J. et al. Nucleic Acids Res. (2012) 40: 6821-33) suggest that the regulation of miR-21 transcription is not straight forward.

miR-21 expression is upregulated by IL-6 and toll-receptor signaling, which activate STAT3 [Yang, C H et al. Pharmaceuticals (2015) 8: 836-847, citing Folini, M. et al. Mol. Cancer (2010) 9: 12; Loffler, D. et al. Blood (2007) 110: 1330-3]. Several lines of evidence indicate that both STAT3 and NFκB signaling pathways regulate miR-21 expression. Type I-interferon (IFN) induces expression of miR-21; this IFN-induction is STAT3-dependent.

miR-21 seems to be strongly associated with renal pathogenesis both in the glomerulus and tubulointerstitium of the kidney [Ichi, O. and Horino, T. J. Toxicol. Pathol. (2018) 31: 23-34]. miR-21 targets several molecules including P53, PDCD4, SMAD7, TEGBR2, TIMP3, CDC25A, CDK6, ERK/MAPK, PTEN, PPARA, MPV17L, DDAHI and RECK [Id.].

miRNA Sorting into Exosomes

miRNAs are not randomly incorporated into exosomes. Guduric-Fuchs et al. analyzed miRNA expression levels in a variety of cell lines and their respective derived exosomes and found that a subset of miRNAs (e.g., miR-150, miR-142-3p, and miR-451) preferentially enter exosomes [Zhang, J. et al. Genomics Proteomics Bioinformatics (2015) 13: 17-24, citing Guduric-Fuchs J., et al. BMC Genomics. (2012) 13: 357]. Similarly, Ohshima et al. compared the expression levels of let-7 miRNA family members in exosomes derived from the gastric cancer cell line AZ-P7a with those from other cancer cell lines, including the lung cancer cell line SBC-3/DMS-35/NCI-H69, the colorectal cancer cell line SW480/SW620, and the stomach cancer cell line AZ-521. As a result, they found that members of the let-7 miRNA family are abundant in exosomes derived from AZ-Pa but are less abundant in exosomes derived from other cancer cells [Id., citing Ohshima K., et al. PLoS One. (2010) 5: e13247]. Moreover, some reports have shown that exosomal miRNA expression levels are altered under different physiological conditions. The level of miR-21 was lower in exosomes from the serum of healthy donors than those glioblastoma patients [Id., citing Skog J., et al. Nat Cell Biol. (2008) 10: 1470-1476]. Levels of let-7f, miR-20b, and miR-30e-3p were lower in vesicles from the plasma of non-small-cell lung carcinoma patients than normal controls [Id., citing Silva J., et al. Eur Respir J. (2011) 37: 617-623]. Different levels of eight exosomal miRNAs, including miR-21 and miR141, were also found between benign tumors and ovarian cancers [Id., citing Taylor D. D., et al. Gynecol Oncol. (2008) 110: 13-21].

There are four potential modes for sorting of miRNAs into exosomes, although the underlying mechanisms remain largely unclear. These include: 1) The neural sphingomyelinase 2 (nSMase2)-dependent pathway. nSMase2 is the first molecule reported to be related to miRNA secretion into exosomes. Kosaka et al. found that overexpression of nSMase2 increased the number of exosomal miRNAs, and conversely inhibition of nSMase2 expression reduced the number of exosomal miRNAs [Id., citing Kosaka N., et al. J Biol Chem. (2013) 288:10849-10859]. 2) The miRNA motif and sumoylated heterogeneous nuclear ribonucleoproteins (hnRNPs)-dependent pathway. Villarroya-Beltri et al. discovered that sumoylated hnRNPA2B1 could recognize the GGAG motif in the 3′ portion of miRNA sequences and cause specific miRNAs to be packed into exosomes [Id., citing Villarroya-Beltri C., et al. Nat Commun. (2013) 4:2980]. Similarly, another two hnRNP family proteins, hnRNPA1 and hnRNPC, can also bind to exosomal miRNAs, suggesting that they might be candidates for miRNA sorting as well. However, no binding motifs have been identified yet [Id., citing Villarroya-Beltri C., et al. Nat Commun. (2013) 4:2980]. 3) The 3′-end of the miRNA sequence-dependent pathway. Koppers-Lalic et al. discovered that the 3′ ends of uridylated endogenous miRNAs were mainly presented in exosomes derived from B cells or urine, whereas the 3′ ends of adenylated endogenous miRNAs were mainly presented in B cells [Id., citing Koppers-Lalic D., et al. Cell Rep. (2014) 8:1649-1658]. The above two selection modes commonly indicate that the 3′ portion or the 3′ end of the miRNA sequence contains a critical sorting signal. 4) The miRNA induced silencing complex (miRISC)-related pathway. It is well known that mature miRNAs can interact with assembly proteins to form a complex called miRISC. The main components of miRISC include miRNA, miRNA-repressible mRNA, GW182, and AGO2. The AGO2 protein in humans, which prefers to bind to U or A at the 5′ end of miRNAs, plays an important role in mediating mRNA:miRNA formation and the consequent translational repression or degradation of the mRNA molecule [Id., citing Frank F., et al. Nature. (2010) 465:818-822]. Recent studies recognized a possible correlation between AGO2 and exosomal miRNA sorting.

The miRNAs in cell-released exosomes can circulate with the associated vehicles to reach neighboring cells and distant cells. After being delivered into acceptor cells, exosomal miRNAs play functional roles. Although it is difficult to completely exclude the effects of other exosomal cargos on recipient cells, miRNAs are considered the key functional elements. The functions of exosomal miRNAs can be generally classified into two types. One is the conventional function, i.e., miRNAs perform negative regulation and confer characteristic changes in the expression levels of target genes. For example, exosomal miR-105 released from the breast cancer cell lines MCF-10A and MDA-MB-231 reduced ZO-1 gene expression in endothelial cells and promoted metastases to the lung and brain [Id., citing Zhou W., et al. Cancer Cell. (2014) 25:501-515]. Exosomal miR-214, derived from the human microvascular endothelial cell line HMEC-1, stimulated migration and angiogenesis in neighboring HMEC-1 cells [Id., citing van Balkom B. W., et al. Blood. (2013) 121:S1-S15]. Exosomal miR-92a, derived from K562 cells, significantly reduced the expression of integrin a5 in the human umbilical vein endothelial (HUVEC) cells and enhanced endothelial cell migration and tube formation [Id., citing Umezu T., et al. Oncogene. (2013) 32: 2747-2755]. The other one is a novel function that has been identified in some miRNAs when they are studied as exosomal miRNAs rather than intracellular miRNAs. Exosomal miR-21 and miR-29a, in addition to the classic role of targeting mRNA, were first discovered to have the capacity to act as ligands that bind to toll-like receptors (TLRs) and activate immune cells [Id., citing Fabbri M., et al. Proc Natl Acad Sci USA. (2012) 109: E2110-E2116].

Exosomal miRNAs can stably exist in the blood, urine, and other body fluids of patients, and exosomes can reflect their tissue or cell of origin by the presence of specific surface proteins [Id., citing Simons M., et al. Curr Opin Cell Biol. (2009) 21:575-581, Mathivanan S., et al. J Proteomics. (2010) 73:1907-1920, Gross J. C., et al. Nat Cell Biol. (2012) 14:1036-1045]. Furthermore, the amount and composition of exosomal miRNAs differ between patients with disease and healthy individuals. Thus, exosomal miRNAs show potential for use as noninvasive biomarkers to indicate disease states. Several previous studies have profiled exosomal miRNAs in different samples. Some exosomal miRNAs can be used to aid in clinical diagnosis [Id., citing Skog J., et al. Nat Cell Biol. (2008) 10:1470-1476; Silva J., et al. Eur Respir J. (2011) 37:617-623; Taylor D. D., et al. Gynecol Oncol. (2008) 110: 13-21; Rabinowits G., et al. Clin Lung Cancer. (2009) 10:42-46]. For example, a set of exosomal miRNAs, including let-7a, miR-1229, miR-1246, miR-150, miR-21, miR-223, and miR-23a, can be used as the diagnostic biomarker of colorectal cancer [Id., citing Ogata-Kawata H., et al. PLoS One. (2014) 9: e92921]. Another set, miR-1290 and miR-375, can be used as the prognostic marker in castration-resistant prostate cancer [Id., citing Huang X., et al. BMC Genomics. (2013) 14:319].

Besides endogenous miRNAs, exogenous miRNAs can also be sorted into exosomes, which has been experimentally confirmed by Pegtel et al. [Id., citing Pegtel D. M., et al. Proc Natl Acad Sci USA. 2010; 107: 6328-6333] and Meckes et al. [Id., citing Meckes, DG, Jr. et al. Proc. Natl. Acad. Sci. USA (2010) 107: 20370-75], who observed that human tumor viruses can exploit exosomes as delivery vectors to transfer their exogenous miRNAs to other non-infected cells [Id., citing Pegtel D. M., et al. Proc Natl Acad Sci USA. 2010; 107:6328-6333, Meckes D. G., Jr., et al. Proc Natl Acad Sci USA. 2010; 107:20370-20375]. Hence, exogenous small RNAs have also been transferred by exosomes by mimicking the molecular mechanism of endogenous miRNAs transportation.

### DNA

Studies have shown that certain EVs may contain DNA fragments [Shao, H. et al. Chem Rev. (2018) 118 (4): 1917-50], citing Balaj, L. et al. Nat. Commun. (2011) 2: 180; Thakur, B K et al. Cell Res. (2014) 24: 766-769; Guescini, M. et al. J. Neural. Trans. (Vienna) (2010) 17: 1-4; Kahlert, C. et al. J. Biol. Chem. (2014) 289: 3869-75; Takahashi, A. et al. Nat. Commun. (2017) 8: 15287]. These DNA are double-stranded fragments which range from 100 base pairs (bp) to 2.5 kbp [Id., citing Thakur, B K et al. Cell Res. (2014) 24: 766-9]. The larger-sized population (>2.5 kbp) was found to be predominately external DNA associated with EVs and smaller-sized population (100 bp-2.5 kbp) as internal DNA confined within EVs. These fragments represent the whole genomic DNA and could be used to identify mutations present in parental tumor cells [Id., citing Thakur, B K, et al., Kahlert, C. et al. J. Biol. Chem. (2014) 289: 3869-75]. The functional roles of these DNAs have yet to be determined.

### Ev Composition:

The heterogeneity of exosomes and/or extracellular vesicles is thought to be reflective of their size, content, functional impact on recipient cells, and cellular origin. During their secretion they acquire surface proteins from their cell of origin. They naturally transport mRNA, miRNA, and proteins between cells.

Biofluids can contain large quantities of EVs that shuttle various molecules from parental cells to other cells, including proteins [Id., citing Graner, M W et al. FASEB J. (2009) 23: 1541-57; Simpson, R J et al. Proteomics (2009) 6: 267-83; Mathivanan, S. et al. Nucleic Acids, Res. (2012) 40: D1241-4], mRNA/miRNA [Id., citing Valadi, H. et al. Nat. Cell Biol. (2007) 9: 654-9; Skog, J. et al. Nat. Cell Biol. (2008) 10: 1470-6] and DNA [Id., citing Balaj, L. et al. Nat. Commun. (2011) 2: 180]. Extracellular vesicles therefore are mediators of near and long-distance intercellular communication in health and disease and affect various aspects of cell biology.

EV composition is determined not only by the cell type but also by the physiological state of the producer cells. The diversity of mechanisms by which EVs are generated and confer effects provides both opportunities and challenges for developing EV-based therapeutics (György B, et al. Annu Rev Pharmacol Toxicol. (2015) 55: 439-464). Many methods are used to isolate EVs, and EV contents and properties overlap with those of the cells of origin and other EV types. Formalizing EV nomenclature and defining attributes is a work in progress. The mechanisms of EV uptake and content delivery (or degradation) vary among EV types and recipient cell types. Elucidating and understanding these processes is critical for harnessing EVs as therapeutic delivery vehicles. Multiple lines of evidence indicate that EVs can transfer biomolecules to modulate recipient cell state in vivo, for example, following bolus injection of purified or concentrated EVs. However, the extent to which such processes naturally shape cellular function and intercellular communication, particularly under homeostatic conditions, remains poorly understood. Moreover, we do not understand the relative importance of EV-mediated transfer between proximal cells, for example, when diffusional barriers lead to local accumulation of secreted EVs rather than transfer of EVs via the circulation, where EV concentrations may be lower. EV-mediated signaling is dose-dependent (Id., citing Yu S, et al. J. Immunol. 2007; 178: 6867-75), so the tuning of EV dose may enable the balancing of potential deleterious and therapeutic effects of EV administration. Understanding the role of EV dose is also important for achieving therapeutic efficacy.

### Ev Binding:

EV binding is mediated by receptors that interact with either universal EV molecules, such as lipids and carbohydrates, or specific peptides present on subsets of EVs. Following initial binding, cells internalize EVs by processes that include receptor-mediated phagocytosis or endocytosis via receptors that include T cell immunoglobulin- and mucin-domain-containing molecule-4 (TIM4), which binds to phosphatidylserine (PS) on EVs; scavenger receptors; integrins; and complement receptors (György B, et al. Annu Rev Pharmacol Toxicol. (2015) 55: 439-464, citing Record M, et al. Biochem. Pharmacol. 2011; 81: 1171-82). How EV cargo is released into the cytoplasm after entry into recipient cells is unclear. Furthermore, uptake of cargo into a cell is not equivalent to cargo functionality. For instance, EVs may potentially pass through cells within the multivesicular body compartment, which could explain how EVs cross the blood-brain barrier (BBB) (i.e., via a transendothelial route). Endocytotic mechanisms must circumvent the lysosomal degradative pathway, and direct fusion between the EV and target cell plasma membrane or endocytotic membrane does not always ensure functionality of the contents. In many cases, EV cargo can be degraded by recipient cells, thereby inhibiting therapeutic delivery but limiting the impact of off-target delivery. In general, the fate of EVs within the body and cells remains poorly understood and requires additional investigation to elucidate how these processes impact functional EV-mediated delivery [Id.].

EVs comprising exosomes are released by most if not all cell types, including platelets, blood cells, dendritic cells, mast cells, T cells, B cells, epithelial cells, endothelial cells, mesenchymal stem cells, smooth muscle cells, neuronal cells and many tumor cells [Zhang, J. et al. Genomics Proteomics Bioinformatics (2015) 13: 17-24, citing Liao J., et al. Int J Mol Sci. 2014; 15: 15530-15551; Kopers-Lalic, D. et al. Adv. Drug delivery rev. (2012) doi: 10.1016/j.addr.2012.07.006, citing Thery V., et al. Nat. rev. Immunol. (2002) 2: 569-79].

Most EVs comprising exosomes share a core set of proteins and lipids; there seems to be a clear conserved protein repertoire in exosomes across cell-types and species [Kopers-Lalic, D. et al. Adv. Drug delivery rev. (2012) doi: 10.1016/j.addr.2012.07.006, citing Simpson, R J, et al. Proteomics (2008) 8: 4083-99]. For example, the endosomal proteins such as Alix and TSG101 have been identified in the majority of the exosomes studied for their protein content thus far. In addition, heat shock proteins, which are involved in protein trafficking, are frequently found in exosomes [Id., citing van Dommelen, S M et al. J. Control. Release (2011) 161: 635-44]. Exosomes are further enriched in tetraspanins, like CD9, CD63, CD81 and CD82, which are important molecules for protein-protein interactions in cellular membranes. Tetraspanins bind many proteins, including integrins and MHC molecules [Id., citing Thery, C., et al. Nat. Rev. Immunol. (2009) 9: 581-593; Escola, J M. J. Biol. Chem. (1998) 273: 20121-20127; Keller, S. et al. Immunol. Lett (2006) 107: 102-108; Stoorvogel, W. et al. Traffic (2002) 3: 321-330]. Specific Rab proteins, a highly conserved family of small GTPases that function as molecular switches and coordinate membrane traffic [Id., citing Stenmark, H. Nat. Rev. Mol. Cell Biol. (2009) 10: 513-25], are often observed in exosomes by mass-spectrometry. Exosomes are also rich in annexins, membrane trafficking proteins that are involved in fusion events. Furthermore, cytoskeletal proteins like myosin, actin and tubulin are present in exosomes. Finally, metabolic enzymes, antigen presentation molecules, ribosomal proteins and signal transduction molecules have been shown to be present in exosomes [Id., citing Mathivanan, S., et al. Proteomics (2008) 8: 4083-99].

Besides selected sets of proteins, EVs comprising exosomes also incorporate (functional) nucleic acids, most notably small RNA molecules [Id., citing Zomer, A. et al. Commun. Integr. Biol. (2010) 3: 447-450; Gibbings, D, Voinnet, O. Trends Cell Biol. (2010) 20: 491-501]. Of all RNA molecules detected in exosomes, the class of 22nt long, non-coding miRNAs has received attention since the discovery that miRNAs can be functionally transferred to recipient cells [Id., citing Pegtel, D M et al. Proc. Nat. Acad. Sci. USA (2010) 107: 6328-6333, Valadi, H. et al. Nat. Cell Biol. (2007) 9: 654-659]. MiRNAs regulate gene expression by binding imperfectly to the 3′ untranslated region of the target mRNA that results in translational repression of the mRNA into protein [Id., citing Bartel, DP. Cell (2004) 116: 281-297; Brennecke, J. et al. PLoS Biol. (2005) 3: e85].

EVs comprising exosomes also contain specific proteins depending on the cell of origin. As examples, exosomes from tumor cells contain tumor antigens, platelet-derived exosomes contain coagulation factors, and exosomes from dendritic cells express toll-like receptor ligands [Zhu, L. et al., Artificial Cells, nanomedicine and biotechnology (2018) 46 (53): S166-S179, citing Jong, A Y et al. J. Extracell. Vesicles (2017) 6: 1294368; Sobo-Vujanovic, A. et al., Cell Immunol. (2014) 289: 119-127]. Exosome-mediated functions vary depending on the condition or the origin of the cells [Id., citing Kim, O Y, et al. Semin. Cell Dev. Biol. (2017) 67: 74-82].

### Trafficking of Exosomes

There are three mechanisms of interaction between EVs comprising exosomes and their recipient cells. First, the transmembrane proteins of exosomes directly interact with the signaling receptors of target cells [Zhang, J. et al. Genomics Proteomics Bioinformatics (2015) 13: 17-24, citing Munich S., Sobo-Vujanovic A., Buchser W. J., Beer-Stolz D., Vujanovic N. L. Dendritic cell exosomes directly kill tumor cells and activate natural killer cells via TNF superfamily ligands. Oncoimmunology. (2012) 1:1074-1083]. Second, the exosomes fuse with the plasma membrane of recipient cells and deliver their content into the cytosol [Id., citing Mulcahy L. A., et al. J Extracell Vesicles. (2014) 3: 10.3402/jev/v3/24641]. Third, the exosomes are internalized into the recipient cells and have two fates. In one case, some engulfed EVs comprising exosomes may merge into endosomes and undergo transcytosis, which will move EVs comprising exosomes across the recipient cells and release them into neighboring cells. In the other case, endosomes fused from engulfed exosomes will mature into lysosomes and undergo degradation [Id., citing Mulcahy L. A., et al. J Extracell Vesicles. (2014) 3: 10.3402/jev/v3/24641, Tian T., et al. J Cell Physiol. (2013) 228:1487-1495]. Some factors influencing internalization of exosomes in recipient cells have been reported. For example, Koumangoye et al. observed that disruption of exosomal lipid rafts resulted in the inhibition of internalization of exosomes and that annexins, which are related to cell adhesion and growth, were essential for the uptake of exosomes in the breast carcinoma cell line BT-549 [Id., citing Koumangoye R. B., et al. PLoS One. (2011) 6: e24234]. Escrevente et al. described a decrease in exosome uptake after the ovarian carcinoma cell line SKOV3 and its derived exosomes were treated with protease K, which indicated that the proteins mediating exosome internalization are presented on the surface of both the cells and the exosomes [Escrevente C., et al. BMC Cancer. (2011) 11:108].

### Exosome Function

EVs comprising exosomes exert their functions through a number of different mechanisms. They can transfer their cargo or membrane constituents from one cell to another, thus transferring functions between cells [Mathieu, M. et al. Nat. Cell Biol. (2019) 21 (10): 9-17]. They carry molecules on their surface, which can act as ligands that stimulate surface receptors in other cells activating intracellular signaling [Maia, J. et al. Front. Cell Dev. Biol. (2018) 6: 18]. These surface molecules can directly perform functions in the extracellular milieu; for example, exosomes may carry remodeling enzymes of their surface, such as matrix metalloproteinases, heparanases, and hyaluronidases [Nawaz, M. et al. Cells (2018) 7 (10) PMC6210724]. Surface proteins on the EV membrane also may capture external molecules or pathogens to neutralize their effects.

It is believed that exosomes can regulate the bioactivities of recipient cells by the transportation of lipids, proteins, and nucleic acids while circulating in the extracellular space [Zhang, J. et al. Genomics Proteomics Bioinformatics (2015) 13: 17-24, citing Liao J., et al. Int J Mol Sci. 2014; 15:15530-15551].

### EVs Play a Role in Several Normal and Disease Physiological Processes.

### Blood Hemostasis.

Molecules on the surface of some EVs, such as tissue factor or phosphatidylserine, work as activators of the coagulation cascade. These EVs, which are available in the blood during injury or endothelial damage are absent from healthy circulating blood. Normal circulating EVs carry plasminogen activators, which induce fibrinolytic activity, preventing thrombus formation. Additionally, exosomes released from platelets under normal conditions inhibit platelet aggregation. (Zarra, M. et al. Intl J. Mol. Sci. (2019) 20 (11 PMX6600675).

### Wound Healing.

With regard to the role of miRNAs in the proliferation and differentiation of MSCs in the setting of wound healing (Guo, L. et al. Exptl Hematol. (2011) 39: 608-616, citing Silo, S., et al. DNA Cell Biol. (2007) 26: 227-37), using a skin excision model, altered expression in a panel of miRNAs, including upregulated expression of miR-31, -21, -223, -142, -205, -203, -18b, -19a, -130b, -16, -26b, -125b, and let-7f, and down regulated expression of miR-133a, -181, -30a-3p, -193b, -30a-5p, -204, -200b, -96, -127, -181c, -182 and -130a was demonstrated at the stage of active granulation formation (Id., citing Zou, Z. et al. Expert Opin. Biol. Thera. (2010) 10: 215-30). Further, TGFβ, a key growth factor elevated in the wound site, was found to stimulate upregulation of miR-21 in MSCs as well as in multipotential C3H10T1/2 cells, and to promote proliferation and differentiation of these cells in vitro [Zou, Z. et al., Expert Opin. Biol. Thera. (2010) 10: 215-30]. Consistently, knockdown of miR-21 in the wound bed delayed the healing process. These results suggest that miR-21 regulates gene expression and, subsequently, the behavior of MSCs in wound healing.

Exosomes derived from bone mesenchymal stem cells preconditioned by stimulation with Fe₃O₄ nanoparticles and a static magnetic field (mag-BMSC-Exos) were reported to enhance wound healing through upregulated miR-21-5p [Wu, D. et al. Intl J. Nanomedicine (2020) 15: 7979-93]. mag-BMSC-Exos were compared to exosomes derived from bone marrow mesenchymal stem cells (BMSC-exo) without preconditioning. Both were isolated by ultracentrifugation. Wound healing in in vitro experiments, including scratch wound assays, transwell assays, and tube formation assays, and an established an in vivo wound healing model were compared, as were the miRNA expression profiles were compared between BMSC-Exos and mag-BMSC-Exos.

In vitro studies have shown that exosomes derived from human adipose mesenchymal stem cells (ASCs-Exos) could be taken up and internalized by fibroblasts to stimulate cell migration, proliferation and collagen synthesis in a dose-dependent manner, with increased genes expression of N-cadherin, cyclin-1, PCNA and collagen I, III. In vivo, tracing experiments demonstrated that ASC-Exos could be recruited to soft tissue wound area in a mouse skin incision model and that they significantly accelerated cutaneous wound healing. Hu, L. et al. Sci. rep. (2016) 6: 32993].

The efficacy of exosomes derived from human umbilical cord blood (UCB-Exos) on wound healing was evaluated by measuring wound closure rates, histological analysis and immunofluorescence examinations. In vitro, quantitative real-time PCR (qRT-PCR) analysis was performed to detect the expression levels of a class of miRNAs that have positive roles in regulating wound healing. The scratch wound assay, transwell assay and cell counting kit-8 analysis were conducted to assess the effects of UCB-Exos on migration and proliferation of human skin fibroblasts and endothelial cells. Tube formation assay was carried out to test the impact of UCB-Exos on angiogenic tube formation ability of endothelial cells. Meanwhile, by using specific RNA inhibitors or siRNAs, the roles of the candidate miRNA and its target genes in UCB-Exos-induced regulation of function of fibroblasts and endothelial cells were assessed.

In vitro, exosomes derived from human umbilical cord blood (UCB-Exos) could promote the proliferation and migration of fibroblasts and enhance the angiogenic activities of endothelial cells. In vivo, the local transplantation of exosomes derived from human umbilical cord blood (UCB-Exos) into full thickness mouse skin wounds resulted in accelerated re-epithelialization, reduced scar widths, and enhanced angiogenesis. It was concluded that accelerated cutaneous wound healing following local transplantation of UCB-Exos occurred through miR-3p-mediated promotion of angiogenesis and fibroblast function. Hu, Y. et al. Theranostics (2018) 8 (10: 169-84).

### Metabolic Functions

EVs play essential metabolic functions by transferring enzymes and metabolites between cells or by performing extracellular metabolic activities. Studies have shown that EVs secreted by liver hepatocytes contain hundreds of enzymes that belong to the different metabolic pathways. When incubated with rat serum, these ECs changed the metabolic profile of the serum [Royo, F. et al., body metabolism. [Royo, F. et al. Sci. Rep. (2017) 7: 42798].

### The Role of EVs in the Host-Pathogen Relationship

The release of pathogen-derived vesicles was observed 40-50 years ago in gram-negative bacteria, Vibrio cholerae [Kuipers, M E et al. Front. Microbiol. (2018) 9: 2182, citing Chatterjee, AN, Das, J. J. Gen. Microbiol. (1967) 49: 1-11] and Neisseria meningitidis [Id., citing Devoe, I W and Gilchrist, JE J. Exp. Med. (1973) 138: 1156-67], the fungus Cryptococcus neoformans [Id., citing Takeo, K. et al. J. Bacteriol. (1973) 113: 1449-54], and the parasites Schistosoma mansoni and Fasciola hepatica [Id., citing Senft, A et al. J. Parasitol. (1961) 47: 217-229; Threadgold, LT. Exp. Cell Res. (1963) 30: 238-42]. These vesicles were initially regarded as artifacts, decades later, studies on gram-negative bacteria showed that their released outer membrane vesicles supported bacterial survival. Bacterial vesicles were reported to contribute to biofilm formation [Id., citing Schooling, S R and Beveridge, TJ J. Bacteriol. (2006) 188: 5945-57] and were able to transfer DNA to other bacteria, thereby sharing genes involved in, for example, antibiotic resistance [Id., citing Renelli, M et al. Microbiology (2004) 150: 2161-69; Mashburn-Warren, L M and Whiteley, M Mol. Microbiol. (2006) 61: 839-46]. Evidence was also provided for release of EV by gram-positive bacteria [Id., citing Dorward, D W and Garon, CF Appl. Environ. Microbiol. (1990) 56: 1960-62], fungi (Rodrigue, M L et al. Eukaryot. Cell (2007) 6: 48-59), parasites [Id., citing Silverman, J M et al. J. Cell Sci. (2010) 123: 842-52], parasite-infected cells, like Plasmodium falciparum-infected red blood cells [Id., citing Mantel, P et al. Cell Host Microbe (2013) 13: 521-34], and the pathogenic protozoa Acanthamoeba castellanii [Id., citing Goncalves D S et al. Virulence (2018) 9: 818-36].

Pathogens can directly utilize EVs in delivery of virulence factors (Id., citing Deo, P et al. PLoS Pathog. (2018) 14: e1006945, Kunsmann, L. et al. Sci. Rep. (2015) 5: 13252; Rivera, J. et al. Proc. Natl Acad. Sci. USA (2010) 107: 19002-109007, disruption of natural barriers, cytotoxicity, and modulation of host cells [Kuipers, M E et al. Front. Microbiol. (2018) 9: 2182; Liu, Y, et al., Front. Microbiol. (2018) 9: 1502]. They also can use their EVs to evade the immune system through several mechanisms, like complement inhibition and antibody degradation. Proteomic studies on bacterial EVs suggested that the protein cargo of these EVs have been loaded selectively from the cytoplasm to perform these functions [Lee, J. et al. Proteomics Clin. Appl. (2016) 10 (9-110): 897-909]. Bacterial EVs also mediate the non-genetic acquisition of antibacterial resistance; for example, EVs can transfer B-lactamase to bacteria that are not genetically expressing it [Lee, J. et al. Antimicrob. Agents Chemother. (2013) 57 (6): 2589-95].

### Viral Infection

Viruses hijack the endosomal pathway or the endosomal machinery for their own benefit, not only to produce new virions in the productive stages, but also for ingenious means of immunoevasion or to facilitate viral spread [Kopers-Lalic, D. et al. Adv. Drug delivery rev. (2012) doi: 10.1016/j.addr.2012.07.006] Besides modifying the proteome and genetic content of exosomes, it appears that viruses exploit a whole range of secreted vesicles [Id., citing Meckes, D G et al. J. Virol. (2011) 85: 12844-54]. HIV for instance exploits the Endosomal sorting complex Required for Transport (ESCRT) machinery for the formation of a particular exosome-like vesicle that is closely involved with viral budding from the plasma membrane [Id., citing Meckes, D G et al. J. Virol. (2011) 85: 12844-12854, Benaroch, P. et al. Retrovirol. (2010) 7: 29; Penchen-Matthews, A., et al. Trends Microbiol. (2004) 12: 310-316] that allows a stealthy infection of neighboring cells i.e. without the use of envelope glycoproteins (Env) and/or retroviral receptors [Id., citing Gould, S J et al. Proc. Nat. Acad. Sci. USA (2003) 100: 10592-10597]. In addition, exosomes released from HIV infected cells may contain co-receptors (CCR5) that when transferred to neighboring/recipient cells may enhance their susceptibility to infection by HIV promoting viral spread [Id., citing Mack, M. et al. Nat. Med. (2000) 6: 769-775]. Although many details are unknown, another interesting observation is the specific release and transport of the HIV Nef protein to neighboring cells via exosomes or ‘nanotubes’ [Id., citing daSilva, LL. et al. J. Virol. (2009) 83: 6578-6590]. Nef is able to alter the endosomal system altogether by increasing the number of endosomes, lysosomes and MVBs [Id., citing Mack, M. et al. Nat. Med. (2000) 6: 769-775; Madrid, R. et al. J. Biol. Chem. (2005) 280: 5032-5044]. Nef is widely considered as an HIV virulence factor and one mechanism maybe the secretion via exosomes that is associated with the induction of apoptosis of responding CD4+T cells [Campbell, T D et al. Ethn. Dis. (2008) 18: S2-S9]. Collectively, these observations link Nef in exosomes to HIV pathogenesis by means of stimulating immuno-evasion.

Virus-infected cells also package virus-encoded RNAs into exosomes that are delivered into non-infected recipient cells [Id.].

The number and contents of exosomes in body fluids may change significantly with disease. For example, there are increased numbers of BAL exosomes in sarcoidosis patients compared with healthy volunteers [Alipoor, S D et al. Mediators of Inflammation (2016) 5628404., citing Qazi, K R, et al. Thorax, (2010) 65 (11): 1016-1024]. BAL exosomes from sarcoidosis patients induce the production of inflammatory cytokines by PBMCs and promote the release of CXCL-8 by airway epithelial cells through delivery of pathogen-associated proinflammatory mediators [Qazi, K R, et al. Thorax (2010) 65 (11): 1016-1024]. In addition, increased numbers of circulating exosomes are associated with disease progression in cancer [Id., citing Masyuk, A I, et al. Journal of Hepatology (2013) 59 (3): 621-625; Skog, J. et al. Nature Cell Biology (2008) 10 (12): 1470-1476]. The exosomal content may also provide valuable information about disease status [Id., citing Tesselaar, M E T et al. Journal of Thrombosis and Haemostasis (2007) 5 (3): 520-527]. For example, pioneering work demonstrated that exosomal cargos changed markedly during hepatitis B viral infection: (1) exosomes can participate directly in viral replication; (2) exosomes modulate immune response during viral infections; (3) exosomal RNAs and proteins might be selected as novel biomarkers for the diagnosis of viral infections; and (4) exosomes can also be designed as therapeutics to attenuate viral replication [Li, S. et al. Biomed. Res. Int. (2019) 2019:2103943. doi: 10.1155/2019/2103943].

Role of miRNAs in Modulating Gene Expression During Viral Infection

MicroRNAs (miRNAs) are implicated in modulating gene expression by interfering with mRNA translation most commonly by destabilizing mRNA thereby facilitating degradation. One miRNA may target a large number of genes, and the targets of an miRNA may belong to a variety of functional groups. In turn, the 3′-UTR of a single mRNA transcript may be the target for several different miRNAs, miRNA-mediated regulation of gene expression has been found to affect many cellular functions, including innate and antiviral responses [Brogaard, L. et al., Sci. Reports (2016) 6:21812].

Members of the miR-29 family are predicted to function as inhibitors of numerous mRNAs involved in ECM production and fibrosis [van Rooij, E. et al. Proc. Natl. Acad. Sci. USA (2008) 105: 13027-32]. These investigators showed that cardiac hypertrophy and heart failure are accompanied by characteristic changes in the expression of a collection of specific microRNAs (miRNAs), which act as negative regulators of gene expression, and that myocardial infarction (MI) in mice and humans also results in the dysregulation of specific miRNAs, which are similar to but distinct from those involved in hypertrophy and heart failure. Among the MI-regulated miRNAs are members of the miR-29 family, which are down-regulated in the region of the heart adjacent to the infarct. The miR-29 family targets a cadre of mRNAs that encode proteins involved in fibrosis, including multiple collagens, fibrillins, and elastin. Thus, down-regulation of miR-29 would be predicted to derepress the expression of these mRNAs and enhance the fibrotic response. Indeed, down-regulation of all three members of the miR-29 family with anti-miRs in vitro and in vivo induced the expression of collagens and enhanced the fibrotic response. This down-regulation remained present even after initial infarct healing had taken place. Downregulation was more pronounced in the border zone than in the remote myocardium. To determine whether overexpression of miR-29 was capable of reducing collagen expression, fibroblasts were exposed to a miR-29b mimic. The level of miR-29b expression in fibroblast cultures increased by as much as 400-fold after 3 days of exposure to miR-29b mimic. miR-29a expression was unaffected and miR-29c expression was increased only slightly by miR-29b mimic. Real-time PCR analysis indicated that the expression of collagen transcripts was diminished in response to miR-29b mimic. However, the magnitude of the decrease in collagen expression was modest compared with the increase in expression of miR-29b, suggesting that miR-29 levels are not the sole determinant of collagen mRNA expression.

Steiner, D F, et al., [Immunity (2011) 35: 169-81], demonstrated that miR-29 is an important regulator of the INF-γ pathway in helper T cells and that this regulation is mediated in part through the Th1 cell transcription factor T-bet. They showed that miR-29 regulates helper T cell differentiation by repressing multiple target genes, including at least two that are independently capable of inducing the Th1 cell gene expression program. miRNA-deficient helper T cells exhibit abnormal INF-γ production and decreased proliferation. Multiple members of the miR-17 and miR-92 families enhanced miRNA-deficient T cell proliferation, whereas miR-29 largely corrected their aberrant INF-γ expression. Repression of INF-γ production by miR-29 involved direct targeting of both the CD4+ T cell T-box family transcription factor T-bet and a closely related T-box family transcription, factor particularly in CD8+ T cells Eomesodermin (Eomes), which are known to induce INF-γ production. Although not usually expressed at functionally relevant amounts in helper T cells, Eomes was abundant in miRNA-deficient cells and was upregulated after miR-29 inhibition in wild type cells.

Differential expression of T bet and Eomes facilitates the cooperative maintenance of the pool of antiviral CD8+ T cells during chronic viral infection [Paley, M A et al., Science (2012) 338: 1220-125]. During chronic infections, T-bet is reduced in virus-specific CD8+ T cells; this reduction correlates with T cell dysfunction. In contrast, Eomes mRNA expression is up-regulated in exhausted CD8+ T cells during chronic infection [Id.].

miR-122 is an indispensable factor in supporting hepatitis C virus (HCV) replication [Li, Y., et al. J. Virol. (2010) 84 (6): 3023-32, citing Jopling, C L, et al., Modulation of hepatitis C virus RNA abundance by a liver-specific microRNA. Science (2005) 309:1577-1581], whereas miR-196 and miR-296 substantially attenuate viral replication through type I interferon (IFN)-associated pathways in liver cells [Id., citing Pedersen, I M, et al, Interferon modulation of cellular microRNAs as an antiviral mechanism. Nature (2007) 449:919-922]. Furthermore, miR-125b and miR-223 directly target human immunodeficiency virus type 1 (HIV-1) mRNA, thereby attenuating viral gene expression in resting CD4+ T cells [Id., citing Huang, J, et al., Cellular microRNAs contribute to HIV-1 latency in resting primary CD4+T lymphocytes. Nat. Med. (2007) 13:1241-1247), and miR-198 modulates HIV-1 replication indirectly by repressing the expression of cont1 [Id., citing Sung, T. L., and A. P. Rice. (2009) miR-198 inhibits HIV-1 gene expression and replication in monocytes and its mechanism of action appears to involve repression of cyclin T1. PLoS Pathog. 5:e1000263], a cellular factor necessary for HIV-1 replication.

The mammalian miRNA pathway has been shown to restrict the replication of infecting viruses and promotes latency [Aqil, M. et al., J. Extracell. Vesicles (2014) 3. doi: 10342/jev.v3.23129]. While miR-323, miR-491 and miR-654 inhibit the replication of H1N1 influenza virus, miR-24 and miR-93 limit vesicular stomatitis virus (VSV) replication, and miR-32 restricts primate foamy virus type 1 (PFV-1) [Id., citing Song L., et al. Silencing suppressors: viral weapons for countering host cell defenses. Protein Cell. (2011) 2: 273-81]. Human miR-28, miR-125b, miR-150, miR-223 and miR-382 target the 3′UTR of HIV-1 transcripts potentially taking productive infection towards latency [Id., citing Wang X, et al. Cellular microRNA expression correlates with susceptibility of monocytes/macrophages to HIV-1 infection. Blood. (2009) 113: 671-4].

Furthermore, viruses may promote their life cycles by modulating the intracellular environment through actively regulating the expression of multiple cellular microRNAs. For example, human T-cell lymphotropic virus type 1 (HTLV-1) modulates the expression of a number of cellular microRNAs in order to control T-cell differentiation [Li, Y., et al., J. Virol. (2010) 84(6): 3023-32, citing Bellon, M. et al. Deregulation of microRNA involved in hematopoiesis and the immune response in HTLV-I adult T-cell leukemia. Blood (2009) 113:4914-4917]. Similarly, human cytomegalovirus (hCMV) selectively manipulates the expression of miR-100 and miR-101 to facilitate its own replication [Id., citing Wang, F Z et al, 2008. Human cytomegalovirus infection alters the expression of cellular microRNA species that affect its replication. J. Virol. (2008) 82: 9065-9074].

MiR-132 has been shown to be highly induced after herpes simplex virus-1 (HSV-1), and human cytomegalovirus (hCMV) infection, and to down-regulate the expression of interferon-stimulated genes thereby facilitating virus replication [Bakre, A. et al., PLos One (2013) 6: e6679., citing Lagos D, et al. Nature Cell Biology (2010) 12: 513-519]. HSV-1 replication is suppressed when miRNA-101 (miR-101) targets a subunit of mitochondrial ATP synthase (ATP5B) [Id., citing Zheng S Q, et al. Antiviral Res (2011) 89: 219-226]. Human immunodeficiency virus type 1 (HIV-1) down-regulates the expression of many cellular miRNAs [Id., citing Yeung M L, et al. Retrovirology (2005) 2: 81], and for miR-17/92, miRNA suppression is required for efficient virus replication [Id., citing Triboulet R, et al. Science (2007) 315: 1579-1582].

### Influenza Virus

Influenza viruses are ubiquitous, causing acute respiratory disease and substantial morbidity and mortality each year [Bakre, A. et al., PLoS One (2013) 6: e66796., citing Thompson W W, et al. (2004) Influenza-associated hospitalizations in the United States. JAMA 292: 1333-1340; Thompson W W, et al. (2003) Mortality associated with influenza and respiratory syncytial virus in the United States. JAMA 289: 179-186; MMWR (2010) Estimates of Deaths Associated with Seasonal Influenza—United States, 1976-2007. Atlanta, GA: Centers for Disease Control and Prevention. pp. 1057-1062]. Influenza viruses belong to the family Orthomyxoviridae, are enveloped, and have an eight segmented, negative-sense, single-stranded RNA genome that encodes up to 11 proteins [Id., citing Palese P S M (2007) Fields Virology; Knipe D M H P, editor. Philadelphia: Raven]. The viral envelope contains the surface glycoproteins and antigenic determinants, hemagglutinin (HA) and neuraminidase (NA), as well as the membrane ion channel protein, M2. Within the virion, the matrix protein (M1) provides structure and secures the viral ribonucleoprotein (vRNP) complexes consisting of viral RNA coupled to nucleoprotein (NP) and the three polymerase proteins (PB1, PB2 and PA). The remaining viral proteins include the nonstructural proteins, NS1 and NS2, and the recently identified PB1-F2 protein found in some virus species. The virus must infect a host cell to co-opt host proteins and pathways for the successful generation of progeny virus. host cell pathways affected. Overlap was identified in pathways used for virus entry [Id., citing Shapira S D, et al. (2009) Cell 139: 1255-1267; Karlas, A. et al., Nature (2010) 463 (7282): 818-22]; Konig R, et al. Human host factors required for influenza virus replication. Nature (2010) 463: 813-817; Brass A L, et al. (2008) Science 319: 921-926], fusion of the endosomal and viral membrane [Id., citing Hao L, et al. (2008) Nature 454: 890-893 Karlas, A. et al., Nature (2010) 463 (7282): 818-22; Konig R, et al. Nature (2010) 463: 813-817; Brass A L, et al. (2008) Science 319: 921-926], transport of the viral components to the nucleus [Id., citing Karlas, A. et al., Nature (2010) 463 (7282): 818-22; Konig R, et al. (2009) Nature 463: 813-817], as well as late events including export of the vRNP complex and RNA into the cytoplasm [Id., citing Brass A L, et al. (2009) Cell 139: 1243-1254; Hao L, et al. (2008) Nature 454: 890-893; Shapira S D, Gat-Viks I, Shum B O, Dricot A, de Grace M M, et al. (2009) Cell 139: 1255-1267; Karlas, A. et al., Nature (2010) 463 (7282): 818-22; Konig R, et al. Nature (2010) 463: 813-817].

A number of miRNAs have been demonstrated to bind to influenza PB1 mRNA and inhibit viral replication in vitro [Brogaard, et al., Sci. Repts. (2016) 6: 21812, citing Song, L. et al., J. Virol. (2010) 84: 8849-60]. Three studies have investigated the role of circulating miRNA during influenza A infection in human patients. Each study employed a different type of sample material, namely whole blood [Id., citing Tambyah, P. et al., PLoS One (2013) 8: e76811], PBMCs [Id., citing Song, H., et al., BMC Infect. Dis. (2013) 13: 257] and serum [Id., citing Zhu, Z. et al. Viruses (2014) 6: 1525-39]. Human miRNAs hsa-miR-29a, 29b, and 29c are known to be expressed in human peripheral blood mononuclear cells. Ahluwalia, J. et al. Retrovirology (2008) 5: 117). One study reported that in PBMCs of critically ill patients with H1N1 infection, expression of hsa-miR-29a, -31, and -148a were all determined individually to have diagnostic potential [Brogaard, et al., Sci. Repts. (2016) 6: 21812, citing Song, H., et al. BMC Infect. Dis. (2013) 13: 257], whereas serum levels of hsa-miR-17, -20a, -106a, and -376c in combination could discriminate between avian influenza infected patients and healthy controls [Id., citing Zhu, Z. et al., Viruses (2014) 6: 1525-39]. Another study employing whole blood reported some overlap with these findings, namely hsa-miR-29a and hsa-miR-17 and -106a, respectively [Id., citing Tambyah, P. et al., PLoS One (2013) 8: e76811]. A porcine model used for demonstration of the temporal dynamics of miRNA expression after influenza A virus challenge from the first days of infection to after the infection had cleared showed that there is a time factor to consider when assessing the relation and involvement of cell-associated circulating miRNAs in response to influenza A virus infection. For example, ssc-miR29a was initially down-regulated (24 h p.i.), but later up-regulated (72 h and 14d pi) in the porcine model, whereas human studies have reported only down-regulation of its homolog hsa-miR29a-3p [Id., citing Tambyah, P. et al., PLoS One (2013) 8: e76811; Song, H., et al., BMC Infect. Dis. (2013) 13: 257; Zhu, Z. et al., Viruses (2014) 6: 1525-39]. Among the targets for hsa-miR-29a-3p is phosphatidylinositol 3,4,5-trisphosphate 3-phosphatase and dual-specificity protein phosphatase (PTEN), a tumor suppressor that acts as a dual-specificity protein phosphatase, dephosphorylating tyrosine-, serine- and threonine-phosphorylated proteins. PTEN was significantly down-regulated at both 24 h and 14d pi. This is consistent with up-regulation of ssc-miR-29a (porceine) at 14d pi, whereas upregulation of ssc-miR-182-another PTEN-targeting miRNA—may be a contributing factor to PTEN down-regulation at 24 h pi given that ssc-miR-29a is itself down-regulated at this time point. In a previous study, inhibition of miR-29a (−3p) in human hepatoma cell lines was shown to lead to up-regulation of PTEN at mRNA and protein levels, whereas overexpression of miR-29a down-regulated PTEN mRNA and protein [Id., citing Kong, G., et al., PLoS One (2011) 6: 1-10]. Moreover, miR-29a inhibition negatively regulated phosphorylation of Akt (protein kinase B), which is an important step in the PI3K/AKT signaling pathway [Id., citing Kong, G., et al., PLoS One (2011) 6: 1-10]. Akt family member AKT2 as well as FOX03A, a pro-apoptotic transcription factor (FoxO) downstream of PTEN and Akt, are also among the targets for hsa-miR-29a-3p. Despite being heavily targeted by up-regulated miRNAs at both 72 h and 14d po, no regulation of these two genes was seen. This is consistent with up-regulation of ssc-miR-29miR-150-5p (porcine), another miRNA identified in human studies as regulated after H1N1 infection. miR-150p was reported by one study to be up-regulated [Id., citing Tambyah, P. et al., PLoS One (2013) 8: e76811], while it was found to downregulated in another [Id., citing Song, H., et al., BMC Infect. Dis. (2013) 13: 257]. In the porcine model, hsa-miR-150p was found to be downregulated in porcine leukocytes 72 h and 114 d after H1N2 challenge in pigs [Id.].

In a previous study [Id., citing Wang, W., BMC Genomics (2012) 13: 278], the chemokine transcripts CCL2, CCL3, and CXCL10 in the lung tissue of the same animals were highly up-regulated at 24 h and 72 h pi. This is in accordance with an expected need for recruitment of various immune cells to the affected lung tissue, in order to combat and contain the influenza A virus (IAV) infection. The results show that leukocytes available for extravasation display an miRNA expression profile specialized toward contributing to the control of cell survival and apoptosis in response to IAV infection.

Viral infection triggers host responses that engage signaling networks which have a fundamental role in the anti-viral response. Previous studies have identified human protein kinases (HPKs) having key functions in influenza biology. By example these include protein kinase C (PKC) which is induced by viral binding to cell surface [Bakre, A. et al., PLoS One (2013) 6: e66796, citing Arora D J, Gasse N (1998) Arch Virol 143: 2029-2037; Kunzelmann K, et al. (2000) Proc Natl Acad Sci USA 97: 10282-10287], the extracellular signal-regulated kinase ERK [Id., citing Pleschka S, et al. (2001) Nature Cell Biology 3: 301-305; Pleschka S (2008) Biol Chem 389: 1273-1282] induced by accumulation of viral HA on the cell surface via PKC and regulating RNP export [Id., citing Marjuki H, et al. (2006) J Biol Chem 281: 16707-16715], and phosphatidylinositol-3 kinase (PI3K)]. The inhibition of this signaling network results in nuclear retention of the vRNP and decrease in influenza virus replication [Id., citing Pleschka S, et al. (2001) Nature Cell Biology 3: 301-305; Ludwig S, et al. (2004) FEBS Letters 561: 37-43]. NF-κB, a key mediator, is induced by accumulation of viral HA, NP and M1 proteins [Id., citing Ludwig S, et al. (2004) FEBS Letters 561: 37-43; Wei L, et al. (2006) J Biol Chem 281: 11678-11684; Wang X, et al. (2000) J Virol 74: 11566-11573; Pinto R, et al. (2011) I Antiviral Res 92: 45-56; Pauli E K, et al. (2008) PLoS Pathog 4: e1000196; Pahl H L, Baeuerle P A (1995) J Virol. 69: 1480-1484; Nimmerjahn F, et al. (2004) J Gen Virol 85: 2347-2356; Ludwig S, Planz O (2008) Biol Chem 389: 1307-1312; Kumar N, et al. (2008) J Virol 82: 9880-9889; Flory E, et al. (2000) J Biol Chem 275: 8307-8314]. Additionally, the presence of viral dsRNA has been shown to activate signaling cascades involving IKK-NF-κB, c-Jun N-terminal kinase (JNK), and P38 mitogen-activated protein kinase (MAPK) cascades all which regulate the expression of antiviral cytokines [Id., citing Majde J A (2000) J Interferon Cytokine Res 20: 259-272; Chu W M, et al. (1999) JNK2 and IKKbeta are required for activating the innate response to viral infection. Immunity 11: 721-731; Ludwig S, et al. (2006) Cell Microbiol 8: 375-386].

Influenza virus infection modulates multiple cellular miRNAs, and miR-323, miR-491, and miR-654 have been shown to inhibit viral replication by binding to the viral PB1 gene [Id., citing Liu H, et al. (2010) MiR26a and miR939 regulate the replication of H1N1 influenza virus in MDCK cells. Wei Sheng Wu Xue Bao 50: 1399-1405]. miR-507 and miR-136 have potential binding sites within the viral PB2 and HA genes [Id., citing 49]. miR-26a and miR-939 regulate the replication of H1N1 influenza virus in MDCK cells [Id., citing Liu H, et al. (2010) Wei Sheng Wu Xue Bao 50: 1399-1405].

Influenza virus A/WSN/33 was chosen for primary and validation screens; because it is a lab-adapted strain, another influenza strain (influenza A/New Caledonia/20/199 was tested to validate the HPK hits. Human kinase genes identified as important for influenza virus replication include: NPR2 (natriuretic peptide receptor B/guanylate cyclase B; MAP3K1 (mitogen-activated protein kinase 1), DYRK3 (dual specificity tyrosine (Y)-phosphorylation regulated kinase 3); EPPHA6 (EPH receptor A6; TPK1 (thiamin pyrophosphokinase 1); PDK2 (pyruvate dehydrogenase kinase, isozyme 2) C9ORF96 (chromosome 9 open reading frame 96); EXOSC10 (exosome component 10); NEKS (never in mitosis gene a-related kinase 8); PLLK4 (polo-like kinase 4); SGK3 (serum glucocorticoid regulated kinase family, member 3); NEK3 (never in mitosis gene a-related kinase 3); PANK4 (pantothenate kinase 4): ITPKB (inositol 1, 4, 5-triphosphate 3-kinase B); CDC2L5 (cell division cycle 2-like 5); CDK3 (cyclin-dependent kinase 3); CALM2 (calmodulin 2); PRKAG3 (protein kinase, AMP-activated, gamma 3 non-catalytic subunit); ERBB4 (v-erb-a erthroblastic leukemia viral oncogene homolog 4); ADK (adenosine kinase); PKN3 (protein kinase N3); HK2 (hexokinase 2). Three (NPR, MAP3K1 and DYRK3 when silenced led to increased viral replication, suggesting they have antiviral activity. The remaining 19 likely are pro-viral and indispensable for viral replication.

The validated HPKs affect critical pathways during influenza infection and replication, Four of the 6 validated HPKs, i.e. CDK13, NEK8, PLK4 and SGK3, have roles in cell cycle regulation. Similarly, two of 3 anti-viral HPKs (MAP3K1, DYRK3) are also implicated in regulation of cell cycle. SGK3 belongs to the three member family of serum glucocorticoid kinases (SGK1, 2 and 3) and has been shown to regulate influenza vRNP nuclear export into the cytoplasm [Id., citing Alamares-Sapuay, J G et al. J. Virol. (2013) 87: 6020-26]. SGK3 has been implicated in regulating cell survival [Liu M, et al. (2012) Serum and glucocorticoid kinase 3 at 8q13.1 promotes cell proliferation and survival in hepatocellular carcinoma. Hepatology 55: 1754-1765]. MAP3K1 is a multifunctional protein and important for induction of IFN-β induction in response to poly I: C challenge via IRF-3 activation [Yoshida R, et al. (2008) TRAF6 and MEKK1 play a pivotal role in the RIG-I-like helicase antiviral pathway. J Biol Chem 283: 36211-36220]. MAP3K1 also inhibits expansion of virus specific CD8+ T cells [Id., citing Labuda T, et al. (2006) MEK kinase 1 is a negative regulator of virus-specific CD8(+) T cells. Eur J Immunol 36: 2076-2084]. DYRK3 belongs to a family of dual specificity tyrosine kinases that activate by auto phosphorylation and catalyze phosphorylation of histone H3 and H2B. DYRK3 phosphorylates and activates sirtuin 1 (SIRT1) turnover, causes deacetylation of p53 and increased apoptosis [Id., citing Guo X, et al. (2010) DYRKIA and DYRK3 promote cell survival through phosphorylation and activation of SIRT1. J Biol Chem 285: 13223-13232]. Influenza virus infection upregulates mTORC1 signaling pathway [Id., citing Mata M A, et al. (2011) Chemical inhibition of RNA viruses reveals REDD1 as a host defense factor. Nat Chem Biol 7: 712-719] and inhibition of mTORC1 can significantly delay mortality due lethal challenge of influenza virus in mice [Id., citing Murray J L, et al. (2012) Inhibition of influenza A virus replication by antagonism of a PI3K-AKT-mTOR pathway member identified by gene-trap insertional mutagenesis. Antivir Chem Chemother 22: 205-215]. DYRK3 has been shown to stabilize P-granule like structures and the mTORC1 pathway during cellular stress. Inactivation of DYRK3 traps mTORC1 inside cytosolic stress granules while activation of DYRK3 promotes dissolution of stress granules and release of mTORC1 [Wippich F, et al. (2013) Dual specificity kinase DYRK3 couples stress granule condensation/dissolution to mTORC1 signaling. Cell 152: 791-805]. CDK13, HK2, NEK8, PANK4, PLK4, SGK3 increased or decreased viral replication of A/New Caladonia/20/99 infection as measured by influenza NP localization and influenza M gene levels. HK2, NEK8, PANK4, PLK4 have been identified as important for influenza virus replication in other influenza genome screens.

miRNAs regulate multiple aspects of the host response to infection. The number of miRNAs that were validated to be affected was limited. miRNAs of HPKs important for influenza replication include the following. Targeting DYRK3, CDK13 and SGK3 did not alter HPK expression or viral replication and were not discussed further. No effect of miR-149* modulation on influenza NP staining was evident suggesting that under the conditions of assay, NEK 8 transcript modulation by miR-149* did not have any effect on viral replication. Indeed, miR-149* induction during influenza infection has been reported to occur only post 72 hrs [Id., citing Loveday, E K et al. J. Virol (2012) 86: 6109-6122]. miR-149 is known to induce apoptosis by repressing Akt1 and E2F1. MAP3K1 transcript expression was significantly up-regulated by miR-548d inhibitor treatment; the mimic down-regulated MAP3K1 transcript expression. miR-29a or miR-138* treatments did not have any appreciable effect on MAP3K1 expression; miR29a has been implicated in regulation of MAPK1, and miR-29b has been implicated in regulation of DUYRK3 and CDK13. miR-548d inhibitor/mimic treatments did not alter MAP3K1 protein expression; similar to the NEK8 findings, MAP3K1 transcript modulation by miR-548d did not alter viral replication.

The most significant effects were observed for miR-34c and the PLK4 gene. While miR-34b and let-7i inhibitor/mimic treatments had no substantial effects on PLK4 transcript and protein expression, miR-34c mimic considerably up-regulated PLK4 transcript and protein expression, as well as influenza NP levels. It was hypothesized that during influenza virus infection, NS1 mediated p53 up-regulation triggers miR-34c activity to regulate cell cycle through Myc, PLK4 and NEK8. miR-34c alters PLK4 activity by modulating the activity of either p53 or by stabilizing PLK4 translation.

Li et al [J. Virol. (2010) 84(6): 3023-32] investigated whether differential expression of cellular microRNAs plays a role in the host response to infection by the 1918 H1N1 influenza virus. Unlike the nonlethal infections of some other H1N1 influenza virus strains, such as A/Texas/36/91 (Tx/91) or A.Kawasaki/173/01 (K173), the r1918 causes severe and lethal pulmonary disease. Functional genomics analyses revealed that the extreme virulence of r1918 was correlated with atypical expression of immune response related genes, including massive induction of cellular genes related to inflammatory response and cell death pathways [Id., citing Kash, J C, et al. Genomic analysis of increased host immune and cell death responses induced by 1918 influenza virus. Nature (2006) 443: 578-581; Kobasa, D., et al. Nature (2007) 445: 319-323].

A systematic profiling of cellular microRNAs in lung tissues from ice infected with r1918 or a nonlethal seasonal influenza virus [Id., citing Kash, J C, et al. Nature (2006) 443: 578-581] was performed to identify miRNAs whose expression patterns differentiated the host response to r1918 and Tx/91 infections, and the potential functions of differentially expressed miRNAs was assessed by analyzing the predicted target genes whose expression was inversely correlated with the expression of these miRNAs.

Because of their high abundance in lung tissue, 18 miRNAs were the focus of the analysis. These miRNAs demonstrated distinct expression patterns between the infections of r1918 and Tx/91 in mouse lungs. For example, miR-193 was strongly downregulated during r1918 infection, while it was upregulated during Tx/91 infection. In contrast, miR-709 was strongly upregulated during r1918 infection, while it was strongly downregulated during Tx/91 infection. miR-223 and miR-21, which were strongly upregulated in r1918 infection, were moderately upregulated only upon Tx/91 infection. On the other hand, while strongly downregulated in r1918 infection, miR-29a and miR-29b were moderately downregulated only upon Tx/91 infection. Finally, the expression levels of miR-200a, miT-34a, and miR-30a were downregulated in r1918 infection but were below the cutoff in Tx/91 infection [Id.].

These microRNAs have been implicated in multiple key functions. MiR-223 and Let-7 have been shown to be involved in immune responses, and miR-223 is a negative modulator of neutrophil activation and neutrophil-mediated killing [Id., citing Jopling, C L M, et al. Science (2006) 309: 1577-81]. A decreased expression level of Let-7 is associated with the activation of NF-κB in response to microbial challenge [Id., citing Hu, G., et al. J. Immunol. (2009) 183z: 1617-24]. Upregulation of miR-21 is closely related to airway inflammation [Id., citing Lu, T X, et al. J. Immunol. (2009) 182: 4994-5002], a symptom of lethal r1918 infection, and miR-34a is associated with tumorigenesis, as the mutual activation of MiR-34a and p53 has been shown both in a human cell line [Id., citing Uamakuchi, M. et al. Proc. Natl Acad. Sci. USA (2008) 105: 13421-26] and in patients [Id., citing Mraz, M. e al. Leukemia (2009) 23: 1159-63]. In addition, a stable expression of miR-200a is critical in maintaining the phenotype of epithelial cells [Id., citing Gregory, P A, et al. 2008. The miR-200 family and miR-205 regulate epithelial to mesenchymal transition by targeting ZEB1 and SIP1. Nat. Cell Biol. 2008) 10:593-6011].

As microRNAs predominately function as repressors of target gene expression, these investigators therefore focused only on the targets whose expression was inversely correlated with the expression of their corresponding microRNAs. MicroRNA and cellular gene expression was directly compared between r1918 and Tx/91 infections to assess expression changes. In the analyses, an upregulation means that a microRNA or a cellular gene was expressed more abundantly during r1918 infection relative to Tx/91 infection, while a downregulation means that a microRNA or a cellular gene was expressed less abundantly during r1918 infection relative to Tx/91 infection [Id.].

The statistical analysis indicated a significant enrichment of the inversely related targets of 18 highly abundant microRNAs during r1918 infection; these microRNAs may regulate target gene expression at different stages of infection. For example, although miR-223 was upregulated during r1918 infection at all three time points, only the hypergeometric (HG) test results from day 1 had a P of ≤0.05. These results suggested that miR-223-mediated regulation of gene expression was predominately elicited on day 1, an early stage of infection. In contrast, although miR-29a was significantly downregulated in r1918 infection at all three time points, the HG test results from days 3 and 5, but not day 1, had P values of ≤0.05; by inference, miR-29a-mediated regulation on target gene expression occurred predominately at a later stage of infection. Furthermore, although miR-34a was downregulated in r1918 infection at all three time points, the HG test results from only day 3 had a P of ≤0.05, suggesting that miR-34a-mediated regulation on gene expression may be transient [Id.].

Gene ontology analysis indicated that the inversely correlated targets of four microRNAs are associated with the immune response. The targets of Let-7f are associated with lymphocyte-mediated immune response, while the targets of miR-200a are associated with viral gene replication and the JAK-STAT signaling pathway, which is closely related to the type I IFN-mediated innate immune response. The inversely correlated miR-34a targets are associated with calcium ion homeostasis, which is critical for immune cell activation. In addition, the targets of miR-27a are associated with regulation of the immune response. The inversely correlated targets of three microRNAs, including miR-652, miR-27a, and miR-27b, are associated with apoptosis and cell death. Intriguingly, atypical expression of immune response-related and cell death-related genes was previously shown to be related to the extreme virulence of r1918 in mouse and macaque models of infection [Id., citing Kash, J C, et al. Nature (2006) 443:578-581, Kobasa, D., et al. Nature (2007) 445:319-323]. Furthermore, the inversely correlated targets of miR-223, miR-29a, miR-29b, and miR-709 are related to cell division and the cell cycle. Finally, the inversely correlated targets of four microRNAs, including miR-200b, -30a, -30d, and -429, are associated with the regulation of fever, a key sign of illness during influenza virus infection.

Two related pathways comprising inversely correlated targets were analyzed to better understand how specific miRNAs affected these biological functions.

Type 1 interferon pathway. The analysis of microRNA targets indicated that the type I IFN pathway was subject to microRNA-mediated regulation, since key genes in this pathway, such as IFNAR1 and STAT2, are direct targets of miR-200a, and their expression was inversely correlated with the expression of this microRNA). The data suggested that the downregulation of miR-200a in r1918-infected lungs may induce the upregulation of key genes in the type I IFN signaling pathway. Indeed, many IFN-stimulated genes demonstrated increased expression levels in r1918-infected lungs compared with those in the Tx/91-infected lungs [Id., citing Kash, J C, et al. Nature (2006) 443:578-581; Kobasa, D., et al. Nature (2007) 445:319-323].

CREB pathway. The cyclic AMP (CAMP) responsive element binding protein, CREB, is a transcription factor that regulates the expression of hundreds of genes. CREB-null mice die immediately after birth from respiratory distress [Id., citing Rudolph, D A, et al. Proc. Natl. Acad. Sci. U.S.A (1998) 95: 4481-4486], as CREB is involved in critical functions, including T-cell development [Id., citing Rudolph, D A, et al. Proc. Natl. Acad. Sci. U.S.A (1998) 95:4481-4486], as CREB is involved in critical functions, including T-cell development) and cell survival [Id., citing Bonni, A, et al. Science (1999) 286:1358-1362]. The activity of CREB is regulated by multiple upstream pathways, including insulin-like growth factor, Ca2+, and G protein-coupled receptor signaling pathways. Many key genes in the CREB upstream pathways are miR-233 targets, and concomitant with the strongly increased expression of miR-233 in r1918-infected lungs, the miR-223 targets in the CREB upstream pathways were significantly downregulated. The data therefore suggested that upregulation of miR-223 may repress the activity of CREB.

The authors concluded that taken together, this study reveals that influenza virus infection induces changes in the cellular microRNAome and that unique patterns of differential expression of microRNAs may contribute to the extreme virulence of r1918 influenza virus infection by regulating the expression of cellular targets involving immune response and other critical cellular functions. Regulating cellular microRNA expression may be a common activity among different influenza viruses; however, the pathogenic capacity of the viruses may eventually be determined by the expression pattern of a specific microRNA or a group of microRNAs.

Role of Cellular microRNAs in the Host Immune Response.

In a further study, a potential role for miR-200a in regulating the immune response was identified. Previously, miR-200a was known only for regulating the epithelial-to-mesenchymal transition by targeting zeb1 and sip1 [Id., citing Park, S M, et al. Genes Dev. (2008) .22: 894-907]. In r1918-infected mice, the investigators not only found inversely correlated expression of miR-200a and sip1 but also found evidence implicating miR-200a in the type I IFN response. The aberrant activation of the type I IFN pathway during r1918 infection may contribute to an unconstrained inflammatory response [Id., citing Kash, J C, et al. Nature (2006) 443: 578-581; Kobasa, D., et al. Nature (2007) 445:319-323]. Increased expression of miR-200a target genes in the type I IFN pathway, including IFN-α receptors and STAT2/4 was found; the upregulation of these cellular genes, owing to the downregulation of miR-200a, may induce profound effects during infection. As these genes are located at the upstream end of the IFN signaling cascade, a moderate expression change could be exponentially amplified at the downstream end of the cascade. As transcription factors, STAT2/4 are able to regulate the expression of a large number of IFN-stimulated genes (ISGs). An increase in the expression of STAT2/4 caused by the downregulation of miR-200a may induce an overwhelming increase of ISG expression. Massive induction of ISG expression has been observed in mice and macaques infected with the r1918 virus [Id., citing Kash, J C, et al. Nature (2006) 443: 578-581; Kobasa, D., et al. Nature (2007) 445: 319-323]).

In addition to directly regulating the expression of transcription factors, microRNAs may also impose indirect regulation. For instance, miR-223 may indirectly repress the activity of the transcription factor CREB by regulating its upstream pathways. In this study, it was demonstrated that miR-223 may downregulate CREB activity by repressing three such upstream pathways, namely the IGF-1 receptor, Ca2+ channel, and GPCR pathways. In addition, miR-223 also repressed the expression of crucial intermediate molecules in these pathways, such as PI3K, PP2A, and PKA. One critical function of CREB is maintaining cell survival and growth in vivo (20). Indirect repression of CREB activity by miR-223 would be expected to result in increased cell death, which is a signature of lethal r1918 infection in vivo Id., citing Kash, J C, et al. Nature (2006) 443:578-581; Kobasa, D., et al. Nature (2007) 445:319-323].

### Human Immunodeficiency Virus (HIV)

There is significant functional interplay between HIV-1 and miRNA mediated silencing in host cells. Human immunodeficiency virus may be targeted by several host miRNAs [Aqil, M. et al., J. Extracell. Vesicles (2014) 3. doi: 10342/jev.v3.23129, citing Jariharan, M. et al., Biochem. Biophys. Res. Commun. (2005) 337: 1214-18]. The HIV-1Tat and Nef proteins inhibit key components of the host miRNA pathway [Id, citing Bennasser, Y. et al., Immunity (2005) 22: 607-19; Bennasser, Y., Jeang, KT. Retrovirology (2006) 3: 95]. Knockdown of the miRNA biogenesis proteins Drosha and Dicer in latently infected cells increases HIV-1 replication, and host miRNAs such as the miR-17/92 cluster indirectly modulate HIV-1 replication through the p300/CBP-associated factor (Id., citing Triboulet, R. et al. Science (2007) 315: 15799-82). miR-29a, which targets the nef-3′UTR, is a potent inhibitor of HIV-1 replication [Id., citing Sunm, G. et al., Nucleic Acids Res. (2012) 40: 2181-96); Ahluwalia, J K, et al., Retrovirology (2008) 5: 117; Nathans, R. et al., Mol. Cell (2009) 34: 696-709]. Another report showed that miR-29a, miR-29b, miR-9, and miR-146a target the SIV/HIV 3′UTR [Id. citing Sisk, J M et al., Retrovirology (2013) 10: 95]. Two cell line models of latent HIV-1 infection (U1, a derivative of U937 monocytic cells, and J1.1, derived from CD4+ Jurkat T cells) [Id. citing Folks, T M. Science (1987) 238: 800-802; Perez, V L, et al. J. Immunol. (1991) 147: 3145-48] were used to show, by qRT-PCR, that miR-29a levels are high during latency and are reduced during active viral replication. Microarray analysis of acutely infected PBMcs showed downregulation of miR-29a and similar results were observed in PBMCs isolated from HIV infected individuals with high viral load [Id., citing Sunm, G. et al., Nucleic Acids Res. (2012) 40: 2181-96; Houzet, L. et al., Retrovirology (2008) 5: 118].

By regulating cellular miRNA, Nef may inhibit Kaposi's sarcoma-associated herpesvirus (KSHV) replication to promote viral latency and contribute to the pathogenesis of AIDS-related malignancies [Yan, Q. et al., J. Virol. 88 (9): 4987-5000]. Kaposi's sarcoma-associated herpesvirus (KSHV) is causally linked to several AIDS-related malignancies, including Kaposi's sarcoma (KS), primary effusion lymphoma (PEL), and multicentric Castleman's disease [Id.]. Bioinformatics and luciferase reporter analyses showed that hsa-miR-1258, a Nef-upregulated miRNA, directly targeted a seed sequence in the 3′ untranslated region (UTR) of the mRNA encoding the major lytic switch protein (RTA), which controls KSHV reactivation from latency. Ectopic expression of hsa-miR-1258 impaired RTA synthesis and enhanced Nef-mediated inhibition of KSHV replication, whereas repression of hsa-miR-1258 has the opposite effect. Mutation of the seed sequence in the RTA 3′ UTR was found to abolish downregulation of RTA by hsa-miR-1258.

HIV-1 Replication and miRNAs

miRNAs affect HIV-1 replication indirectly by affecting host cell HIV-1 dependency factors that regulate HIV-1 integration and transcription, and directly by binding to viral transcripts and inhibiting translation [Frattari, G. et al. The role of miR-29a in HIV-1 replication and latency. J. Virus Eradication (2017) 3: 185-191, citing Swaminathan, G. et al. J. Mol. Biol. (2014) 426: 1178-97]. Specifically, miR-144 targets cellular LEDGF/p75, which plays a critical role in guiding reverse-transcribed genomes to the intronic regions of highly expressed genes [Id., citing Ruelas, DS, Green, WC. Cell (2013) 155: 519-29]. At the transcriptional level, miRNAs can reinforce HIV-1 latency through their regulation of constitutively expressed factors that control cellular proliferation regardless of the cell's infection status. It has been hypothesized that miRNAs known to target cyclin T1, whose association with CDK9 in the POTEFb complex is essential for hIV-1 transcription (miR-27b, miR-29b, miR-150, miR-198, and miR-223) might play a key role in HIV-1 latency regulation [Id. citing Chiang, K. et al. J. Virol (2012) 86: 3244-52]. miR-17-5p and miR-20a are involved in the epigenetic control of HIV-1 replication. They affect the cellular levels of p300/CBP-associated factor (PCAF) [Id., citing Triboulket, R. et al. Science (2007) 315: 1579-82], a histone acetyltransferase that enhances HIV-1 transcription by acetylating both the histone proteins and the p65 component of NF-κB [Id., citing Ruelas, DS, Green, WC. Cell (2013) 155: 519-29]. miR-155, another regulator of NF-κB activity, inhibits the ubiquitinating effect of TRIM-32 on IκBα, this enhancing the availability of IκB and the following sequestration of NFκB in the cytoplasm [Id. citing Ruelas, D S, et al. J. Biol. Chem. (2015) 290: 13736-48].

Conversely, some miRNAs can enhance HIV-1 infection by inhibiting cellular repressors of viral replication. For example, miR-34a and miR-217 downregulate SIRT-1, a p65 and Tat deacetylase, thus enhancing their efficiency under HIV-1 transcription [Id., citing Zhang, H S et al., FEBS Lett. (2012) 586: 4203-7; Zhang, H S, et al., Biochim. Biophys. Acta (2012) 1823: 1017-23].

Host miRNAs also can bind directly to viral transcripts. miR-28, miR-125b, miR-150, miR-223, and miR-382 were shown to downregulate transcripts containing a 1.2 kb fragment from HIV-1 3′UTR harboring target sequences for these miRNAs; mutations in these putative miRNA targets relieved the inhibition [Id., citing Huang, J. et al. Nat. Med. (2007) 13: 1241-47]. Virus transcript downregulation was reported to take place in resting, but not stimulated CD4+ T cells. Inhibition of the five miRNAs simultaneously was reported to cause reactivation of HIV-1 infection in CD4 T cells from HIV-1 on combined retroviral therapy [Id. citing Huang, J. et al. Nat. Med. (2007) 13: 1241-47].

Interactions Between miR-29a and HIV Replication

The following in vitro experiments suggested an existing association between cellular miR-29a levels and HIV-1 protein expression. Experiments conducted in cell lines transfected with HIV-1 plasmids (mainly pNL4-3 or its luciferase variant pNL4-3LucR-E) showed that overexpression of miR-29a downregulates HIV-1 virus production [Id., citing Ahluwalia, J. et al. Retrovirology (2008) 5: 117, Nathans, R. et al. Mol. Cell (2009) 34: 696-709; Sun, G. et al, Nucleic Acid Res. (2012) 40: 2181-96] and reduces the infectivity of resulting viruses (Id., citing Nathans, R. et al. Mol. Cell (2009) 34: 696-709). In a similar experiment, cells harboring artificial constructs that mimic miR-29a in structure and function show decreased HIV-1 production [Id., citing Nathans, R. et al. Mol. Cell (2009) 34: 696-709]. In a reciprocal experiment, inhibition of endogenous miR-29a was shown to reactivate latent provirus in HIV-1 latently infected Jurkat E6 cells (J1.1 cells) [Id., citing Patel, P. et al., Retrovirology (2014) 11: 108].

A putative binding site for miR-29a, located in a highly conserved region of the Nef gene that also serves as 3′UTR for HIV-1 transcripts, has been reported to exist in the HIV-1 genome [Id., citing Hariharan, M. et al., Biochem. Biophys. Res. Comun. (2005) 337: 1214-18, confirmed by Nathans, R. et al. Mol. Cell (2009) 34: 696-709]. Experiments suggested that miR-29a allows the RISC to bind HIV-1 mRNA, and that the miR-29a-HIV-1-mRNA-RISC complex then associates with P-bodies, the cytoplasmic substructures where Ago-proteins, miRNAs and untranslated mRNAs accumulate, together with other enzymes involved in mRNA turnover and translational repression [Id. citing Winter, J. et al. Nat. Cell Biol. (2008) 11: 228-34], where mRNA translational repression takes place.

Evidence from in vitro studies [Id., citing Ahluwalia, J. et al. Retrovirology (2008) 5: 117; Nathans, R. et al. Mol. Cell (2009) 34: 696-709; Patel, P. et al., Retrovirology (2014) 11: 108] indicates that miR-29a interacts with HIV-1 transcripts, silencing viral production and infectivity, but results from two other studies have questioned whether this interaction may exist in vitro [Id., citing Sun, G. et al. Nucleic Acid Res. (2012) 40: 2181-96, Whisnant, A W et al. MBio (2013) 4: e000193], and suggested that the virus can escape miR-29a-mediated restriction when cellular miR-29a expression is at physiological concentrations.

Evidence suggests that miR-29a downregulates Nef protein levels in infected cells [Id., citing Ahluwalia, J. et al. Retrovirology (2008) 5: 117; Patel, P. et al., Retrovirology (2014) 11: 108]. The ability to target Nef mRNA is common for the miR-29 family but stronger for miR-29a [Id., citing Ahluwalia, J. et al. Retrovirology (2008) 5: 117]; such targeting has been hypothesized to play a role in regulating HIV-1 pathogenesis, given that functional Nef is essential for in vivo viral pathogenesis [Id., citing Zou, W., et al., Retrovirology (2012) 9: 44]. Although miR-29a also mediated HIV-1 gag mRNA association with Ago2 proteins [Id., citing Nathans, R. et al. Mol. Cell (2009) 34: 696-709], direct 3′UTR targeting of whole length HIV-1 mRNAs by miR-29a could be sufficient to cause the inhibitory effects observed [Id.].

### Exosome Co-Option by Retroviruses

The exosomal membrane-sorting pathway can be co-opted by retroviruses for the generation of Trojan virions [Willis, G R et al., Front. Cardiovasc. Med. (2017) 4: article 63]. Further, some miRNAs are selectively retained in cells, while others are preferentially secreted.

The human immunodeficiency virus (HIV) encodes prototypic retroviral proteins (Gag, Pol, Env) as regulatory (Rev, Tat) and accessory (Nef, Vif, Vpr, Vpu/Vpx) proteins, the last group being dispensable for virus replication in vitro, but important for persistence and disease in an immunocompetent host [Aqil, M. et al., J. Extracell. Vesicles (2014) 3. doi: 10342/jev.v3.23129, citing Das S R, Jameel S. Indian J Med Res. (2005) 121: 315-32]. The nef gene is located at the 3′-end of the viral genome, partially overlapping the env gene and the U3 region in the 3′ long terminal repeat (LTR). Nef is the largest of the four HIV accessory proteins, is expressed early in infection and at far higher levels than the other early proteins, Tat and Rev [Id., citing Landi A, et al Curr HIV Res. (2011) 9: 496-504]. It is primarily localized at cellular membranes, which include endosomal membranes, the perinuclear region and the inner surface of plasma membrane. Nef is also released from cells, either in microvesicles (MVs) [Id., citing Campbell T D, et al. Ethn Dis. (2008) 18: S2-14-93; Lenassi M, et al. Traffic. (2010) 11: 110-22] or as a soluble protein, and has effects on bystander cells [Id., citing Raymond A D, et al. AIDS Res Hum Retroviruses. (2011) 27:167-78; Fujii Y, et al. FEBS Lett. (1996) 393:93; Campbell P E, et al. J Mol Model. (2012) 18:4603-13].

The first evidence of extracellular Nef came from reports on the detection of anti-Nef antibodies in the sera of AIDS patients [Id., citing Ameisen J C, et al. N Engl J Med. 1989; 320:251-2]. Later, Nef was also found in exosome-like vesicles released from HEK293 cells expressing a Nef-green fluorescent protein (Nef-GFP) fusion protein [Id., citing Campbell T D, et al. Ethn Dis. (2008) 18: S2-14-9]. These vesicles were shown to be taken up by Jurkat CD4+ T cells, in which the Nef-GFP fusion protein localized mainly to the cytoplasm as punctate structures [Id., citing Campbell T D, et al. Ethn Dis. 2008; 18: S2-14-9]. The vesicles are likely to enter recipient cells via endocytosis, which had been reported in other systems [Id., citing Svensson K J, et al. J Biol Chem. (2013) 288: 17713-24]. Nef exosomes could also fuse with Nef-deficient HIV-1 virions and restore the infectivity of mature particles [Id., citing Campbell, T D et al. Ethn. Dis. (2008) 18: S2-14-9]. Lastly, in vitro evidence obtained using U937/Nef-EYFP cells and exosomes purified from these cells suggests that Nef mediates the redistribution of miRNAs between cells and exosomes to aid in viral replication and persistence.

Nef-expressing U937 cells secreted on an average about 60% more exosomes than control cells. Nef exosomes are enriched in miRNAs that can target proinflammatory cytokines and other genes involved in key pathways like JAK-STAT signaling, MAPK signaling and apoptosis. Further, an overwhelming percentage of miRNAs that can potentially target HIV-1 are secreted out of Nef-expressing cells into exosomes. As reported earlier for other cell types, U937 cells expressing Nef also showed increased exosome secretion. There was differential expression of about 50% of detected miRNAs under the influence of Nef. We observed significant changes in the levels of several miRNAs that regulate innate immune responses, especially the proinflammatory cytokines. These include miR-16, miR-125b, miR-146a, miR-146b-3p and miR-181a, which are reduced in Nef-expressing U937 cells.

Host miRNAs also target viral transcripts and limit replication. MiR-18, miR-19a, miR-20a, miR-21 and miR-29b were found to be downregulated in Nef expressing monocytes. A comparison of miRNA expression patterns in resting and activated CD4+ T cells and use of specific antagomirs concluded that miR-28, miR-125b, miR-150, miR-223 and miR-382 target the nef/3′LTR region and contribute to HIV latency in resting CD4+ T cells; similar data were also reported for monocytes and macrophages [Id., citing Wang X, et al. Blood. (2009) 113: 671-4; Sun G, et al. Nucleic Acids Res. 2012; 40:2181-96]. Profiling demonstrated reduced levels of miR-125b and miR-223 in Nef-expressing monocytes, whereas miR-382 was not detected.

Based on in silico analyses, miR-29a, miR-29b, miR-149, miR-324-5p and miR-378 have been reported to target conserved regions of the HIV-1 genome, including the nef gene [Id., citing Sun G, et al. Nucleic Acids Res. (2012) 40:2181-966]. Of these, miR-29b was found to be downregulated in Nef-expressing cells. A majority of miRNAs that inhibit HIV replication, including miR-17, miR-19a, miR-19b, miR-20a, miR-26a, miR-28, miR-29a, miR-29b, miR-29c, miR-92a, miR-125b, miR-149, miR-150, miR-223, miR-324-5p, miR-378 and miR-382 were present at 1.5-folds or higher levels in Nef exosomes. Further, when correlated with the reported in silico analysis of miRNA target sites in the HIV genome, an overwhelming majority of miRNAs that can potentially target HIV-1 genomes were present at increased levels in exosomes secreted by Nef-expressing cells. 47 miRNAs were found to be present at increased levels in Nef exosomes despite being present at reduced levels in Nef-expressing cells. Of these selectively secreted miRNAs, 21 also had target sites on HIV-1 genomes. Thus, Nef expression reduced cellular levels of several host miRNAs that target innate immune responses and viral transcripts by exosome-mediated export, which likely modifies the host cell environment to favor virus replication.

### Exosome Modulation of the Immune Response to Viral Infection

Generally, when challenged by a viral infection, pattern recognition receptors (PRRs), such as Toll-like receptors and RIG-I-like receptors, sense viral components called pathogen-associated molecular patterns (PAMPs) and trigger signals to induce innate immune responses. Studies have revealed that EVs released from virus-infected cells deliver viral RNA to dendritic cells and macrophages, thereby activating PRRs in recipient cells, which results in the expression of type I interferon and pro-inflammatory cytokines. EVs transfer not only viral RNA but also host microRNAs to recipient cells. Infection of hepatocytes with hepatitis B virus (HBV) was shown to affect microRNA levels in EVs released from virus-infected cells, leading to attenuation of host innate immune response [Kouwaki, T. et al. Int. J. Mol. Sci. (2017) 18 (30): 666].

T cells can recruit major histocompatibility complex class II-containing DC exosomes secreted in the extracellular milieu during cognate DC-T-cell interactions. Recruitment of these exosomes required T-cell activation and was dependent on leukocyte function-associated antigen-1 (LFA-1) rather than on T-cell receptor specificity. Indeed, inducing a high-affinity state of LFA-1 on resting T cells was sufficient to provoke exosome binding. These results were interpreted to imply that DC exosomes secreted in the extracellular milieu during cognate T-cell-DC interactions are targeted to T cells activated in that microenvironment [Zhang, J. et al. Genomics Proteomics Bioinformatics (2015) 13: 17-24, citing Nolte-'t Hoen, E N et al. Blood. (2009) 113:1977-1981].

Exosomes can modulate the immune response during viral infection by increasing the function of macrophages and NK cells, and delivering antiviral molecules among cells, as well as by inhibiting immune responses directly or indirectly and influencing cytokine-mediated signaling pathways and cytokine production. For example, exosomes released from DCs laden with pathogen-derived antigens can protect against infection [Alipoor, S D et al. Mediators Inflamm. (2016) 2016: 5628404., citing A. I. Masyuk, T. V. Masyuk, and N. F. LaRusso, “Exosomes in the pathogenesis, diagnostics and therapeutics of liver diseases,” Journal of Hepatology, (2013) vol. 59, no. 3, pp. 621-625]. In contrast, exosomes can also contain exogenous viral RNAs and be involved in the spreading of infection [Id., citing H. S. Chahar, X. Bao, and A. Casola, “Exosomes and their role in the life cycle and pathogenesis of RNA viruses,” Viruses (2015) 7 (6): 3204-3225]. On the other hand, due to their biological properties, exosomes have also been proposed as possible delivery vectors for therapeutic purposes [Id., citing E. van der Pol, A. N. Böing, P. Harrison, A. Sturk, and R. Nieuwland, “Classification, functions, and clinical relevance of extracellular vesicles,” Pharmacological Reviews (2012) 64 (3): 676-705] and vaccine delivery vehicles [Id., citing N. A. Kruh-Garcia, L. M. Wolfe, and K. M. Dobos, “Deciphering the role of exosomes in tuberculosis,” Tuberculosis (2015) 95 (1): 26-30, P. K. Anand, E. Anand, C. K. E. Bleck, E. Anes, and G. Griffiths, “Exosomal hsp70 induces a pro-inflammatory response to foreign particles including mycobacteria,” PLoS ONE (2010) 5 (4): Article ID e10136]. Natural killer (NK) cells are innate immune lymphocytes that destroy infecting or transformed cells without the need for activation, in contrast to T and B cells.

Human natural killer (NK) cells have been reported to release exosomes in both resting and activated condition [Fais, S. Oncoimmunology (2013) 2: 1, e: 2237, citing Lugini, L. et al. J. Immunol. (2012) 189: 2833-42]. The NK cell-derived exosomes not only express both typical NK markers (i.e., CD56) and killer proteins (i.e., FASL and perforin) but also exert antitumor and immune homeostatic activities. These findings demonstrate that—at odds with T and B cells [Id., citing Raposo, G. et al. J. Exp. Med. (1996) 183: 1161-72; Peters, P J et al. Eur. J. Immunol. (1989) 19: 1469-75]—NK cells secrete exosomes in a constitutive way and independently from their activation status. These in vitro results were strongly supported by ex vivo findings on circulating exosomes obtained from healthy donors, showing expression of NK markers, such as CD56 and perforin, and exerting exosome-induced cytotoxicity. Thus, it appears conceivable that NK cell-derived exosomes control immune responses both in a paracrine fashion and systemically. Together with CD56, NK cell-derived express detectable amounts of the activating receptor NKG2D, whereas natural cytotoxicity receptors (NCRs), the other NK-cell receptors that mediate cytotoxic functions (i.e., NKp30, NKp46 and NKp44), are less expressed. Perforin was detected in exosomes purified from both NK-cell culture supernatants and the plasma of healthy individuals, whereas FASL was undetectable in plasmatic exosomes [Id., citing Lugini, L. et al. J. Immunol. (2012) 189: 2833-42]. Moreover, perforin-containing plasmatic exosomes were exclusively associated with NK-cell but not CD8+ T-cell markers; this is in line with a previous report showing that perforin is highly expressed by resting NK cells, but not by resting CD8+ T lymphocytes [Id., citing Obata-Onai, A., et al. Intl. Immunol. (2002) 14: 1085-98].

It has also been demonstrated that exosomes can serve as vectors, e.g., for gene delivery. While adeno-associated virus (AAV)-containing exosomes can be used for delivering genes to the cardiomyocytes and to the heart, they are susceptible to blocking by AAV neutralizing antibodies. AAV exosomes purified so as to minimize contamination with free AAVs were used to deliver the sarcoplasmic reticulum calcium ATPase gene (SERCA2a) in a mouse model with myocardial infarction. AAVExo vectors showed a significant enhancement in gene transduction compared to free AAVs and were more resistant to neutralizing antibodies than AAV both in vitro and in vivo. AAVExo-SERCA2a outperformed conventional AAV vectors in preserving cardiac function in presence and absence of neutralizing antibodies [Liang, Y. et al. Circulation (2017) 136: A15439].

### Disease Processes

Cancer. Several reports have demonstrated that exosomes play important roles in tumor progression. Exosomes derived from platelets that were treated with thrombin and collagen stimulated proliferation and increased chemoinvasion in the lung adenocarcinoma cell line A549 [Id., citing Janowska-Wieczorek A., et al. Int J Cancer. (2005) 113:752-760]. Exosomes derived from SGC7901 promoted the proliferation of SGC7901 and another gastric cancer cell line, BGC823 [Id., citing Qu J. L., et al. Dig Liver Dis. (2009) 41:875-8]. In addition, CD147-positive exosomes derived from epithelial ovarian cancer cells promoted angiogenesis in endothelial cells in vitro [Id., citing Millimaggi D., et al. Neoplasia. (2007) 9:349-357]. Webber et al. incubated exosomes derived from a mesothelioma cell line, a prostate cancer cell line, a bladder cancer cell line, a colorectal cancer cell line, and a breast cancer cell line with primary fibroblasts in vitro and found that fibroblasts could be transformed into myofibroblasts [Id., citing Webber J., et al. Cancer Res. (2010) 70:9621-9630]. A similar phenomenon was also observed by Cho et al., who described that tumor-derived exosomes converted mesenchymal stem cells within the stroma of the tumor tissue into cancer-associated myofibroblasts [Id., citing Cho J. A., et al. Int J Oncol. (2012) 40: 130-138].

### Fibrosis as a Pathology

Fibrosis represents the formation or development of excess fibrous connective tissue in an organ or tissue, which is formed as a consequence of the normal or abnormal/reactive wound healing response leading to a scar. Although the fibrogenic response may have adaptive features in the short term, when it progresses over a prolonged period of time, parenchymal scarring and ultimately cellular dysfunction and organ failure ensue [Rockey D C et al., N Engl J Med. (2015) 372 (12): 1138-49]. Fibrosis is characterized by, for example, without limitation, an aberrant deposition of an extracellular matrix protein, an aberrant promotion of fibroblast proliferation, an aberrant induction of differentiation of a population of fibroblasts into a population of myofibroblasts, an aberrant promotion of attachment of myofibroblasts to an extracellular matrix, or a combination thereof.

There are four major phases of the fibrinogenic response. First is initiation of the response, driven by primary injury to the organ. The second phase is the activation of effector cells, and the third phase is the elaboration of extracellular matrix, both of which overlap with the fourth phase, during which the dynamic deposition (and insufficient resorption) of extracellular matrix promotes progression to fibrosis and ultimately to end-organ failure (Id.).

The fact that diverse diseases in different organ systems are associated with fibrotic changes suggests common pathogenic pathways (Id.). This “wounding response” is orchestrated by complex activities within different cells in which specific molecular pathways have emerged. Cellular constituents include inflammatory cells (e.g., macrophages and T cells), epithelial cells, fibrogenic effector cells, endothelial cells, and others. Many different effector cells, including fibroblasts, myofibroblasts, cells derived from bone marrow, fibrocytes, and possibly cells derived from epithelial tissues (epithelial-to-mesenchymal transition) have been identified; there is some controversy regarding the identity of specific effectors in different organs. Beyond the multiple cells essential in the wounding response, core molecular pathways are critical; for example, the transforming growth factor beta (TGF-β) pathway is important in virtually all types of fibrosis (Id.).

As fibrosis progresses, myofibroblasts proliferate and sense physical and biochemical stimuli in the local environment by means of integrins and cell-surface molecules; contractile mediators trigger pathological tissue contraction. This chain of events, in turn, causes physical organ deformation, which impairs organ function. Thus, the biology of fibrogenesis is dynamic, although the degree of plasticity appears to vary from organ to organ (Id.).

Acute and chronic inflammation often trigger fibrosis (Id.). Inflammation leads to injury of resident epithelial cells and often endothelial cells, resulting in enhanced release of inflammatory mediators, including cytokines, chemokines, and others. This process leads to the recruitment of a wide range of inflammatory cells, including lymphocytes, polymorphonuclear leukocytes, eosinophils, basophils, mast cells, and macrophages. These inflammatory cells elicit the activation of effector cells which drive the fibrogenic process [Id., citing Wynn T A. Nat Rev Immunol (2004) 4: 583-94]. In addition, macrophages can play a prominent role in interstitial fibrosis, often driven by the TGF-β pathway [Id., citing Meng X M, et al. Nat Rev Nephrol (2014) 10:493-503]. However, some inflammatory cells may be protective. For example, certain populations of macrophages phagocytose apoptotic cells that promote the fibrogenic process and activate matrix-degrading metalloproteases [Id., citing Ramachandran P, Iredale J P. J Hepatol (2012) 56: 1417-9]. Fibroblasts and myofibroblasts have been identified as key fibrosis effectors in many organs, and as such are responsible for the synthesis of extracellular matrix proteins [Id., citing Hinz B, et al. Am J Pathol (2007); 170: 1807-16].

The matrix proteins that compose the fibrotic scar, which are highly conserved across tissues, consist predominantly of interstitial collagens (types I and III), cellular fibronectin, basement-membrane proteins such as laminin, and other, less abundant elements. In addition, myofibroblasts, which by definition are cells that express smooth-muscle proteins, including actin (ACTA2), are contractile (Id., citing Rockey D C, et al. J Clin Invest (1993) 92: 1795-804). The contraction of these cells contributes to the distortion of parenchymal architecture, which can promote disease pathogenesis and tissue failure. However, myofibroblasts also contribute to the normal wound healing process by contracting the edges of the wound and synthesizing and depositing extracellular matrix components [Hinz B. Curr Res Transl Med. (2016) October-December; 64(4): 171-177; Darby I A, et al. Clin Cosmet Investig Dermatol. (2014) 7: 301-311].

### Pro-Inflammatory Mediators

Accumulating evidence has suggested that polypeptide mediators known as cytokines, including various lymphokines, interleukins, and chemokines, are important stimuli to collagen deposition in fibrosis. Released by resident tissue cells and recruited inflammatory cells, cytokines are thought to stimulate fibroblast proliferation and increased synthesis of extracellular matrix proteins, including collagen. For example, an early feature in the pathogenesis of idiopathic pulmonary fibrosis is alveolar epithelial and/or capillary cell injury. This promotes recruitment into the lung of circulating immune cells, such as monocytes, neutrophils, lymphocytes and eosinophils. These effector cells, together with resident lung cells, such as macrophages, alveolar epithelial and endothelial cells, then release cytokines, which stimulate target cells, typically fibroblasts, to replicate and synthesize increased amounts of collagen. Breakdown of extracellular matrix protein also may be inhibited, thereby contributing to the fibrotic process [Coker and Laurent, Eur Respir J. (1998) 11: 1218-1221].

Numerous cytokines have been implicated in the pathogenesis of fibrosis, including, without limitation, transforming growth factor-β (TGF-β), tumor necrosis factor-α (TNF-α), platelet-derived growth factor (PDGF), insulin-like growth factor-1 (IGF-1), endothelin-1 (ET-1) and the interleukins, interleukin-1 (IL-1), interleukin-6 (IL-6), interleukin-8 (IL-8), and interleukin-17 (IL-17). Chemokine leukocyte chemoattractants, including the factor Regulated upon Activation in Normal T-cells, Expressed and Secreted (RANTES), are also thought to play an important role. Elevated levels of pro-inflammatory cytokines, such as Interleukin 8 (IL-8), as well as related downstream cell adhesion molecules (CAMs) such as intercellular adhesion molecule-1 (ICAM-1) and vascular cell adhesion molecule-1 (VCAM-1), matrix metalloproteinases such as matrix metalloproteinase-7 (MMP-7), and signaling molecules such as S100 calcium-binding protein A12 (S100A12, also known as calgranulin C), in the peripheral blood have been found to be associated with mortality, lung transplant-free survival, and disease progression in patients with IPF [Richards et al, Am J Respir Crit Care Med. (2012) 185: 67-76].

The molecular processes driving fibrosis are wide-ranging and complex. The TGF-β cascade, which plays a major role in fibrosis, involves the binding of a ligand to a serine-threonine kinase type II receptor that recruits and phosphorylates a type I receptor. This type I receptor subsequently phosphorylates SMADs, which function as downstream effectors, typically by modulating target gene expression. TGF-β, which is a potent stimulator of the synthesis of extracellular matrix proteins in most fibrogenic cells and is synthesized and secreted by inflammatory cells and by effector cells, thereby functioning in both an autocrine and paracrine fashion (Id.). The TGF-β family of proteins has a potent stimulatory effect on extracellular matrix deposition, and in fact has been used in constructing induced animal models of fibrosis through gene transfer. In vitro studies show that TGF-β1, secreted as a latent precursor, promotes fibroblast procollagen gene expression and protein synthesis. The data suggest that the other mammalian isoforms, TGF-β2 and TGF-β3, also stimulate human lung fibroblast collagen synthesis and reduce breakdown in vitro. In animal models of pulmonary fibrosis, enhanced TGF-β1 gene expression is temporally and spatially related to increased collagen gene expression and protein deposition. TGF-β1 antibodies reduce collagen deposition in murine bleomycin-induced lung fibrosis, and human fibrotic lung tissue shows enhanced TGF-β1 gene and protein expression.

TNF-α can stimulate fibroblast replication and collagen synthesis in vitro, and pulmonary TNF-α gene expression rises after administration of bleomycin in mice. Soluble TNF-α receptors reduce lung fibrosis in murine models, and pulmonary overexpression of TNF-α in transgenic mice is characterized by lung fibrosis. In patients with IPF or asbestosis (a chronic inflammatory and fibrotic medical condition affecting the parenchymal tissue of the lungs caused by the inhalation and retention of asbestos fibers), bronchoalveolar lavage (BAL) fluid-derived macrophages release increased amounts of TNF-α compared with controls.

Platelet-derived growth factor (PDGF), connective-tissue growth factor (CTGF), and vasoactive peptide systems (especially angiotensin II and endothelin-1) play important roles [Id., citing Wynn T A. J Clin Invest (2007) 117: 524-9]. Among vasoactive systems, endothelin plays a role in fibrosis in virtually all organ systems, acting through G-protein-coupled endothelin-A or endothelin-B cell-surface receptors or both [Id., citing Khimji A K, Rockey D C. Cell Signal (2010) 22: 1615-25]. Endothelin (ET-1) also fulfills the criteria for a profibrotic cytokine. This molecule promotes fibroblast proliferation and chemotaxis and stimulates procollagen production. It is present in the lungs of patients with pulmonary fibrosis, and a recent report suggests that bosentan, an ET-1 receptor antagonist, ameliorates lung fibrosis when administered to experimental animals. Furthermore, angiogenic pathways may be important in fibrosis [Id., citing Johnson A, DiPietro L A. FASEB J (2013); 27: 3893-901]. Integrins, which link extracellular matrix to cells, are considered critical in the pathogenesis of fibrosis [Id., citing Levine D, et al. Am J Pathol (2000) 156: 1927-35; Henderson N C, et al. Nat Med (2013) 19: 1617-24].

### Regenerative Cells of the Lungs

The lung is a highly quiescent tissue, previously thought to have limited reparative capacity and a susceptibility to scarring. It is now known that the lung has a remarkable reparative capacity, when needed, and scarring or fibrosis after lung injury may occur infrequently in scenarios where this regenerative potential is disrupted or limited [Kotten, D. N. and Morrisey, E. E., “Lung regeneration: mechanisms, applications and emerging stem cell populations,” Nat. Med. (2014) 20 (8): 822-832, citing Beers, M F and Morrisey, EE, “The 3 R's of lung health and disease-repair, remodeling and regeneration,” J. Clin. Invest. (2011) 121: 2065-2073; and Wansleeben, C. et al, “Stem cells of the adult lung: their development and role in homeostasis, regeneration and disease,” Wiley Interdiscip. Rev. Dev. Biol. (2013) 2: 131-148]. Thus, the tissues of the lung may be categorized as having facultative progenitor cell populations that can be induced to proliferate in response to injury as well as to differentiate into one or more cell types.

The adult lung comprises at least 40-60 different cell types of endodermal, mesodermal, and ectodermal origin, which are precisely organized in an elaborate 3D structure with regional diversity along the proximal-distal axis. In addition to the variety of epithelial cells, these include cartilaginous cells of the upper airways, airway smooth muscle cells, interstitial fibroblasts, myofibroblasts, lipofibroblasts, and pericytes as well as vascular, microvascular, and lymphatic endothelial cells, and innervating neural cells. The regenerative ability of lung epithelial stem/progenitor cells in the different regions of the lung are thought to be determined not only by their intrinsic developmental potential but also by the complex interplay of permissive or restrictive cues provided by these intimately associated cell lineages as well as the circulating cells, soluble and insoluble factors and cytokines within their niche microenvironment [McQualter & Bertoncello., Stem Cells. (2012) May; 30(5); 811-816].

The crosstalk between the different cell lineages is reciprocal, multidirectional, and interdependent. Autocrine and paracrine factors elaborated by mesenchymal and endothelial cells are required for lung epithelial cell proliferation and differentiation [Yamamoto et al., Dev Biol. 2007 Aug. 1; 308(1): 44-53; Ding et al., Cell. 2011 Oct. 28; 147(3): 539-553], while endothelial and epithelial cell-derived factors also regulate mesenchymal cell proliferation and differentiation, extracellular matrix deposition and remodeling, and adhesion-mediated signaling [Crivellato. Int J Dev Biol. (2011) 55(4-5): 365-375; Grinnell & Harrington. Pulmonary endothelial cell interactions with the extracellular matrix. In: Voelkel N F, Rounds S, eds. The Pulmonary Endothelium: Function in Health and Disease. Chichester, West Sussex: Wiley-Blackwell, (2009): 51-72]. Chemotactic factors elaborated by these cell lineages also orchestrate the recruitment of inflammatory cells, which participate in the remodeling of the niche and the regulation of the proliferation and differentiation of its cellular constituents [McQualter & Bertoncello. Stem Cells. (2012) May; 30(5); 811-816].

### Stem Cell Niches

Adult tissue compartments contain endogenous niches of adult stem cells that are capable of differentiating into diverse cell lineages of determined endodermal, mesodermal or ectodermal fate depending on their location in the body. For example, in the presence of an appropriate set of internal and external signals, bone marrow-derived adult hematopoietic stem cells (HSCs) have the potential to differentiate into blood, endothelial, hepatic and muscle cells; brain-derived neural stem cells (NSCs) have the potential to differentiate into neurons, astrocytes, oligodendrocytes and blood cells; gut- and epidermis-derived adult epithelial stem cells (EpSCs) have the potential to give rise to cells of the epithelial crypts and epidermal layers; adipose-derived stem cells (ASCs) have the potential to give rise to fat, muscle, cartilage, endothelial cells, neuron-like cells and osteoblasts; and bone-marrow-derived adult mesenchymal stem cells (MSCs) have the potential to give rise to bone, cartilage, tendon, adipose, muscle, marrow stroma and neural cells.

Endogenous adult stem cells are embedded within the ECM component of a given tissue compartment, which, along with support cells, form the cellular niche. Such cellular niches within the ECM scaffold together with the surrounding microenvironment contribute important biochemical and physical signals, including growth factors and transcription factors required to initiate stem cell differentiation into committed precursors cells and subsequent precursor cell maturation to form adult tissue cells with specialized phenotypic and functional characteristics.

### Lung Mesenchymal Stem/Progenitor Cells

Tracheal and distal embryonic lung mesenchyme have been demonstrated to have inductive properties for the regional specification of the embryonic epithelium [Shannon & Deterding. Epithelial-mesenchymal interactions in lung development. In: McDonald J A, ed. Lung Biology in Health and Disease. Vol. 100. New York: Marcel Dekker Inc, (1997), pp. 81-118]. During lung development, mesenchymal stromal cells at the distal tip of the branching epithelium are known to secrete fibroblast growth factor (FGF)-10, which influences the fate and specificity of early lung epithelial progenitor cells [Bellusci et al. Development. (1997) December; 124(23): 4867-4878; Ramasamy et al. Dev Biol. 2007 Jul. 15; 307(2): 237-247]. FGF-10 is a component of a multifaceted epithelial-mesenchymal cell signaling network involving BMP, Wnt, and Shh pathways which coordinate the proliferation and differentiation of progenitor cells in the developing lung (reviewed in Morrisey & Hogan. Dev Cell. 2010 Jan. 19; 18(1): 8-23]. Lineage tracing studies have also revealed that FGF-10+ mesenchymal cells residing at the branching tip of the epithelium function as stem/progenitor cells for smooth muscle cells, which become distributed along the elongating airways [De Langhe et al., Dev Biol. 2006 Nov. 1; 299(1): 52-62; Mailleuix et al., Development. (2005) May; 132(9): 2157-2166]. In other studies, mesenchymal stromal cells adjacent to the trachea and extrapulmonary bronchi have also been shown to give rise to bronchiolar smooth muscle cells [Shan et al., Dev Dyn. (2008) 237: 750-755]. Collectively, these studies suggest that at least two distinct populations of mesenchymal stromal cells endowed with epithelial modulating properties emerge during development.

Several studies have identified resident mesenchymal stromal cells in adult lungs with the capacity for adipogenic, chondrogenic, osteogenic, and myogenic differentiation. These cells have been clonally expanded from heterogeneous populations of mixed lineage cells defined by their ability to efflux Hoechst 33342 [Giangreco et al., Am J Physiol Lung Cell Mol Physiol. (2004) 286: L624-L630; Summer et al., Am J Respir Cell Mol Biol. (2007); 37: 152-159], by their capacity for outgrowth from lung explant cultures [Hoffman et al., Stem Cells Dev. (2011); 20: 1779-1792], or by their characteristic expression of Sca-1 [McQualter et al., Stem Cells. (2009); 27: 612-22; Hegab et al, Stem Cells Dev. 2010; 19: 523-536]. In addition, further enrichment of CD45-CD31-Sca-1+ mesenchymal stromal cells has been achieved based on their lack of EpCAM expression, which selectively labels epithelial lineage cells [McQualter et al. Proc Natl Acad Sci USA (2010) 107: 1414-1419]. Resolution of the mesenchymal and epithelial lineages has revealed that the endogenous lung mesenchymal stromal cell population is necessary and sufficient to support the proliferation and differentiation of bronchiolar epithelial stem/progenitor cells in coculture [Id.]. This suggests that adult mesenchymal stromal cells share similar epithelial inductive properties to their embryonic counterparts and are an important element of the epithelial stem/progenitor cell niche in the adult lung. This concept is also supported by in vivo studies showing that following naphthalene injury of club cells, parabronchial mesenchymal cells secrete FGF-10 to support epithelial regeneration from surviving epithelial stem/progenitor cells [Volckaert et al., J Clin Invest. (2011) 121: 4409-4419].

### Lung Endothelial Progenitor Cells

Endothelial-epithelial cell interactions and angiogenic and angiocrine factors elaborated in the lung epithelial stem/progenitor cell microenvironment also play a role in the regulation of endogenous lung epithelial stem/progenitor cell regeneration and repair [Yamamoto et al., Dev Biol. 2007 Aug. 1; 308(1): 44-53; Ding et al., Cell. 2011 Oct. 28; 147(3): 539-553; Crivellato. Int J Dev Biol. (2011) 55(4-5): 365-375; Grinnell & Harrington. Pulmonary endothelial cell interactions with the extracellular matrix. In: Voelkel N F, Rounds S, eds. The Pulmonary Endothelium: Function in Health and Disease. Chichester, West Sussex: Wiley-Blackwell, (2009): 51-72]. For example, it has been reported that the coculture of human vascular endothelial cells with a human bronchial epithelial cell line promotes the generation of branching bronchioalveolar epithelial structures in a 3D culture system (Frazdottir et al. Respir Res. (2010) 11: 162]. While considerable progress has been made in understanding the heterogeneity, functional diversity, and pathophysiological behavior of lung vascular and microvascular endothelial cells, the immunophenotypic profiling, quantitation, and functional analysis of lung endothelial progenitor cells (EPC) lags far behind. As for EPC derived from human umbilical cord blood, bone marrow, and mobilized peripheral blood [Timmermans et al., J Cell Mol Med. (2009) 13: 87-102], the rarity of EPC in the lung, their lack of distinguishing markers, and the inability to discriminate circulating EPC and tissue resident EPC have been major impediments in assessing the contribution of endogenous lung EPC in lung vascular repair, and lung regeneration and remodeling [Thebaud & Yoder. Pulmonary endothelial progenitor cells. In: Voelkel N F, Rounds S, eds. The Pulmonary Endothelium: Function in Health and Disease. Chichester, West Sussex: Wiley, (2009) 203-216; Yoder. Proc Am Thorac Soc. (2011) 8: 466-470].

Lung macrovascular and microvascular endothelial cells can be resolved on the basis of their preferential binding to the lectins Helix pomatia and Griffonia simplicifolica, respectively [King et al., Microvasc Res. (2004) 67: 139-151], but there are no other cell surface markers that can discriminate mature lung endothelial cells and EPC [Yoder. Proc Am Thorac Soc. (2011) 8: 466-470]. In addition, the rarity of EPC has necessitated the ex vivo expansion and passaging of adherent heterogeneous rat (Alvarez et al., Am J Physiol Lung Cell Mol Physiol. (2008) 294: L419-L430] or mouse [Schniedermann et al., BMC Cell Biol. (2010) 11: 50] lung endothelial cells in liquid culture prior to quantitation and flow cytometric and functional analysis of lung-derived EPC in in vitro assays. These assays suggest that the lung microvasculature is a rich source of EPC. However, the incidence, immunophenotypic and functional properties of EPC in the primary explanted endothelial cells compared with their ex vivo manipulated, selected, and expanded counterparts remains indeterminate. The ability of these endogenous lung EPCs to contribute to vascular repair and remodeling in vivo is also unproven [Yoder. Proc Am Thorac Soc. (2011) 8: 466-470]. Studies suggest it is likely that both circulating EPC and resident lung EPC contribute to endothelial cell regeneration and repair [Balasubramian et al., Am J Physiol Lung Cell Mol Physiol. (2010) 298: L315-L323; Duong et al., Angiogenesis. (2011) 411-422; Chamoto et al. Am J Respir Cell Mol Biol. (2012) March; 46(3): 283-289].

Exosomes are released by a wide range of cell types present within the lung including endothelial cells, stem cells, epithelial cells, alveolar macrophage, and tumor cells, although epithelial cells are reported to be the main source of lung-derived exosomes [Alipoor, S D et al. Mediators Inflamm. (2016) 2016: 5628404, citing Y. Fujita, N. Kosaka, J. Araya, K. Kuwano, and T. Ochiya, “Extracellular vesicles in lung microenvironment and pathogenesis,” Trends in Molecular Medicine (2015) vol. 21, no. 9, pp. 533-542]. Exosomes released by airway epithelial cells contain mucins and alpha 2,6-linked sialic acid which have a neutralizing effect on human influenza virus infection [Id., citing N. T. Eissa, “The exosome in lung diseases: message in a bottle,” Journal of Allergy and Clinical Immunology (2013) vol. 131, no. 3, pp. 904-905]. Membrane-tethered mucins within epithelial cell-derived exosomes affect the structural properties, conformation, and surface charge of exosomes. The properties of exosomes contribute to mucociliary defense by the lung's innate immune system [Id., citing M. C. Rose and J. A. Voynow, “Respiratory tract mucin genes and mucin glycoproteins in health and disease,” Physiological Reviews (2006) 86 (1):. 245-278; Bourdonnay, Z. Zasłona, L. R. K. Penke et al., “Transcellular delivery of vesicular SOCS proteins from macrophages to epithelial cells blunts inflammatory signaling,” The Journal of Experimental Medicine (2015) 212 (5): 729-742].

Repair of Lung Tissue after Viral Infection or Mechanical Trauma

The repair of lung tissue after infection or mechanical trauma normally occurs in a controlled series of events beginning with damage signals sent from infected cells, which recruit inflammatory cells, which then induce secretion of growth factors, which activates basement membrane repair and finally leads to the replacement of injured tissue. Under normal circumstances, the wound healing response is downregulated once the injury is repaired. However, when either the infectious burden overwhelms the system (e.g., with SARS-CoV) or there is persistent damage (e.g., with hepatitis C virus infection), the wound healing response can become dysregulated, resulting in scarring and fibrosis. When fibrosis occurs, it leads to reduced lung function, resulting in a low quality of life and often death. There are limited treatment options for pulmonary fibrosis. Traditionally, corticosteroids are used for the treatment of acute respiratory distress syndrome (ARDS) and pulmonary fibrosis. However, during a viral infection, these interventions dampen the immune response and often result in worsened disease. Due to this lack of therapeutic options, there is a critical need to understand the molecular pathways involved in the development of fibrosis, thus helping to identify novel targets for therapy.

### Pulmonary Fibrosis

Pulmonary fibrosis (PF) occurs in association with a wide range of diseases, including scleroderma (systemic sclerosis), sarcoidosis, and infection, and as a result of environmental exposures (e.g., silica dust or asbestos), but in most patients it is idiopathic and progressive. Pulmonary fibrosis is characterized by parenchymal honeycombing (meaning the characteristic appearance of variably sized cysts in a background of densely scarred lung tissue. Microscopically, enlarged airspaces surrounded by fibrosis with hyperplastic or bronchiolar type epithelium are present. (From https://emedicine.medscape.com/article/2078590-overview), reduced lung compliance, and restrictive lung function (meaning a decreased lung capacity or volume, so a person's breathing rate often increases to meet the oxygen needs on inhalation). Fibrosis of the interstitial spaces (meaning the walls of the air sacs of the lungs (alveoli) and the spaces around blood vessels and small airways) hinders gas exchange, culminating in abnormal oxygenation and clinical dyspnea (meaning shortness of breath, inability to take a deep breath, or chest tightness). Progressive pulmonary fibrosis also leads to pulmonary hypertension, right-sided heart failure, and ultimately respiratory failure (Id.).

Alterations in the WNT signaling pathways are known to contribute to cellular (dys)functions in pulmonary fibrosis [Martin-Medina A, et al. Am J Respir Crit Care Med. 2018 Jul. 25, citing Konigshoff M, et al. PLoS One 2008; 3: e2142; Chilosi M, et al. Am J Pathol (2003); 162: 1495-1502; Selman M, et al. PLoS medicine (2008) 5: e62] and it has been demonstrated that secreted WNT proteins can be transported by EVs to exert their intercellular communication [Id., citing Gross J C, et al. Nat Cell Biol (2012) 14: 1036-1045]. The vast majority of research has focused on the role of the WNT/β-catenin pathway in pulmonary fibrosis, which has been linked to disturbed lung epithelial cell function and impaired repair [Id., citing Konigshoff M, et al. PLoS One (2008) 3: e2142; Chilosi M, et al. Am J Pathol (2003) 162: 1495-1502; Selman M, et al. PLoS medicine (2008) 5: e62; Baarsma H A, Konigshoff M. Thorax (2017); 72: 746-759]. β-catenin independent WNT signaling in lung fibrosis is much less studied. The WNT protein WNT-5A is largely known to exert its effects β-catenin independent and has been found upregulated in IPF fibroblasts [Id., citing Vuga L J, et al. Am J Respir Cell Mol Biol. (2009); 41(5): 583-9].

One study showed that lung fibroblasts are a source of EVs and demonstrate autocrine effects of EVs on fibroblast proliferation, which was enhanced by TGF-β (Id.). Similarly, MSC-derived exosomes were found to induce dermal fibroblast proliferation [Id., citing McBride J D, et al. Stem Cells Dev. (2017) 26 (19): 1384-1398]. Fibroblast-derived EVs did not promote myofibroblast differentiation, but rather decreased mRNA levels of myofibroblast markers. MSC-EVs have also been reported to suppress myofibroblast differentiation [Id., citing Fang S, et al. Stem Cells Transl Med. (2016); 5 (10): 1425-1439]. The proliferative effect of EVs on fibroblasts was to a large extent mediated by WNT-5A, as it was demonstrated that this effect could not only be attenuated by siRNA-mediated WNT-5A knockdown, but also by antibody-mediated neutralization of WNT-5A on EVs or upon destruction of EV structure (Id.). WNT transport on EVs has important implications with respect to the signaling range of WNT proteins, which is generally thought to be rather short and limited to close neighboring cells. EV-mediated transport can contribute to a larger signaling range of WNT proteins and thus determine the signaling outcome on other cells. WNT-5A has also been reported to promote processes as fibroblast adhesion [Id., citing Kawasaki A, et al. Cell Signal. (2007); 19 (12): 2498-2506] or invasion [Id., citing Wäster P, et al. Int J Oncol. (2011); 39 (1): 193-202], as well as epithelial-mesenchymal transition [Id., citing Gujral T S, et al. Cell. (2014); 159 (4): 844-856]. WNT-5A bound EVs in IPF bronchoalveolar lavage fluid (BALF) were shown to contribute to the functional effects, thus suggesting that fibroblast derived EVs can be found in IPF BALF. This work further raises the more general question whether EVs promote lung fibrosis development or might have a protective role in vivo (Id.).

### Idiopathic Pulmonary Fibrosis (IPF)

Idiopathic Pulmonary fibrosis (IPF, also known as cryptogenic fibrosing alveolitis, CFA, or Idiopathic Fibrosing Interstitial Pneumonia) is defined as a specific form of chronic, progressive fibrosing interstitial pneumonia of uncertain etiology that occurs primarily in older adults, is limited to the lungs, and is associated with the radiologic and histological pattern of usual interstitial pneumonia (UIP) [Raghu G. et al., Am J Respir Crit Care Med. (2011) 183(6): 788-824; Thannickal, V. et al., Proc Am Thorac Soc. (2006) 3(4): 350-356]. It may be characterized by abnormal and excessive deposition of fibrotic tissue in the pulmonary interstitium. On high-resolution computed tomography (HRCT) images, UIP is characterized by the presence of reticular opacities often associated with traction bronchiectasis. As IPF progresses, honeycombing becomes more prominent [Neininger A. et al., J Biol. Chem. (2002) 277(5): 3065-8]. Pulmonary function tests often reveal restrictive impairment and reduced diffusing capacity for carbon monoxide [Thomas, T. et al., J Neurochem. (2008) 105(5): 2039-2052]. Studies have reported significant increases in TNF-α and IL-6 release in patients with idiopathic pulmonary fibrosis (IPF) [Zhang, Y, et al. J. Immunol. (1993) 150(9): 4188-4196], which has been attributed to the level of expression of IL-1B [Kolb, M., et al. J. Clin. Invest, (2001) 107(12): 1529-1536]. The onset of IPF symptoms, shortness of breath and cough, are usually insidious but gradually progress, with death occurring in 70% of patients within five years after diagnosis. This grim prognosis is similar to numbers of annual deaths attributable to breast cancer [Raghu G. et al., Am J Respir Crit Care Med. (2011) 183(6): 788-824].

IPF afflicts nearly 130,000 patients in the United States, with approximately 50,000 new patients annually and nearly 40,000 deaths each year worldwide [Raghu G. et al., Am J Respir Crit Care Med. (2011) 183 (6): 788-824]. While these data are notable, a recent study reported that IPF may be 5-10 times more prevalent than previously thought, perhaps due to increasing prevalence or enhanced diagnostic capabilities [Thannickal, V. et al., Proc Am Thorac Soc. (2006) 3(4): 350-356]. Lung transplantation is considered a definitive therapy for IPF, but the five year survival post lung transplantation is less than 50%. Accordingly, even lung transplantation cannot be considered a “cure” for IPF. In addition to the physical and emotional toll on the patient, IPF is extremely expensive to treat and care for, with national healthcare costs in the range of $2.8 billion dollars for every 100,000 patients annually.

Previous studies have suggested that superimposed environmental insults may be important in the pathogenesis of IPF. In most reported case series, up to 75 percent of index patients with IPF are current or former smokers. In large epidemiologic studies, cigarette smoking has been strongly associated with IPF. In addition, many of the inflammatory features of IPF are more strongly linked to smoking status than to the underlying lung disease. Thus, cigarette smoking may be an independent risk factor for IPF. Latent viral infections, especially those of the herpes virus family, have also been reported to be associated with IPF.

Histopathologically, IPF can be described as accumulation of activated myofibroblasts (or mesenchymal cells) in fibroblastic foci [Thannickal, V. et al., Proc Am Thorac Soc. (2006) 3(4): 350-356]. Impaired apoptosis of myofibroblasts may result in a persistent and dysregulated repair process that culminates in tissue fibrosis. Arguably, inflammation also plays a critical role in IPF, perhaps through cyclic acute stimulation of fibroblasts. These findings point to potential targets for therapeutic intervention.

### Pathogenesis of Idiopathic Pulmonary Fibrosis (IPF)

While pathogenic mechanisms are incompletely understood, the currently accepted paradigm proposes that injury to the alveolar epithelium is followed by a burst of pro-inflammatory and fibroproliferative mediators that invoke responses associated with normal tissue repair. For unclear reasons, these repair processes never resolve and progressive fibrosis ensues [Selman M, et al., Ann Intern Med (2001) 134(2): 136-151; Noble, P. and Homer R., Clin Chest Med. (2004) 25(4): 749-58; Strieter, R., Chest (2005) 128 (5 Suppl 1): 526S-532S].

Aside from lung transplantation, potential IPF treatments have included corticosteroids, azathioprine, cyclophosphamide, anticoagulants, and N-acetylcysteine [Raghu G. et al., Am J Respir Crit Care Med. (2011) 183(6): 788-824]. In addition, supportive therapies such as oxygen therapy and pulmonary rehabilitation are employed routinely. However, none of these have definitely impacted the long term survival of IPF patients, which further highlights the unmet medical need for treatment options in IPF. As an example, despite mixed clinical program results, InterMune's oral small-molecule Esbriet® (pirfenidone) received European and Japanese approvals for patients with IPF. Esbriet® thus became the first medication specifically indicated for the treatment of IPF; due to equivocal trial outcomes and drug side effects, the drug's utility is viewed with skepticism in the United States and did not receive an FDA approval based on the data submitted at that time. A large, double-blind, placebo-controlled phase 3 clinical trial to assess the safety and efficacy of pirfenidone in patients with IPF was completed in 2017.

### Cardiac Fibrosis

The heart undergoes extensive structural and functional remodeling (meaning a group of molecular, cellular and interstitial changes that manifest clinically as changes in size, mass, geometry and function of the heart) in response to injury, central to which is the hypertrophy (meaning an increase in size of the individual muscle cells without changing their total number) of cardiac myocytes, with excessive deposition of extracellular matrix [Rockey D C et al., N Engl J Med. 2015 Mar. 19; 372(12): 1138-1149, citing Hill J A, Olson E N. N Engl J Med (2008) 358: 1370-1380]. Myocardial fibrosis is commonly categorized as one of two types: reactive fibrosis or replacement fibrosis. Reactive fibrosis occurs in perivascular spaces (meaning the fluid-filled space that surrounds a blood vessel or organ) and corresponds to similar fibrogenic responses in other tissues; replacement fibrosis occurs at the site of myocyte loss.

In the heart, fibrosis is attributed to cardiac fibroblasts, the most abundant cell type in the myocardium, the middle muscular layer of the heart wall. These cells are derived from fibroblasts that are native to the myocardium, from circulating fibroblasts, and from fibroblasts that emerge from epithelial-to-mesenchymal transition [Id., citing Zeisberg E M, et al. Nat Med (2007) 13: 952-61; Moore-Morris T, et al. J Clin Invest (2014) 124: 2921-2934]. All these cell types proliferate and differentiate into myofibroblasts in response to injury, a process that is driven by classic factors such as TGF-β1, endothelin-1, and angiotensin II [Id., citing Burchfield J S, et al. Circulation (2013) 128: 388-400]. Crosstalk and feedback also occurs between cells—in this case, between activated fibroblasts and cardiomyocytes—which further fuels fibrogenesis [Id., citing Martin M L, Blaxall B C. J Cardiovasc Transl Res (2012) 5: 768-782].

Cardiac fibrosis contributes to both systolic and diastolic dysfunction and to perturbations of electrical excitation; it also disrupts repolarization [Id., citing Spinale F G. Physiol Rev (2007) 87: 1285-1342]. Proarrhythmic effects (meaning worsening of existing arrhythmias) are the most prominent. Collagenous septa in the failing heart contribute to arrhythmogenesis by inducing a discontinuous slowing of conduction [Id., citing Spach M S, Boineau J P. Pacing Clin Electrophysiol (1997) 20: 397-413]. Areas of arrhythmogenic fibrosis slow conduction through junctions in the heterocellular gap (meaning intercellular channels that allow direct diffusion of ions and small molecules between adjacent cells) that couple fibroblasts and cardiomyocytes [Id., citing Miragoli M, et al. Circ Res (2006) 98: 801-810]. Conduction of vasoconstrictor and vasodilator responses in the microcirculation involves electrical coupling through gap junction channels among cells of the vascular wall [Sandow S L, et al. Cardiovasc. Res. (2003); 60 (3): 643-653]. Endocardial breakthrough of microreentrant circuits (meaning small areas of continuous circulating electricity in which an impulse reenters and repetitively excites a region of the heart; are the basis of most clinical arrhythmias and occurs as a result of the heterogeneous spatial distribution of fibrosis and the triggering of activity caused by the depolarization of myocytes by electrically coupled myofibroblasts [Rockey D C et al., N Engl J Med. 2015 Mar. 19; 372 (12): 1138-1149, citing Tanaka K, et al. Circ Res (2007) 101: 839-47; Miragoli M, et al. Circ Res (2007) 101: 755-758].

Fibrotic scarring in the heart correlates strongly with an increased incidence of arrhythmias and sudden cardiac death [Id., citing Wu K C, et al. J Am Coll Cardiol (2008) 51: 2414-2421]. For example, a 3% increase in the extracellular volume fraction of fibrous tissue (measured by means of magnetic resonance imaging after the administration of gadolinium) is associated with a 50% increase in the risk of adverse cardiac events [Id., citing Wong T C, et al. Circulation (2012) 126: 1206-1216].

### Hepatic Fibrosis

The liver is made up of two lobes, each of which is made up of thousands of hexagonally shaped lobules. Each lobule is made up of numerous liver cells, called hepatocytes, that are cuboidal epithelial cells that line up in radiating rows and make up the majority of cells in the liver. Hepatocytes perform most of the liver's functions-metabolism, storage, digestion, and bile production. Between each row are sinusoids, which are small blood vessels lined by hepatocytes that diffuse oxygen and nutrients through their capillary walls into the liver cells. The lobules are connected to small bile ducts that connect with larger ducts to ultimately form the hepatic duct. Hepatic biliary cells, which line the bile ducts, are targets of liver injury, but also orchestrate liver repair. They undergo extensive morphogenesis to form a complex network of intrahepatic biliary ducts. This network functions to drain the bile produced by hepatocytes to the intestine. Hepatic stellate cells exist in the space between parenchymal cells and sinusoidal endothelial cells of the hepatic lobule and store 80% of retinoids in the whole body as retinyl palmitate in lipid droplets in the cytoplasm. In physiological conditions, these cells play pivotal roles in the regulation of retinoid homeostasis, which contributes to many diverse functions including vision, inflammatory/immune response, adipogenesis, cell differentiation, and insulin sensitivity. In pathological conditions such as liver fibrosis, hepatic stellate cells lose retinoids and synthesize a large amount of extracellular matrix (ECM) components including collagen, proteoglycan, and adhesive glycoproteins [Senoo H. Med Electron Microsc. (2004) 37(1): 3-15]. Healthy sinusoidal endothelial cells maintain hepatic stellate cell quiescence, thus inhibiting their vasoconstrictive effect [Poisson J, et al. J Hepatol. (2017) 66(1): 212-227].

Hepatic fibrosis typically results from an inflammatory process that affects hepatocytes or biliary cells. Inflammation leads to the activation of effector cells, which results in the deposition of extracellular matrix. Although a variety of effectors synthesize extracellular matrix in the liver, hepatic stellate cells appear to be the primary source of extracellular matrix. Abundant evidence suggests that the stellate cell is pericyte-like (pericytes are spatially isolated contractile cells on capillaries which control blood flow), undergoing a transformation into a myofibroblast in response to injury [Rockey D C et al., N Engl J Med. (2015) 372(12): 1138-1149, citing Rockey D C, et al. J Clin Invest (1993) 92: 1795-804].

In the liver, multiple cell types, including stellate cells, endothelial cells, Kupffer cells (the resident macrophages of the liver), bile-duct cells, and immune cells, orchestrate the cellular and molecular response to injury [Id., citing Rockey D C. Clin Gastroenterol Hepatol (2013) 11(3): 224-231]. Numerous molecular pathways, similar to those found in other organs, are involved. A pathway that appears to be unique to the liver involves toll-like receptor 4 (TLR4); TLR4 is activated on the surface of stellate cells by intestinal bacterial lipopolysaccharides derived from the gut (i.e., translocated bacteria), triggering cell activation and fibrogenesis and thereby linking fibrosis to the microbiome [Id., citing Seki E, et al. Nat Med (2007) 13: 1324-1332; Fouts D E, et al. J Hepatol (2012) 56: 1283-92]. TLR4 expression is associated with portal inflammation and fibrosis in patients with fatty liver disease [Id., citing Vespasiani-Gentilucci U, et al. Liver Int (2015) 35: 569-581].

The end result of hepatic fibrogenesis is cirrhosis, an ominous parenchymal lesion that underlies a wide range of devastating complications that have adverse effects on survival. Portal hypertension (meaning an increase in the pressure within the portal vein, which carries blood from the digestive organs to the liver), a devastating result of injury, develops during the fibrogenic response after disruption of the normal interaction between sinusoidal endothelial cells and hepatic stellate cells; the resulting activation and contraction of pericyte-like stellate cells leads to sinusoidal constriction (sinusoidal capillaries are a special type of capillary that have a wide diameter) and increased intrahepatic resistance (meaning the resistance in the liver vascular bed to the flow that reaches the liver via the portal vein, which can be assessed experimentally, based on Ohm's law, by measuring portal pressure changes when an increasing portal venous flow is applied). This increase in resistance in turn activates abnormal signaling by smooth-muscle cells in mesenteric vessels. An increase in angiogenesis and collateral blood flow follows, resulting in an increase in mesenteric blood flow (meaning blood flow to the intestines) and a worsening of portal hypertension [Id., citing Sanyal A J, et al. Gastroenterology (2008) 134: 1715-1728]. The major clinical sequelae of portal hypertension, variceal hemorrhage (varices are dilated blood vessels caused by portal hypertension; they cause no symptoms unless they rupture and bleed, which can be life threatening) and ascites (meaning an abnormal accumulation of protein-containing fluid within the abdomen), emerge relatively late, after the portal pressure rises to a hepatic venous pressure gradient of more than 12 mm Hg (Id.).

### Renal Fibrosis

Events that initiate renal fibrosis are diverse, ranging from primary renal injury to systemic diseases [Id., citing Liu Y. Nat Rev Nephrol (2011) 7: 684-696; Kaissling B, et al. Biochim Biophys Acta (2013) 1832: 931-939). The kidneys are susceptible to hypertension and diabetes, the two leading causes of renal fibrosis. As is true in other organs, fibrosis of the kidney is mediated by cellular elements (e.g., inflammatory cells) and molecular elements (e.g., cytokines, TGF-β1, CTGF, PDGF, and endothelin-1) (Id., citing Liu Y. Nat Rev Nephrol (2011) 7: 684-696; Kaissling B, et al. Biochim Biophys Acta (2013) 1832: 931-939; Chen J, et al. J Am Soc Nephrol (2012) 23: 215-224; Mezzano S A, et al. Hypertension (2001) 38: 635-638]. The intrarenal renin-angiotensin-aldosterone axis (a signaling pathway that regulates the body's blood pressure by homeostatic control of arterial pressure, tissue perfusion, and extracellular volume) is particularly important in hypertension-induced fibrosis [Id., citing Mezzano S A, et al. Hypertension (2001) 38: 635-638].

The kidney's unique cellular architecture consists of the glomeruli (meaning a tuft formed of capillary loops at the beginning of each nephiric tubule in the kidney; this tuft with its capsule (Bowman's capsule) constitutes the Malpighian body), tubules (meaning the portion that extends from the Bowman capsule in the kidney cortex (meaning the outer part of the kidney between the renal capsule and the renal medulla) into the kidney medulla (meaning the innermost part of the kidney), interstitium (meaning the intratubular, exxtraglomerular, extravascular space of the kidney), and capillaries. Injury at any of these sites triggers the deposition of extracellular matrix [Id., citing Burchfield J S, et al. Circulation (2013) 128: 388-400]. The location of the initial injury is an important determinant of the clinical consequences. Injuries that initially target glomeruli elicit patterns of disease that are different from those that are elicited by injuries to the tubular-interstitial environment. For example, NSAIDs, urinary obstruction, polycystic kidney disease, and infections can provoke tubulointerstitial fibrosis (a progressive detrimental connective tissue deposition on the kidney parenchyma), whereas glomerular immune deposition (e.g., the deposition of IgA in the glomeruli) leads to glomerulonephritis (meaning acute inflammation of the kidney, typically caused by an immune response) [Id., citing Miragoli M, et al. Circ Res (2007) 101: 755-758; Wu K C, et al. J Am Coll Cardiol (2008) 51: 2414-2421]. Glomeruli and podocytes (highly specialized cells of the kidney glomerulus that wrap around capillaries and that neighbor cells of the Bowman's capsule, see Reiser J and Altintas M M. Podocytes. F1000Research (2016) 5(F1000 Faculty Rev): 114) are sensitive to systemic and local immunologic insults; high glomerular capillary pressure, exacerbated by systemic hypertension and diabetes, leads to proteinuria (meaning the presence of abnormal quantities of protein in the urine), the activation of cytokines and complement, and the infiltration of immune cells, resulting in epithelial cell and interstitial fibrosis [Id., citing Wong T C, et al. Circulation (2012) 126: 1206-1216; Rockey D C. Clin Gastroenterol Hepatol (2013) 11(3): 224-231]. Podocytes cooperate with mesangial cells (contractile cells that constitute the central stalk of the glomerulus) to support the structure and function of the glomerulus (see, e.g., Pavenstadt, H, Am. J. Physiol. Renal Physol. (2000) 278 (2): F173-F179). Mesangial cells have characteristics of a modified smooth muscle cell but also are capable of generation of prostaglandins and mediators of inflammation; production and breakdown of basement membrane and other biomatrix material; synthesis of cytokines, and uptake of macromolecules, including immune complexes [see Schlndorff D., FASEB J. (1987) 1(4): 272-281].

Glomerular fibrosis, regardless of the cause, diminishes renal blood flow, which leads to hypoxia and the activation of hypoxia-inducible factor 1, a dimeric protein complex that plays an integral role in the body's response to low oxygen concentrations, or hypoxia, which in turn triggers nephron collapse and fibrotic replacement by means of rarefaction (meaning a decrease in the capillary density) [Id., citing Seki E, et al. Nat Med (2007) 13: 1324-1332]. The renal interstitium and capillaries contribute substantially to tubulointerstitial disease, as peritubular pericytes migrate into the interstitium, where they are transformed into myofibroblasts [Id., citing Fouts D E, et al. J Hepatol (2012) 56: 1283-1292].

Regardless of the initiating insult, renal fibrosis leads to loss of function and organ failure. Homeostasis can be maintained with a glomerular filtration rate as low as approximately 10% of the normal rate. As the mechanisms maintaining homeostasis are progressively disrupted, anemia develops and the regulation of electrolyte balance and pH is disrupted (Id.).

### Radiation Fibrosis

Patients with cancer often receive external beam ionizing radiation therapy either alone or in combination with surgery and/or chemotherapy. Ionizing radiation induces damage not only in rapidly proliferating tumor cells but also in normal tissue in the radiation field. A significant contributor to patient morbidity is radiation-induced fibrosis (RIF), which may occur in the skin and subcutaneous tissue, lungs, gastrointestinal and genitourinary tracts, as well as any other organs in the treatment field. Radiation injury triggers inflammation and ultimately stimulates transdifferentiation of fibroblasts into myofibroblasts. In addition to their excessive proliferation, these myofibroblasts produce excess collagen and other extracellular matrix (ECM) components, which is compounded by a reduction in remodeling enzymes. Subsequent fibrosis reduces tissue compliance and—in a majority of cancer patients and particularly those with head and neck cancer—causes cosmetic and functional impairment that significantly impacts quality of life [Straub J M, et al. J Cancer Res Clin Oncol. (2015) 141(11): 1985-1994].

RIF usually occurs 4-12 months after radiation therapy and progresses over several years. It affects almost every part of the body that is exposed to radiation. The clinical presentation depends on the type of tissue exposed to irradiation. In general, RIF may manifest as skin induration and thickening, muscle shortening and atrophy, limited joint mobility, lymphedema, mucosal fibrosis, ulceration, fistula, hollow organ stenosis, and pain [Id., citing Dorr W, Hendry J H. Radiother Oncol J Eur Soc Ther Radiol Oncol. (2001) 61: 223-231].

The mechanism of RIF is similar to that of any chronic wound healing process. An initial injury incites an acute response that leads to inflammation, followed by fibroblast recruitment and activation with extracellular matrix deposition. Radiation is energy in the form of waves or high-speed particles. The term “ionizing” indicates that said energy is strong enough to displace bound electrons. Ionizing radiation refers to three types of emissions—alpha, beta, and gamma—with therapeutic radiation being predominantly gamma ([Id., citing Harrison J D, Stather J W. J Anat. (1996) 189(Pt 3): 521-530]. Radiation injury results from two primary mechanisms: direct DNA damage and the generation of reactive oxygen species (ROS) [Id., citing Travis E L. Semin Radiat Oncol. (2001) 11(3): 184-196]. The latter is more prominent in RIF and involves the interaction of ionizing radiation with water molecules to form free radicals, including superoxide, hydrogen peroxide, and hydroxyl radical [Id., citing Tak J K, Park J W. Free Radic Biol Med. (2009) 46: 1177-1185], the last of which accounts for 60-70% of the total damage [Id., citing Terasaki Y, et al. Am J Physiol Lung Cell Mol Physiol. (2011) 301: L415-L426; Zhao W, Robbins M E. Curr Med Chem. (2009) 16: 130-143]. Reactive nitrogen species (RNS) also likely play a role in radiation injury, as treatment with the inducible nitric oxide synthase (iNOS) inhibitor, L-nitroarginine methyl ester (L-NAME), prevented acute lung injury in rats [Id., citing Khan M A, et al. Radiother Oncol J Eur Soc Ther Radiol Oncol. (2003) 66: 95-102]. Free radicals damage all components of cells, including proteins, nucleic acids, and lipids [Id., citing Terasaki Y, et al. Am J Physiol Lung Cell Mol Physiol. (2011) 301: L415-L426; Zhao W, Robbins M E. Curr Med Chem. (2009) 16: 130-143]. Superoxide dismutase, catalase, and glutathione peroxidase are responsible for controlling free radical damage [Id., citing Greenberger J S, Epperly M W. In vivo. (2007) 21: 141-146]. A deficiency in these enzymes or excess ROS/RNS leads to oxidative stress in tissues [Id., citing Chaudiere J, Ferrari-Iliou R. Food Chem Toxicol. (1999) 37: 949-962; Darley-Usmar V, Halliwell B. Pharm Res. (1996) 13: 649-662; Evans P, Halliwell B. Ann N Y Acad Sci. (1999) 884: 19-40]. Injured cells release chemoattractant molecules that trigger nonspecific inflammation [Id., citing Denham J W, Hauer-Jensen M. Radiother Oncol J Eur Soc Ther Radiol Oncol. (2002) 63: 129-145; Travis E L. Semin Radiat Oncol. (2001) 11 (3): 184-96; Williams J P, et al. Curr Drug Targets. (2010) 11: 1386-1394]. Furthermore, thrombosis and ischemia exacerbate local injury leading to further release of inflammatory chemokines and cytokines [Id., citing Boerma M, Hauer-Jensen M. Curr Drug Targets. (2010) 11: 1405-1412; Lefaix J L, Daburon F. Health Phys. 1998; 75: 375-384].

Neutrophils are the first inflammatory cells to arrive at the site of injury [Id., citing Abreu M T, et al. J Immunol. (2005) 174: 4453-4460]. Increased expression of intercellular adhesion molecule 1 (ICAM-1) [Id., citing Hallahan D E, et al. J Natl Cancer Inst. (2002) 94: 733-741] and platelet endothelial cell adhesion molecule 1 (PECAM-1) [Id., citing Quarmby S, et al. Arterioscler Thromb Vasc Biol. (1999) 19: 588-597] on disrupted endothelial surfaces contributes to neutrophil extravasation and transmigration into tissues [Id., citing Lefaix J L, Daburon F. Health Phys. (1998) 75: 375-384]. When these cells come into contact with collagen fragments and fibronectin, they release proinflammatory cytokines like tumor necrosis factor alpha (TNF-α), IL-1, and IL-6 that perpetuate the development of ROS and lead to even greater local inflammation [Id., citing Calveley V L, et al. Int J Radiat Biol. (2005) 81: 887-899; Finkelstein J N, et al. Environ Health Perspect. (1997) 105(Suppl 5): 1179-1182; Olman M A, et al. Chest. (2002) 121: 69S-70S; Porter D W, et al. Inhalation Toxicol. (2002) 14: 349-367; Sedgwick J B, et al. J Allergy Clin Immunol. (2002) 110: 752-756]. The next cells to arrive are the monocytes and lymphocytes [Id., citing Haston C K, Travis E L. Cancer Res. (1997) 57: 5286-5291; Sharplin J, Franko A J. Radiat Res. (1989) 119: 1-14], which interact with each other to lead to the differentiation of monocytes into two subsets of macrophages [Id., citing Gordon S, Martinez F O. Immunity. (2010) 32: 593-604; Sica A, Mantovani A. J Clin Investig. (2012) 122: 787-795; Varin A, Gordon S. Immunobiology. (2009) 214: 630-641]: classically activated pro-inflammatory MI or alternatively activated anti-inflammatory M2 [Id., citing Ford A Q, et al. BMC Immunol. (2012) 13: 6; Zhang H, et al. J Radiat Res. (2011) 52: 717-726]. Platelet-derived growth factor (PDGF) secreted from the M2 subset promotes neoangiogenesis and stimulates the migration of fibroblasts into the injured tissue from either the surrounding stroma or from circulating mesenchymal stem cells [Id., citing Li M, Jendrossek V, Belka C. Radiat Oncol. (2007) 2: 5; Mathew M, Thomas S M. In: Li X, editor. Squamous cell carcinoma. InTech; (2012) pp. 163-174]. They also secrete TGF-β, which is heavily implicated in RIF [Id., citing Li M O, et al. Annu Rev Immunol. 2006; 24: 99-146]. Indeed, TGF-β is responsible for a number of functions that contribute to the pathogenesis of this condition, including the production of fibroblasts from bone marrow progenitors [Id., citing Campana F, et al. J Cell Mol Med. (2004) 8: 109-116; Rodemann H P, Bamberg M. Radiother Oncol J Eur Soc Ther Radiol Oncol. (1995) 35: 83-90] and the differentiation of fibroblasts into myofibroblasts [Id., citing Yarnold J, Brotons M C. Radiother Oncol J Eur Soc Ther Radiol Oncol. (2010) 97: 149-161], whereby a phenotypic change in the fibroblasts results in increased expression of alpha-smooth muscle actin (α-SMA), followed by subsequent transformation into protomyofibroblasts and eventual maturation into myofibroblasts [Id., citing Tomasek J J, et al. Nat Rev Mol Cell Biol. (2002) 3: 349-363]. These myofibroblasts may also derive from circulating bone marrow-derived progenitor cells known as fibrocytes or from epithelial cells undergoing epithelial-mesenchymal transition (EMT) [Id., citing Darby I A, Hewitson T D. Int Rev Cytol. (2007) 257: 143-179]. In response to TGF-β, myofibroblasts secrete excess collagen, fibronectin, and proteoglycans [Id., citing Chithra P, et al. J Ethnopharmacol. (1998) 59: 179-186], and in doing so they are responsible for the increased stiffness and thickening of the tissue [Id., citing Lefaix J L, Daburon F. Health Phys. (1998) 75: 375-384; Martin M, et al. Int J Radiat Oncol Biol Phys. (2000) 47: 277-290]. Furthermore, TGF-β promotes decreased matrix metalloproteinase (MMP) activity (especially MMP-2 and MMP-9) and increased activity of tissue inhibitors of metalloproteinases (TIMPs), compounding the already excessive ECM deposition [Id., citing Pardo A, Selman M. Proc Am Thorac Soc. (2006) 3 (4): 383-]). Lastly, although myofibroblasts promote endothelial cell proliferation and angiogenesis through the secretion of basic fibroblast growth factor (bFGF) [Id., citing Finlay G A, et al. J Biol Chem. (2000) 275: 27650-27656], excess collagen reduces vascularity over time [Id., citing Lefaix J L, Daburon F. Health Phys. (1998) 75: 375-384]. This makes fibrotic areas susceptible to physical trauma and gradual ischemia, which may lead to loss of function, tissue atrophy, reduction in the number fibroblasts, or necrosis [Id., citing Burger A, et al. Int J Radiat Biol. (1998) 73: 401-408; Delanian S, et al. Radiother Oncol J Eur Soc Ther Radiol Oncol. (1998) 47: 255-261; Delanian S, et al. Radiother Oncol J Eur Soc Ther Radiol Oncol. (2001) 58: 325-331; Denham J W, Hauer-Jensen M. Radiother Oncol J Eur Soc Ther Radiol Oncol. (2002) 63: 129-145; Rudolph R, et al. Plast Reconstr Surg. (1988) 82: 669-677; Toussaint O, et al. Mech Ageing Dev. (2002) 123: 937-946].

### Other Forms of Fibrosis

Fibrosis also occurs, for example, in the spine (epidural fibrosis), skeletal muscle, the joints, bone marrow, brain, eyes, intestines, peritoneum and retroperitoneum, pancreas, and skin.

Epidural fibrosis is defined as nonphysiologic scar formation, usually at the site of neurosurgical access into the spinal canal [Maswpust, V. et al. Clinical J. Pain (2009) 25 (7): 600-6]. From its onset, it behaves as a reparative inflammation. Epidural fibrosis and recurrent or residual disk herniation are among the common causes of failed back surgery syndrome which is characterized by intractable pain and various degrees of functional incapacitation after removal of herniated lumbar intervertebral disc and/or bone. Its reported incidence ranges from 105 to 40% [Bundschuh, C V et al. AJNR (1988) 9: 169-78, citing Burton, C V et al. Clin. Ortho. (1981) 157: 191-99; Burton, CV. Spine (1978) 3 (1): 24-30]. Reoperation on scar tissue generally leads to a poor surgical result, in contradistinction to removal of the herniated disk [Id., citing Jorgensen, J. et al. Neuroradiology (1975) 9: 133-44; Law, J D et al. J. Neurosurg. (1978) 48: 259-63].

Skeletal muscle subjected to different types of injuries undergoes degeneration with inflammatory cellular infiltration [Mahdy, Mohamed A. A. Cell & Tissue Res. (2019) 375: 575-88, citing Karalaki, M. et al. In vivo (2009) 23: 779-96; Mahdy, MA Cell Tissue Res. (2018) 374: 233-41]; Mann, et al. (2011)]. The quiescent satellite cells (SCs) activate, proliferate and differentiated forming new myotubes with production of new ECM, blood vessels and nerves (Laumonier and Menetrey (2016); Mann, C J et al. Skeletal Muscle (2011) 4: 21; Saclier, M. et al. FEBS J. (2013) 280: 4118-30; Yin, H. et al Physiol. Rev. (2013) 93: 23-67). The myotubes mature into myofibers and ECM undergoes remodeling (Id., citing Alameddine, H S and Morgan, JE J. Neuromuscul. Dis. (2016) 3: 455-73; Lei, H et al. Am. J. Physiol. Cell Physiol. (2013) 305: C529-38). The regenerated muscle in normal conditions resembles undamaged muscle in morphological as well as functional states [Id., citing Charge, S B and Rudnicki, MA. Physiol. Rev. (2004) 84: 209-38].

The high regeneration ability of skeletal muscle compromised, for example, in muscular dystrophies [Id., citing Pessina, P et al. Skeletal Muscle (2014) 4: 7], a group of inherited skeletal muscle disease caused by mutations in genes controlling stability and viability of muscle fibers [Id., citing Bersini, S, et al. Adv. Drug Deliv. Rev. (2018) 129: 64-77], myopathies, and severe injuries (Id., citing Uezumi, et al. (2014). The excessive deposition of fibrous tissue impairs muscle function (Id., citing Delaney, K et al. Cell Biol. Int. (2017) 41: 706-715; Jarvinen, T A et al. Am. J. Sports Med. (2005) 33: 745-764; Uezumi et al., 2014), affects muscle fiber regeneration after injury and increases muscle susceptibility to reinjury [Id., citing Prazeres, PHDM et al. Int. J. Biochem. Cell Biol. (2018) 5: 109-113]. It is considered a major cause of muscle weakness. Fibrosis of skeletal muscle is a hallmark of muscular dystrophies, aging and severe muscle injury.

Muscle fibrosis is closely associated and overlapping with inflammation. In response to muscle injury, neutrophils are recruited to the injury site to phagocytose damaged cells and initiate regeneration [Id., citing Bersini, S, et al. Adv. Drug Deliv. Rev. (2018) 129: 64-77; Tidball, JG (2005) Am. J. Physiol. Regul. Integr. Comp. Physiol. (2005) 288: R345-R353; Tidball, J G and Welc, SS. Mol. Ther. (2015) 23: 1134-1135]. The recruited neutrophils release chemoattractant cytokines [Id., citing Soehnlein, O et al. Blood (2009) 114: 4613-4623], which promote further infiltration of monocytes and macrophages [Id., citing Mann, C J et al. Skeletal Muscle (2011) 4: 21; Saclier, M et al. FEBS J. (2013) 280: 4118-4190; Serrano, A L et al. Curr. Topics Dev. Biol. (2011) 96: 167-201]. The classically activated M1 phenotype produces proinflammatory cytokines, such as TNF-α and IL-6, which activates fibroblast proliferation, while the alternatively activated M2 subtype produces TGF-β1 and fibronectin. Disturbance of the balance between M1 and M2 macrophage activation increases the expression of TGF-β1, which activates resident fibroblasts and inhibits fibroadipogenic progenitors (FAPs) apoptosis, and induces their differentiation into fibrogenic lineage leading to excessive ECM deposition and fibrosis [Id., citing Lemos, D R et al. Nat. Med. (2015) 21: 786-794)].

### Aged Muscle:

Aged muscle is characterized by loss of muscle mass (sarcopenia) [Id., citing Ciciliot, S and Schiaffino, S. Curr. Pharm. Des. (2010) 16: 906-914]. Sarcopenia is associated with decreased muscle force and endurance together with increased fibrosis. Age-related fibrosis is mediated through different factors, such as defects in cell populations, alteration in cell signaling and changes in growth factors regulation (Id., citing Serrano, A L et al. Curr. Top. Dev. Biol. (2011) 96: 167-201, which in turn lead to change in muscle microenvironment (Id., citing Zhou, Y. et al. Tissue Eng. Part C. Methods (2017) 23: 1012-1021).

The myogenic ability of satellite cells decreases in aged muscles due to increased levels of IL-6 [Id., citing Forcina, L. et al. Cytokine Growth Factor Rev. (2018) 41: 1-9]. On the other hand, aged SCs as well as myoblasts, have an increased tendency to convert to a fibrogenic lineage [Id., citing Brack, A S et al. Science (2007) 317: 807-810; Ciciliot, S. and Schiaffino, S. Curr. Pharm. Des. (2010) 16: 906-914; von Malzahn, J. et al. Trends Cell Biol (2012) 22: 602-609]. This myogenic-fibrogenic conversion is under the control of the Wnt signaling pathway [Id., citing Biressi, S. et al. Sci. Transl. Med. (2014) 6: (267): 267ra76; Brack, A S et al. Science (2007) 317: 807-810], and compositional changes of ECM in aged muscle [Id., citing Parker, MH Front. Genetics (2015) 6: 59; Stearns-Reider, K M et al. Aging Cell (2017) 16: 518-528]. The Wnt signaling pathway mediates the myogenic-fibrogenic conversion of SCs through upregulation of TGF-β2 expression [Id., citing Biressi, S. et al. Sci. Transl. Med. (2014) 6: (267): 267ra76].

A crosstalk between Wnt/β-catenin signaling and TGF-β signaling has been shown in the pathogenesis of fibrosis. TGF-β signaling upregulates the expression of Wnt/β-catenin and vice versa [Id., citing Guo, Y. et al. Physiol. Res. (2012) 61: 337-346]. It has been shown that TGF-β is upregulated in aged myogenic cells together with an increased level of phosphorylated Smad2/3, β-catenin, and collagen I [Id., citing Rajasekaran, M R et al. Am. J. Physiol. Gastrointest. Liver Physiol. (2017) 313: G581-88; Serrano, A L et al Curr. Top. Dev. Biol. (2011) 96: 167-201]. On the other hand, fibroblasts isolated from aged muscle show an increased level of TGF-β, collagen IVa2, laminin [Id., citing Thorley, M et al. J. Neuromuscl. Dis. (2015) 2: 205-217] and tissue inhibitors of metalloproteinase (TIMP)-1 and 2, inhibitors of ECM degradation [Id., citing Stearns-Reider, K M et al. Aging Cell (2017) 16: 518-528], indicating that collagen deposition increases in intact muscle with advancement of age [Serrano, A L et al Curr. Top. Dev. Biol. (2011) 96: 167-201].

Early studies have indicated that there is considerable musculoskeletal dysfunction in some patients with COVID-19, although long-term follow-up studies have not yet been conducted [Disser, N P et al. J. Bone Joint Surg. Am. (2020) 102: 1197-1204].

### Retroperitoneal Fibrosis:

Retroperitoneal fibrosis is a rare condition characterized by inflammation and fibrosis in the retroperitoneal space; most cases are idiopathic, but secondary causes include drugs, infections, autoimmune and inflammatory stimuli, and radiation. Patients may present with pain, and the major clinical sequelae of this condition are related to its involvement with structures in the retroperitoneum, including arteries (leading to acute and chronic renal failure) and ureters (leading to hydronephrosis, the swelling of a kidney due to a build-up of urine). Currently, there is no treatment available for this primary fibrosing disorder. In certain cancers, fibrosis is linked to TGF-β-integrin signaling [Rockey D C et al., N Engl J Med. (2015) 19; 372 (12): 1138-1149, citing Margadant C, Sonnenberg A. EMBO Rep (2010) 11: 97-105]. TGF-β affects integrin-mediated cell adhesion and migration by regulating the expression of integrins, their ligands and integrin-associated proteins. Conversely, several integrins directly control TGF-β activation. In addition, a number of integrins can interfere with both Smad-dependent and Smad-independent TGF-β signaling in different ways, including the regulation of the expression of TGF-β signaling pathway components, the physical association of integrins with TGF-β receptors and the modulation of downstream effectors. Reciprocal TGF-β-integrin signaling is implicated in normal physiology, as well as in a variety of pathological processes including systemic sclerosis, idiopathic pulmonary fibrosis, chronic obstructive pulmonary disease and cancer [Margadant C, Sonnenberg A. EMBO Rep. (2010) 11(2): 97-105)] In scleroderma, the prototypical fibrosing skin disease, skin fibroblasts and myofibroblasts are activated through the TGF-β-SMAD signaling pathway [Rockey D C et al., N Engl J Med. (2015) 372 (12): 1138-1149, citing Jinnin M. J Dermatol (2010) 37: 11-25]. Nephrogenic systemic fibrosis, a debilitating condition that is marked by widespread organ fibrosis, occurs in patients with renal insufficiency who have been exposed to gadolinium-based contrast material. Initial systemic inflammatory-response reactions and the reaction of gadolinium (Gd3+) ions with circulating proteins and heavy metals lead to the deposition of insoluble elements in tissue [Id., citing Swaminathan S, et al. N Engl J Med (2007) 357: 720-722]. Since no effective therapies have been identified, prevention is key (Id.). A recently recognized IgG4-related disease appears to involve autoimmune-driven inflammation that provokes fibrosis in multiple organs, including the pancreas, retroperitoneum, lung, kidney, liver, and aorta [Id., citing Umehara H, et al. Mod Rheumatol (2012) 22: 1-14].

Role of miRNAs in Pain Conditions

The analysis and validation of miRNAs in different tissue and pain conditions have been extensively reported [Tan-P-H et al. Acta Anaesthesiologica Taiwanica 51 (2013) 171-176]. Among the miRNAs dysregulated in the dorsal root ganglion (DRG), miR-21 expression is consistently shown to increase after multiple types of peripheral nerve injury [Id., citing Strickland, L T et al. PLoS One (2011) 6: e23423; Wu et al., Neuroscience (2011) 190: 386-397]. miR-124a has been shown to be involved in inflammatory nociception by regulation of relevant target proteins [Id., citing Kynast, K L, et al. Pain (2013) 154: 368-376]. miR-143 was shown to be expressed in nociceptive neurons; it has been suggested that miR-143 could selectively contribute to mRNA regulation in specific populations of nociceptors [Id., citing Tam, S. et al. Ell Tissue Res. (2011) 346: 163-167]. A functional study showed that miR-103 is downregulated in neuropathic animals and that intrathecal applications of miR-103 successfully relieve pain [Id., citing Favereaux, A. et al. EMBO J. (2011) 30: 3830-3841]. miRNA functions have also been investigated in animal models of chronic pelvic pain including of bladder pain syndrome (BPS) and irritable bowel syndrome (IBS). These studies indicate that miRNAs are involved in the onset and progression of neural sensitization and play an important role in inflammatory, neuropathic and visceral nociception. Therefore, these studies provided targets miRNAs for treatment of inflammatory, neuropathic, and visceral pain. Using cell-based models, 31 differentially expressed miRNAs were identified in bladder pain syndrome (BPS) patients and a direct correlation demonstrated between miR-449b, miR-500, miR-328, and miR-320 and a downregulation of NK1 receptor mRNA and/or protein levels [Id., citing Sanchez Freire, et al. (2010) Am. J. Pathol. 176: 288-303]. Defects in urothelial integrity resulting in leakage and activation of underlying sensory nerves are possible causative factors of bladder pain syndrome [Id.]. A possible link between miR-199a-5p expression and the control of urothelial permeability in bladder pain syndrome has been suggested [Id., citing Monastyrskaya, K. et al. Am. J. Pathol. (2013) 182: 431-448]. It has also been suggested that upregulation of miR-199a-5p and concomitant downregulation of its multiple targets might determine the impact of a tight urothelial barrier, leading to chronic bladder pain syndrome [Id., citing Monastyrskaya, K. et al. Am. J. Pathol. (2013) 182: 431-448]. In IBS patients with increased intestinal membrane permeability, increased expression of miR-29a was found in blood microvesicles, small bowel, and colon tissues. miR-29a has a complementary site in the 3′-UTRs of the glutamate-ammonia ligase gene that leads to decreased glutamine synthetase levels, increased intestinal permeability and chronic visceral pain in IBS patients. Suppressing the expression of miR-29a in vitro restored intestinal permeability [Id., citing Zhou, Q. et al. Gut (2010) 59: 775-784].

Has-miR-29a expression was reduced in lingual nerve neuromas pf patients with higher pain visual analogue scare (VAS) scores (painful group), compared with patients with lower pain VAS scores (non-painful group. A statistically significant negative correlation was observed between the expression of both hsa-miR-29a and hsa-miR-500a, and the pain VAS score, indicating that reduced levels of both of these miRNAs are associated with the presence of pain [Tavares-Ferreira, D. et al. Molecular Pain (2019) 15: 1-16].

### MSC EVs in Treatment of Organ Fibrosis

MSC-derived EVs have shown protective effects in several models of organ injury and fibrosis. In murine models of kidney injury, MSC-derived EVs protected against renal injury by reducing levels of creatinine, uric acid, lymphocyte response and fibrosis through shuttling miR-let7c to induce renal tubular cell proliferation [Kusuma G D, et al. Front Pharmacol. 2018; 9: 1199, citing Wang B, et al. Mol Ther. (2016) 24(7): 1290-1301]. In a murine model of carbon tetrachloride-induced hepatic injury, concurrent treatments of MSC-EVs attenuated the injury by increasing the proliferation, survival and prevented the apoptosis of hepatocytes [Id., citing Tan C Y, et al. Stem Cell Res Ther. (2014) 5 (3): 76]. In animal models of lung injury, MSC and hAEC-EVs have been shown to reduce pulmonary inflammation, improved lung tissue recovery and supported the proliferation of alveolar type II and bronchoalveolar stem cells [Id., citing Rubenfeld G D, et al. N Engl J Med. (2005) 353 (16): 1685-1693; Cruz F F, et al. Stem Cells Transl Med. (2015) 4 (11): 1302-1316; Monsel A, et al. Am J Respir Crit Care Med. (2015) 192 (3): 324-336; Tan J L, et al. Stem Cells Transl Med. (2018) 7 (2): 180-196]. In models of stroke, MSC-EVs delivery of miR-133b directly to neurite cells reportedly enhanced the outgrowth of neurites resulting in increased proliferation of neuroblasts and endothelial cells [Id., citing Xin H, et al. Stem Cells. (2013) 31 (12): 2737-2746]. Additionally, Anderson et al. showed through a comprehensive proteomic analysis that MSC-derived EVs mediated angiogenesis via NF-κB signaling [Anderson J D, et al. Stem Cells. (2016) 34 (3): 601-613], while Zhang et al [Stem Cells Transl Med. (2015) 4 (5): 513-522] showed that UC MSC-EVs mediated angiogenesis via the Wnt4/β-catenin pathway. However, EV composition is determined not only by the cell type but also by the physiological state of the producer cells. This diversity of mechanisms by which EVs are generated and confer effects provides both opportunities and challenges for developing EV-based therapeutics [György B, et al. Annu Rev Pharmacol Toxicol. (2015) 55: 439-464].

### Emerging Viruses

Coronaviruses (CoVs), a large family of single-stranded RNA viruses, can infect a wide variety of animals, causing respiratory, enteric, hepatic and neurological diseases [Yin, Y., Wunderink, RG, Respirology (2018) 23 (2): 130-137, citing Weiss, SR, Leibowitz, IL, Coronavirus pathogenesis. Adv. Virus Res. (2011) 81: 85-164]. Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma- and delta-groups; the beta group is further composed of A, B, C and D subgroups [Xia, S. et al. Sci. Adv. (2019) 5: eaav4580].

CoVs are enveloped with a non-segmented, positive sense, single strand RNA, with size ranging from 26,000 to 37,000 bases; this is the largest known genome among RNA viruses [Yang, Y. et al., J. Autoimmunity (2020) doi.org/10.1016/j.jaut.2020.102434, citing Weiss, S R et al. Microbiol. Mol. Biol. Rev. (2005) 69 (4): 635-664]. The viral RNA encodes structural proteins, and genes interspersed within the structural genes, some of which play important roles in viral pathogenesis [Yang, Y. et al., J. Autoimmunity (2020) doi.org/10.1016/j.jaut.2020.102434, citing Fehr, AR, Perlman, S. Methods Mol. Biol. (2015) 1282: 1-23; Zhao, L. et al. Cell Host Microbe (2012) 11 (6): 607-616]. The spike protein(S) is responsible for receptor binding and subsequent viral entry into host cells; it consists of S1 and S2 subunits. The membrane (M) and envelope (E) proteins play important roles in viral assembly; the E protein is required for pathogenesis [Id., citing DeDiego, M L, et al. J. Virol. (2007) 81 (4): 1701-1713; Nieto-Torres, J L et al. PLoS Pathog. (2014) 10 (5): e1004077]. The nucleocapsid (N) protein contains two domains, both of which can bind virus RNA genomes via different mechanisms and is necessary for RNA synthesis and packaging the encapsulated genome into virions Y. et [Yang, al., J. Autoimmunity (2020) doi.org/10.1016/j.jaut.2020.102434, citing Fehr, AR, Perlman, S. Methods Mol. Biol. (2015) 1282: 1-23; Song, Z. et al. Viruses (2019) 11(1): 59; Chang, C K et al., J. Biomed. Sci. (2006) 13(1): 59-72; Hurst, K R, et al. J. Virol. (2009) 83 (14): 7221-7234]. The N protein also is an antagonist of interferon and viral encoded repressor (VSR) of RNA interference (RNAi), which benefits viral replication [Id., citing Cui, L. et al. J. Virol. (2015) 89 (17): 9029-9043].

Before December 2019, six coronavirus species had been identified to infect humans and cause disease. Among them, infections caused by H-CoV-229E and HCoV-NL63 in the alpha group, HCoV-OC43 and HCoV-HKU1 in beta subgroup A are frequently mild, mostly causing common cold symptoms [Xu, X. et al. Eur. J. Nuclear Medicine & Molec. Imaging (2020) doi.org/10.1007/s00259-020-04735-9, citing Su, S. et al. Trends Microbiol. (2016) 24: 490-502]. The other two species, severe acute respiratory syndrome coronavirus (SARS-CoV) in beta subgroup B and Middle East respiratory syndrome coronavirus (MERS-CoV) in beta subgroup C, have a different pathogenicity and have caused fatal illness [Id., citing Cui, J. et al. Nat. Rev. Microbiol. (2019) 17: 181-192]. Human-to-human transmission of SARS-CoV and MERS-CoV occurs mainly through nosocomial transmission: from 43.5-100% of MERS patients in individual outbreaks were linked to hospitals, [Id., citing Hunter, I C et al. Transmission of Middle East respiratory syndrome coronavirus infections in healthcare settings, Abu Dhabi. Emerg. Infect. Dis. (2016) 22: 647-656. Osong Public Health Res. Perspect. (2015) 6: 269-78], which was similar in SARS patients [Anderson, R M et al. Philos. Trans. R. Soc. Lond. B. Biol. Sci. (2004) 359: 1091-1105]. Severe Acute Respiratory Syndrome Coronavirus 2 (SARS-CoV-2) is the seventh member of the coronaviruses that infects humans [Zhu, N. et al. N. Engl. J. Med. (2020) 382: 727-733].

The coronavirus-19 disease (COVID-19) pandemic caused by SARS-CoV-2 has exceeded 11 million cases worldwide and caused more than 500,000 deaths in 216 countries [Kuri-Cervantes, L. et al. Sci. Immunol. (2020) 10.1126/sciimmuol.abd7114]. Due to efficient person-to-person transmission, the SARS-CoV-2 pandemic is still evolving. The extent of the disease, its epidemiology, pathophysiology and clinical manifestations are being documented on an ongoing basis [Guan w. et al. N. Engl. J. Med. (2020) 382: 1708-1720; Yang, Y. et al., J. Autoimmunity (2020) doi.org/10.1016/j.jaut.2020.102434].

COVID-19 can present as an asymptomatic carrier state, acute respiratory disease, and pneumonia. Adults represent the population with the highest infection rate; however, neonates, children, and elderly patients can also be infected by SARS-CoV-2. In addition, nosocomial infection of hospitalized patients and healthcare workers, and viral transmission from asymptomatic carriers are possible. The most common finding on chest imaging among patients with pneumonia was ground-glass opacity with bilateral involvement.

The severity of COVID-19 can be roughly categorized into three groups based on the severity of the initial infection. Mild COVID-19, which, along with asymptomatic COVID-19 comprises the majority of cases, is characterized by symptoms such as fever, shortness of breath, gastrointestinal distress, malaise, headaches and a loss of taste and small. Severely ill patients require hospitalization for treatment of the infection, because of respiratory issues. Critical patients are a subset of the severely ill patients who experience respiratory failure that requires mechanical ventilation support. The percentages of patients vary, but mild patients are reported to be approximately 80%, severe cases are 14%, and critical cases are 6%. As many countries prioritize testing only for hospitalized patients, determining the exact percentages of patients in the general population is challenging [Disser, N P et al. J. Bone Joint Surg. Am. (2020) 102: 1197-1204]. Severe cases are more likely to be older patients with underlying comorbidities compared to mild cases. Indeed, age and disease severity may be correlated with the outcomes of COVID-19.

Currently, effective infection control intervention is the primary way to prevent the spread of SARS-CoV-2. As a result of Operation Warp Speed, two mRNA-based vaccines now have been approved for distribution in the United States, with more vaccines awaiting approval [Fortner, A. and Schumacher, D. Discoveries (Vraiova) (2021) 9(1): e122]. Parameters of the immunity conferred undoubtedly will be determined on an ongoing basis.

SARSCoV-2 uses the SARS-CoV receptor ACE2 to gain entry into host cells and the serine protease TMPRSS2 for S protein priming [Hoffman, M. et al. Cell (2020) 181 (2): 271-280]. One mechanism for SARS-CoV-2 entry occurs when the spike protein on the surface of SARS-CoV-2 binds to an ACE2 receptor followed by cleavage at two cut sites (“priming”) that causes a conformational change allowing for viral and host membrane fusion [Shrimp, J H et al. ACS Pharmacol. Trans. Sci. (2020) 3 (5): 997-1007]. Angiotensin converting enzyme 2 (ACE2) and dipeptidyl peptidase 4 (DPP4) are known host receptors for SARS-CoV and MERS-CoV respectively [Yang, Y. et al., J. Autoimmunity (2020) doi.org/10.1016/j.jaut.2020.102434, citing Kuhn, J H, et al. Cell Mol. Life Sci. (2004) 61 (21): 2738-2743; Raj, V S, et al. Nature (2013) 495 (7440): 251-254].

Although the respiratory system is a primary target of SARS-CoV-2, multiple nonpulmonary manifestations and complications of COVID-19 are being documented on an ongoing basis. For example, bioinformatics analysis of single-cell transcriptosome datasets of lung, esophagus, gastric, ileum and colon tissue reveal that the digestive system is also a potential route of entry for COVID-19; Cardiovascular complications are rapidly emerging as a key threat in COVID-19 [Varga, Z. et al. The Lancet (2020) doi.org/10.1016/S0140-6736 (20) 30937-5]. Endothelial cell involvement across vascular beds of different organs has been demonstrated in a series of patients with COVID-19 [Varga, Z. et al. The Lancet (2020) doi.org/10.1016/S0140-6736 (20) 30937-5]. Early studies also have indicated that there is considerable musculoskeletal dysfunction in some patients with COVID-19, although long-term follow-up studies have not yet been conducted [Disser, N P et al. J. Bone Joint Surg. Am. (2020) 102: 1197-1204; Lopez. M. et al. Am. J. Physical Med. & Rehab. 99 (8) 669-673]. CoVs, which are neuroinvasive and neurotropic, can also be neurovirulent, causing illnesses such as meningitis and encephalitis. In addition, brain tissue is reported to contain ACE2 receptors [Lopez. M. et al. Am. J. Physical Med. & Rehab. 99 (8) 669-673]. Thrombotic complications have been reported, including pulmonary embolism. Skin manifestations also have been documented [Lopez. M. et al. Am. J. Physical Med. & Rehab. 99 (8) 669-673].

Epidemiological evidence and experimental studies suggest that aging is the most important risk factor for chronic diseases. These data provided the foundation for the geroscience concept, which posits that the fundamental processes underlying aging also underlie, in part or in whole, the development and/or progression of many chronic diseases. Among these, chronic respiratory diseases impose an enormous global health burden, particularly in the elderly. Despite important advances in therapy for many of these diseases, there remains a major unmet need for the development of safe and effective disease-modifying treatments. Current drug treatments do not reduce disease progression or mortality in chronic obstructive pulmonary disease (COPD), which now affects about 12% of people older than 65 years. The newest treatments for patients with idiopathic pulmonary fibrosis (IPF) also have modest long-term clinical impact [W. Merkt, M. Bueno, A. L. Mora, D. Lagares, Senotherapeutics: Targeting senescence in idiopathic pulmonary fibrosis. Semin Cell Dev Biol. (2020) 101, 104-110]. Aging of the lung and airway are also involved in COPD and IPF as well as asthma, bronchiectasis, pulmonary hypertension, lung infections, and adult patients with cystic fibrosis [K. Ascher, S. J. Elliot, G. A. Rubio, M. K. Glassberg, Lung Diseases of the Elderly: Cellular Mechanisms. Clin Geriatr Med (2017) 33, 473-490; D. M. E. Bowdish, The Aging Lung: Is Lung Health Good Health for Older Adults? Chest (2019) 155, 391-400]. Increased understanding of aging pathways in chronic respiratory diseases will identify new therapeutic targets, novel disease-modifying therapies, and lead to improved survival.

The geroscience concept has stimulated investigations of the complex molecular pathways involved in aging and in chronic disease. For example, aging is associated with processes including macromolecular damage, impaired mitochondria! bioenergetics, accumulation of senescent cells and a low-level increase in pro-inflammatory mediators. This low-level chronic inflammation leads to down-regulation of components of the immune response and increased susceptibility to infection, as well as exacerbation of most, if not all, chronic diseases of aging, including, but not limited to, COPD, cardiovascular diseases, type 2 diabetes, cancers and chronic kidney disease. As such, inflammation has been recognized as one of the fundamental pillars of aging [D. Furman et al., Chronic inflammation in the etiology of disease across the life span. Nature Medicine (2019) 25, 1822-1832].

Exosomes target inflammation relevant to age-related lung diseases. MSC-derived exosomes suppress pro-inflammatory processes in a variety of experimental models of inflammatory lung diseases, including asthma, acute lung injury (ALI), acute respiratory distress syndrome (ARDS), and pneumonias. While the exact mechanisms of exosome action are under intense study, it is known that exosomes deliver cytokines, immunomodulatory proteins, mRNAs and miRNAs; they have been shown to activate autophagy and/or inhibit apoptosis, necrosis and oxidative stress in injured hepatocytes, neurons, retinal cells, lung, gut and renal epithelial cells [C. R. Harrell, N. Jovicic, V. Djonov, N. Arsenijevic, V. Volarevic, Mesenchymal Stem Cell-Derived Exosomes and Other Extracellular Vesicles as New Remedies in the Therapy of Inflammatory Diseases. (2019) Cells 8, 1605]. Specific anti-inflammatory cytokines, such as interleukin (IL)-18 binding protein, IL-13, neurotrophin 3 (NT-3) factor, ciliary neurotrophic factor (CNTF) and IL-10 have all been found in exosomes [F. J. Vizoso, N. Eiro, S. Cid, J. Schneider, R. Perez-Fernandez, Mesenchymal Stem Cell Secretome: Toward Cell-Free Therapeutic Strategies in Regenerative Medicine. (2017) Int J Mol Sci 18]. Therefore, it is not surprising that immunomodulation may be one of the effects of the MSC-secretome. Recently, freeze dried formulated MSC-secretome including exosomes were shown to inhibit pro-inflammatory interferon (IFN)-y release by phytohemagglutinin-activated PBMCs while increasing anti-inflammatory IL-10 release, with an efficacy equal to whole MSCs [E. Bari et al., Freeze-dried and GMP-compliant pharmaceuticals containing exosomes for acellular mesenchymal stromal cell immunomodulant therapy. Nanomedicine (2019) 14, 753-765].

Exosomes also effect resolution of inflammation in vitro [X.-D. Tang et al., Mesenchymal Stem Cell Microvesicles Attenuate Acute Lung Injury in Mice Partly Mediated by Ang-1 mRNA. (2017) STEM CELLS 35, 1849-1859], and attenuate oxidative stress, as demonstrated using exosomes derived from transfected placental cells (chorion and decidua) [S. Y. Kim et al., Placenta Stem/Stromal Cell-Derived Extracellular Vesicles for Potential Use in Lung Repair. (2019) PROTEOMICS 19, 1800166]. They also modulate or ameliorate inflammation and injury in a range of preclinical lung injury models [S. C. Abreu, D. J. Weiss, P. R. Rocco, Extracellular vesicles derived from mesenchymal stromal cells: a therapeutic option in respiratory diseases? Stem Cell Res Ther (2016) 7, 53; R. Y. Mahida, S. Matsumoto, M. A. Matthay, Extracellular Vesicles: A New Frontier for Research in Acute Respiratory Distress Syndrome. American Journal of Respiratory Cell and Molecular Biology (2020) 63, 15-24; J. Phelps, A. Sanati-Nezhad, M. Ungrin, N. A. Duncan, A. Sen, Bioprocessing of Mesenchymal Stem Cells and Their Derivatives: Toward Cell-Free Therapeutics. Stem Cells Int (2018) 2018, 9415367] including influenza, severe pneumonia and traumatic and acute lung injury [X.-D. Tang et al., Mesenchymal Stem Cell Microvesicles Attenuate Acute Lung Injury in Mice Partly Mediated by Ang-1 mRNA. (2017) STEM CELLS 35, 1849-1859, M. Khatri, L. A. Richardson, T. Meulia, Mesenchymal stem cell-derived extracellular vesicles attenuate influenza virus-induced acute lung injury in a pig model. Stem Cell Res Ther (2018) 9, 17; J. H. Lee, J. Park, J. W. Lee, Therapeutic use of mesenchymal stem cell-derived extracellular vesicles in acute lung injury. Transfusion (2019) 59, 876-883; A. Monsel et al., Therapeutic Effects of Human Mesenchymal Stem Cell-derived Microvesicles in Severe Pneumonia in Mice. American journal of respiratory and critical care medicine (2015) 192, 324-336; J. Park, S. Jeong, K. Park, K. Yang, S. Shin, Expression profile of microRNAs following bone marrow-derived mesenchymal stem cell treatment in lipopolysaccharide-induced acute lung injury. Exp Ther Med (2018) 15, 5495-5502].

The present disclosure will utilize exosome therapy to target age- and organ-specific inflammation while also monitoring mitochondria! dysfunction and immune aging [D. Furman et al., Chronic inflammation in the etiology of disease across the life span. Nature Medicine (2019) 25, 1822-1832, B. K. Kennedy et al., Geroscience: linking aging to chronic disease. Cell (2014) 159, 709-713]. We will develop and test a new cell-based exosome therapy approach for currently incurable age-related diseases, focusing on the lung, although potentially applicable to multiple organs. We will also specifically address sexual dimorphism related to age-related lung disease, in an attempt to provide a therapy with broad applicability.

### SUMMARY OF THE INVENTION

According to one aspect, the present disclosure provides a composition comprising a purified population of potent exosomes derived from extracellular vesicles derived from mesenchymal stem cells (MSCs), wherein the population of exosomes comprise an identity signature comprising expression of biomarkers CD9, CD63, and CD81; the population of exosomes comprise total protein of about 1 mg; the population of exosomes comprise total RNA content greater than 20 μg; and the population of exosomes comprise a cargo comprising one or more, miRNAs; wherein the miRNA cargo is tunable by exposure to heat, light, or both, and wherein lot to lot and batch to batch variability relative to prior lots of the composition is below a significance level of ≤0.05.

In some embodiments of the composition, the MSCs originate from a tissue or a body fluid of a mammalian subject. In some embodiments of the composition, the mammalian subject is a human subject.

In some embodiments of the composition, (a) the MSCs originate from placental tissue, adipose tissue, umbilical cord tissue, lung tissue, heart tissue, or dental pulp; or (b) the mesenchymal stem cells originate from bone marrow of normal healthy subjects aged 21-40 years old; or (c) the body fluid is blood, amniotic fluid or urine.

In some embodiments of the composition, the exosomes originate from mesenchymal cells obtained from adipose tissue (ASCs); or the exosomes derived from MSCs obtained from placental tissue originate from one or more of chorionic membrane (CM), chorionic trophoblast without villi (CT-V), chorionic villi (CV), or decidua (DC); or the blood is umbilical cord blood or peripheral blood.

In some embodiments of the composition, the miRNA cargo of the population of exosomes is tunable by exposure of the MSCs first to a heat shock followed by exposure to red light, near infrared light, UV light or a combination thereof or by exposure of the MSCs first to a heat shock, and isolation of the population of exosomes from the heat-shocked MSCs followed by exposure of the isolated heat-shocked exosomes to red light, near infrared light, UV light, or a combination thereof.

In some embodiments of the composition, the heat shock comprises exposure to a temperature of from about 41 °C to about 43 °C, inclusive; the red light is light of a wavelength from about 620 nanometers (nm) to about 750 nanometers, (nm), inclusive; the near infrared light is light of a wavelength from about 700 nm to about 1400 nm, inclusive; and the ultraviolet light is light of a wavelength from about 10 nm to about 400 nm, inclusive.

In some embodiments of the composition, the red light exposure is at a wavelength of 630 nm, the near infrared light exposure is at a wavelength of 850 nm, the near infrared light exposure is at a wavelength of 940 nm, or exposure is to a combination thereof.

In some embodiments of the composition, the population of exosomes derived from tuned MSCs, when compared to a non-heat-shocked and non-irradiated (native) control: comprise tuned exosomes containing unique miRNA species compared to the miRNA species in native exosomes; comprise one or more downregulated miRNA species selected from miR-21, miR-145, miR-23, and miR-31 compared to the miRNA species in native exosomes, comprise one or more upregulated miRNA species selected from miR-29, miR-196a, and miR-9a; or a combination thereof.

In some embodiments of the composition, an improvement in wound healing by the population of exosomes comprising the tuned miRNAs is by at least one of improved collagen production; improved cell proliferation; improved immune regulation; or improved angiogenesis, compared to a native control.

In some embodiments of the composition, the composition is a pharmaceutical composition comprising a therapeutic amount of the isolated tuned potent exosomes and a pharmaceutically acceptable carrier.

In some embodiments of the composition, the population of exosomes is derived from at least 1×1012 EVs comprising exosomes per isolation.

In some embodiment of the composition s, the pharmaceutical composition is formulated for administration by inhalation, topical administration, or intravenous administration.

In some embodiments of the composition, an effective amount of the purified, tuned potent exosomes comprises at least 1×10E8 exosomes.

In some embodiments of the composition, the population of exosomes is tuned to treat an age-related chronic disease, which if left untreated, comprises at least one of a progressive injury, progressive inflammation, progressive fibrosis of a tissue or a combination thereof.

In some embodiments of the composition, the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle.

In some embodiments of the composition, the age-related chronic disease is a chronic lung disease, chronic inflammation and immune dysfunction, mitochondrial dysfunction, organ transplantation dysfunction, organ resuscitation and rejuvenation, a viral infection, neuropathic pain; neurofibrosis, neurodegeneration, connective tissue dysfunction, musculoskeletal repair, dysfunction of the gut microbiome, or age-related decline.

In some embodiments of the composition, the chronic lung disease is a fibrotic lung disease.

In some embodiments of the composition, the chronic lung disease is due to chronic smoking or a severe viral infection.

In some embodiments of the composition, the severe lung infection is due to a severe coronavirus infection.

In some embodiments of the composition, the age-related chronic lung disease comprises reduced forced vital capacity compared to a normal healthy control.

According to another aspect, the present disclosure provides a method for treating a wound comprising, in order a) tuning an miRNA cargo derived from extracellular vesicles (EVs) that originate from a population of mesenchymal stem cells (MSCs) that originate from a tissue or a body fluid of a mammalian subject by:
(i) isolating the population of MSCs from the tissue or the body fluid of the human subject;
(ii) adapting the isolated population of MSCs in vitro to culture conditions at 37 °C to form a cultured population of MSCs at 37 °C;
(iii) heat shocking the cultured population of MSCs at 37 °C by exposing the cultured population of MSCs to a temperature of about 41 °C to about 43 °C, inclusive, to form a heat-shocked population of MSCs comprising a population of exosomes comprising a heat-shocked miRNA cargo; and
(iv) irradiating the heat-shocked population of MSCs of (iii); or isolating from the heat-shocked population of MSCs the population of exosomes comprising the heat shocked miRNA cargo and irradiating the isolated population of heat-shocked exosomes; wherein the irradiating comprises exposing the heat-shocked population of MSCs or the isolated population of heat-shocked exosomes to red light, near infrared light, UV light or a combination thereof; and
b) administering by inhalation, topically or parenterally the pharmaceutical composition comprising the population of heat-shocked and irradiated exosomes comprising a tuned cargo of miRNAs.

In some embodiments, the mammalian subject is a human subject.

In some embodiments of the method, the tissue comprises: (a) placental tissue, adipose tissue, umbilical cord tissue, lung tissue, heart tissue, or dental pulp; or (b) bone marrow of normal healthy subjects aged 21-40 years old.

In some embodiments of the method, the body fluid is blood, amniotic fluid or urine.

In some embodiments of the method, (i) the tissue is adipose tissue, (ii) the population of MSCs are a population of adipose-derived mesenchymal stem cells (ASCs); and (iii) the population of exosomes comprising the tuned cargo of miRNAs comprise one or more unique miRNA species compared to the miRNA species in native exosomes.

In some embodiments of the method, the tuned population of heat-shocked and red light irradiated exosomes comprise one or more upregulated miRNA species selected from miR-29, miR-196a, and miR-9a compared to the miRNA species in native exosomes; and comprise one or more downregulated miRNA species selected from miR-21, miR-145, miR-23, and miR-31 compared to the miRNA species in native exosomes.

In some embodiments of the method, the population of exosomes comprising the tuned cargo of miRNAs treats an age-related chronic disease, which if left untreated, comprises at least one of a progressive injury, progressive inflammation, progressive fibrosis of a tissue or a combination thereof.

In some embodiments of the method, the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle.

In some embodiments of the method, the age-related chronic disease is a chronic lung disease, chronic inflammation and immune dysfunction, mitochondrial dysfunction, organ transplantation dysfunction, organ resuscitation and rejuvenation, a viral infection, neuropathic pain; neurofibrosis, neurodegeneration, connective tissue dysfunction, musculoskeletal repair, dysfunction of the gut microbiome, or age-related decline.

In some embodiments of the method, the chronic lung disease is a fibrotic lung disease.

In some embodiments of the method, the chronic lung disease is due to chronic smoking or a severe viral infection.

In some embodiments of the method, the chronic lung disease comprises reduced forced vital capacity compared to a normal, healthy control.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, FIG. 1B, FIG. 1C, FIG. 1D, FIG. 1E, FIG. 1F, FIG. 1G, and FIG. 1H shows that old human adipose stem cells (ASCs) displayed increased mitochondrial activity compared to adult ASCs. Adult (39 years) (Row 1) and older (65 years) (Row 2) human male adipose derived stem cells (ASCs) were incubated with Dapi (FIG. 1A, FIG. 1E) or Mito Tracker Green (to show mitochondrial number, FIG. 1B, 1F) or Red (to show mitrochondrial activity, FIG. 1C, 1G). Localization and activity were merged using confocal microscopy. (FIG. 1D, FIG. 1H).

FIG. 2 shows that catalase protein expression decreases in old human female ASCs (lanes 4-6) compared to adult human female ASCs (lanes 1-3).

FIG. 3A, FIG. 3B, and FIG. 3C shows that modulation of antioxidant catalase activity in old and adult ASC directly determines their ability to ameliorate BLM-induced pulmonary fibrosis. FIG. 3A shows representative photomicrographs (4×, 20× and 40× magnification) of lung sections from mice infused with adult+catalase inhibitor (panels 4-6) or old transfected with catalase activator (panels 10-12) ASCs. Adult control ASCs (panels 1-3) or old-control ASCs (panels 7-9) transfected with control scrambled plasmids were also infused. Lung tissue sections were stained with Masson's-Trichrome. FIG. 3B shows collagen content (g/mg tissue) for adult control, adult+catalase inhibitor and adult+catalase activator and old control, old+catalase inhibitor and old+catalase activator. Adult+inhibitor increased lung hydroxyproline (collagen accumulation). FIG. 3C shows TNFα mRNA expression for adult control, adult+inhibitor, old control and old+activator. Old+activator reduced collagen and TNFα. n=5-8 mice/group, *P<0.05 compared to corresponding control transfected ASCs. Data in FIG. 3B, FIG. 3C were analyzed using one-way Mann Whitney test.

FIG. 4A and FIG. 4B shows that wound healing capacity of ASCs is age- and catalase dependent. At baseline adult ASCs promote wound healing and old ASCs inhibit wound healing of ex vivo human wounds. FIG. 4A is a graph representing quantification of wound healing using histomorphometric analyses 4 days after wounding. Catalase inhibitor reduced the capability of adult ASCs to promote wound closure, while catalase activation rescued ability of old ASCs to stimulate wound closure (n=3 biological replicates from 2-3 experimental ASC isolates/group). Data are graphed as mean±SEM % of vehicle **P<0.01, ***P<0.001 Data were analyzed using one-way Kruskal-Wallace and Mann-Whitney tests. FIG. 4B shows representative ex vivo wounds stained with hematoxylin and eosin. White arrows indicate wound edges after initial wounding, whereas red arrowheads point at the epithelialized edges of the migrating fronts 4 days after wounding; scale bar=200 μm.

FIG. 5A shows a transmission electron micrograph of isolated exosomes. Scale bar=50 nm. FIG. 5B shows a representative Western analysis confirming that the isolated exosomes express CD63 and HSP70.

FIG. 6 shows the biodistribution of human ASC exosomes. Shown are representative in vivo bioluminescence images to study the biodistribution of ExoGlow™ labeled adult and old exosomes in 16-month-old C57BL/6 mice. Time points are 5 min, 30 min, 60 min, 90 min, 2, 4, 6, and 24 hr. Inset shows PBS injected mice at 5 min, 6 hr and 24 hr. Intensity of luminescence seen in bar from lowest (red) to highest (yellow).

FIG. 7A and FIG. 7B shows a transmission electron micrograph revealing exosomes labeled with gold nanoparticles in alveolar type I epithelial cells (AEC I) and alveolar type II cells (AEC II) (red arrows).

FIG. 8A, FIG. 8B, FIG. 8C, FIG. 8D, FIG. 8E, and FIG. 8F shows that human adipose mesenchymal stem cell (ASC) exosomes administered 10 days after bleomycin-instillation reduce severity of pulmonary fibrosis and collagen content in old mice. Histological sections of lung tissue were stained with Masson's-Trichrome. Representative photomicrographs (40× magnification) of lung sections from (FIG. 8A) bleomycin-treated mice receiving plasmalyte vehicle or (FIG. 8B) adult ASC exosomes, (FIG. 8C) old ASC exosomes, and (FIG. 8D) ASCs. (FIG. 8E) shows the degree of pulmonary fibrosis on histological sections as measured by semi-quantitative Ashcroft score. (FIG. 8F) shows hydroxyproline (collagen content). Intratracheal bleomycin instillation increased lung collagen content as measured by hydroxyproline assays Data are graphed as mean±standard error of the mean (n=6-10/group) Data were analyzed using one-way Kruskal-Wallace and Mann-Whitney tests. * P<0.05: “P<0.01.

FIG. 9 shows that adult ASC exosomes increase SPC-1 cells (alveolar type 2 cells, yellow color) in lungs isolated from 18-month-old male mice with bleomycin-induced lung injury. Yellow color: SPC-1 positive cells; purple color: Dapi.

FIG. 10 shows that ASC exosomes, similar to ASC whole cells, promote wound closure in human ex vivo wound model. Gross photos show visual signs of wound closure and correspond to histology assessments. White dashed line indicates initial wound edge; white arrowhead indicates initial wounding edge in the H&E stained sections; red arrowhead is pointing to epithelial tongue location at day 4 post-wounding.

FIG. 11A and FIG. 11B shows bleomycin-induced lung injury evidenced by micro computed tomography (μCT) 7 days post-instillation. (FIG. 11A) shows representative pCT transverse and coronal lung sections acquired from old (22-month) male C57BL/6 mice at baseline (left) and 7 days following intratracheal bleomycin (BLM, 2.0 units/kg) administration (right) demonstrating increased lung density and loss of airspaces. (FIG. 11B) shows that saline treatment did not result in evidence of lung injury on pCT scan at baseline (left) or 7 days post-instillation (right). For both (FIGS. 11A and 11B), transverse (top panels) and frontal (Bottom panels) CT views are shown.

FIG. 12 shows that lung punches (shown in tissue culture dish) have normal histology (Trichrome staining 10×mag) and structure by EM (500 mag). AEC=alveolar epithelial cell.

FIG. 13 shows that lung matrix metalloproteinase-2 (MMP-2) activity is decreased in fibrotic bleomycin (BLM) lung punches only after treatment with adult exosomes. Representative ezymography is shown. M=marker. Data are mean±SD, *P<0.05 analyzed by student T test. n=3/group.

FIG. 14 is a schematic depiction of a smoking protocol whereby 14 month old male and female mice will be exposed to 6 months of mainstream and sidestream cigarette smoke (CS).

FIG. 15 shows an experimental scheme for human 3D lung punch experiments.

FIGS. 16A, FIG. 16B, FIG. 16C, and FIG. 16D shows that activated fibrotic pathways are induced by myofibroblast-derived exosomes from patients with fibrotic lung disease. Lung punches were injected with media, myofibroblast-derived exosomes from patients with lung disease or age-matched fibroblast-derived exosomes from control subjects (without lung disease). Punches were collected 4 days later. The diseased exosomes transferred the diseased phenotype characterized by increased integrin (FIG. 16A), collagen type I mRNA (FIG. 16B), increased ERα (FIG. 16C) and decreased Cav-1 (FIG. 16D) protein. *P<0.05 compared to media, **P<0.01, compared to control exosomes. P values calculated by Mann Whitney test; each point represents an individual punch, n=3 individual exosome/group.

FIG. 17 depicts the “four core genotypes” (FCG) mouse model, which has emerged as a major model testing if sex differences in phenotypes are caused by sex chromosome complement (XX vs. XY) or gonadal hormones or both. The model involves deletion of the testis-determining gene Sry from the Y chromosome and insertion of an Sry transgene onto an autosome [Itoh, Y. et al. BMC Res. Notes (2015) 8: 69]. It produces XX and XY mice with testes, and XX and XY mice with ovaries. Because the sex of the gonads is no longer based on sex chromosome complement (XX vs. XY), the FCG model produces XX and XY gonadal males (XXM, XYM) and XX and XY gonadal females (XXF, XYF).

FIG. 18A, FIG. 18B, and FIG. 18C shows characterization of exosome products processed from three daily conditioned media harvests. Exosome products were subjected to the nanosight nanoparticle analysis to record the mean particle size (FIG. 18A), and the sample's particle number concentration (FIG. 18B). The particle number concentration was then multiplied by the total volume of the sample to calculate the total particle yield. (FIG. 18C).

FIG. 19A, FIG. 19B, and FIG. 19C shows flow cytometry characterization of exosome products. (FIG. 19A) shows that CD63 magnetic selection beads enabled the flow cytometry detection of exosomes from a representative product. (FIG. 19B) shows gating based on the PE and PC5.5 isotype controls. (FIG. 19C) shows sample analysis revealing positive expression of CD63, CD9 and negative expression of CD105.

FIG. 20 shows bioreactor reservoir medium bottle tops sterilized prior to use, with foil loosely covering the inflow and outflow tube ends to maintain sterility during handling and assembly of the bioreactor.

FIG. 21 shows a fully assembled bioreactor in a two-cartridge configuration, wherein the second cartridge is obscured from view by the circulating pump.

FIG. 22A and FIG. 22B shows Zeta View® Nanoparticle Tracking Analysis (NTA) of exosomes derived from adipose-derived stem cells (ASCs) indicating a particle diameter of 121.9 nm and a mode diameter of 97.6 nm, with a concentration of 1.3×10E+11 particles/mL. FIG. 22A shows the particle size distribution based on the relative number of particles detected at each size. FIG. 22B shows the particle size distribution weighted by volume.

FIG. 23 is a representative microscopy image showing the transfer of DiD-stained ASC-derived exosomes to human primary dermal fibroblasts (DFs) in culture. The exosomes stained with DiD (a far-red fluorescent carbocyanine dye) were incubated with DFs for 24 hours at 37 °C, followed by nuclear staining with Hoechst to enumerate the cells. The image was collected on an ArrayScan® XTI high-content analysis system.

FIG. 24 is an image of a western blot and quantified western blot data demonstrating increased HSP70 protein content in exosomes isolated from a heat-shocked (42 °C) bioreactor compared to a control that was maintained at 37 °C. Actin was used as an internal control. Band intensities were measured using a ChemiDoc™ XRS+ System.

FIG. 25 is a bar graph demonstrating particle size as determined by nanoparticle tracking analysis (NTA) of ASC-derived exosomes treated with heat shock (HS)+200 ng/ml platelet derived growth factor-BB (PDGF-BB) compared to untreated ASCs. The NTA analysis shows a mean particle size of 163.6±25.4 nm and a mode particle size of 116.9±11.5 nm.

FIG. 26 is a bar graph demonstrating tetraspanin distribution on ASC-derived exosomes treated with heat shock (HS)+200 ng/mL platelet derived growth factor-BB (PDGF-BB) compared to untreated ASCs. CD9 and CD63 abundance was observed in EVs isolated from HS+200 ng/mL PDGF-BB treated cells, whereas a reduction in CD81 was observed in EVs isolated from HS+200 ng/mL PDGF-BB treated cells compared to untreated control cells.

FIG. 27A, FIG. 27B, and FIG. 27C are bar graphs demonstrating surface antigen expression of CD9, CD63, and CD81 on exosomes derived from adipose-derived mesenchymal stem cells (MSCs) isolated from subcutaneous adipose tissue, as analyzed using the MACSPlex™ Human Exosome Kit. Expression of CD9, CD63, and CD81 was differentially modulated across treatment groups. FIG. 27A is a bar graph demonstrating CD9 expression on exosomes from adipose-derived MSCs treated with DMEM/F12, 5% human platelet lysate (HPL), heat shock, 10 μM β-lapachone, 50 ng/mL TNF-α, or 100 ng/mL epidermal growth factor (EGF). FIG. 27B is a bar graph demonstrating CD63 expression on exosomes from adipose-derived MSCs treated with DMEM/F12, 5% HPL, heat shock, 10 μM β-lapachone, 50 ng/mL TNF-α, or 100 ng/mL EGF. FIG. 27C is a bar graph demonstrating CD81 expression on exosomes from adipose-derived MSCs treated with DMEM/F12, 5% HPL, heat shock, 10 μM β-lapachone, 50 ng/mL TNF-α, or 100 ng/mL EGF.

FIG. 28 shows tunable resistive pulse sensing (TRPS) analysis of the size distribution of exosomes isolated from human ASCs in a representative sample using a NP150 nanopore membrane at a 47 mm stretch, demonstrating a mean particle diameter of 144.4 nm, with a large population of particles between 100 and 120 nm. The inset is a scanning electron microscopy (SEM) image demonstrating that the exosome preparations contain discrete particles.

FIG. 29 shows a three-dimensional graph depicting the miRNA profile of exosomes isolated from human ASCs, as analyzed using the MACSPlex® miRNA kit, across four different cell types: (1) MSCs; (2) adipose cells; (3) astrocytes; and (4) neurons, revealing differential miRNA expression among the cell types.

FIG. 30 is a schematic depiction of a method for keratinocyte exposure to UVB light and treatment with extracellular vesicles (EVs) isolated from human ASCs. Keratinocytes are pretreated with EVs and subsequently exposed to 40 mJ/cm² of UVB. Culture media is harvested at 6 hours and 24 hours post-exposure for cytokine ELISA analysis, high-content imaging, and DNA damage analysis.

FIG. 31 is a bar graph demonstrating interleukin-8 (IL-8) concentration in culture media harvested 6 hours after keratinocyte exposure to UVB and treatment with EVs isolated from human ASCs at the indicated concentrations.

FIG. 32A and FIG. 32B are bar graphs demonstrating interleukin-8 (IL-8) and tumor necrosis factor-alpha (TNF-α) concentrations in culture media harvested 24 hours after keratinocyte exposure to UVB and treatment with EVs isolated from human ASCs at the indicated concentrations. FIG. 32A is a bar graph demonstrating IL-8 concentration in culture media harvested 24 hours after keratinocyte exposure to UVB and treatment with EVs at the indicated concentrations. FIG. 32B is a bar graph demonstrating TNF-α concentration in culture media harvested 24 hours after keratinocyte exposure to UVB and treatment with EVs at the indicated concentrations.

FIG. 33A, FIG. 33B, FIG. 33C, FIG. 33D, FIG. 33E, and FIG. 33F show detection of DNA damage in human keratinocytes following UVB irradiation. Human keratinocytes were cultured to confluence and exposed to 40 mJ/cm² UVB, with unexposed cells serving as a control. Following exposure, cells were fixed and immunostained for DNA double-strand breaks using a phospho-γ-H2AX antibody and Hoechst nuclear stain. FIG. 33A shows the nuclei of cells exposed to UVB. FIG. 33B shows the DNA damage of cells exposed to UVB. FIG. 33C shows the merged image of nuclei (blue) and DNA damage (green) of cells exposed to UVB. FIG. 33D shows the nuclei of unexposed control cells. FIG. 33E shows the DNA damage of unexposed control cells. FIG. 33F shows the merged image of nuclei (blue) and DNA damage (green) of unexposed control cells, demonstrating markedly fewer cells exhibiting DNA damage compared to UVB-exposed cells.

FIG. 34A, FIG. 34B, FIG. 34C, FIG. 34D, FIG. 34E, FIG. 34F, FIG. 34G, FIG. 34H, and FIG. 34I show immunofluorescent imaging analysis and quantification of human keratinocytes dosed with 1 J/cm² UV light. Cells were monitored for toxicity and ROS induction after 4 hours of recovery using high content image analysis (HCA). FIG. 34A shows nuclei labeled with Hoechst stain in keratinocytes that were not exposed to UV irradiation. FIG. 34B shows compromised membrane integrity labeled with ImageIT™ Dead Stain in keratinocytes that were not exposed to UV irradiation. FIG. 34C shows ROS activity labeled with CellROX™ dye in keratinocytes that were not exposed to UV irradiation. FIG. 34D shows the merged image of nuclei (blue), compromised membrane integrity (green), and ROS activity (red) in keratinocytes that were not exposed to UV irradiation. FIG. 34E shows nuclei labeled with Hoechst stain in keratinocytes that were exposed to UV irradiation. FIG. 34F shows compromised membrane integrity labeled with ImageIT™ Dead Stain in keratinocytes that were exposed to UV irradiation. FIG. 34G shows ROS activity labeled with CellROX™ dye in keratinocytes that were exposed to UV irradiation. FIG. 34H shows the merged image of nuclei (blue), compromised membrane integrity (green), and ROS activity (red) in keratinocytes that were exposed to UV irradiation. FIG. 34I is a bar graph demonstrating the quantification of the immunofluorescent images shown in FIG. 34A, FIG. 34B, FIG. 34C, FIG. 34D, FIG. 34E, FIG. 34F, FIG. 34G and FIG. 34H, indicating robust induction of ROS activity after UV exposure. 25 images per condition were analyzed and total intensity per cell was quantified.

### DETAILED DESCRIPTION OF THE INVENTION

### Definitions

As used herein, the term “about” means plus or minus 20% of the numerical value of the number with which it is being used. Therefore, about 50% means in the range of 40%-60%.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “adaptive immunity” as used herein refers to a specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

The term “adipose stem cell,” “adipose-derived stem cell,” or “ASC” as used herein refers to pluripotent stem cells, mesenchymal stem cells, and more committed adipose progenitors and stroma obtained from adipose tissue.

“Administering” when used in conjunction with a therapeutic means to give or apply a therapeutic directly into or onto a target organ, tissue or cell, or to administer a therapeutic to a subject, whereby the therapeutic positively impacts the organ, tissue, cell, or subject to which it is targeted. Thus, as used herein, the term “administering”, when used in conjunction with EVs or compositions thereof, can include, but is not limited to, providing EVs into or onto the target organ, tissue or cell; or providing EVs systemically to a patient by, e.g., intravenous injection, whereby the therapeutic reaches the target organ, tissue or cell. “Administering” may be accomplished by parenteral, oral or topical administration, by inhalation, or by such methods in combination with other known techniques.

The term “alveolus” or “alveoli” as used herein refers to an anatomical structure that has the form of a hollow cavity. Found in the lung, the pulmonary alveoli are spherical outcroppings of the respiratory sites of gas exchange with the blood. The alveoli contain some collagen and elastic fibers. Elastic fibers allow the alveoli to stretch as they fill with air when breathing in. They then spring back during breathing out, in order to expel the carbon dioxide-rich air.

The term “amniotic stem cells” as used herein refers to pluripotent stem cells, multipotent stem cells, and progenitor cells derived from amniotic membrane, which can give rise to a limited number of cell types in vitro and/or in vivo under an appropriate condition, and expressly includes both amniotic epithelial cells and amniotic stromal cells.

The term “angiotensin II” or “Ang-2” as used herein refers to a vasoactive octapeptide produced by the action of angiotensin-converting enzyme on angiotensin 1; it produces stimulation of vascular smooth muscle, promotes aldosterone production, and stimulates the sympathetic nervous system.

The terms “animal,” “patient,” and “subject” as used herein include, but are not limited to, humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals.

Antigen presenting cells. T cells recognize antigens on the surface of other cells and mediate their functions by interacting with, and altering, the behavior of antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products and carry them via the lymph to local lymph nodes or gut associated lymphoid organs. The encounter with a pathogen induces the dendritic cell to mature from an antigen-capturing cell to an antigen-presenting cell (APC) that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated. (“Chapter 24: The adaptive immune system,” Molecular Biology of the Cell, Alberts, B. et al., Garland Science, NY, 2002).

The term “antioxidant” as used herein refers to any substance that can prevent, reduce, or repair the ROS-induced damage of a target biomolecule. In ROS biology and medicine, the target molecules usually include proteins, lipids, and nucleic acids, among others. There are three major modes of action for antioxidants: (i) antioxidants that directly scavenge ROS already formed; (ii) antioxidants that inhibit the formation of ROS from their cellular sources; and (iii) antioxidants that remove or repair the damage or modifications caused by ROS [Li, R., et al. React. Oxyg. Species (Apex) (2016): 1 (1): 9-21].

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligomerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway [Loberg, R D, et al., J. Biol. Chem. 277 (44): 41667-673 (2002)]. It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “aquaporins” as used herein refers to water-specific membrane channel proteins. Aquaporin 5 (AQP5) is found in airway epithelial cells, type I alveolar epithelial cells and submucosal gland acinar cells in the lungs where it plays a key role in water transport [Hansel, N N et al. PLoS One (2010) doi.10.1371/journal.pone.0014226, citing Verkman, A S et al. Am. J. Physiol. Lung Cell Mol. Physiol. 278 (5): L867-79]. Decreased expression of human AQP5 has been associated with mucus overproduction in the airways of subjects with COPD and lower lung function [Id., citing Wang, K. et al. Acta Pharmacol. Sin. (2007) 28 (8): 1166-74]. Furthermore, smoking has been shown to attenuate the expression of AQP5 in submucosal glands of subjects with COPD [Id., citing Id, citing Wang, K. et al. Acta Pharmacol. Sin. (2007) 28 (8): 1166-74].

The term “Argonaute 2” or “AGO2” as used herein refers to an RNA binding protein that can shuttle between the cytoplasm and nucleus in a context-dependent fashion [Sharma, N R et al. J. Biol. Chem. (2016) 291: 2302-9] and is a key effector of RNA-silencing pathways. It is a major component of the RNA-induced silencing complex RISC).

The term “Ashcroft scale for the evaluation of bleomycin-induced lung fibrosis” refers to the analysis of stained histological samples by visual assessment. A modified Ashcroft scale precisely defines the assignment of grades from 0 to 8 for the increasing extent of fibrosis in lung histological samples [Hubner, R-H et al. Biotechniques (2008) 44 (4): 507-11].

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bleomycin-induced pulmonary fibrosis model” as used herein refers to an animal model of pulmonary fibrosis in rodents. It causes inflammatory and fibrotic reactions within a short period of time, even more so after intratracheal instillation. The initial elevation of pro-inflammatory cytokines (interleukin-1, tumor necrosis factor-α, interleukin-6, interferon-γ) is followed by increased expression of pro-fibrotic markers (transforming growth factor-β1, fibronectin, procollagen-1), with a peak around day 14 [Moeller, A. et al. Int. J. Biochem. Cell Biol. (2008) 40 (3): 362-82]. The “switch” between inflammation and fibrosis appears to occur around day 9 after bleomycin [Id., citing Chaudhary, N I et al. Am. J. Respir. Crit. Care Med. (2006) 173 (7): 769-76]. While the bleomycin model has the advantage that it is quite easy to perform, widely accessible and reproducible, and therefore fulfills important criteria expected from a good animal model. The bleomycin model has significant limitations in regard to understanding the progressive nature of human IPF. While bleomycin causes an inflammatory response, triggered by overproduction of free radicals, with induction of pro-inflammatory cytokines and activation of macrophages and neutrophils, thus resembling acute lung injury in some way, the subsequent development of fibrosis, however, is at least partially reversible, independent from any intervention [Izbicki, G et al. Intl J. Exp. Pathol. (2002) 83 (3): 111-19], and the aspect of slow and irreversible progression of IPF in patients is not reproduced in the bleomycin model [Chua, F. et al. Am. J. Respir. Cell Mol. Biol. (2005) 33 (1): 9-13].

The term “bronchoalveolar lavage” (BAL) is used herein to refer to a medical procedure in which a bronchoscope is passed through the mouth or nose into the lungs and fluid is squirted into a small part of the lung and then collected for examination. “Bronchoalveolar lavage fluid” (BALF) is used herein to refer to the fluid collected from a BAL procedure. Bronchoalveolar lavage (BAL), performed during fiberoptic bronchoscopy is a useful adjunct to lung biopsy in the diagnosis of nonneoplastic lung diseases. BAL is able to provide cells and solutes from the lower respiratory tract and may provide important information about diagnosis and yield insights into immunologic, inflammatory, and infectious processes taking place at the alveolar level. BAL has been helpful in elucidating the key immune effector cells driving the inflammatory response in IPF [Costabel and Guzman Curr Opin Pulm Med, 7 (2001), pp. 255-261]. Increase in polymorphonuclear leukocytes, neutrophil products, eosinophils, eosinophil products, activated alveolar macrophages, alveolar macrophage products, cytokines, chemokines, growth factors for fibroblasts, and immune complexes have been noted in BAL of patients with IPF [Id.].

The term “cargo” as used herein refers to a load or that which is conveyed. With respect to exosomes and/or extracellular vesicles, the term cargo refers to a substance encapsulated in the exosome and/or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or extracellular vesicle.

The term “cargo profile” as used herein refers to measurements of cargo components that characterize a population of extracellular vesicles

The term “caveolins (Cavs)” as used herein refers to integrated plasma membrane proteins that are complex signaling regulators with numerous partners and whose activity is highly dependent on cellular context [Boscher, C, Nabi, IR. Adv. Exp. Med. Biol. (2012) 729: 29-50]. Cavs are both positive and negative regulators of cell signaling in and/or out of caveolae, invaginated lipid raft domains whose formation is caveolin expression dependent. Caveolins and rafts have been implicated in membrane compartmentalization; proteins and lipids accumulate in these membrane microdomains where they transmit fast, amplified and specific signaling cascades. The term “caveolin 1 (CAV1)”, refers to a scaffolding protein that links integrin subunits to the tyrosine kinase FYN, an initiating step in coupling integrins to the Ras-ERK pathway and promoting cell cycle progression.

The term “chorion” as used herein refers to the outer fetal membrane that surrounds the amnion, the embryo, and other membranes and entities in the womb. A spongy layer of loosely arranged collagen fibers separates the amniotic and chorionic mesoderm. The chorionic membrane consists of mesodermal and trophoblastic regions. Chorionic and amniotic mesoderm are similar in composition. A large and incomplete basal lamina separates the chorionic mesoderm from the extravillous trophoblast cells. The latter, similar to trophoblast cells present in the basal plate, are dispersed within the fibrinoid layer and express immunohistochemical markers of proliferation. The Langhans fibrinoid layer usually increases during pregnancy and is composed of two different types of fibrinoid: a matrix type on the inner side (more compact) and a fibrin type on the outer side (more reticulate). At the edge of the placenta and in the basal plate, the trophoblast interdigitates extensively with the decidua [Cunningham, F. et al., The placenta and fetal membranes, Williams Obstetrics, 20th ed. Appleton and Lange, 1997, 95-125; Benirschke, K. and Kaufmann, P. Pathology of the human placenta. New York, Springer-Verlag, 2000, 42-46, 116, 281-297]. The chorion, which interfaces maternal tissues, consists of four layers. These are, from within outward: (F) the cellular layer, a thin layer consisting of an interlacing fibroblast network, which is frequently imperfect or completely absent; (G) a reticular layer, which consists of a reticular network, the fibers of which tend to be parallel, along with a few fibroblasts and many Hofbauer cells; (H) a pseudo-basement membrane, which is a layer of dense connective tissue firmly adherent to the reticular layer above, and which sends anchoring and branching fibers down into the trophoblast; and (I) a trophoblast layer, which is the deepest layer of the chorion consisting of from two to 10 layers of trophoblast cells in contact, on their deeper aspect, with maternal decidua. This layer contains the chorionic villi [Bourne, GL, Am. J. Obstet. & Gynec. (1960) 79 (6): 1070-1073].

The term “chromophore” as used herein refers to a molecule or section of a larger compound that absorbs and reflects specific electromagnetic radiation. Any visible light that is reflected by the molecule is observed as color. Radiation that is absorbed can activate ground state electrons in the chromophore into its excited state (if the energy is sufficient), which can induce conformational changes via electronic transitions.

“Cluster of Differentiation” or “cluster of designation” (CD) molecules are utilized in cell sorting using various methods, including flow cytometry. Cell populations usually are defined using a “+” or a “−” symbol to indicate whether a certain cell fraction expresses or lacks a particular CD molecule.

The term “CD9” as used herein refers to a member of the tetraspanin protein family whose crystal structure shows a reversed cone-like molecular shape, which generates membrane curvature in the crystalline lipid layers [Umeda, R. et al. Nature Communic. (2020) 11: article 1606].

The term “CD29” as used herein refers to integrin β1.

The term “CD37” as used herein refers to a member of the tetraspanin protein family exclusively expressed on immune cells [Zuidscherwoude, M. et al. Scientific Reports (2015) 5: 12201].

The term “CD44” as used herein refers to a cell adhesion molecule (HCAM) found on monocytes, neutrophils, fibroblasts and memory T cells, which is involved in lymphocyte homing.

The term “CD63” as used herein refers to a member of the tetraspanin protein family, the C-terminal domain of which interacts with several subunits of adaptor protein (AP) complexes, linking the traffic of this tetraspanin to clathrin-dependent pathways [Andreu, Z. & Yanez-Mo, M., citing Rous, B A et al. Mol. Biol. Cell (2002) 13 (3): 1071-1082]. Among intracellular interacting proteins, CD63 was shown to directly bind to syntenin-1, a double PDZ domain-containing protein [Id., citing Latysheva, N. et al. Mol. Cell Biol. (2006) 26 (20): 7707-7718]. A major role in exosome biogenesis has been reported for Syntenin-1 [Id., citing Baietti, M F et al. Nat. Cell Biol. (2012) 14 (7): 677-685].

The term “CD81” as used herein refers to a member of the tetraspanin protein family whose crystal structure shows a reversed teepee-like arrangement of the four transmembrane I helices, which create a central pocket in the intramembranous region that appears to bind cholesterol in the central cavity [Zimmerman, B. et al. Cell (2016) 167: 1041-1051]. During development, CD81 regulates the trafficking of CD19, an essential co-stimulatory molecule of lymphoid B cells and a well-characterized CD81 partner, along the secretory pathway [Shoham, T. et al. J. Imunol. (2003) 171: 4062-4072]. CD9 and CD81 have been shown to regulate several cell-cell fusion processes [Charrin, S. et al. J. Cell Science (2014) 127: 3641-3648].

The term “CD82” as used herein refers to a member of the tetraspanin protein family that has been implicated in the regulation of protein sorting into EVs and in antigen presentation by antigen presenting cells [Andreu, Z. and Yanez-Mo, M. Front. Immunol. (2014) doi.org/10.3389/fimmu.2014.00442].

The term “CD105” refers to endoglin, a cell membrane glycoprotein and part of the transforming growth factor-β receptor complex, which plays a role in angiogenesis.

The term “cell reprogramming” as used herein refers to a process by which transcription factors inducing cells to revert to an earlier stage of development. It includes reverting mature differentiated cells into immature stem or progenitor cells and then differentiating those stem or progenitor cells; a process of converting somatic cells into other cell types without the need for an intermediate pluripotent state (direct cell reprogramming); or direct conversion of one differentiated cell type into another, also known as transdifferentiation [Wilmut, I. et al. Philos. Trans. R. Soc. London B. Biol. Sci. (2011) 366 (1575): 2183-97].

The term “cellular senescence” as used herein refers to a process that results from a variety of stresses and leads to a state of irreversible growth arrest. A variety of cell-intrinsic and -extrinsic stresses can activate the cellular senescence program. These stressors engage various cellular signaling cascades but ultimately activate p53, p16Ink4a, or both. Cells exposed to mild damage that can be successfully repaired may resume normal cell-cycle progression. On the other hand, cells exposed to moderate stress that is chronic in nature or that leaves permanent damage may resume proliferation through reliance on stress support pathways (green arrows). This phenomenon (termed “assisted cycling”) is enabled by p53-mediated activation of p21. Thus, the p53-p21 pathway can either antagonize or synergize with p16Ink4a in senescence depending on the type and level of stress. Cells undergoing senescence induce an inflammatory transcriptome regardless of the senescence inducing stress [van Deursen, J M. Nature (2014) 509 (7501): 439-446]. Senescent cells accumulate in tissues and organs with age [Id., citing Lawless, C. Exp. Gerontol. (2010) 45: 772-778; Krishnamurthy, J. et al Nature (2006) 443: 453-457; Jeyapalan, J C et al. Mech Ageing Dev. (2007) 128: 36-44] as well as at sites of tissue injury and remodeling [Id., citing Rajagopalan, S and Long, E O. Proc. Natl. Acad. Sci. USA (2012) 109: 20596-20601; Munoz-Espin, D. et al. Cell (2013) 155: 1104-1118; Storer, M. et al. Cell (2013) 155: 1119-1130; Jun, J I and Lau, LF. Nature Cell Biol. (2010) 12: 676-685; Krizhanovsky, V. et al. Cell (2008) 134: 657-667].

The term “Chronic Obstructive Pulmonary Disease” as used herein refers to a lung disease that causes obstructed airflow in the lungs and results in breathing problems. It is used to describe such lung diseases as emphysema, chronic bronchitis, and severe asthma. COPD affects 9-10% of the adult population in the United States; it is the third leading cause of death and the 12th leading cause of morbidity. By 2030, it is expected to be the 4th leading cause of death worldwide, representing over 4.5 million deaths annually.

The term “contact” and its various grammatical forms as used herein refers to a state or condition of touching or of immediate or local proximity.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

As used herein, the term “derived from” is meant to encompass any method for receiving, obtaining, or modifying something from a source of origin.

As used herein, the terms “detecting”, “determining”, and their other grammatical forms, are used to refer to methods performed for the identification or quantification of a biomarker, such as, for example, the presence or level of miRNA, or for the presence or absence of a condition in a biological sample. The amount of biomarker expression or activity detected in the sample can be none or below the level of detection of the assay or method.

The terms “disease” or “disorder” as used herein refer to an impairment of health or a condition of abnormal functioning. The term “fibrotic disease” as used herein refers to a condition marked by an increase of interstitial fibrous tissue. The terms “lung tissue disease” or “lung disease” as used herein refers to a disease that affects the structure of the lung tissue, for example, without limitation, pulmonary interstitium. Scarring or inflammation of lung tissue makes the lungs unable to expand fully (“restrictive lung disease”). It also makes the lungs less capable of taking up oxygen (oxygenation) and releasing carbon dioxide. Examples of lung tissue diseases include, but are not limited to, idiopathic pulmonary fibrosis (IPF), acute lung injury (ALI), radiation-induced fibrosis in the lung, a fibrotic condition associated with lung transplantation, and sarcoidosis, a disease in which swelling (inflammation) occurs in the lymph nodes, lungs, liver, eyes, skin, or other tissues. According to some embodiments, pulmonary fibrosis is due to acute lung injury caused by viral infection, including, without limitation, influenza, SARS-CoV, MERS, COVID-19, and other emerging respiratory viruses.

The term “dispersion”, as used herein, refers to a two-phase system, in which one phase is distributed as droplets in the second, or continuous phase. In these systems, the dispersed phase frequently is referred to as the discontinuous or internal phase, and the continuous phase is called the external phase and comprises a continuous process medium. For example, in course dispersions, the particle size is 0.5 μm. In colloidal dispersions, size of the dispersed particle is in the range of approximately 1 nm to 0.5 μm. A molecular dispersion is a dispersion in which the dispersed phase consists of individual molecules; if the molecules are less than colloidal size, the result is a true solution.

The term “dry powder inhaler” or “DPI” as used herein refers to a device similar to a metered-dose inhaler, but where the drug is in powder form. The patient exhales out a full breath, places the lips around the mouthpiece, and then quickly breathes in the powder. Dry powder inhalers do not require the timing and coordination that are necessary with MDIs.

The term “Drosha” as used herein refers to a nuclear RNase III that cleaves primary miRNAs to release hairpin-shaped pre-miRNAs that are subsequently cut by the cytoplasmic RNase III Dicer to generate mature miRNAs.

The term “endogenous photosensitizers” (ePS) as used herein refers to a diverse group of molecules that absorb ultraviolet radiation (UVR, 200-400 nm) and/or visible light (VL, 400-750 nm), forming electronic excited states that transfer energy/electron to surrounding molecules. ePS can have specific physiological functions (e.g., flavins, carotenoids, melanin, porphyrins) or just accumulate as a subproduct of cell aging or oxidative stress (e.g., lipofuscin, which is a product of the incomplete oxidation/degradation of lipids, proteins, pigments and iron that accumulates inside dysfunctional lysosomes) [Tonolli, P N et al. J. Photochemistry and Photobiology (2021) 7: 100044].

The term “Endosomal Sorting Complexes required for transport” (ESCRTs) refers to components involved in multivesicular body (MVB) and intraluminal vesicle (ILV) biogenesis. ESCRTs consist of approximately twenty proteins that assemble into four complexes (ESCRT-0, -I, -II and -III) with associated proteins (VPS4, VTA1, ALIX), which are conserved from yeast to mammals [Colombo, M. et al. J. Cell Science (2013) 126: 5553-5565, citing Henne, W. M., et al. (2011). Dev. Cell 21, 77-91; Henne et al. (2011) Roxrud, I. et al. (2010). ESCRT & Co. Biol. Cell 102, 293-318]. The ESCRT-0 complex recognizes and sequesters ubiquitylated proteins in the endosomal membrane, whereas the ESCRT-I and -II complexes appear to be responsible for membrane deformation into buds with sequestered cargo, and ESCRT-III components subsequently drive vesicle scission [Id., citing Hurley, J. H. and Hanson, P. I. (2010). Nat. Rev. Mol. Cell Biol. 11, 556-566; Wollert, T. et al. Nature (2009) 458: 172-177]. ESCRT-0 comprises HRS protein that recognizes the mono-ubiquitylated cargo proteins and associates in a complex with STAM, Eps15 and clathrin. HRS recruits TSG101 of the ESCRT-I complex, and ESCRT-I is then involved in the recruitment of ESCRT-III, through ESCRT-II or ALIX, an ESCRT-accessory protein. Finally, the dissociation and recycling of the ESCRT machinery requires interaction with the ATPase associated with various cellular activities (AAA-ATPase) Vps4; Vps4 releases ESCRT-III from the MVB membrane for additional sorting events. It is unclear whether ESCRT-II has a direct role in ILV biogenesis or whether its function is limited to particular cargo [Id., citing Bowers, K. et al. (2006) J. Biol. Chem. 281, 5094-5105; Malerød, L. et al. Traffic 8, 1617-1629].

Concomitant depletion of ESCRT subunits belonging to the four ESCRT complexes does not totally impair the formation of MVBs, indicating that other mechanisms may operate in the formation of ILVs and thereby of exosome and or extracellular vesicles [Id., citing Stuffers, S. et al., (2009) Traffic 10, 925-937]. One of these pathways requires a type II sphingomyelinase that hydrolyses sphingomyelin to ceramide [Id., citing Trajkovic, K. et al. (2008) Science 319, 1244-1247]. Although the depletion of different ESCRT components does not lead to a clear reduction in the formation of MVBs and in the secretion of proteolipid protein (PLP) associated to exosomes and/or extracellular vesicles, silencing of neutral sphingomyelinase expression with siRNA or its activity with the drug GW4869 decreases exosome and/or extracellular vesicle formation and release. However, whether such dependence on ceramides is generalizable to other cell types producing exosomes and or extracellular vesicles and additional cargos has yet to be determined. The depletion of type II sphingomyelinase in melanoma cells does not impair MVB biogenesis [Id., citing van Niel, G. et al., (2011) Dev. Cell 21, 708-721] or exosome and or extracellular vesicle secretion, but in these cells the tetraspanin CD63 is required for an ESCRT-independent sorting of the luminal domain of the melanosomal protein PMEL [van Niel et al., (2011) Dev. Cell 21, 708-721]. Moreover, tetraspanin-enriched domains have been proposed to function as sorting machineries allowing exosome and or extracellular vesicle formation [Perez-Hernandez, D. et al. J. Biol. Chem. (2013) 288, 11649-11661]

Despite evidence for ESCRT-independent mechanisms of exosome and or extracellular vesicle formation, proteomic analyses of purified exosomes and or extracellular vesicles from various cell types have identified ESCRT components (TSG101, ALIX) and ubiquitylated proteins [Id., citing Buschow, S. et al., (2005) Blood Cells Mol. Dis. 35, 398-403; Théry, C. et al., (2006). Curr. Protoc. Cell Biol. Chapter 3, Unit 3.22]. It has also been reported that the ESCRT-0 component HRS could be required for exosome and/or extracellular vesicle formation and/or secretion by dendritic cells (DCs) and thereby impact on their antigen-presenting capacity [Id., citing Tamai, K. et al., (2010) Biochem. Biophys. Res. Commun. 399, 384-390]. The transferrin receptor (TfR) in reticulocytes that is generally fated for exosome and/or extracellular vesicle secretion, although not ubiquitylated, interacts with ALIX for MVB sorting [Id., citing Geminard, C. et al., (2004). Traffic 5, 181-193]. It was also shown that ALIX is involved in exosome and/or extracellular vesicle biogenesis and exosomal sorting of syndecans through its interaction with syntenin [Id., citing Baietti, M F et al., (2012). Nat. Cell Biol. 14, 677-685]. Silencing of genes for two components of ESCRT-0 (HRS, STAMI) and one of ESCRT-I (TSG101), as well as a late acting component (VPS4B) induced consistent alterations in exosome and/or extracellular vesicle secretion [Colombo, M. et al. J. Cell Sci. (2013) 126: 5553-5565].

As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

As used herein, the term “expression” and its various grammatical forms refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product”. If the polynucleotide is derived from genomic DNA, expression may include splicing of the mRNA in a eukaryotic cell. Expression may also refer to the post-translational modification of a polypeptide or protein.

The term “extracellular vesicles” or “EVs” as used herein includes exosomes and microvesicles that carry bioactive molecules, such as proteins, RNAs and microRNAs, that may be released into and influence the extracellular environment. Microvesicles are small membrane-enclosed sacs thought to be generated by the outward budding and fission of membrane vesicles from the cell surface. Exosomes originate predominantly from preformed multivesicular bodies that are released upon fusion with the plasma membrane.

The term “exosomes” as used herein refers to extracellular bilayered membrane-bound vesicles of endosomal origin in a size range of ~40 to 160 nm in diameter (~100 nm on average) generated by all cells that are actively secreted.

When used to describe the expression of a gene or polynucleotide sequence, the terms “down-regulation”, “disruption”, “inhibition”, “inactivation”, and “silencing” are used interchangeably herein to refer to instances when the transcription of the polynucleotide sequence is reduced or eliminated. This results in the reduction or elimination of RNA transcripts from the polynucleotide sequence, which results in a reduction or elimination of protein expression derived from the polynucleotide sequence (if the gene comprised an ORF). Alternatively, down-regulation can refer to instances where protein translation from transcripts produced by the polynucleotide sequence is reduced or eliminated. Alternatively, down-regulation can refer to instances where a protein expressed by the polynucleotide sequence has reduced activity. The reduction in any of the above processes (transcription, translation, protein activity) in a cell can be by about 20%, 30%, 40%, 50%, 60%, 70%, 80%, 90%, 95%, or 100% relative to the transcription, translation, or protein activity of a suitable control cell. Down-regulation can be the result of a targeting event as disclosed herein (e.g., indel, knock-out), for example.

The term “clinical efficacy” as used herein refers to the therapeutic effectiveness of a drug or therapeutic in humans using appropriate outcome measures.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

The term “extracellular vesicles (EVs)” as used herein refers to nanosized, membrane-bound vesicles released from cells that can transport cargo—including DNA, RNA, and proteins—between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo [Zaborowski, M P et al. BioScience (2015) 65 (8): 783-797, citing Heijnen, H F et al. Blood (1999) 94: 3791-3799]. Another EV subtype, exosomes, is formed by the fusion between multivesicular bodies and the plasma membrane, by which multivesicular bodies release smaller vesicles (exosomes) whose diameters range from 40 to 120 nm [Id., citing El Andaloussi, S. et al. Nature Reviews Drug Discovery (2013) 12: 347-357; Cocucci, E. and Meldolesi J. Trends in Cell Biology (2015) 25: 364-372]. Dying cells, release vesicular apoptotic bodies (50 nm-2 μm) that can be more abundant than exosomes and or extracellular vesicles or MVs under specific conditions and can vary in content between biofluids [Id., citing Thery, C. et al. J. Immunology (2001) 1666: 7309-7318; El Andaloussi, S. et al. Nature Reviews Drug Discovery (2013) 12: 347-357]. Membrane protrusions can also give rise to large EVs, termed oncosomes (1-10 μm), which are produced primarily by malignant cells in contrast to their nontransformed counterparts [Id., citing Di Vizio, D. et al. Am. J. Pathol. (2012) 181: 1573-1584; Morello, M. et al. Cell Cycle (2013) 12: 3526-3536].

The term “forced vital capacity” as used herein refers to the maximal volume of gas that can be exhaled from full inhalation by exhaling as forcefully and rapidly as possible.

The term “fluence” refers to the total radiant energy delivered per unit area of a surface, expressed in joules per square centimeter (J/cm²). Fluence is calculated as the product of irradiance and the duration of the exposure.

The term “free radical” is defined as any chemical species capable of independent existence that contains one or more unpaired electrons. An unpaired electron refers to the one that occupies an atomic or molecular orbital by itself. Examples of oxygen radicals include superoxide, hydroxyl, peroxyl, and alkoxyl radicals.

The term “growth factor” as used herein refers to extracellular polypeptide molecules that bind to a cell-surface receptor triggering an intracellular signaling pathway, leading to proliferation, differentiation, or other cellular response that stimulate the accumulation of proteins and other macromolecules, e.g., by increasing their rate of synthesis, decreasing their rate of degradation, or both. Exemplary growth factors include fibroblast growth factor (FGF), insulin-like growth factor (IGF-1), transforming growth factor beta (TGF-β), and vascular endothelial growth factor (VEGF).

Fibroblast Growth Factor (FGF). The fibroblast growth factor (FGF) family currently has over a dozen structurally related members. FGF1 is also known as acidic FGF; FGF2 is sometimes called basic FGF (bFGF); and FGF7 sometimes goes by the name keratinocyte growth factor. Over a dozen distinct FGF genes are known in vertebrates; they can generate hundreds of protein isoforms by varying their RNA splicing or initiation codons in different tissues. FGFs can activate a set of receptor tyrosine kinases called the fibroblast growth factor receptors (FGFRs). Receptor tyrosine kinases are proteins that extend through the cell membrane. The portion of the protein that binds the paracrine factor is on the extracellular side, while a dormant tyrosine kinase (i.e., a protein that can phosphorylate another protein by splitting ATP) is on the intracellular side. When the FGF receptor binds an FGF (and only when it binds an FGF), the dormant kinase is activated, and phosphorylates certain proteins within the responding cell, activating those proteins.

FGFs are associated with several developmental functions, including angiogenesis (blood vessel formation), mesoderm formation, and axon extension. While FGFs often can substitute for one another, their expression patterns give them separate functions. For example, FGF2 is especially important in angiogenesis, whereas FGF8 is involved in the development of the midbrain and limbs.

The term “hepatocyte growth factor” (or HGF) as used herein refers to a pleiotrophic growth factor, which induces cellular motility, survival, proliferation, and morphogenesis, depending upon the cell type. In the adult, HGF has been demonstrated to play a critical role in tissue repair, including in the lung. Administration of HGF protein or ectopic expression of HGF has been demonstrated in animal models of pulmonary fibrosis to induce normal tissue repair and to prevent fibrotic remodeling. HGF-induced inhibition of fibrotic remodeling may occur via multiple direct and indirect mechanisms including the induction of cell survival and proliferation of pulmonary epithelial and endothelial cells, and the reduction of myofibroblast accumulation [Panganiban, R A M and Day, R M, Acta Pharmacol. Sin. (2011) 32 (1): 12-20].

Insulin-Like Growth Factor (IGF-1). IGF-1, a hormone similar in molecular structure to insulin, has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 can regulate cell growth and development, especially in nerve cells, as well as cellular DNA synthesis.

IGF-1 was shown to increase the expression levels of the chemokine receptor CXCR4 (receptor for stromal cell-derived factor-1, SDF-1) and to markedly increase the migratory response of MSCs to SDF-1 [Li, Y, et al. Biochem. Biophys. Res. Communic. (2007) 356(3): 780-784]. The IGF-1-induced increase in MSC migration in response to SDF-1 was attenuated by PI3 kinase inhibitor (LY294002 and wortmannin) but not by mitogen-activated protein/ERK kinase inhibitor PD98059. Without being limited by any particular theory, the data indicate that IGF-1 increases MSC migratory responses via CXCR4 chemokine receptor signaling which is PI3/Akt dependent.

The term “platelet derived growth factor” or “PDGF” as used herein refers to a major mitogen for connective tissue cells and certain other cell types. It is a dimeric molecule consisting of disulfide-bonded, structurally similar A- and B-polypeptide chains, which combine to homo- and heterodimers. The PDGF isoforms exert their cellular effects by binding to and activating two structurally related protein tyrosine kinase receptors, denoted the alpha-receptor and the beta-receptor. Activation of PDGF receptors leads to stimulation of cell growth, but also to changes in cell shape and motility; PDGF induces reorganization of the actin filament system and stimulates chemotaxis, i.e., a directed cell movement toward a gradient of PDGF. In vivo, PDGF has important roles during the embryonic development as well as during wound healing. Moreover, overactivity of PDGF has been implicated in several pathological conditions [Helden, C H and Westermark, B. Physiol. Rev. (1999) 79 (4): 1283-316].

Transforming Growth Factor Beta (TGF-β). There are over 30 structurally related members of the TGF-β superfamily, and they regulate some of the most important interactions in development. The proteins encoded by TGF-β superfamily genes are processed such that the carboxy-terminal region contains the mature peptide. These peptides are dimerized into homodimers (with themselves) or heterodimers (with other TGF-β peptides) and are secreted from the cell. The TGF-β superfamily includes the TGF-β family, the activin family, the bone morphogenetic proteins (BMPs), the Vg-1 family, and other proteins, including glial-derived neurotrophic factor (GDNF, necessary for kidney and enteric neuron differentiation) and Müllerian inhibitory factor, which is involved in mammalian sex determination. TGF-β family members TGF-β1, 2, 3, and 5 are important in regulating the formation of the extracellular matrix between cells and for regulating cell division (both positively and negatively). TGF-β1 increases the amount of extracellular matrix epithelial cells make both by stimulating collagen and fibronectin synthesis and by inhibiting matrix degradation. TGF-βs may be critical in controlling where and when epithelia can branch to form the ducts of kidneys, lungs, and salivary glands.

The term “tumor necrosis factor-alpha” (“TNF-α”) as used herein refers to a potent pro-inflammatory cytokine exerting pleiotropic effects on various cell types and plays a critical role in the pathogenesis of chronic inflammatory diseases, Transmembrane TNF-α, a precursor of the soluble form of TNF-α, is expressed on activated macrophages and lymphocytes as well as other cell types. After processing by TNF-α-converting enzyme (TACE), the soluble form of TNF-α is cleaved from transmembrane TNF-α and mediates its biological activities through binding to Types 1 and 2 TNF receptors (TNF-R1 and -R2) of remote tissues. Accumulating evidence suggests that not only soluble TNF-α, but also transmembrane TNF-α is involved in the inflammatory response [Horiuchi, T. et al. Rheumatology (Oxford) (2010) 49 (7): 1215-28].

Vascular Endothelial Growth Factor (VEGF). VEGFs are growth factors that mediate numerous functions of endothelial cells including proliferation, migration, invasion, survival, and permeability. The VEGFs and their corresponding receptors are key regulators in a cascade of molecular and cellular events that ultimately lead to the development of the vascular system, either by vasculogenesis, angiogenesis, or in the formation of the lymphatic vascular system. VEGF is a critical regulator in physiological angiogenesis and also plays a significant role in skeletal growth and repair.

VEGF's normal function creates new blood vessels during embryonic development, after injury, and to bypass blocked vessels. In the mature established vasculature, the endothelium plays an important role in the maintenance of homeostasis of the surrounding tissue by providing the communicative network to neighboring tissues to respond to requirements as needed. Furthermore, the vasculature provides growth factors, hormones, cytokines, chemokines and metabolites, and the like, needed by the surrounding tissue and acts as a barrier to limit the movement of molecules and cells.

The term “healthy control” as used herein refers to a subject in a state of physical well-being without signs or symptoms of a fibrotic disease or process.

The term “heat shock response” as used herein refers to an evolutionarily conserved mechanism that protects cells or organisms from the harmful effects of various stressors such as heat, chemicals toxins, UV radiation, and oxidizing agents. These stressors cause various types of damage, both reversible and irreversible, at cellular and molecular levels. Such damage can alter the protein structure, leading to damage or degradation that affect cell functions [Singh, M K et al. Int. J. Mol. Sci. (2024) 25 (8): 4209, citing Morimoto, RI and Santoro, MG. Nat. Biotechnol. (1998): 16: 833-838]. When the heat shock response is initiated, protein transcription and translation are halted, possibly to reduce the burden of protein damage. This process is regulated by a subset of transcription factors known as heat shock factors (HSFs), which bind to the promoter region of the heat shock gene and induce its expression until the cells are normalized. In vertebrates, HSF1 mainly responds to heat shock or elevated temperature, while HSF2 and HSF4 are essential for development and differentiation processes. HSF3 is an avian-specific transcriptional factor, co-expressed and co-activated with HSF1 by chemical and physiological stress [Id., citing Mizrahi, T. et al. Cell Stress Chaperones (2012) 17: 639-645; Pirkkala, L. etal. FASEB J. (2001) 15: 1118-1131]. HSF2 can modulate HSF1-mediated expression of heat-responsive genes under certain circumstances, suggesting that HSF2 can also participate in transcriptional regulation of the heat shock response [Id., citing Ostling, P. et al. J. Biol. Chem. (2007) 282: 7077-7086]. HSF3 also interacts with other transcriptional factors, such as the MYB oncogene, directly and without stress [Id., citing Kanei-Ishii, C. et al. Science (1997) 277: 246-248]. Thus, HSFs play an essential role in regulation of the induced expression of heat shock proteins. At the translational level, these chaperones bind transiently and non-covalently to nascent polypeptides, and can unfold or unassembled proteins, aiding in protein biogenesis in two general ways: either by blocking non-productive protein-protein interactions or maintaining protein folding in their native state by sequestering folding intermediates, allowing coordinated folding by domains and assembly of oligomers [Id., citing Craig, E A et al. Microbiol. Rev. (1993) 57: 402-414].

The heat shock response triggers the expression of a specific set of genes and proteins known as heat shock genes/proteins or molecular chaperones, including HSP100, HSP90, HSP70, HSP60, and small HSPs. Heat shock proteins (HSPs) play a crucial role in thermotolerance and aiding in protecting cells from harmful insults of stressors. HSPs are involved in essential cellular functions such as protein folding, eliminating misfolded proteins, apoptosis, and modulating cell signaling.

The term “HSP70” as used herein refers to a system of 70 kDa heat shock proteins. They are ubiquitous molecular chaperones that act in a large variety of cellular protein folding and remodeling processes. They function virtually at all stages of the life of proteins from synthesis to degradation and are thus crucial for maintaining protein homeostasis, with direct implications for human health. A large set of co-chaperones comprising J-domain proteins and nucleotide exchange factors regulate the ATPase cycle of Hsp70s, which is allosterically coupled to substrate binding and release. Moreover, Hsp70s cooperate with other cellular chaperone systems including Hsp90, Hsp60 chaperonins, small heat shock proteins and Hsp100 AAA+ disaggregases, together constituting a dynamic and functionally versatile network for protein folding, unfolding, regulation, targeting, aggregation and disaggregation, as well as degradation. Rosenzweig, R. et al. Nature Reviews Molecular Cell Biol. (2019) 20: 665-680].

Hydroxyproline assay. Collagen content is assessed by quantifying hydroxyproline, an amino acid present in appreciable quantities in collagen.

The term “high throughput screening” or “HTS” as used herein refers to the use of automated equipment to rapidly test thousands to millions of samples for biological activity at the model organism, cellular, pathway, or molecular level.

The term “immune system” as used herein refers to a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other but rather work together to elicit effective immune responses.

The term “inflammation” as used herein refers to the physiologic process by which vascularized tissues respond to injury [See, e.g., FUNDAMENTAL IMMUNOLOGY, 4th Ed., William E. Paul, ed. Lippincott-Raven Publishers, Philadelphia (1999) at 1051-1053, incorporated herein by reference]. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity. The term “innate immunity” as used herein refers to a nonspecific fast response to pathogens that is predominantly responsible for an initial inflammatory response before adaptive immunity is induced. These include such mechanisms as anatomical barriers, antimicrobial peptides, the complement system and the chemokine/cytokine system; macrophages and neutrophils carrying nonspecific pathogen-recognition receptors, and a number of specialized cell types, including innate lymphoid cells (ILCs, including natural killer (NK) cells) mast cells and dendritic cells (DCs). Innate immunity is present in all individuals at all times, does not increase with repeated exposure to a given pathogen, and discriminates between groups of similar pathogens, rather than responding to a specific pathogen.

The term “infuse” and its other grammatical forms as used herein refers to introduction of a fluid other than blood into a vein.

The term “inhalation delivery device” as used herein refers to a machine/apparatus or component that produces small droplets or an aerosol from a liquid or dry powder aerosol formulation and is used for administration through the mouth in order to achieve pulmonary administration of a drug, e.g., in solution, powder, and the like. Examples of inhalation delivery device include, but are not limited to, a nebulizer, a metered-dose inhaler, and a dry powder inhaler (DPI).

The term “insufflation” as used herein refers to the act of delivering air, a gas, or a powder under pressure to a cavity or chamber of the body. For example, nasal insufflation relates to the act of delivering air, a gas, or a powder under pressure through the nose.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

The term “interleukin” as used herein refers to a cytokine secreted by white blood cells as a means of communication with other white blood cells. For example, interleukin-8 (or “IL-8”) is produced by phagocytes and mesenchymal cells exposed to inflammatory stimuli (e.g., interleukin-1 or tumor necrosis factor) and activates neutrophils inducing chemotaxis, exocytosis and the respiratory burst. In vivo, IL-8 elicits a massive neutrophil accumulation at the site of injection.

The term “interleukin-1 receptor associated kinase” or IRAK-1″ IRAK-4, refer to protein kinases that are part of the intracellular signaling pathways leading from TLRs.

The term “irradiance” refers to the power of electromagnetic radiation per unit area of a surface, expressed in watts per square centimeter (W/cm²) or milliwatts per centimeter (mW/cm²). Irradiance describes the instantaneous rate at which radiant energy is delivered to a surface.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “JAG1” refers to the gene that encodes protein jagged-1. Jagged-1 is the ligand for multiple Notch receptors and is involved in the mediation of Notch signaling.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression [Zampetaki, A. et al. Front. Physiol. (2018) doi.org/10.3389/fphys.2018.01201, citing Yoon, J H et al. Semin. Cell Dev. Bio. (2014) 34: 9-14; Ballantyne, M D et al. Clin. Pharmacol. Ther. (2016) 99: 494-501]. Finally, lncRNAs may contain embedded miRNA sequences and serve as a source of miRNAs [Id., citing Piccoli, M T et al. Cir. Res. (2017) 121: 575-583].

The terms “lung function” or “pulmonary function” are used interchangeably to refer to the process of gas exchange called respiration (or breathing). In respiration, oxygen from incoming air enters the blood, and carbon dioxide, a waste gas from the metabolism, leaves the blood. A reduced lung function means that the ability of lungs to exchange gases is reduced.

The terms “lung interstitium” or “pulmonary interstitium” are used interchangeably herein to refer to an area located between the airspace epithelium and pleural mesothelium in the lung. Fibers of the matrix proteins, collagen and elastin, are the major components of the pulmonary interstitium. The primary function of these fibers is to form a mechanical scaffold that maintains structural integrity during ventilation.

The abbreviation “MAPK” as used herein refers to Mitogen-Activated Protein Kinase (MAPK) signaling which activates a three-tiered cascade with MAPK kinase kinases (MAP3K) activating MAPAK kinases (MAP2K) and finally MAPK. MAPKs are protein Ser/Thr kinases that convert extracellular stimuli into a wide range of cellular responses [Cargnello, M. and Roux, PP, Microbiol. Mol. Biol. Rev. (2011) 75(1): 50-83]. The major MAPK pathways involved in inflammatory diseases are extracellular regulating kinase (ERK), p38 MAPK, and c-Jun NH2-terminal kinase (JNK). All three MAPK pathways may be activated by TGF-β, and signaling through these cascades can further regulate the expression of Smad proteins and mediate Smad-independent TGF-β responses. These three MAPK pathways are all involved in TGF-β-induced fibrosis [He, W. and Dai, C. Curr. Pathobiol. Rep. (2015) 3: 183-92, citing Tsou, P S et al. Am. J. Physiol. Cell Physiol. (22014) 307: C2-13; Kamato, D. et al. Cell Signal (2013) 25: 2017-2024; Pannu, J. et al. J. Biol. Chem. (2007) 282: 10405-10413; Yu, L. et al. Biol. Chem. (2002) EMBO J. 21: 3749-3759].

Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). TGF-β can signal in a noncanonical manner via the MAPK family.

The term “matrix metalloproteinases” as used herein refers to a collection of zinc-dependent proteases involved in the breakdown and the remodeling of extracellular matrix components [Guiot, J. et al. Lung (2017) 195(3): 273-280, citing Oikonomidi et al. Curr Med Chem. 2009; 16(10): 1214-1228]. MMP-1 and MMP-7 seem to be primarily overexpressed in plasma of IPF patients compared to hypersensitivity pneumonitis, sarcoidosis and COPD with a possible usefulness in differential diagnosis [Id., citing Rosas I O, et al. PLoS Med. 2008; 5(4): e93]. They are also involved in inflammation and seem to take part to the pathophysiological process of pulmonary fibrosis [Id., citing Vij R, Noth I. Transl Res. 2012; 159(4): 218-227; Dancer R C A, et al. Eur Respir J. 2011; 38(6): 1461-1467]. The most studied is MMP-7, which is known as being significantly increased in epithelial cells both at the gene and protein levels and is considered to be active in hyperplastic epithelial cells and alveolar macrophages in IPF [Id., citing Fujishima S, et al. Arch Pathol Lab Med. 2010; 134(8): 1136-1142]. There is also a significant correlation between higher MMP-7 concentrations and disease severity assessed by forced vital capacity (FVC) and diffusing capacity of the lungs for carbon monoxide (DLCO) [Id., citing Rosas I O, et al. PLoS Med. 2008; 5(4): e93]. Higher levels associated to disease progression and worse survival (>4.3 ng/ml for MMP-7) (Id.). The MMP2 gene provides instructions for making matrix metallopeptidase 2. This enzyme is produced in cells throughout the body and becomes part of the extracellular matrix, which is an intricate lattice of proteins and other molecules that forms in the spaces between cells. One of the major known functions of MMP-2 is to cleave type IV collagen, which is a major structural component of basement membranes, the thin, sheet-like structures that separate and support cells as part of the extracellular matrix.

MMPs play a critical role in neuroinflammation through the cleavage of ECM proteins, cytokines and chemokines [Ji. R-R et al, US Neurology, Touch Briefings (2008) 71-74]. MMP-2 is constitutively expressed and normally present in brain and spinal cord tissues. In contrast, MMP-9 is normally expressed at low levels, but upregulated in many injury and disease states such as spinal cord injury and brain trauma [Id., citing Rosenberg, GA. Glia (2002) 39: 279-291]; it is also induced in the crushed sciatic nerve and causes demyelination, a condition associated with neuropathic pain, by the cleavage of myelin basic protein [Id., citing Chattopadhyay, S. et al. Brain Behav. Immun. (20007) 21: 561-568]. Besides targeting matrix, because MMPs can process a variety of growth factors and other extracellular cytokines and signals, they may contribute to the neurovascular remodeling that accompanies chronic CNS injury [Id., citing Zhao, B Q, et al. Nat. Med. (2006) 12: 441-445].

The term “mean” as used herein refers to the sum of all values in a data set divided by the number of values in the data set. It is also called the arithmetic average.

The term “median” as used herein refers to a middle value for sorted data that divides the data into two halves.

The term “mesenchymal stem cells” (MSCs) (also known as bone marrow stromal stem cells or skeletal stem cells) are non-blood adult stem cells found in a variety of tissues. They are characterized by their spindle-shape morphologically; by the expression of specific markers on their cell surface; and by their ability, under appropriate conditions, to differentiates along a minimum of three lineages (osteogenic, chondrogenic, and adipogenic) [Najar M. et al., “Mesenchymal stromal cells and immunomodulation: A gathering of regulatory immune cells”, Cytotherapy (2016) Vol. 18(2): 160-171]. No single marker that definitely delineates MSCs in vivo has been identified due to the lack of consensus regarding the MSC phenotype, but it generally is considered that MSCs are positive for cell surface markers CD105, CD166, CD90, and CD44 and that MSCs are negative for typical hematopoietic antigens, such as CD45, CD34, and CD14. Studies have reported that populations of bone marrow-derived MSCs have the capacity to develop into terminally differentiated mesenchymal phenotypes both in vitro and in vivo, including bone, cartilage, tendon, muscle, adipose tissue, and hematopoietic supporting stroma. Studies using transgenic and knockout mice and human musculoskeletal disorders have reported that MSC differentiate into multiple lineages during embryonic development and adult homeostasis [Najar M. et al., “Mesenchymal stromal cells and immunomodulation: A gathering of regulatory immune cells”, Cytotherapy (2016) Vol. 18(2): 160-171].

It has been reported that MSCs from different placental layers have different proliferation rates and differentiation potentials [Choio, Y S et al. “Different characteristics of mesenchymal stem cells isolated from different layers of full term placenta.” PLoS One (2017) 12 (2): e0172642], i.e., MSCs from chorionic membrane (CM), chorionic trophoblast without villi (CT-V), chorionic villi (CV), and decidua (DC) had better population doubling time and multi-lineage differentiation potentials compared to those from other layers [See also, Wu, M. et al. “Comparison Of The Biological Characteristics Of Mesenchymal Stem Cells Derived From The Human Placenta And Umbilical Cord.” Scientific Repts (2018) 8: 5014, comparing MSCs derived from amniotic membrane (AM), chorionic plate (CP), decidua parietalis (CP) and umbilical cord (UC)].

The term “metered-dose inhaler”, “MDI”, or “puffer” as used herein refers to a pressurized, hand-held device that uses propellants to deliver a specific amount of medicine (“metered dose”) to the lungs of a patient. The term “propellant” as used herein refers to a material that is used to expel a substance usually by gas pressure through a convergent, divergent nozzle. The pressure may be from a compressed gas, or a gas produced by a chemical reaction. The exhaust material may be a gas, liquid, plasma, or, before the chemical reaction, a solid, liquid or gel. Propellants used in pressurized metered dose inhalers are liquefied gases, traditionally chlorofluorocarbons (CFCs) and increasingly hydrofluoroalkanes (HFAs). Suitable propellants include, for example, a chlorofluorocarbon (CFC), such as trichlorofluoromethane (also referred to as propellant 11), dichlorodifluoromethane (also referred to as propellant 12), and 1,2-dichloro-1,1,2,2-tetrafluoroethane (also referred to as propellant 114), a hydrochlorofluorocarbon, a hydrofluorocarbon (HFC), such as 1,1,1,2-tetrafluoroethane (also referred to as propellant 134a, HFC-134a, or HFA-134a) and 1,1,1,2,3,3,3-heptafluoropropane (also referred to as propellant 227, HFC-227, or HFA-227), carbon dioxide, dimethyl ether, butane, propane, or mixtures thereof. In other embodiments, the propellant includes a chlorofluorocarbon, a hydrochlorofluorocarbon, a hydrofluorocarbon, or mixtures thereof. In other embodiments, a hydrofluorocarbon is used as the propellant. In other embodiments, HFC-227 and/or HFC-134a are used as the propellant.

The term “microRNA” (or “miRNA” or “miR”) as used herein refers to a class of small, 18- to 28-nucleotide-long, noncoding RNA molecules.

The term “mode” as used herein refers to the most frequent value or item of a data set.

The term “modulate” as used herein means to regulate, alter, adapt, or adjust to a certain measure or proportion.

The term “neuropathic” as used herein refers to relating to any disorder affecting the nervous system. The term “neuropathic pain” as used herein refers to pain derived from injury to the peripheral nervous system (e.g., peripheral nerves) or the CNS, which may result from major surgeries, e.g., amputation and thoracotomy, diabetic neuropathy, viral infection, chemotherapy, spinal cord injury, stoke, etc. Neuropathic pain is often characterized by spontaneous pain, described as shooting, lancinating, or bringing pain, and also by evoked pain, such as hyperalgesia (increased responsiveness to noxious stimuli) to mechanical and thermal stimuli. Mechanical allodynia, meaning painful responses to normally innocuous tactile stimuli may be the most distinct symptom of neuropathic pain. There are at least two phases of neuropathic pain in animal models: an early phase (first several days) when neuropathic pain is developed, and late phase (from a week to months and even years) when neuropathic pain is maintained. Animal model experiments have shown that MMP-9 induces early-phase neuropathic pain by activating IL-1β and microglia in the early phase [Ji. R-R et al, US Neurology, Touch Briefings (2008) 71-74]. MMP-2 inhibition experiments showed that MMP-2 contributes to late-phase neuropathic pain development by activating IL-1ß and astrocytes in the late phase [Id.]. Apart from their pathological roles, MMP-9 and MMP-2 aalso play a physiological roles in regulating development and regeneration; depending on whether functional or dysfunctional remodeling occurs, the result might be recovery or the induction of aberrant neuronal circuits [Ji, R-R et al, Trends Pharmacol. Sci. (2009) 30 (7): 336-340]. Using a rat adjuvant-induced arthritis model, it was shown that the Chinese medicine crocin may alleviate neuropathic pain in AIA rats by inhibiting the expression of pain-related molecules through the Wnt5a/β-catenin pathway [Wang, J-F et al. Neural Plasticity (2020) 4297483]. Although it was long known that crocin can effectively alleviate pain sensitization in rat pain models, its mechanism was unknown. Crocin significantly increased the mechanical thresholds of adjuvant-induced arthritis in rats, suggesting that crocin can alleviate neuropathic pain. Crocin significantly decreased the levels of pain-related factors and glial activation. Foxy5, activator of Wnt5a, inhibited these effects of crocin in AIA rats. In addition, intrathecal injection of a Wnt5a inhibitor significantly decreased hyperalgesia in AIA rats.

The term “nerve” as used herein refers to a whitish fiber or bundle of fibers that transmits impulses of sensation to the brain or spinal cord, and impulses from the brain or spinal cord to the muscles and organs.

The term “nervous system” as used herein refers to the network of nerve cells and fibers which transmits nerve impulses between parts of the body. The central nervous system (CNS) is that part of the nervous system that consists of the brain and spinal cord. It is one of the two major divisions of the nervous system. The other is the peripheral nervous system (PNS) which is outside the brain and spinal cord. The peripheral nervous system (PNS) connects the central nervous system (CNS) to sensory organs (such as the eye and ear), other organs of the body, muscles, blood vessels and glands. The peripheral nerves include the 12 cranial nerves, the spinal nerves and roots, and the autonomic nerves of the autonomic nervous system (ANS), meaning the part of the nervous system responsible for control of the bodily functions not consciously directed, such as breathing, the heartbeat, and digestive processes.

The abbreviation “NFκB” as used herein refers to which is a proinflammatory transcription factor. It switches on multiple inflammatory genes, including cytokines, chemokines, proteases, and inhibitors of apoptosis, resulting in amplification of the inflammatory response [Barnes, PJ, (2016) Pharmacol. Rev. 68: 788-815]. The molecular pathways involved in NF-κB activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ (or NFκB essential modulator [Id., citing Hayden, MS and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, SC. (2012) Immunol. Rev. 246: 125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-189].

The term “Notch” refers to a signaling pathway that has been implicated in abnormal differentiation of respiratory epithelial cells in progressive IPF or secondary pulmonary fibrosis [He, W. and Dai, C. Curr. Pathobiol. Resp. (2015) 3: 183-192, citing Plantier, L. et al. Thorax (2011) 66: 651-657]. Notch proteins are single-pass transmembrane receptors with conserved expression among animal species during evolution. Their principal function is the regulation of many developmental processes, including proliferation, differentiation, and apoptosis. Mammals possess four different Notch receptors, referred to as Notch 1-4. The Notch receptor consists of an extracellular domain, which is involved in ligand binding, and an intracellular domain that works in signal transduction. Notch ligands also are single-pass transmembrane proteins named Jagged (Jag1 and 2) and Delta (D111, 3, and 4) [Id., citing Sharma, S. et al. Curr. Opin. Nephrol Hypertens. (2011) 20: 56-61; Bray, SJ. Nat. Rev. Mol. Cell Biol. (2006) 7: 678-689]. Activation of this signaling pathway requires cell-cell contact. Interaction of ligands with the Notch receptors triggers a series of proteolytic cleavages, by a metalloprotease of the ADAM family (TACE; tumor necrosis factor-α-converting enzyme) and finally by the γ-secretase complex. The final cleavage leads to the release of Notch intracellular domain (NICD), which travels to the nucleus and binds to other transcriptional regulators (mainly of the CBF1/RBP-Jκ, SU(H), Lag1 family) to trigger the transcription of the target genes, classically belonging to the Hes and Hey family. This core signal transduction pathway is used in most Notch-dependent processes and is known as the canonical Notch pathway [Id., citing Sharma, S. et al. Curr. Opin. Nephrol. Hypertens. (2011) 20: 56-61; Kavian N. et al. Open Rheumatol. J (2012) 6: 96-102; Fortini, ME. Dev. Cell (2009) 16: 633-647]. During the past few years, activation of Notch signaling has shown fibrogenic effects in a wide spectrum of diseases, including systemic sclerosis (SSc) [Id., citing Dees, C. et al. Ann. Rheum. Dis. (2011) 70: 1304-1310; Kavian, N. et al. Arthritis Rheum. (2010) 62: 3477-3487], scleroderma, idiopathic pulmonary fibrosis (IPF) [Id., citing Plantier, L. et al. Thorax (2011) 66: 651-657], kidney fibrosis [Id., citing Sharma, S. et al. Curr. Opin. Nephrol. Hpertens. (2011) 20: 56-61; Niranjan, T. et al. Nat. Med. (2008) 14: 290-298; Murea, M. et al. Kidney Int. (2010) 78: 514-522], and cardiac fibrosis [Id., citing Kavian, N. et al. Open Rheumatol J. (2012) 6: 96-102].

The term “organ” as used herein refers to a differentiated structure consisting of cells and tissues and performing some specific function in an organism.

The term “oxidative stress” as used herein refers to a condition where the levels of ROS significantly overwhelm the capacity of antioxidant defenses, leading to potential damage in a biological system. Oxidative stress can be caused by either increased ROS formation or decreased activity of antioxidants or both. Not all increases in ROS levels in a biological system are associated with injury. Under certain circumstances, small transient increases in ROS levels can be employed as a signaling mechanism, leading to physiological cellular responses [Li, R. et al. React. Oxyg. Species (Apex) (2016) 1 (1): 9-21].

As used herein, the term “paracrine signaling” refers to short range cell-cell communication via secreted signal molecules that act on adjacent cells.

The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques.

The term “particles” as used herein refers to refers to an extremely small constituent (e.g., nanoparticles, microparticles, or in some instances larger) in or on which is contained the composition as described herein.

The term “particulate” as used herein refers to fine particles of solid or liquid matter suspended in a gas or liquid.

The term “pathogen” as used herein refers to a causative agent of disease. It includes, without limitation, viruses, bacteria, fungi and parasites.

The term “pathogenesis” as used herein refers to the pathologic, physiologic or biochemical mechanism resulting in the development of a disease.

The term “pharmaceutical composition” is used herein to refer to a composition that is employed to prevent, reduce in intensity, cure or otherwise treat a target condition or disease. The terms “formulation” and “composition” are used interchangeably herein to refer to a product of the described invention that comprises all active and inert ingredients.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

“Phosphopinositide 3-Kinase Pathway, AKT/mTOR and PAK2/c-Abl”. The phosphoatidylinositol 3-kinase (PI3K) pathway is a non-Smad pathway contributing to TGF-β induced fibrosis. It induces two profibrotic pathways: Akt-mammalian target of rapamycin (mTOR) and p21-activated kinase 2 (PAK2)/Abelson kinase (c-Abl) [He, W and Dai, C. Curr. Pathobiol. Rep. (2015) 3: 183-192, citing Biernacka, A, et al. Growth Factors (2011) 29: 196-202; Tsou, P S et al. Am. J. Physiol. Cell Physiol. (2014) 307: C2-C13; Kamato, D. et al. Cell Signal (2013) 25: 2017-2024; Wilkes, M C et al. Cancer Res. (2005) 65: 10431-10440]. The phosphatidylinositol-3-kinase (PI3K)/Akt and the mammalian target of rapamycin (mTor) signaling pathways are crucial to many aspects of cell growth and survival [Porta, C. et al., “Targeting PI2K/Akt/mTor signaling in cancer. Frontiers] in Oncology (2014) doi.10.3389/fpmc.2014.00064]. They are so interconnected that they could be regarded as a single pathway that, in turn, heavily interacts with many other pathways, including that of hypoxia inducible factors (HIFs).

PI3Ks constitute a lipid kinase family characterized by the capability to phosphorylate inositol ring 3′-OH group in inositol phospholipids [Id., citing Fruman, D A et al., Phosphoinositide kinases. Annu. Rev. Biochem. (1998) 67: 481-507]. Class I PI3Ks are heterodimers composed of a catalytic (CAT) subunit (i.e., p110) and an adaptor/regulatory subunit (i.e., p85). This classis further divided into two subclasses: subclass IA (PI3Kα, β, and δ), which is activated by receptors with protein tyrosine kinase activity, and subclass IB (PI3Kγ), which is activated by receptors coupled with G proteins [Id., citing Fruman, D A et al., Phosphoinositide kinases. Annu. Rev. Biochem. (1998) 67: 481-507].

Activation of growth factor receptor protein tyrosine kinases results in autophosphorylation on tyrosine residues. P13K is then recruited to the membrane by directly binding to phosphotyrosine consensus residues of growth factor receptors or adaptors through one of the two SH2 domains in the adaptor subunit. This leads to allosteric activation of the CAT subunit. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) [Id., citing Fruman, D A et al., Phosphoinositide kinases. Annu. Rev. Biochem. (1998) 67: 481-507, Fresno-Vara, J A, et al., PI3K/Akt signaling pathway and cancer. Cancer Treat. Rev. (2004) 30: 193-204]. Akt/PKB, on its own, regulates several cell processes involved in cell survival and cell cycle progression.

Akt. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor [Shimamura, H, et al., J. Am. Soc. Nephrol. (2003) 14: 1427-1434; Datta K, Franke T F, Chan T O, Makris A, Yang S I, Kaplan D R, Morrison D K, Golemis E A, Tsichlis P N, Mol Cell Biol (1995) 15: 2304-2310; Kulik G, Klippel A, Weber M J, Mol Cell Biol (1997) 17: 1595-1606; Yao R, Cooper G M, Science (1995) 267: 2003-2006]. Stimulation of Akt has been shown to be dependent on phosphatidylinositol 3-kinase (PI3-kinase) activity [Fruman D A, Meyers R E, Cantley L C, Annu Rev Biochem (1998) 67: 481-507; Choudhury G, Karamitsos C, Hernandez J, Gentilini A, Bardgette J, Abboud H E, Am J Physiol (1997) 273: F931-938, Franke T F, Yang S I, Chan T O, Datta K, Kazlauskas A, Morrison D K, Kaplan D R, Tsichlis P N, Cell (1995) 81: 727-736; Franke T F, Kaplan D R, Cantley L C, Cell (1997) 88: 435-437, 1997].

Akt has been shown to act as a mediator of survival signals that protect cells from apoptosis in multiple cell lines [Brunet A, Bonni A, Zigmond M J, Lin M Z, Juo P, Hu L S, Anderson M J, Arden K C, Blenis J, Greenberg M E, Cell (1999) 96: 857-868; Downward J, Curr Opin Cell Biol (19998) 10: 262-267]. For example, phosphorylation of the pro-apoptotic Bad protein by Akt was found to decrease apoptosis by preventing Bad from binding to the anti-apoptotic protein Bcl-XL [Dudek H, Datta S R, Franke T F, Birnbaum M J, Yao R, Cooper G M, Segal R A, Kaplan D R, Greenberg M E, Science (1997) 275: 661-665; Datta S R, Dudek H, Tao X, Masters S, Fu H, Gotoh Y, Greenberg M E, Cell (1997) 91: 231-241]. Akt was also shown to promote cell survival by activating nuclear factor-kB (NF-kB) [Cardone M H, Roy N, Stennicke H R, Salvesen G S, Franke T F, Stanbridge E, Frisch S, Reed J C, Science (1998) 282: 1318-1321; Khwaja A, Nature (1999) 401: 33-34] and inhibiting the activity of the cell death protease caspase-9 [Kennedy S G, Kandel E S, Cross T K, Hay N, Mol Cell Biol (1999) 19: 5800-5810].

mTOR signaling pathway: Mechanistic target of rapamycin (mTOR) is an atypical serine/threonine kinase that is present in two distinct complexes. The first, mTOR complex 1 (mTORC1), is composed of mTOR, Raptor, GBL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumour-suppressor genes mutated in the tumour syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1) [Huang, J. Manning B D, Biochem J. (2008) 412 (2): 179-90]. Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GBL, Sin1, PRR5/Protor-1, and DEPTOR. mTORC2 promotes cellular survival by activating Akt, regulates cytoskeletal dynamics by activating PKCα, and controls ion transport and growth via SGK1 phosphorylation. Aberrant mTOR signaling is involved in many disease states

PI3K also acts as a branch point in response to TGF-β, leading to activation of PAK2/c-Abl, which stimulates collagen gene expression in normal fibroblasts, and induces fibroblast proliferation, thereby increasing the number of myofibroblast precursors [He, W and Dai, C. Curr. Pathobiol. Rep. (2015) 3: 183-192, citing Wilkes, M C and Leof, EB. J. Biol. Chem. (2006) 281: 27846-27854]. PAK2/c-Abl promotes fibrosis through its downstream mediators, including PKCδ/Fli-1 and early growth response (Egr)-1, -2, and -3 {Id., citing Tsou, P S et al. A. J. Physiol. Cell Physiol. (2014) 307: C2-C13; Bhattacharyya, S. et al. J. Pathol. (2013) 229: 286-297; Fang, F. et al. Am. J. Pathol. (2013) 183: 1197-1208].

The term “Plasma-Lyte” or Plasma-Lyte 148″ as used herein refers to an isotonic, buffered intravenous crystalloid solution with a physiochemical composition that closely reflects human plasma.

The term “potency” and its various grammatical forms as used herein, refers to power or strength of a formulation.

The term “pulmonary compliance” as used herein refers to the change in lung volume per unit change in pressure. Dynamic compliance is the volume change divided by the peak inspiratory transthoracic pressure. Static compliance is the volume change divided by the plateau inspiratory pressure. Pulmonary compliance measurements reflect the elastic properties of the lungs and thorax and are influenced by factors such as degree of muscular tension, degree of interstitial lung water, degree of pulmonary fibrosis, degree of lung inflation, and alveolar surface tension [Doyle D J, O'Grady K F. Physics and Modeling of the Airway, D, in Benumof and Hagberg's Airway Management, 2013]. Total respiratory system compliance is given by the following calculation:

C=ΔV/AP
where ΔV=change in lung volume, and ΔP=change in airway pressure

This total compliance may be related to lung compliance and thoracic (chest wall) compliance by the following relation:
where CT=total compliance (e.g., 100 mL/cm H2O)
CL=lung compliance (e.g., 200 mL/cm H2O)
CTh=thoracic compliance (e.g., 200 mL/cm H2O)

The values shown in parentheses are some typical normal adult values that can be used for modeling purposes (Id.). It has been reported that soon after onset of respiratory distress from COVID, patients initially retain relatively good compliance despite very poor oxygenation [Marini, J J and Gattinoni, L., JAMA Insights (2020) doi: 10.1001/jama.2020.6825, citing Grasselli, G. et al., JAMA (2020) doi: 10.1001/jama.2020.5394; Arentz, M. et al. JAMA (2020) doi: 10.1001/jama.2020.4326]. Minute ventilation is characteristically high. Infiltrates are often limited in extent and, initially, are usually characterized by a ground-glass pattern on CT that signifies interstitial rather than alveolar edema. Many patients do not appear overtly dyspneic. These patients can be assigned, in a simplified model, to “type L,” characterized by low lung elastance (high compliance), lower lung weight as estimated by CT scan, and low response to PEEP [Id., citing Gattinoni, L. et al. Intensive Care Med. (2020) doi: 10.1007/s00134-020-06033-2]. For many patients, the disease may stabilize at this stage without deterioration while others, either because of disease severity and host response or suboptimal management, may transition to a clinical picture more characteristic of typical ARDS. These can be defined as “type H,” with extensive CT consolidations, high elastance (low compliance), higher lung weight, and high PEEP response. Types L and H are the conceptual extremes of a spectrum that includes intermediate stages, in which their characteristics may overlap.

The term “potency” as used herein and its various grammatical forms is an expression of the activity of a drug in terms of the concentration or amount of the drug required to produce a defined effect.

The term “precision medicine” as used herein refers to an approach for disease treatment and prevention that takes into account individual variability in genes, environment and lifestyle. A precision medicine approach allows for a more accurate prediction of which treatment and prevention strategies for a particular disease will work in which groups of patients. This is in contrast to a one-size-fits-all approach, in which disease treatment and prevention strategies are developed for the average person with less consideration for differences between individuals.

The term “progression” as used herein refers in medicine to the course of a disease as it becomes worse or spreads in the body.

The term “purification” and its various grammatical forms as used herein refers to the process of isolating or freeing from foreign, extraneous, or objectionable elements.

The term “reactive oxygen species” as used herein refers to oxygen-containing reactive species. It is a collective term to include superoxide (O2▪-), hydrogen peroxide (H2O2), hydroxyl radical (OH▪-), singlet oxygen (1O2), peroxyl radical (LOO▪), alkoxyl radical (LO-▪), lipid hydroperoxide (LOOH), peroxynitrite (ONOO—), hypochlorous acid (HOCl), and ozone (O3), among others [Li, R. et al. React. Oxyg. Species (Apex) (2016) 1 (1): 9-21].

The term “recombinant” as used herein refers to an artificial combination of two otherwise separated segments of sequence, e.g., by chemical synthesis or by the manipulation of isolated segments of nucleic acids by genetic engineering techniques.

The term “rejuvenate” and its various grammatical forms as used herein refer to making young or youthful again. The term “resuscitate” as used herein refers to being restored to life or being revived.

The term “RISC” or RNA-induced silencing complex, as used herein, refers to a multiprotein complex that incorporates one strand of a small interfering RNA (siRNA) or micro RNA (miRNA). RISC uses the siRNA or miRNA as a template for recognizing complementary mRNA. When it finds a complementary strand, it activates RNase and cleaves the RNA. This process is important both in gene regulation by microRNAs and in defense against viral infections, which often use double-stranded RNA as an infectious vector.

Redox signaling refers to a physiological process, where ROS act as second messengers to mediate responses that are required for proper function and survival of the cell. On the other hand, redox modulation (or redox regulation) refers to a process wherein ROS alter the activity or function of the redox-sensitive molecular targets, including signaling proteins and metabolic enzymes, leading to either physiological or pathophysiological responses. When pathophysiological responses occur, it is also known as oxidative stress [Li, R. et al. React. Oxyg. Species (Apex) (2016) 1 (1): 9-21].

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function.

The term “reverse” as used herein refers to turning backward or in an opposite direction.

The term “senolytic” or “senolyte” as used herein refers to an agent or compound that selectively induces death or clearance of senescent cells, which are cells that have entered a state of irreversible growth arrest.

The term “signal transducers and activators of transcription” or “STATs” refers to a family of seven transcription factors activated by many cytokine and growth factor receptors. There are seven STATs (1-4, 5a, 5b, and 6), which reside in the cytoplasm in an inactive form until activated by cytokine receptors. Before activation, most STATS form homodimers, due to a specific homotypic interaction between domains present at the amino termini of the individual STAT proteins. The receptor specificity of each STAT is determined by the recognition of the distinctive phosphotyrosine sequence on each activated receptor by the different SH2 domains within the various STAT proteins. Recruitment of a STAT to the activated receptor brings the STAT close to an activated Janus kinase (JAK), which can then phosphorylate a conserved tyrosine residue in the carboxy terminus of the particular STAT. This leads to a rearrangement, in which the phosphotyrosine of each STAT protein binds to the SH2 domain of the other STAT, forming a configuration that can bind DNA with high affinity. Activated STATS predominantly form homodimers, with cytokine typically activating one type of STAT. For example, IFN-gamma activates STAT1 and generates STAT 1 homodimers, while IL-4 activates STAT6, generating STAT1 homodimers. Other cytokine receptors can activate several STATS, and some STAT heterodimers can be formed. The phosphorylated STAT dimer enters the nucleus, where it acts as a transcription factor to initiate the expression of selected genes that can regulate growth and differentiation of particular subsets of lymphocytes [Janeway's Immunobiology, 9^{th} Ed., Murphy K. & Weaver, C. Eds. Garland Science, New York (2017) at 110-111].

The term “signature” as used herein refers to a specific and complex combination of biomarkers that reflect a biological state.

The term “Sirt1” as used herein refers to a member of the sirtuin family. Sirtuins are evolutionarily conserved proteins that use nicotinamide adenine dinucleotide (NAD+) as a co-substrate in their enzymatic reactions. There are seven proteins (SIRT1-7) in the human sirtuin family, among which SIRT1 is the most conserved and characterized. Sirt1 is a nicotinamide adenosine dinucleotide (NAD)-dependent deacetylase that removes acetyl groups from several transcription factors and regulatory proteins that are involved in inflammation, antioxidant expression, DNA repair, mitochondrial function, proteostasis, including autophagy. It inhibits cellular senescence and PI3K-mTOR signaling and restores defective autophagy. More specifically, Sirt1 activates FOXo3a, which regulates antioxidants (superoxide dismutases and catalase), activates PGC-1α, a transcription factor that maintains mitochondrial function, inhibits p53 induced senescence, and inhibits NF-κB thereby suppressing the senescence-associated secretory phenotype (SASP). The SASP response is activated by p21^{CIP1}, which results in activation of p38 mitogen activated protein (MAP) kinase and Janus-activated kinases (JAK), which results in the activation of NF-κB and secretion of proinflammatory cytokines (e.g., IL-1β, IL-6, TNFα), growth factors (e.g., VEGF, TGF-β), chemokines (e.g., CXCL1, CXCL8, CCL2) and matrix metalloproteinases (e.g., MMP-2, MMP-9), which are all increased in age-related diseases, including COPD [Barnes, P J et al. Am L. Respir. Crit. Care Med. (2019) 200 (5): 556-564]. Plasminogen activator inhibitor-1 (PAI-1), another characteristic SASP protein, is increased in the sputum, sputum macrophages, and alveoli of patients with COPD [Id., citing To, M. et al. Chest (2013) 144: 515-521] and in IPF [Id., citing Schlliga, M. et al. Int. J. Biochem. Cell Biol. (2018) 97: 108-117].

Sirt1 has been implicated in a broad range of physiological functions, including control of gene expression, metabolism and aging [Rahman, S. and Islam, R. Cell Communication & Signaling (2011) article 11, citing Michan, S. & Sinclair, D. Biochem. J (2007) 404: 1-13; Haigis, M C and Guarente, LP. Genes Dev. (2006) 20: 2913-2921; Yamamoto, H. et al. Mol. Endocrinol. (2007) 21: 1745-1755]. Whereas an increase in the expression of the SIRT1 protein has been observed in cancer [Elibol, B. and Kilic, U. Front. Endocrinol. (Laussane) (2018) 9: 614, citing Chen, W Y et al. Cell (2005) 123: 437-448; Wang, C. et al. Nat. Cell Biol. (2006) 8: 1025-1031], reductions in the SIRT1 level are more common in other diseases such as Alzheimer's Diseases (AD), Parkinson Disease (PD), obesity, diabetes, and cardiovascular diseases [Id., citing Lutz, M I et al. Neuromol. Med. (2014) 16: 405-414; Costa dos Santos, C. et al. Obes. Surg. (2010) 20: 633-639; Singh, P. et al. BMC Neurosci. (2017) 18: 46; Chan, S H et al. Redox Biol. (2017) 13: 301-309; Aditya, R. et al. Curr. Phar. Des. (2017) 23: 2299-2307]. Recent developments elucidated the relation between downregulation of SIRT1 levels and disease progression as an increase in oxidative stress and inflammation [Id., citing Singh, P. et al. BMC Neurosci. (2017) 18: 46; Chan, S H et al. Redox Biol. (2017) 13: 301-309]. The list of Sirt1 substrates is continuously growing and includes several transcription factors: the tumor suppressor protein p53, members of the FoxO family (forkhead box factors regulated by insulin/Akt), HES1 (hairy and enhancer of split 1), HEY2 (hairy/enhancer-of-split related with YRPW motif 2), PPARγ (peroxisome proliferator-activated receptor gamma), CTIP2 [chicken ovalbumin upstream promoter transcription factor (COUPTF)-interacting protein 2], p300, PGC-1α (PPARγ coactivator), and NF-κB (nuclear factor kappa B) [Rahman, S. and Islam, R. Cell Communication & Signaling (2011) article 11, citing Michan S. and Sinclair D. Biochem. J. (2007) 404: 1-13; Haigis, M C and Guarente, LP. Genes Dev. (2006) 20: 2913-2921; Yamamoto, H. et al. Mol. Endocrinol. (2007) 21: 1745-1755].

The term “skeletal muscle satellite cells” as used herein refers to myogenic stem cells residing between the myofiber plasmalemma and basal lamina that can self-renew and produce differentiated progeny. Skeletal muscle satellite cells may be identified by the specific expression of the paired box transcription factor Pax-7 [Yablonka-Reuveni, Z. J. Histochem. Cytochem. (2011) 59 (12): 1041-1059].

The term “slow” as used herein refers to holding back progress or development.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. Among the acceptable vehicles and solvents that may be employed are water, Ringer's solution, and isotonic sodium chloride solution.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “stem cells” refers to undifferentiated cells having high proliferative potential with the ability to self-renew that can generate daughter cells that can undergo terminal differentiation into more than one distinct cell phenotype. The term “renewal” or “self renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

The term “adult (somatic) stem cells” as used herein refers to undifferentiated cells found among differentiated cells in a tissue or organ. Their primary role in vivo is to maintain and repair the tissue in which they are found. Adult stem cells, which have been identified in many organs and tissues, including brain, bone marrow, peripheral blood, blood vessels, skeletal muscles, skin, teeth, gastrointestinal tract, liver, ovarian epithelium, and testis, are thought to reside in a specific area of each tissue, known as a stem cell niche, where they may remain quiescent (non-dividing) for long periods of time until they are activated by a normal need for more cells to maintain tissue, or by disease or tissue injury. Mesenchymal stem cells are an example of adult stem cells.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one a composition of the described invention, unless the context and usage of the phrase indicates otherwise.

The terms “surfactant protein A (SP-A)” and “surfactant protein D (SP-D)” refer to hydrophobic, collagen-containing calcium-dependent lectins, with a range of nonspecific immune functions at pulmonary and cardiopulmonary sites. SP-A and SP-D play crucial roles in the pulmonary immune response, and are secreted by type II pneumocytes, nonciliated bronchiolar cells, submucosal glands, and epithelial cells of other respiratory tissues, including the trachea and bronchi. SP-D is important in maintaining pulmonary surface tension, and is involved in the organization, stability, and metabolism of lung parenchyma (Wang K, et al. Medicine (2017) 96 (23): e7083). An increase of 49 ng/ml (1 SD) in baseline SP-A level was associated with a 3.3-fold increased risk of mortality in the first year after presentation. SP-A and SP-D are predictors of worse survival in a one year mortality regression model (Guiot, J. et al. Lung (2017) 195(3): 273-280).

The term “suspension” as used herein refers to a dispersion (mixture) in which a finely divided species is combined with another species, with the former being so finely divided and mixed that it doesn't rapidly settle out. In everyday life, the most common suspensions are those of solids in liquid.

The term “symptom” as used herein refers to a sign or an indication of disorder or disease, especially when experienced by an individual as a change from normal function, sensation, or appearance.

The term “TERT activating pathways” as used herein refer to networks of signals in cells that turn on the telomerase reverse transcriptase (TERT) gene. The TERT gene creates telomerase, an enzyme that maintains structures, called telomeres, which are composed of repeated segments of DNA found at the ends of chromosomes. Telomeres protect chromosomes from abnormally sticking together or degrading. In most cells, telomeres become progressively shorter as the cell divides. After a certain number of cell divisions, the telomeres become so short that they trigger the cell to stop dividing or to undergo apoptosis. Telomerase counteracts the shortening of telomeres by adding small repeated segments of DNA to the ends of chromosomes each time the cell divides. [Medline Plus (internet) Bethesday, MD, National Library of Medicine (US) update 2020 Jun. 2, TERT Gene] Abnormal activation of telomerase which facilitates escaping from cell senescence occurs in most cancer types. [Liu, M. et al. Cell Death & Disease (2024) 15: 90].

The TERT promoter (TERTp) region contains GC boxes that bind the zinc finger transcription factor Sp1, which increases TERT transcription, and E-boxes that bind both transcriptional enhancers and repressors. TERTp lacks a TATA box but it contains binding sites for many different transcription factors [Dratwa, M. et al. Front. Immunol. (2020) 11: 5899299, citing Takakura, M. et al. Cancer Res. (1999) 59: 551-557].

TERTp contains binding sites for a huge number of transcriptional activators and repressors that directly or indirectly regulate gene expression. Multiple pathways, such as RAS/RAF/MEK/MAPK, PI3K/Akt/mTOR, IKK/NF-κB, transforming growth factor β/Smads, PKC, and the JAK-STAT pathway regulate TERT expression and telomerase enzymatic activity [Id., citing Yamada, O. and Kawauchi, K. JAKSTAT (2013) 2: e25256]. Canonical positive regulators of TERT transcription include the oncogene c-MYC, Sp1, NF-κB, STAT family of proteins, AP-2, and GSC. c-MYC activates telomerase by inducing expression of TERT [Id., citing Zheng, L. et al. Cancer Sci (2018) 109: 395-402; Wu, K J et al. Nat. Genet. (1999) 21: 220-224]. In addition, TERT is responsible for maintenance of c-MYC levels and regulates c-MYC proteasomal degradation [Id., citing Pestana, A. et al. J. Mol. Endocrinol. (2017) 58: R129-R146].

The core promoter of TERT also contains specificity protein 1 (Sp1) binding sites that are necessary for TERT expression. Sp1 belongs to the family of nuclear proteins called Sp/KLF (specificity protein/Krüppel-like factor) that binds GC-(GGGGCGGGG) and GT-(GGTGTGGGG) rich elements [Id., citing Chang, W C and Hung, JJ. J. BioMed Sci. (2012) 19: 94; Vellingiri, B. et al. Intl J. Mol. Sci. (2020) 21: 1153]. Sp1 regulates processes such as inflammation, carcinogenesis, senescence, hormonal activation, apoptosis and angiogenesis [Id., citing Chang, WC and Hung J J. J. Biomed Sci. (2012) 19: 94]. Transcriptional activity of Sp1 is regulated by a few post-translational (glycosylation, acetylation, phosphorylation) and by direct interaction with other proteins, including other transcription factors, nuclear factors, oncogenes, and tumor suppressors. Sp1-silencing completely inhibits telomerase activity by suppressing TERT expression, leading to apoptosis. Furthermore, mutations in Sp1 binding sites (GC-boxes) significantly decrease transcriptional activity of TERTp, suggesting that Sp1 protein is involved in TERT transcription [Id., citing Jie, M M et al. Cell Commun. Signal (2019) 17: 63].

NF-κB is well known for playing a major role in inflammation, tumorigenesis, cytokine and chemokine expression, stress regulation, cell division and transformation [Id., citing Li, Y. et al. Nat. Cell Biol. (2015) 17: 1327-1328; Tong, L. et al. Biosci Rep. (2014) 34: e00115]. The NF-κB signaling pathway is a master regulator of TERT activation in cancer cells. It initiates expression of TERT by binding to either of two potential motifs in TERTp [Id., citing Li, Y. et al. Nat. Cell Biol. (2015) 17: 1327-1338]. Additionally, TERT can directly regulate expression of NF-κB-dependent genes through binding to the p65 subunit. Studies have demonstrated that telomerase can directly regulate recruitment to promoters of NF-κB target genes, such as those encoding interleukin-6 (IL-6) and tumor necrosis factor alpha (TNF-α) that are critical for inflammation and cancer progression [Id., citing Ding, D. ete al. FEBS J. (2013) 280: 3205-3211].

TERTp contains STAT3 binding sites and is overexpressed in prostate, breast, head, neck, and hematologic cancers [Id., citing Chau, M N et al. Carcinogenesis (2007) 28: 2282-2290].

The adipocyte protein 2 (AP-2) family of transcription factors contains five isoforms: AP-2α, AP-2β, AP-2γ, AP-2δ, and AP-2ϵ [Id., citing Wei, C W et al. Acta Pharmacol. Sin (2009) 30: 1297-1306; Deng, W G et al. J. Biol. Chem. (2007) 282: 26460-26470], which are encoded by the FABP4 gene. These isoforms have a major role in gene regulation and have different biological functions. They are required for morphogenesis during embryonic development Id., citing Wei, C W et al. Acta Pharmacol. Sin. (2009) 30: 1297-1306]. AP-2B specifically binds to the TERTp and activates telomerase in human cancer cells, but not normal cells. Two E-box sites in a 320-bp region of TERTp (320 bp upstream of the translational ATG site) have been observed to regulate promoter activity in human rhabdomyosarcoma cells [Id., citing Deng, W B et al. J. Biol. Chem. (2007) 282: 26460-26470].

Goosecoid homebox protein (GSC) is a TERT activator and was variously expressed in both thyroid cancer and normal thyroid cells. Additionally, GSC was overexpressed in thyroid GSC is a TERT activator and was variously expressed in both thyroid cancer and normal thyroid cells. Additionally, GSC was overexpressed in thyroid cancer [Id., citing Avin, B A et al. Genes Chromosomes Cancer (2019) 58: 530-540].

Transcriptional repressors that have been shown to downregulate TERT transcription, include MAD1/2, p53, WT1, CTCF, and MZF-2. [Id.]

Mitotic arrest deficient 1 (MAD1) is a major element of the mitotic checkpoint, and it recruits its binding partner MAD2 to nuclear pores [Id., citing Wan, J. et al. Nat. Commun. (2019) 10: 1540; Raich, N. et al. Open Biol. (2018) 8: 180166]. During mitosis, MAD1 localizes to unattached kinetochores, where it serves as a docking site for MAD2. Kinetochore-bound MAD1-MAD2 act as a catalyst for conformational change of free MAD2 [Id., citing Luo, Y. et al. Front. Cell Dev. Biol. (2018) 6: 51; Ryan, S D et al. Proc. Natl Acad. Sci USA (2012) 109: E2205-E2214]. MAD1 upregulation serves as a marker of poor prognosis, as it tends to be overexpressed in cancers. Upregulation of MAD1 leads to chromosomal instability and resistance to microtubule poisons that are currently used as chemotherapeutic agents [Id., citing Ryan, S D et al. Proc. Nat. Acad. Sci. USA (92012) E2205-2214]. MAD1 is recognized as an important cellular antagonist of c-MYC [Id., citing Lluscher, B. Eur. J. Cell Biol. (2012) 91: 506-514; Fujiki, T. et al. Cytotechnology (2010) 62: 333-339]. In addition, c-MYC and MAD1 are involved in regulation of TERT expression because they bind to the same promoter sites (E-box) to activate TERT expression [Meeran, S M et al. Cancer Prev. Res. (Phila.) (2011) 4: 1243-1254]. There are two E-boxes in TERTp, both of which constitute binding sites for c-MYC/MAX or MAD1/MAX heterodimers [Id., citing Luscher, B. Eur. J. Cell Biol. (2012) 91: 506-514; Xu, D. et al. Proc. Natl Acad. Sci. USA (2001) 998: 3826-3831]. A switch from c-MYC/MAX to MAD1/MAX, triggers decrease in H3 and H4 histone acetylation at TERTp [Id., citing Meeran, S M et al. Cancer Pre. Res. (Phila) 2011) 4: 1243-1254; Xu, D. et al. Proc. Natl Acad. Sci. USA (2001) 998: 3826-3831].

Several findings showed that p53 suppresses telomerase activity by inhibiting TERT expression This inhibition of TERT may be caused indirectly by an interaction between Sp1 and overexpressed p53 [Id., citing Cong, Y-S et al. Microbiol. Mol. Biol. Rev. (2002) 66: 407-425; Horikawa, I. et al. Cytotechnolgy (2004) 45: 23-32] and could be possibly independent of other p53 functions, such as those associated with apoptosis [Id., citing Cong, Y-S et al. Microbiol. Mol. Biol. Rev. (2002) 66: 407-425].

Another protein implicated in inhibition of TERT is the Wilms' tumor 1 (WT1) tumor suppressor [Id., citing Cong, Y-S et al. Microbiol. Mol. Biol. Rev. (2002) 66: 407-425]. It contains four zinc fingers and an RNA-binding protein that directs the development of several organs (heart, diaphragm) and genitourinary tissues ([Id., citing Toska, E. and Roberts, SGE, Biochem. J. (2014) 461: 15-32; Eozenou, C. et al. Proc. Natl Acad Sci. USA (2020) 117: 13680-13688]. It is normally expressed in kidney, testes, ovaries, and spleen [Id., citing Liu, L. et al. Gene (2004) 340: 1-10]. Most neoplasms, including lung carcinomas, renal cell carcinoma, pediatric sarcomas, and breast, ovarian, colon, melanoma, and pancreas cancers, and exhibit a possible oncogenic activity of WT1 [Id., citing Ariyaratana, S. and Loeb, DM. Expert Rev. Mol Med. (2007) 9: 1-17]; Han, Y. et al. Breast Cancer Res. (2007) 9: R43]. In addition, it is overexpressed in most acute myeloid leukemia patients and is considered to be an independent marker of minimal residual disease [Id., citing Ayatollahi, H. et al. J. Res. Med. Sci. (2017) 22: 54. Furthermore, WT1 binding to TERTp suppresses c-MYC level at both protein, and mRNA level [Id., citing Daniel, M. et al. Gene (2012) 498: 135-146; Ramllee, M K et al. Genes (Basel (2016) 7:50].

CCCTC-binding factor (CTCF) is a zinc finger transcription factor which is ubiquitously expressed in human [Id., citing Renaud, S. et al. Nucleic Acids Res. (2005) 33: 6850-6860]. Its binding sites are located in the first two exons of the TERT gene and are located in a CpG island. Earlier studies showed that CTCF does not bind to TERT in telomerase-positive cells, which is correlated with methylation of exon 1 in these cells [Id., citing Renaud, S. et al. Nucleic Acids Res. (2007) 35: 1245-1256]. Hypermethylation in this exonic region is common in most cancers, and CTCF is considered a major TERT repressor in normal cells.

The myeloid zinc finger protein (MZF)-2 is a Krüppel-like C2H2 zinc finger protein expressed predominantly in myeloid progenitor cells and involved in growth, differentiation, and tumorigenesis [Id., citing Ogawa, H. et al. J. Biol. Vhem. (2003) 278: 2921-2927]. There are multiple binding sites for MZF-2 within the TERTp region, and upregulation of MZF-2 inhibits TERTp activity. This suggests a role for MZF-2 in transcriptional downregulation of TERT [Id., citing Cong, Y-S et al. Microbiol. Mol. Biol. Rev. (2002) 66: 407-425; Fujimoto, K. et al. Nucleic Acids Res. (2007) 28: 2557-2562].

Various miRNAs are known as regulators of TERT. In particular let-7g-3p, miR-128, miR-133a, miR-138-5p, miR-498, miR-541-3p, and miR-1182 downregulate expression of TERT and telomerase activation [Id., citing Leao, R. et al. J. BioMed Sci. (2018) 25: 22; Schrank, Z. et al. Molecules (2018) 23: 2267]

As used herein, the term “therapeutic agent” or “active agent” refers to refers to the ingredient, component or constituent of the compositions of the described invention responsible for the intended therapeutic effect.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

The term “therapeutic signature” as used herein refers to a specific and complex combination of biomarkers that reflect a biological state that leads to a specific therapeutic effect.

As used herein, the term “tissue” refers to a collection of similar cells and the intercellular substances surrounding them. For example, adipose tissue is a connective tissue consisting chiefly of fat cells surrounded by reticular fibers and arranged in lobular groups or along the course of smaller blood vessels. Connective tissue is the supporting or framework tissue of the body formed of fibrous and ground substance with numerous cells of various kinds. It is derived from the mesenchyme, and this in turn from the mesoderm. The varieties of connective tissue include, without limitation, areolar or loose; adipose; sense, regular or irregular, white fibrous; elastic; mucous; lymphoid tissue; cartilage and bone.

The term “tissue inhibitors of metalloproteases” or “TIMPs” as used herein refers to key regulators of the metalloproteinases that degrade the extracellular matrix and shed cell surface molecules [Brew, K. and Nagase, H. Biochim. Biophys. Acta (2010) 1803 (1): 55-71]. TIMPs can undergo changes in molecular dynamics induced by their interactions with proteases. TIMPs also have biological activities that are independent of metalloproteinases; these include effects on cell growth and differentiation, cell migration, anti-angiogenesis, anti- and pro-apoptosis, and synaptic plasticity. The human genome has 4 paralogous genes encoding TIMPs (TIMPs-1 to -4). All four TIMPs inhibit MMPs, but with affinities that vary for different inhibitor-protease pairs. The four human TIMPs are, in general terms, broad-spectrum inhibitors of the 23 MMPs found in humans, but there are some differences in specificity among them. TIMP-1 is more restricted in its inhibitory range than the other three TIMPs, having a relatively low affinity for the membrane-type MMPs, MMP-14, MMP-16, and MMP-24 as well as for MMP-19. There are some relatively subtle differences between the affinities of different TIMPs for other MMPs. For example, TIMPs-2 and -3 are weaker inhibitors than TIMP-1 for MMP-3 and MMP-7, contrasting with their affinities for other MMPs [Id., citing Hamze, Abe t al. Protein Sci. (2007) 16: 1905-13]. TIMP-3 is unique among the mammalian TIMPs in inhibiting a broader array of metalloproteinases including several members of the aggrecanase ADAM and ADAMTS families [Id., citing Amour, A. et al. FEBS Lett. (1998) 435: 39-44; Kashiwagi, M. et al. J. Biol. Chem. (2001) 276: 12501-12504; Amour, A. et al. FEBS Lett. (2000) 473: 275-279; Hashimoto, G. et al. FEBS Lett. (2001) 494: 192-195; Wang, W M et al. Biochem. J. (2006) 398: 515-519; Jacobssen, J. et al. Biochemistry (2008) 47: 537-547]. Other TIMPs have limited inhibitory activities for ADAMs: TIMP-1 and TIMP-2 inhibit ADAM10 [Id., citing Amour, A. et al. FEBS Lett. (2000) 473: 275-279] and ADAM12 [Id., citing Jacobsen, J. et al. Biochemistry (2008) 47: 537-547], respectively. TIMP-3 and N-TIMP-4, but not full-length TIMP-4, inhibit ADAM17 [Id., citing Lee, M H et al. J. Biol. Chem. (2005) 280: 15967-15975]. TIMP-4 was also reported to inhibit ADAM28 [Mochizuki, S. et al. Biochem. Biophys. Res. Commun. (2004) 315: 79-84]. ADAM metalloproteinases differ from the MMPs in domain structures and are highly divergent in catalytic domain sequences: ADAMs are membrane-bound enzymes containing disintegrin, cysteine-rich, EGF-like and transmembrane domains C-terminal to their catalytic domains [Id., citing Edwards, D R et al. Mol. Aspects Med. (2008) 29: 2582-89]; and ADAMTS (disintegrin-metalloproteinases with thrombospondin motifs) are secreted proteins with a disintegrin domain and variable numbers of thrombospondin type 1 motifs and other domains in their C-terminal regions [Id., citing Porter, S. et al. Biochem. J. (2005) 386: 15-27].

The term “toll-like receptor” or “TLRs” as used herein refers to innate receptors on macrophages, dendritic cells and some other cells that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

Smad-dependent pathway for TGF-signaling. The classical Smad-dependent pathway for transforming growth factor-β (TGFβ) signaling occurs when TGF-β receptor type 2, which is constitutively active, transphosphorylates and forms a complex with the TGF-β-bound TGF-β receptor type 1. This complex then phosphorylates serine residues of cytoplasmic receptor-activated Smad (R-Smad), a complex of Smad2 and Smad3. These two heterodimerize and bind to the common mediator Smad (Co-Smad) Smad4, and the whole complex translocates across the nuclear membrane to interact with specific cis-acting elements in the regulatory regions of its target genes [He, W. and Dai, C. Curr. Pathobiol. Rep. (2015) 3(2): 183-192, citing Tsou, P S et al. Am. J. Physiol. Cell Physiol. (2014) 307: C2-13], recruiting coactivators such as p300 and CBP; corepressors such as c-Ski, SnoN, transforming growth-inhibiting factor, and Smad nuclear-interacting protein 1; or transcription factors such as AP-1 and Sp1 to modulate gene expression [Id., citing Biernacka, A. et al. Growth Factors (2011) 29: 196-202]. Inhibitory Smad (I-Smad) Smad6 or Smad7, acting as negative regulators, not only antagonizes the TGF-β/Smad pathway by binding to TGF-β1 or competing with activated R-Smad for binding to Co-Smad, but also recruits the E3 ubiquitin-protein ligases Smurf1 and Smurf2, which target Smad proteins for proteasomal degradation, thereby blocking Smad2/3 activation, facilitating receptor degradation, and eventually terminating Smad-mediated signaling.

The term “transcriptome” as used herein refers to the full range of messenger RNA (or mRNA) molecules expressed by an organism. The term “transcriptome” also refers to the array of mRNA transcripts produced in a particular cell or tissue type.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tumor necrosis factor receptor-associated factor 6” or TRAF6″ as used herein refers to an E3 ligase that produces a K63 polyubiquitin signaling scaffold in TLR-4 signaling to activate the NFκB pathway.

The term “tumor susceptibility gene 101” or Tsg101″ as used herein refers to a housekeeping gene highly conserved between mouse and human for which significant variations in high or low protein expression levels in normal tissues or cancer cells are likely a consequence of post-transcriptional or post-translational mechanisms. It has been suggested to function as a negative regulator of ubiquitin-mediated protein degradation [Ferraiuolo, R-M, et al., Cancers (Basel) (2020) 12 (2): 450, citing Koonin, E V and Abagyan, RA. Nat. Genet. (1997) 75: 467-469) as well as a mediator for the intracellular movement of ubiquinated proteins [Id., citing Katzmann, D J et al. Cell (2001) 106: 145-155].

The term “tunable” as used herein refers to that which can be adjusted or modified in terms of its properties, settings, or characteristics.

The term “WNT1 (Wnt Family Member 1)” as used herein refers to a protein coding gene and a member of the WNT gene family. Protein Wnt-1 is encoded by the WNT1 gene and is very conserved in evolution. It acts in the canonical Wnt signaling pathway by promoting beta-catenin-dependent transcriptional activation. Activation of Wnt/β catenin signaling has been reported in skin, kidney, liver, lung and cardiac fibrosis [He, W and Dai, C. Curr. Pathobiol. Rep (2015) 3: 183-192, citing Wynn, T A and Ramalingam, TR. Nat. Med. (2012) 18: 1028-1040; Lam, A P and Gottardi, CJ. Curr. Opin. Rheumatol. (2011) 23: 562-567]. Wnt proteins deliver their signal across the plasma membrane by interacting with Fizzled receptors and coreceptors. Once Wnts bind to their receptors/coreceptors, they initiate a chain of downstream signaling events leading to dephosphorylation of β-catenin [Id., citing Liu, Y. J. Am. Soc. Nephrol. (2010) 21: 212-222]. Escaping from degradation mediated by the ubiquitin/proteasome system stabilized β catenin accumulates in the cytoplasm and translocates into the nucleus, where it interacts with its DNA-binding partner known as T cell factor (TCF)/lymohpcyte enhancer-binding factor 1 (LEF1) to stimulate the transcription of Wnt target genes [Id., citing Lam, A P and Gottardi, CJ. Curr. Opin. Rheumatol. (2011) 23: 562-567; Liu, Y. J. Am. Soc. Nephrol. (2010) 21: 212-222; Huang, H. & He, X. Curr. Opin. Cell Biol. (2008) 20: 119-125].

The term “wound healing” refers to the process by which the body repairs trauma to any of its tissues, especially those caused by physical means and with interruption of continuity.

A wound-healing response often is described as having three distinct phases-injury, inflammation and repair. Generally speaking, the body responds to injury with an inflammatory response, which is crucial to maintaining the health and integrity of an organism. If, however, it goes awry, it can result in tissue destruction.

Although these three phases are often presented sequentially, during chronic or repeated injury, these processes function in parallel, placing significant demands on regulatory mechanisms [Wilson and Wynn, Mucosal Immunol., 2009, 3(2): 103-121].

### Phase I: Injury

Injury caused by factors including, but not limited to, autoimmune or allergic reactions, environmental particulates, chronological aging (meaning the time that has passed since a person's birth, measured in years, months and days), or infection or mechanical damage, often results in the disruption of normal tissue architecture, initiating a healing response. Damaged epithelial and endothelial cells must be replaced to maintain barrier function and integrity and prevent blood loss, respectively. Acute damage to endothelial cells leads to the release of inflammatory mediators and initiation of an anti-fibrinolytic coagulation cascade, temporarily plugging the damaged vessel with a platelet and fibrin-rich clot. For example, lung homogenates, epithelial cells or bronchoalveolar lavage fluid from idiopathic pulmonary fibrosis (IPF) patients contain greater levels of the platelet-differentiating factor, X-box-binding protein-1, compared with chronic obstructive pulmonary disease (COPD) and control patients, suggesting that clot-forming responses are continuously activated. In addition, thrombin (a serine protease required to convert fibrinogen into fibrin) is also readily detected within the lung and intra-alveolar spaces of several pulmonary fibrotic conditions, further confirming the activation of the clotting pathway. Thrombin also can directly activate fibroblasts, increasing proliferation and promoting fibroblast differentiation into collagen-producing myofibroblasts. Damage to the airway epithelium, specifically alveolar pneumocytes, can evoke a similar anti-fibrinolytic cascade and lead to interstitial edema, areas of acute inflammation, and separation of the epithelium from the basement membrane.

Platelet recruitment, degranulation and clot formation rapidly progress into a phase of vasoconstriction with increased permeability, allowing the extravasation (movement of white blood cells from the capillaries to the tissues surrounding them) and direct recruitment of leukocytes to the injured site. The basement membrane, which forms the extracellular matrix underlying the epithelium and endothelium of parenchymal tissue, precludes direct access to the damaged tissue. To disrupt this physical barrier, zinc-dependent endopeptidases, also called matrix metalloproteinases (MMPs), cleave one or more extracellular matrix constituents allowing extravasation of cells into, and out of, damaged sites.

### Phase II: Inflammation

Once access to the site of tissue damage has been achieved, chemokine gradients recruit inflammatory cells. Neutrophils, eosinophils, lymphocytes, and macrophages are observed at sites of acute injury with cell debris and areas of necrosis cleared by phagocytes.

The early recruitment of eosinophils, neutrophils, lymphocytes, and macrophages providing inflammatory cytokines and chemokines can contribute to local TGF-β and IL-13 accumulation. Following the initial insult and wave of inflammatory cells, a late-stage recruitment of inflammatory cells may assist in phagocytosis, in clearing cell debris, and in controlling excessive cellular proliferation, which together may contribute to normal healing. Late-stage inflammation may serve an anti-fibrotic role and may be required for successful resolution of wound-healing responses. For example, a late-phase inflammatory profile rich in phagocytic macrophages, assisting in fibroblast clearance, in addition to IL-10-secreting regulatory T cells, suppressing local chemokine production and TGF-β, may prevent excessive fibroblast activation.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses) and influence the response of innate cells to invading pathogens. Endogenous danger signals also can influence local innate cells and orchestrate the inflammatory cascade.

The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. Fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1β), interleukin-6 (IL-6), tumor necrosis factor alpha (TNF-α), transforming growth factor beta (TGF-β), and platelet-derived growth factors (PDGFs)). Each of these cytokines has been shown to exhibit significant pro-fibrotic activity, acting through the recruitment, activation and proliferation of fibroblasts, macrophages, and myofibroblasts.

### Phase III: Tissue Repair and Contraction

The closing phase of wound healing consists of an orchestrated cellular reorganization guided by a fibrin (a fibrous protein that is polymerized to form a “mesh” that forms a clot over a wound site)-rich scaffold formation, wound contraction, closure and re-epithelialization. The vast majority of studies elucidating the processes involved in this phase of wound repair have come from dermal wound studies and in vitro systems.

Myofibroblast-derived collagens and smooth muscle actin (α-SMA) form the provisional extracellular matrix, with macrophage, platelet, and fibroblast-derived fibronectin forming a fibrin scaffold. Collectively, these structures are commonly referred to as granulation tissues. Primary fibroblasts or alveolar macrophages isolated from IPF patients produce significantly more fibronectin and α-SMA than control fibroblasts, indicative of a state of heightened fibroblast activation. It has been reported that IPF patients undergoing steroid treatment had similar elevated levels of macrophage-derived fibronectin as IPF patients without treatment. Thus, similar to steroid resistant IL-13-mediated myofibroblast differentiation, macrophage-derived fibronectin release also appears to be resistant to steroid treatment, providing another reason why steroid treatment may be ineffective. From animal models, fibronectin appears to be required for the development of pulmonary fibrosis, as mice with a specific deletion of an extra type III domain of fibronectin (EDA) developed significantly less fibrosis following bleomycin administration compared with their wild-type counterparts.

In addition to fibronectin, the provisional extracellular matrix consists of glycoproteins (such as PDGF), glycosaminoglycans (such as hyaluronic acid), proteoglycans and elastin. Growth factor and TGF-β-activated fibroblasts migrate along the extracellular matrix network and repair the wound. Within skin wounds, TGF-β also induces a contractile response, regulating the orientation of collagen fibers. Fibroblast to myofibroblast differentiation, as discussed above, also creates stress fibers and the neo-expression of α-SMA, both of which confer the high contractile activity within myofibroblasts. The attachment of myofibroblasts to the extracellular matrix at specialized sites called the “fibronexus” or “super mature focal adhesions” pull the wound together, reducing the size of the lesion during the contraction phase. The extent of extracellular matrix laid down and the quantity of activated myofibroblasts determines the amount of collagen deposition. To this end, the balance of matrix metalloproteinases (MMPs) to tissue inhibitor of metalloproteinases (TIMPs) and collagens to collagenases vary throughout the response, shifting from pro-synthesis and increased collagen deposition towards a controlled balance, with no net increase in collagen. For successful wound healing, this balance often occurs when fibroblasts undergo apoptosis, inflammation begins to subside, and granulation tissue recedes, leaving a collagen-rich lesion. The removal of inflammatory cells, and especially α-SMA-positive myofibroblasts, is essential to terminate collagen deposition. Interestingly, in IPF patients, the removal of fibroblasts can be delayed, with cells resistant to apoptotic signals, despite the observation of elevated levels of pro-apoptotic and FAS-signaling molecules.

Several studies also have observed increased rates of collagen-secreting fibroblast and epithelial cell apoptosis in IPF, suggesting that yet another balance requires monitoring of fibroblast apoptosis and fibroblast proliferation. From skin studies, re-epithelialization of the wound site re-establishes the barrier function and allows encapsulated cellular re-organization. Several in vitro and in vivo models, using human or rat epithelial cells grown over a collagen matrix, or tracheal wounds in vivo, have been used to identify significant stages of cell migration, proliferation, and cell spreading. Rapid and dynamic motility and proliferation, with epithelial restitution from the edges of the denuded area, occur within hours of the initial wound. In addition, sliding sheets of epithelial cells can migrate over the injured area assisting wound coverage. Several factors have been shown to regulate re-epithelialization, including serum-derived transforming growth factor alpha (TGF-α), and matrix metalloproteinase-7 (MMP-7) (which itself is regulated by TIMP-1).

Collectively, the degree of inflammation, angiogenesis, and amount of extracellular matrix deposition all contribute to ultimate development of a fibrotic lesion.

### Embodiments

### Compositions

According to one aspect, the present disclosure provides a composition comprising an isolated population of extracellular vesicles (EVs) comprising a purified, enriched population of potent exosomes derived from mesenchymal stem cells (MSCs), wherein
(a) the EVs comprise an identity signature of three or more biomarkers selected from CD9, CD63, CD81, or Tsg101+;
(b) the EVs comprise total protein of about 1 mg;
(c) the EVs comprise total RNA content greater than 20 μg;
(d) the exosomes comprise a cargo comprising a therapeutic signature of one or more miRNAs selected from miR-29a, miR-10a, miR-34a, miR-125, miR-181a, miR-181c, miR-Let-7a, miR-Let-7b, miR-Let-7d, miR-146a, miR-145, miR-21, miR-101, and miR-199; and
(e) size of the exosomes is 50-130 nm, inclusive;
wherein lot to lot and batch to batch variability relative to prior lots of the composition is below a significance level of ≤0.05; and wherein expression of the miRNA cargo is configured to treat an age-related chronic disease.

According to some embodiments, extracellular vesicles are derived from a biological sample. According to some embodiments, the source of MSCs is a tissue autologous to the recipient subject. According to some embodiments, the source of the MSCs is a tissue allogeneic to the recipient subject. According to some embodiments, the tissue is mammalian. According to some embodiments, the tissue is human. According to some embodiments, the source of the MSCs is placental tissue obtained from one or more areas, including both material and fetal tissue, e.g., chorionic membrane (CM), chorionic trophoblast without villi (CT-V), chorionic villi (CV), or decidua (DC). According to some embodiments, the source of MSCs is adipose tissue. According to some embodiments, the adipose tissue is subcutaneous white adipose tissue. According to some embodiments, the source of MSCs is bone marrow, umbilical cord tissue, lung tissue, heart tissue, or dental pulp. According to some embodiments, the source of the MSCs is a body fluid. According to some embodiments, the body fluid is peripheral blood, umbilical cord blood, urine, or amniotic fluid.

Placenta. Structure and development. The placenta and associated extraembryonic membranes are formed from the zygote at the start of each pregnancy and thus have the same genetic composition as the fetus. The two principal tissue sources are the trophectoderm that forms the wall of the blastocyst, and the underlying extraembryonic mesoderm. The trophectoderm differentiates into trophoblast, which in turn forms the epithelial covering of the placenta and also gives rise to the subpopulation of invasive extravillous trophoblast cells. The extraembryonic mesoderm forms the stromal core of the placenta, from which originate the fibroblasts, vascular network and resident macrophage population [Burton, GJ, Fowden, A L. Philos. Trans. R. Soc. Lond. B. Biol. Sci. (2015) 370 (1663): 20140066].

The surfaces of the mature placenta are the chorionic plate that faces the fetus and to which the umbilical cord is attached, and the basal plate that abuts the maternal endometrium. Between these plates is a cavity, the intervillous space, into which 30-40 elaborately branched fetal villous trees project. Each villous tree arises from a stem villus attached to the deep surface of the chorionic plate, and branches repeatedly to create a globular lobule 1-3 cm in diameter. The center of a lobule is located over the opening of a maternal spiral artery through the basal plate. Maternal blood released at these openings percolates between the villous branches before draining into openings of the uterine veins and exiting the placenta. Each lobule thus represents an independent maternal-fetal exchange unit.

The final branches of the villous trees are the terminal villi. These present a surface area of 12-14 m² at term and are richly vascularized by a fetal capillary network. The capillaries display local dilations, referred to as sinusoids, which bring the endothelium into close approximation to the covering of trophoblast. This is locally thinned, and the diffusion distance between the maternal and fetal circulations may be reduced to as little at 2-3 μm. The morphological resemblance of these structures, termed vasculosyncytial membranes, to the alveoli of the lung has led to the assumption that they are the principal sites of maternal-fetal exchange. Terminal villi are formed primarily from 20 weeks of gestation onwards, and elaboration of the villous trees continues until term [Id., citing Simpson, R A et al. Placenta (1992) 13: 501-512].

The epithelial covering of the villous tree is the syncytiotrophoblast, a true multinucleated syncytium that presents no intercellular clefts to the intervillous space. This arrangement may assist in preventing the vertical transmission of pathogens from the maternal blood [Id., citing Robbins, J R et al. PLoS Pathog. (2010) 6: e1000732] but may also facilitate regional specializations of the syncytiotrophoblast. Because of its location, the syncytiotrophoblast is involved in many of the functions of the placenta, such as the synthesis and secretion of large quantities of steroid and peptide hormones, protection against xenobiotics and active transport. Hence, it has a high metabolic rate, and accounts for approximately 40% of the total oxygen consumption of the feto-placental unit [Id., citing Carter, AM (2000) Placenta (2000) 21 (Suppl. A): S31-S37]. Interposing such an active tissue between the maternal and fetal circulations potentially reduces the oxygen available for the fetus, and so the syncytiotrophoblast shows regional variations in thickness around the villous surface, being very thin and devoid of organelles at the site of vasculosyncytial membranes and thicker over non-vascular parts of the villous surface. Having no lateral cell boundaries may facilitate flow of the syncytioplasm and so help to optimize oxygen supply to the fetus [Id., citing Burton, GJ, Tham, SW. J. Dev. Physiol. (1992) 18: 43-47].

The syncytiotrophoblast is a highly polarized epithelium, bearing a dense covering of microvilli on its apical border. The projections provide a surface amplification factor of 5-7× for insertion of receptor and transporter proteins. At the base of each microvillus is a clathrin-coated pit, which is capable of forming a coated vesicle for the transport of macromolecules across the syncytiotrophoblast [Id., citing Ockleford, CD, Whyte, et al. J. Cell Si. (1977) 25: 293-312].

The syncytiotrophoblast is a terminally differentiated tissue, and its expansion during pregnancy is achieved by the fusion and incorporation of underlying mononuclear progenitor cytotrophoblast cells that rest on the underlying basement membrane. Fusion is a complex event that is still not fully understood but involves exit of the progenitor from the cell cycle, the formation of gap junctions with the syncytiotrophoblast, externalization of phosphatidylserine and the expression of two endogenous retroviral proteins that entered the primate genome 25 and more than 40 million years ago [Id., citing Gauster, M., Huppertz, B. (2010) Placenta 31: 82-88; Rote, N S et al. Placenta (2010) 31: 89-96].

### Placental Tissue Matrix

The placenta is considered one of the most important sources of stem cells and has been studied extensively. It fulfills two main desiderata of cell therapy: a source of a high as possible number of cells and the use of non-invasive methods for their harvesting. Their high immunological tolerance supports their use as an adequate source in cell therapy [Mihu, C. et al., 2008, Romanian Journal of Morphology and Embryology, 2008, 49(4): 441-446].

The fetal adnexa is composed of the placenta, fetal membranes, and umbilical cord. The term placenta is discoid in shape with a diameter of 15-20 cm and a thickness of 2-3 cm. The fetal membranes, amnion and chorion, which enclose the fetus in the amniotic cavity, and the endometrial decidua extend from the margins of the chorionic disc. The chorionic plate is a multilayered structure that faces the amniotic cavity. It consists of two different structures: the amniotic membrane (composed of epithelium, compact layer, amniotic mesoderm, and spongy layer) and the chorion (composed of mesenchyme and a region of extravillous proliferating trophoblast cells interposed in varying amounts of Langhans fibrinoid, either covered or not by syncytiotrophoblast).

Villi originate from the chorionic plate and anchor the placenta through the trophoblast of the basal plate and maternal endometrium. From the maternal side, protrusions of the basal plate within the chorionic villi produce the placental septa, which divide the parenchyma into irregular cotyledons [Parolini, O. et al., 2008, Stem Cell, 2008, 26:300-311].

Some villi anchor the placenta to the basal plate, whereas others terminate freely in the intervillous space. Chorionic villi present with different functions and structure. In the term placenta, the stem villi show an inner core of fetal vessels with a distinct muscular wall and connective tissue consisting of fibroblasts, myofibroblasts, and dispersed tissue macrophages (Hofbauer cells). Mature intermediate villi and term villi are composed of capillary vessels and thin mesenchyme. A basement membrane separates the stromal core from an uninterrupted multinucleated layer, called the syncytiotrophoblast. Between the syncytiotrophoblast and its basement membrane are single or aggregated Langhans cytotrophoblastic cells, commonly called cytotrophoblast cells [Parolini, O. et al., 2008, Stem Cell, 2008, 26:300-311].

Four regions of fetal placenta can be distinguished: an amniotic epithelial region, an amniotic mesenchymal region, a chorionic mesenchymal region, and a chorionic trophoblastic region.

### Amniotic Membrane

Fetal membranes continue from the edge of the placenta and enclose the amniotic fluid and the fetus. The amnion is a thin, avascular membrane composed of an inner epithelial layer and an outer layer of connective tissue that, and is contiguous, over the umbilical cord, with the fetal skin. The amniotic epithelium (AE) is an uninterrupted, single layer of flat, cuboidal and columnar epithelial cells in contact with amniotic fluid. It is attached to a distinct basal lamina that is, in turn, connected to the amniotic mesoderm (AM). In the amniotic mesoderm closest to the epithelium, an acellular compact layer is distinguishable, composed of collagens I and III and fibronectin. Deeper in the AM, a network of dispersed fibroblast-like mesenchymal cells and rare macrophages are observed. It has been reported that the mesenchymal layer of amnion indeed contains two subfractions, one having a mesenchymal phenotype, also known as amniotic mesenchymal stromal cells, and the second containing monocyte-like cells.

### Chorionic Membrane

A spongy layer of loosely arranged collagen fibers separates the amniotic and chorionic mesoderm. The chorionic membrane (chorion leave) consists of mesodermal and trophoblastic regions. Chorionic and amniotic mesoderm are similar in composition. A large and incomplete basal lamina separates the chorionic mesoderm from the extravillous trophoblast cells. The latter, similar to trophoblast cells present in the basal plate, are dispersed within the fibrinoid layer and express immunohistochemical markers of proliferation. The Langhans fibrinoid layer usually increases during pregnancy and is composed of two different types of fibrinoid: a matrix type on the inner side (more compact) and a fibrin type on the outer side (more reticulate). At the edge of the placenta and in the basal plate, the trophoblast interdigitates extensively with the decidua [Cunningham, F. et al., The placenta and fetal membranes, Williams Obstetrics, 20th ed. Appleton and Lange, 1997, 95-125; Benirschke, K. and Kaufmann, P. Pathology of the human placenta. New York, Springer-Verlag, 2000, 42-46, 116, 281-297].

### Amnion-Derived Stem Cells

The amniotic membrane itself contains multipotent cells that are able to differentiate in the various layers. Studies have reported their potential in neural and glial cells, cardiac repair and also hepatocyte cells. Studies have shown that human amniotic epithelial cells express stem cell markers and have the ability to differentiate toward all three germ layers. These properties, the ease of isolation of the cells, and the availability of placenta, make amniotic membrane a useful and noncontroversial source of cells for transplantation and regenerative medicine.

Amniotic epithelial cells can be isolated from the amniotic membrane by several methods that are known in the art. According to one such method, the amniotic membrane is stripped from the underlying chorion and digested with trypsin or other digestive enzymes. The isolated cells readily attach to plastic or basement membrane-coated culture dishes. Culture is established commonly in a simple medium such as Dulbecco's Modified Eagle's Medium (DMEM) supplemented with 5%-10% serum and epidermal growth factor (EGF), in which the cells proliferate robustly and display typical cuboidal epithelial morphology. Normally, 2-6 passages are possible before proliferation ceases. Amniotic epithelial cells do not proliferate well at low densities.

Amniotic membrane contains epithelial cells with different surface markers, suggesting some heterogeneity of phenotype. Immediately after isolation, human amniotic epithelial cells express very low levels of human leukocyte antigen (HLA)-A, B, C; however, by passage 2, significant levels are observed. Additional cell surface antigens on human amniotic epithelial cells include, but are not limited to, ATP-binding cassette transporter G2 (ABCG2/BCRP), CD9, CD24, E-cadherin, integrins α6 and 1, c-met (hepatocyte growth factor receptor), stage-specific embryonic antigens (SSEAs) 3 and 4, and tumor rejection antigens 1-60 and 1-81. Surface markers thought to be absent on human amniotic epithelial cells include SSEA-1, CD34, and CD133, whereas other markers, such as CD117 (c-kit) and CCR4 (CC chemokine receptor), are either negative or may be expressed on some cells at very low levels. Although initial cell isolates express very low levels of CD90 (Thy-1), the expression of this antigen increases rapidly in culture [Miki, T. et al., Stem Cells, 2005, 23: 1549-1559; Miki, T. et al., Stem Cells, 2006, 2: 133-142].

In addition to surface markers, human amniotic epithelial cells express molecular markers of pluripotent stem cells, including octamer-binding protein 4 (OCT-4) SRY-related HMG-box gene 2 (SOX-2), and Nanog [Miki, T. et al., Stem Cells, 2005, 23: 1549-1559]. Previous studies also have shown that human amnion cells in xenogeneic, chimeric aggregates, which contain mouse embryonic stem cells, can differentiate into all three germ layers and that cultured human amniotic epithelial cells express neural and glial markers, and can synthesize and release acetylcholine, catecholamines, and dopamine. Hepatic differentiation of human amniotic epithelial cells also has been reported. Studies have reported that cultured human amniotic epithelial cells produce albumin and α-fetoprotein and that albumin and α-fetoprotein-positive hepatocyte-like cells could be identified integrated into hepatic parenchyma following transplantation of human amniotic epithelial cells into the livers of severe combined immunodeficiency (SCID) mice. The hepatic potential of human amniotic epithelial cells was confirmed and extended, whereby in addition to albumin and α-fetoprotein production, other hepatic functions, such as glycogen storage and expression of liver-enriched transcription factors, such as hepatocyte nuclear factor (HNF) 3γ and HNF4α, CCAAT/enhancer-binding protein (CEBP α and β), and several of the drug metabolizing genes (cytochrome P450) were demonstrated. The wide range of hepatic genes and functions identified in human amniotic epithelial cells has suggested that these cells may be useful for liver-directed cell therapy [Parolini, O. et al., 2008, Stem Cell, 2008, 26:300-311].

Differentiation of human amniotic epithelial cells to another endodermal tissue, pancreas, also has been reported. For example, it was shown that human amniotic epithelial cells cultured for 2-4 weeks in the presence of nicotinamide to induce pancreatic differentiation, expressed insulin. Subsequent transplantation of the insulin-expressing human amniotic epithelial cells corrected the hyperglycemia of streptozotocin-induced diabetic mice. In the same setting, human amniotic mesenchymal stromal cells were ineffective, suggesting that human amniotic epithelial cells, but not human amniotic mesenchymal stromal cells, were capable of acquiring B-cell fate [Parolini, O. et al., 2008, Stem Cell, 2008, 26:300-311].

Mesenchymal Stromal Cells from Amnion and Chorion: HAMSC and hCMSC

Human amniotic mesenchymal cells (hAMSC) and human chorionic mesenchymal cells (hCMSC) are thought to be derived from extraembryonic mesoderm. hAMSC and hCMSC can be isolated from first-, second-, and third-trimester mesoderm of amnion and chorion, respectively. For hAMSC, isolations are usually performed with term amnion dissected from the deflected part of the fetal membranes to minimize the presence of maternal cells. For example, homogenous hAMSC populations can be obtained by a two-step procedure, whereby: minced amnion tissue is treated with trypsin to remove hAEC and the remaining mesenchymal cells are then released by digestion (e.g., with collagenase or collagenase and DNase). The yield from term amnion is about 1 million hAMSC and 10-fold more hAEC per gram of tissue [Casey, M. and MacDonald P., Biol Reprod, 1996, 55: 1253-1260].

hCMSCs are isolated from both first- and third-trimaster chorion after mechanical and enzymatic removal of the trophoblastic layer with dispase. Chorionic mesodermal tissue is then digested (e.g., with collagenase or collagenase plus DNase). Mesenchymal cells also have been isolated from chorionic fetal villi through explant culture, although maternal contamination is more likely [Zhang, X., et al., Biochem Biophys Res Commun. (2006) 340: 944-952; Soncini, M. et al., J Tissue Eng Regen Med (2007) 1: 296-305; Zhang et al., Biochem Biophys Res Commun (2006) 351: 853-859].

The surface marker profile of cultured hAMSC and hCMSC, and mesenchymal stromal cells (MSC) from adult bone marrow are similar. All express typical mesenchymal markers (Table 2) but are negative for hematopoietic (CD34 and CD45) and monocytic markers (CD14). Surface expression of SSEA-3 and SSEA-4 and RNA for OCT-4 has been reported [Wei J. et al., Cell Transplant (2003) 12: 545-552; Wolbank, S. et al., Tissue Eng. (2007) 13: 1173-1183; Alviano, F. et al., BMC Dev Biol. (2007) 7: 11; Zhao, P. et al, Transplantation, (2005) 79: 528-535]. Both first- and third trimester hAMSC and hCMSC express low levels of HLA-A, B, C but not HLA-DR, indicating an immunoprivileged status [Portmann-Lanz, C. et al, Am J Obstet Gynecol. (2006) 194: 664-673; Wolbank, S. et al., Tissue Eng. (2007) 13: 1173-1183].

Table 2 provides surface antigen expression profile at passages 2-4 for amniotic mesenchymal stromal and human chorionic mesenchymal stromal stem cells.

TABLE 2
Specific surface antigen expression for amniotic mesenchymal
stromal cells and human chorionic mesenchymal stromal cells

| Positive (≥95%) | Negative (≤2%) |
|---|---|
| CD90 | CD45 |
| CD73 | CD34 |
| CD105 | HLA-DR |

Both hAMSCs and hCMSCs differentiate toward “classic” mesodermal lineages (osteogenic, chondrogenic, and adipogenic) and differentiation of hAMSC to all three germ layers-ectoderm (neural), mesoderm (skeletal muscle, cardiomyocytic and endothelial), and endoderm (pancreatic) was reported [Int'Anker, P. et al., Stem Cells (2004) 22: 1338-1345; Portmann-Lanz, C. et al, Am J Obstet Gyneco (2006) 194: 664-673; Wolbank, S. et al., Tissue Eng. (2007) 13: 1173-1183; Soncini, M. et al., J Tissue Eng Regen Med. (2007) 1: 296-305; Alviano, F., BMC Dev Biol. (2007) 7: 11].

Human amniotic and chorionic cells successfully and persistently engraft in multiple organs and tissues in vivo. Human chimerism detection in brain, lung, bone marrow, thymus, spleen, kidney, and liver after either intraperitoneal or intravenous transplantation of human amnion and chorion cells into neonatal swine and rats was indeed indicative of an active migration consistent with the expression of adhesion and migration molecules (L-selectin, VLA-5, CD29, and P-selectin ligand 1), as well as cellular matrix proteinase (MMP-2 and MMP-9) [Bailo, M. et al., Transplantation, 2004, 78: 1439-144]).

### Umbilical Cord

Two types of umbilical stem cells can be found, namely hematopoietic stem cells (UC-HS) and mesenchymal stem cells, which in turn can be found in umbilical cord blood (UC-MS) or in Wharton's jelly (UC-MM). The blood of the umbilical cord has long been in the focus of attention of researchers as an important source of stem cells for transplantation, for several reasons: (1) it contains a higher number of primitive hematopoietic stem cells (HSC) per volume unit, which proliferate more rapidly, than bone marrow; (2) there is a lower risk of rejection after transplantation; (3) transplantation does not require a perfect HLA antigen match (unlike in the case of bone marrow); (4) UC blood has already been successfully used in the treatment of inborn metabolic errors; and (5) there is no need for a new technology for collection and storage of the mononuclear cells from UC blood, since such methods are long established.

Umbilical cord (UC) vessels and the surrounding mesenchyma (including the connective tissue known as Wharton's jelly) derive from the embryonic and/or extraembryonic mesodermis. Thus, these tissues, as well as the primitive germ cells, are differentiated from the proximal epiblast, at the time of formation of the primitive line of the embryo, containing MSC and even some cells with pluripotent potential. The UC matrix material is speculated to be derived from a primitive mesenchyma, which is in a transition state towards the adult bone marrow mesenchyma [Mihu, C. et al., 2008, Romanian Journal of Morphology and Embryology (2008) 49 (4): 441-446].

The blood from the placenta and the umbilical cord is relatively easy to collect in usual blood donation bags, which contain anticoagulant substances. Mononuclear cells are separated by centrifugation on Ficoll gradient, from which the two stem cell populations will be separated: (1) hematopoietic stem cells (HSC), which express certain characteristic markers (CD34, CD133); and (2) mesenchymal stem cells (MSC) that adhere to the culture surface under certain conditions (e.g., modified McCoy medium and lining of vessels with Fetal Bovine Serum (FBS) or Fetal Calf Serum (FCS)) [Munn, D. et al., Science (1998) 281: 1191-1193; Munn, D. et al., J Exp Med (1999) 189: 1363-1372]. Umbilical cord blood MSCs (UC-MS) can produce cytokines, which facilitate grafting in the donor and in vitro HSC survival compared to bone marrow MSC [Zhang, X et al., Biochem Biophys Res Commun. (2006) 351: 853-859].

MSCs from the umbilical cord matrix (UC-MM) are obtained by different culture methods depending on the source of cells, e.g., MSCs from the connective matrix, from subendothelial cells from the umbilical vein or even from whole umbilical cord explant. They are generally well cultured in DMEM medium, supplemented with various nutritional and growth factors; in certain cases, prior treatment of vessels with hyaluronic acid has proved beneficial [Baban, B. et al., J Reprod Immunol. (2004), 61: 67-77].

### Bone Marrow

Human bone marrow can be obtained from the iliac crest of patients after having obtained their written consent. BM is collected aseptically into K2EDTA tubes. The buffy coat is isolated by centrifugation (450×g, 10 min), suspended in 1.5 mL PBS, and used for culture. The separated buffy coat is layered onto equal volume of Ficoll (GE Health Care, USA) and centrifuged (400×g, 20 min). Cells at the interface are removed and washed twice in sterile PBS.

Human bone marrow progenitor cells are cultured on tissue treated culture plates in DMEM medium supplemented with 10% FBS and penicillin/streptomycin (50 U/mL and 50 mg/mL, respectively). The plates are maintained at 37 °C in a humidified atmosphere containing 5% CO₂ for 48 h. To exchange the medium, the plates are washed with PBS in order to remove non-adhered cells and the medium is replaced. The remaining cells have a heterogeneous fibroblastic-like appearance and exhibit colony formation. The cultures can be maintained for an additional week with one medium exchange.

### Adipose Tissue

In comparison to BM-MSC, MSC from adipose tissue, the adipose-derived stromal/stem cells (ASCs), occur at a 100-1000-fold higher frequency within adipose tissue on a volume basis [Aust L, et al., Cytotherapy (2004) 6(1): 7-14]. Harvesting adipose tissue is also minimally invasive and less painful than bone marrow tissue. Conventional enzymatic methods, using enzymes such as collagenase, trypsin, or dispase, are widely used for MSC isolation from adipose tissue. Although the isolation techniques for adipose tissue-derived cells are rather diverse, they follow a certain standard procedure. Differences lie mainly in numbers of washing steps, enzyme concentrations, centrifugation parameters, erythrocyte lysis methods as well as in filtration, and eventually culture conditions [Oberbauer E, et al., Cell Regen (Lond). (2015) 4: 7, citing Zuk P A, et al., Mol Biol Cell. (2002) 13(12): 4279-4295; Gimble J, Guilak F. Cytotherapy (200); 5(5): 362-369; Carvalho P P, et al., Tissue Eng Part C Meth. (2013); 19(6): 473-478].

An exemplary protocol for isolating MSCs from adipose tissue includes the steps of obtaining adipose tissue by surgical resection or lipoaspiration; washing the tissue 3-5 times for 5 minutes in PBS each wash, discarding the lower phase until clear; adding collagenase and incubating 1-4 hr at 37 °C on a shaker; adding 10% FBS to neutralize the collagenase; centrifuging the digested fat at 800×g for 10 min; aspirating floating adipocytes, lipids and liquid, leaving the stromal vascular fraction (SVF) pellet; resuspending the SVF pellet in 160 mM NH₄Cl and incubating for 10 minutes at room temperature; centrifuging at 400×g for 10 min at room temperature; layering cells on Percoll or Histopaque gradient; centrifuging at 1000×g for 30 minutes at room temperature; washing cells twice with PBS and centrifuging at 400×g for 10 min between each wash; resuspending the cell pellet in PBS and filtering cells through a 100 μM nylon mesh; passing the cells through a 400 μM nylon mesh; centrifuging at 400×g for 10 minutes; resuspending the cell pellet in 40% FBS/DMEM culture medium and plating the cells. The plastic-adherent cell fraction, including ASCs, can be obtained after passaging or cryopreservation or further cultivated for expansion for a more homogeneous ASC population (Id.).

An exemplary protocol for expansion and subculture of human MSCs includes the following steps: Precoating a tissue culture vessel with 5 μg/mL of PRIME-XV MatrIS F or PRIME-XV Human Fibronectin for 3 hr at room temperature or overnight at 2-8 °C; prewarming PRIME-XV MSC Expansion SFM to 37 °C for no more than 30 min; removing spent media from T-75 flask culture and gently rinsing cells once with 10 mL of PBS for each T-75 flask; adding 3 mL of room temperature TrupLE™ Express to each T-75 flask, and tilting the flask in all directions to disperse the TrypLE™ Express evenly over the cells; incubating the cells at 37 °C, 5% CO₂ to allow the cells to detach; adding 5 mL of PRIME-XV MSC expansion SFM to the flask and dispersing the cells by pipetting the media over the entire growing surface of the flask; transferring the contents to a 15 ml conical tube; centrifuging the cells at 400×g for 5 min and aspirating the supernatant; resuspending the cell pellet in a small amount of pre-warmed PRIME-XV MSC Expansion SFM and counting the cells; resuspending 4.5-5.0×10⁵ cells into 20 mL of the pre-warmed PRIME-XV MSC Expansion SFM for each pre-coated T-75 flask; gently aspirating off PRIME-XV attachment substrate solution from the flask and slowly adding the cell suspension to a T-75 flask; and incubating the cells at 37 °C, 5% CO₂. Spent media is removed and discarded and the cells fed with pre-warmed PRIME-XV MSC Expansion SFM every two days.

### Dental Pulp

Similar to adipose tissue, generating stem cells from dental pulp is a relatively noninvasive and noncontroversial process. Deciduous teeth may be sterilized, and the dental pulp tissue separated from the pulp chamber and root canal, revealed by cutting around the cementoenamel junction using sterilized dental burs [Tsai A I, et al., Biomed Res Int. 2017: 2851906]. After separation, the dental pulp may be isolated using, for example, a barbed broach or a sharp excavator (Id.). MSCs may be isolated enzymatically or non-enzymatically as described above for adipose tissue.

### Lung or Heart Tissue

MSCs may be cultured from tissue biopsies or transplanted tissues. A study in heart transplant patients demonstrated that MSCs present in transplanted hearts were all of donor origin [Hoogduijn M J, et al., Am J Transplant. (2009] January; 9(1): 222-30]. No MSCs of recipient origin were found, not even many years after transplantation. Similar data were found in lung transplant patients [Lama V N, et al., J Clin Invest. 2007 April; 117(4): 989-996]. These data suggest that MSCs do not migrate between tissues, not even under inflammatory conditions as found in transplanted organs [Eggenholfer E, et al., Front Immunol. (2014); 5: 148].

For the isolation of lung or heart tissue-derived MSCs, tissues are minced into pieces and digested with a culture medium containing 0.2% collagenase (Wako) at 37 °C for 30 min. The collagenase is removed by washing twice with 1×PBS. The cell suspension is filtered through a cell strainer (40-μm) and collected in a 50-ml tube. Red blood cells are removed by incubating cells in 1×RBC lysis buffer (BioLegend™) for 5 min at room temperature. Then, 2×10⁷ cells are seeded onto a collagen I-coated, 10-cm dish using MesenCult™ medium containing 1× MesenPure™ and 10 nM of a Rock inhibitor. MSCs may be cultured for up to three passages to reduce any artefacts potentially introduced by long-term culture.

### Blood

Umbilical cord blood MSCs are obtained from 40 mL of UCB with citrate phosphate dextrose (Sigma-Aldrich, St. Louis, MO) as anticoagulant and centrifuged through Ficoll-Paque (1.077 g/cm³) according to the manufacturer's instructions. MSC fractions are washed with PBS, counted using trypan blue exclusion staining and plated onto fibronectin-coated tissue culture flasks (Becton Dickinson) in MSC expansion medium (Iscove modified Dulbecco medium (IMDM, Life Technologies) and 20% FBS supplemented with 10 ng/ml recombinant human bFGF (Peprotech™, Rocky Hill NJ), 100 U penicillin, 100 U streptomycin and 2 mM L-Glutamine (Life Technologies®/Gibco®). Cells are allowed to adhere overnight and nonadherent cells washed out with medium changes.

In an exemplary protocol for obtaining MSCs from whole blood, a diluted mixture of PBS and peripheral blood is layered in a 50 ml centrifuge tube on top of Ficoll-Paque® and centrifuged at 400×g for 30-40 minutes at 20 °C in a swinging-bucket rotor without a brake. The upper layer is aspirated, leaving the mononuclear cell layer (lymphocytes, monocytes and thrombocytes) undisturbed at the interface. The mononuclear cell layer is carefully transferred into a new 50 ml centrifuge tube. Cells are washed with PBS (pH 7.2) containing 2 mM EDTA, centrifuged at 300×g for 10 min at room temperature and the supernatant discarded. For removal of platelets, the cell pellet is resuspended in 50 mL buffer and centrifuged at 200×g for 10-15 minutes at room temperature. The supernatant containing the platelets is removed. This step is repeated. The cell pellet is resuspended in DMEM, 20% FBS and 1% antibiotic-antimycotic. Cultures are maintained at 37 °C in a humidified atmosphere containing 5% CO₂. Suspended cells are discarded after 5-7 days of culture and adherent cells left to grow on the flask surface. Culture medium is changed every 3 days.

### Amniotic Fluid

Amniotic fluid is formed at 2 weeks after fertilization in the amniotic cavity of early gestation [Kim E Y, et al., BMB Rep. (2014) March; 47(3): 135-140]. Amniotic fluid keeps the fetus safe and supports organ development. The first progenitor cells derived from amniotic fluid was reported in 1993 by Torricelli et al [Ital J Anat Embryol. (1993) April-June; 98(2): 119-26). Many studies have identified amniotic fluid (AF) as a source of MSCs. These AF-MSCs express the pluripotent marker Oct-4 in almost 90% of the active condition, and they also have multiple differentiation capacity like amniotic membrane MSCs [Tsai M S, et al., Hum Reprod. (2004) June; 19(6): 1450-6; De Coppi P, et al., Nature Biotechnol. (2007) January; 25(1): 100-106]. AF is also routinely used to perform the standard evaluation of karyotyping, and genetic and molecular tested for diagnostic purposes. After prenatal diagnostic testing, AF cells can be used as a source of fetal progenitor cells or otherwise discarded [Prusa A R, et al., Med Sci Monit. (2002) November; 8(11): RA253-7]. Use of these cells could minimize ethical objections, have a high renewal activity, and maintain genetic stability [(Kim E Y, et al., BMB Rep. 2014 March; 47(3): 135-140]. AF-MSCs are easily isolated and offer advantages of non-tumorigenicity and low immunogenic activity. (Id.).

Amniotic fluid samples are obtained by amniocentesis performed between 16 and 20 weeks of gestation for fetal karyotyping. A two-stage culture protocol can be used for isolating MSCs from amniotic fluid [Tsai M S, et al., Hum Reprod. 2004 June; 19(6): 1450-1456]. For culturing amniocytes (first stage), primary in situ cultures are set up in tissue culture-grade dishes using Chang medium (Irvine Scientific, Santa Ana, CA). Metaphase selection and colony definition is based on the basic requirements for prenatal cytogenetic diagnosis in amniocytes [Moertel C A, et al. (1992) Prenat Diagn 12, 671-683]. For culturing MSCs (second stage), non-adhering amniotic fluid cells in the supernatant medium are collected on the fifth day after the primary amniocytes culture and kept until completion of fetal chromosome analysis. The cells are then centrifuged and plated in 5 ml of α-modified minimum essential medium (α-MEM; Gibco-BRL) supplemented with 20% fetal bovine serum (FBS; Hyclone, Logan, UT) and 4 ng/mL basic fibroblast growth factor (bFGF; R&D systems, Minneapolis, MN) in a 25 cm² flask and incubated at 37 °C with 5% humidified CO₂ for MSC culture. Similar to MSCs from umbilical cord blood and first-trimester fetal tissues, surface antigens such as SH3, SH4, CD29, CD44 and HLA-A,B,C (MHC class I) may be found, and CD10, CD11b, CD14, CD34, CD117, HLA-DR,DP,DQ (MHC class II) and EMA are absent [Tsai M S, et al., Hum Reprod. (2004) June; 19(6): 1450-6; Pittenger M F, et al., Science 284, 143-147; Colter D C, et al., Proc Natl Acad Sci USA 98, 78415; Young H Y, et al., Anat Rec 264, 51-62].

According to some embodiments, to characterize the adherent MSCs, osteoblastic differentiation is induced by culturing confluent human MSCs for 3 weeks in osteoblastic differentiation media (all from Sigma) and after three weeks, the cells are stained by Alizarin. To induce adipocyte differentiation, confluent MSCs are cultured for 1 to 3 weeks in differentiation medium, and lipid droplet staining is carried out by S Red Oil (Sigma).

According to some embodiments, flow cytometry can be used to characterize cell markers expressed on the surface of the isolated MSCs. According to some embodiments, the phenotype of the adherent MSCs is CD73+, CD90+, CD105+, CD34−, CD45−.

According to some embodiments, the MSCs are derived from a biological sample, wherein the biological sample is a tissue or a body fluid of a human subject. According to some embodiments, the tissue is placental tissue, adipose tissue, umbilical cord tissue, lung tissue, heart tissue or dental pulp; or bone marrow of normal healthy subjects aged 21-40 years old; or the body fluid is blood, amniotic fluid or urine. According to some embodiments, the MSCs derived from placental tissue are derived from one or more of chorionic membrane (CM), chorionic trophoblast without villi (CT-V), chorionic villi (CV), or decidua (DC). According to some embodiments, the blood is umbilical cord blood or peripheral blood.

According to some embodiments, extracellular vesicles derived from a biological sample, e.g., human bone marrow, will be isolated from bone marrow-derived mesenchymal stem cells of an adult healthy donor (21-40 years old) who will have met strict donor acceptance criteria including current and past medical history in addition to passing highly sensitive nucleic acid testing for hepatitis B, hepatitis C, and HIV.

According to some embodiments, the MSCs are obtained from a human subject. According to some embodiments, identity of the hMSCs can be quickly confirmed by their expression of at least three cell surface markers selected from CD29, CD44, CD90, CD73, and CD105.

An exemplary protocol is as follows. Human mesenchymal stem cells used to generate the extracellular vesicle (EV) product originate from a single donor bone marrow source of adherent stromal progenitor cells expanded under GTP/cGMP compliant conditions. In sort, the MSC product is expanded in a 3D bioreactor until 80% confluence. Expansion media is then washed with PBS and replaced with serum-free DMEM. Conditioned media is collected from the waste outlet of the bioreactor and subjected to ultracentrifugation for exosome precipitation. This collection procedure is performed daily for up to 96 hours. EVs are isolated by differential filtration through nylon membrane filters of defined pore size. A first filtration through a large pore size retains cellular fragments and debris; a subsequent filtration through a smaller pore size retains EV and purifies them from smaller size contaminants. Tangential flow filtration (TFF) is utilized to concentrate the fluid containing the EVs. 0.22μ filtration is first completed, followed by TFF with pore size of either 100 kD molecular weight cut off (MWCO) or 300 kD MWCO is used for concentration and diafiltration. EVs are further purified using size exclusion chromatography by loading the concentrated clarified conditioned media on a PBS-equilibrated Chroma S-200 column (Clontech) using a 70 nm pore size, eluting with PBBS and collecting final bulk drug substance. This is sterile filtered and provided in Dulbeccos' Phosphate Buffered Saline (DPBS) without calcium or magnesium added to sterile vials at concentration of ≥80 billion particles per vial, capped packaged, frozen at −80 °C.

The population of exosomes resulting from this isolation process is a purified enriched population of potent exosomes. Primary exosome product characterization analysis includes, without limitation, nanoparticle size and concentration analysis; flow cytometry analysis of exosome specific protein markers (CD63, CD9), and pro-angiogenic and pro-migratory potency. Other exemplary analytics include, without limitation:cytokine testing using a 9-panel human angiogenesis multiplex ELISA comprising: Ang-2, FGF, HGF, IL-8, PDGF, TIMP-1, TIMP-2, and VEGF at standard dilutions of 1:2, 1:20, 1:200 and 1:800; Mycoplasma testing by multiplex detection by polymerase chain reaction; sterility testing for media by membrane filtration; and metabolite and pH analysis on the NOVA Flex 2 for Gln, Glu, Gluc, Lac, NH₄+, Na+, K+, Ca++, pH, pCO₂, pO₂ and osmolality.

The product comprising the purified enriched population of potent exosomes will be tested for sterility and is manufactured in FDA-registered facilities that meet Current Good Manufacturing Practices (cGMP). The exosome product will be filtered [pore size] and purified prior to filling, and all containers will be visually inspected prior to cryopreservation to ensure normal appearance, intact container closure, and the absence of visible extraneous particulate matter. Product will be released for clinical use only if all safety, identity, quality, and purity specifications are met and all applicable GMPs are appropriately followed as determined by Zen-Bio Quality Assurance.

### Method for Tuning a Signature Cargo in Extracellular Vesicles

According to some embodiments, the purified enriched population of potent exosomes comprises a cargo. According to some embodiments, the cargo comprises a potency signature including expression of one or more, two or more, three or more, four or more, five or more, six or more, seven or more, eight or more of angiopoietin 2 (Ang-2), fibroblast growth factor (FGF), hepatic growth factor (HGF), interleukin 8 (IL-8), a tissue inhibitor of metalloproteinases (TIMP), vasculoendothelial growth factor (VEGF), platelet derived growth factor (PDGF), or tumor necrosis factor alpha (TNFα). According to some embodiments, the tissue inhibitor of metalloproteinases is TIMP1, TIMP2, or TIMP1 and TIMP2.

According to some embodiments, exposure of mesenchymal stem cells (MSCs) cultured at 37 °C and 5% CO₂ to one or more environmental stressors followed by isolation of the population of exosomes derived from the MSCs produces a population of tuned exosomes, wherein the cargo of the exosomes comprising a protein signature, an miRNA signature, or a combination thereof, is modulated compared to non-exposed MSCs (“native MSCs”).

According to some embodiments, the isolated population of exosomes derived from the MSCs are exposed to one or more environmental stressors to produce a population of tuned exosomes, wherein the cargo of the exosomes comprising a protein signature, an miRNA signature, or a combination thereof, is modulated compared to native MSCs. According to some embodiments, the one or more environmental stressors includes exposure to light, exposure to extreme temperatures, exposure to growth factors, or a combination thereof.

According to some embodiments, exposure of the MSCs to the light is in culture at 37 °C in 5% CO₂, in a bioreactor at specified temperature, pH, and gas conditions, or directly to a population of MSC-derived exosomes.

According to some embodiments, MSCs are exposed to light at one or more wavelengths in the visible spectrum, the near-infrared spectrum, the ultraviolet spectrum, or a combination thereof. In some embodiments, MSCs are exposed to light at one or more wavelengths in the visible spectrum. In some embodiments, MSCs are exposed to light at one or more wavelengths in the near-infrared spectrum. In some embodiments, MSCs are exposed to light at one or more wavelengths in the ultraviolet spectrum.

According to some embodiments, the light is provided by one or more light-emitting diodes (LEDs), one or more lasers, natural sunlight, fluorescent lighting, incandescent lighting, halogen lighting, or a combination thereof.

According to some embodiments, the exposure to light comprises exposure to visible light at a wavelength of about 380 nm to about 750 nm, inclusive. According to some embodiments, the visible light comprises violet light at a wavelength of about 380 nm to about 450 nm, inclusive. According to some embodiments, the visible light comprises blue light at a wavelength of about 450 nm to about 495 nm, inclusive. According to some embodiments, the visible light comprises green light at a wavelength of about 495 nm to about 570 nm, inclusive. According to some embodiments, the visible light comprises yellow light at a wavelength of about 570 nm to about 590 nm, inclusive. According to some embodiments, the visible light comprises orange light at a wavelength of about 590 nm to about 620 nm, inclusive. According to some embodiments, the visible light comprises red light at a wavelength of about 620 nm to about 750 nm, inclusive. According to some embodiments, the light exposure comprises exposure to near-infrared light at a wavelength of about 750 nm to about 1440 nm, inclusive. According to some embodiments, the light exposure comprises exposure to ultraviolet light at a wavelength of about 100 nm to about 380 nm, inclusive. In some embodiments, the light exposure comprises exposure to ultraviolet A (UVA) light at a wavelength of about 315 nm to about 380 nm, inclusive. In some embodiments, the light exposure comprises exposure to ultraviolet B (UVB) light at a wavelength of about 280 nm to about 315 nm, inclusive. In some embodiments, the light exposure comprises exposure to ultraviolet C (UVC) light at a wavelength of about 100 nm to about 280 nm, inclusive.

According to some embodiments, the MSCs are exposed to light for a duration of about 1 minute to about 24 hours, inclusive, i.e., about 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, or 24 hours.

According to some embodiments, the light exposure is at a fluence (meaning the number of particles, such as photons or neutrons, incident on a given area, or the total energy of those particles received by that area in joules per square centimeter (J/cm²) of about 1 J/cm² to about 100 J/cm², inclusive, i.e., about 1 J/cm², 2 J/cm², 4 J/cm², 6 J/cm², 8 J/cm², 10 J/cm², 12 J/cm², 14 J/cm², 16 J/cm², 18 J/cm², 20 J/cm², 22 J/cm², 24 J/cm², 26 J/cm², 28 J/cm², 30 J/cm², 32 J/cm², 34 J/cm², 36 J/cm², 38 J/cm², 40 J/cm², 42 J/cm², 44 J/cm², 46 J/cm², 48 J/cm², 50 J/cm², 52 J/cm², 54 J/cm², 56 J/cm², 58 J/cm², 60 J/cm², 62 J/cm², 64 J/cm², 66 J/cm², 68 J/cm², 70 J/cm², 72 J/cm², 74 J/cm², 76 J/cm², 78 J/cm², 80 J/cm², 82 J/cm², 84 J/cm², 86 J/cm², 88 J/cm², 90 J/cm², 92 J/cm², 94 J/cm², 96 J/cm², 98 J/cm², or 100 J/cm².

According to some embodiments, the light exposure is at an irradiance (defined as the radiant flux (optical power) received by a surface per unit area measured in watts per square meter (W/m²) of about 1 mW/cm² to about 500 mW/cm², inclusive, i.e., about 1 mW/cm², 5 mW/cm², 10 mW/cm², 15 mW/cm², 20 mW/cm², 25 mW/cm², 30 mW/cm², 35 mW/cm², 40 mW/cm², 45 mW/cm², 50 mW/cm², 55 mW/cm², 60 mW/cm², 65 mW/cm², 70 mW/cm², 75 mW/cm², 80 mW/cm², 85 mW/cm², 90 mW/cm², 95 mW/cm², 100 mW/cm², 105 mW/cm², 110 mW/cm², 115 mW/cm², 120 mW/cm², 125 mW/cm², 130 mW/cm², 135 mW/cm², 140 mW/cm², 145 mW/cm², 150 mW/cm², 155 mW/cm², 160 mW/cm², 165 mW/cm², 170 mW/cm², 175 mW/cm², 180 mW/cm², 185 mW/cm², 190 mW/cm², 195 mW/cm², 200 mW/cm², 205 mW/cm², 210 mW/cm², 215 mW/cm², 220 mW/cm², 225 mW/cm², 230 mW/cm², 235 mW/cm², 240 mW/cm², 245 mW/cm², 250 mW/cm², 255 mW/cm², 260 mW/cm², 265 mW/cm², 270 mW/cm², 275 mW/cm², 280 mW/cm², 285 mW/cm², 290 mW/cm², 295 mW/cm², 300 mW/cm², 305 mW/cm², 310 mW/cm², 315 mW/cm², 320 mW/cm², 325 mW/cm², 330 mW/cm², 335 mW/cm², 340 mW/cm², 345 mW/cm², 350 mW/cm², 355 mW/cm², 360 mW/cm², 365 mW/cm², 370 mW/cm², 375 mW/cm², 380 mW/cm², 385 mW/cm², 390 mW/cm², 395 mW/cm², 400 mW/cm², 405 mW/cm², 410 mW/cm², 415 mW/cm², 420 mW/cm², 425 mW/cm², 430 mW/cm², 435 mW/cm², 440 mW/cm², 445 mW/cm², 450 mW/cm², 455 mW/cm², 460 mW/cm², 465 mW/cm², 470 mW/cm², 475 mW/cm², 480 mW/cm², 485 mW/cm², 490 mW/cm², 495 mW/cm², 500 mW/cm².

According to some embodiments, the exposure to light modulates the miRNA cargo of the population of exosomes derived from the population of MSCs to include a signature of upregulated and downregulated miRNAs relative to non-irradiated (native) controls. According to some embodiments, the exposure to light upregulates one or more of miR-29, miR-196a, or miR-9a in the exosome cargo of the population of MSCs. According to some embodiments, the exposure to light downregulates one or more of miR-21, miR-145, miR-23, or miR-31 in the exosome cargo. In some embodiments, exposure to light modulates the miRNA cargo of a population of exosomes derived from the population of MSCs to include a signature of miRNAs that are not present in non-irradiated (native) controls.

According to some embodiments, the exposure to light modulates a protein cargo of the population of exosomes derived from the population of MSCs. According to some embodiments, the exposure to light modulates the expression of one or more tetraspanins selected from CD9, CD63, CD81, or a combination thereof on the surface of a purified enriched population of exosomes relative to a non-irradiated (native) control.

According to some embodiments, the population of MSCs is exposed to an elevated temperature. According to some embodiments, the elevated temperature comprises a heat shock. According to some embodiments, the exposure of the population of MSCs to an elevated temperature comprises exposing the population of MSCs to a temperature of about 30 °C to about 50 °C, inclusive. According to some embodiments, the elevated temperature is about 30 °C, about 31 °C, about 32 °C, about 33 °C, about 34 °C, about 35 °C, about 36 °C, about 37 °C, about 38 °C, about 39 °C, about 40 °C, about 41 °C, about 42 °C, about 43 °C, about 44 °C, about 45 °C, about 46 °C, about 47 °C, about 48 °C, about 49 °C, or about 50 °C.

According to some embodiments, the exposure of the population of MSCs to an elevated temperature comprises a duration of about 10 minutes to about 6 hours, inclusive. According to some embodiments, the duration is about 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, or 6 hours.

According to some embodiments, the elevated temperature modulates the protein cargo of the population of exosomes derived from the population of MSCs. According to some embodiments, the elevated temperature modulates the protein cargo of the population of exosomes derived from the population of MSCs to include increased expression of one or more heat shock proteins. According to some embodiments, the heat shock protein is HSP70. According to some embodiments, the exposure to elevated temperatures modulates the expression of one or more tetraspanins on the surface of the exosomes. According to some embodiments, the exposure to elevated temperatures modulates the expression of CD9, CD63, CD81, or a combination thereof on the surface of the population of exosomes derived from the population of MSCs relative to a non-exposed (native) control.

According to some embodiments, the exposure to the elevated temperature modulates the miRNA cargo of the population of exosomes derived from the population of MSCs to include a signature of upregulated and downregulated miRNAs relative to non-exposed (native) controls. According to some embodiments, the exposure to elevated temperature upregulates one or more of miR-29, miR-196a, or miR-9a in the cargo of the population of exosomes. According to some embodiments, the exposure to elevated temperature downregulates one or more of miR-21, miR-145, miR-23, or miR-31 in the cargo of the population of exosomes. In some embodiments, exposure to elevated temperature modulates the miRNA cargo of the population of exosomes derived from the population of MSCs to include a signature of miRNAs that are not present in non-exposed (native) controls.

In some embodiments, the population of MSCs is exposed to elevated temperatures in a controlled environment, for example, a chamber, a water bath, an incubator, or a bioreactor. In some embodiments, cellular stress induced by the exposure to elevated temperatures is measured by assessing the expression of heat shock proteins, including, but not limited to one or more of HSP40, HSP60, HSP70, and HSP90.

According to some embodiments, the population of MSCs is exposed to a reduced temperature to modulate the protein cargo, miRNA cargo, or a combination thereof of the population of exosomes derived from the MSCs. According to some embodiments, the reduced temperature comprises a cold shock. According to some embodiments, the exposure to reduced temperature comprises exposing the MSCs to a temperature of about 0 °C to about 32 °C, inclusive. According to some embodiments, the reduced temperature is about 0 °C, about 1 °C, about 2 °C, about 3 °C, about 4 °C, about 5 °C, about 6 °C, about 7 °C, about 8 °C, about 9 °C, about 10 °C, about 11 °C, about 12 °C, about 13 °C, about 14 °C, about 15 °C, about 16 °C, about 17 °C, about 18 °C, about 19 °C, about 20 °C, about 21 °C, about 22 °C, about 23 °C, about 24 °C, about 25 °C, about 26 °C, about 27 °C, about 28 °C, about 29 °C, about 30 °C, about 31 °C, or about 32 °C.

According to some embodiments, the exposure to reduced temperature comprises exposing the population of MSCs to a reduced temperature for a duration of about 10 minutes to about 6 hours, inclusive. According to some embodiments, the duration is about 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, or 6 hours.

According to some embodiments, the reduced temperature modulates the expression of one or more proteins in the cargo of the population of exosomes derived from the population MSCs to include a signature cargo of upregulated and downregulated proteins relative to non-exposed (native) controls. According to some embodiments, the reduced temperature modulates the expression of one or more miRNAs of the cargo of the population of exosomes derived from the population MSCs to include a signature cargo of upregulated and downregulated miRNAs relative to non-exposed (native) controls. In some embodiments, the reduced temperature modulates the expression of one or more proteins, one or more miRNAs, or a combination thereof in the cargo includes one or more proteins, miRNAs, or a combination thereof that is not present in a non-exposed (native) control.

In some embodiments, the MSCs are exposed to reduced temperatures in a controlled environment, for example, a refrigerator, a freezer, a chamber, a water bath, an incubator, or a bioreactor. In some embodiments, cellular stress induced by the exposure to reduced temperatures is measured by assessing the expression of cold shock proteins (meaning multifunctional RNA/DNA binding proteins, characterized by the presence of one or more cold shock domains [Lindquist, J A and Mertens, PR, Cell Communication & Signaling (2018) 16: article 63], including, but not limited to one or more of RNA-binding motif protein 3 (RBM3), cold-inducible RNA-binding protein (CIRP), Y-box binding protein (YB-1), LIN28, calcium regulated heat-stable protein 1 (CARHSP1).

According to some embodiments, an additional environmental stressor comprises exposure of the MSCs to one or more growth factors. According to some embodiments, the growth factor is platelet-derived growth factor (PDGF), epidermal growth factor (EGF), vascular endothelial growth factor (VEGF), fibroblast growth factor (FGF), insulin-like growth factor (IGF).

According to some embodiments, the population of MSCs is exposed to the growth factor is at a concentration of about 5 ng/mL to about 500 ng/mL, inclusive, i.e., about 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, 250 ng/mL, 255 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, 400 ng/mL, 405 ng/mL, 410 ng/mL, 415 ng/mL, 420 ng/mL, 425 ng/mL, 430 ng/mL, 435 ng/mL, 440 ng/mL, 445 ng/mL, 450 ng/mL, 455 ng/mL, 460 ng/mL, 465 ng/mL, 470 ng/mL, 475 ng/mL, 480 ng/mL, 485 ng/mL, 490 ng/mL, 495 ng/mL, or 500 ng/mL.

According to some embodiments, the exposure to the one or more growth factors modulates the protein cargo, miRNA cargo, or a combination thereof of the population of exosomes derived or originated from the population of MSCs. According to some embodiments, the exposure to the one or more growth factors modulates the expression of one or more proteins in the exosome cargo includes a signature of upregulated and downregulated proteins relative to controls not exposed to the one or more growth factors (native controls). According to some embodiments, the exposure to the one or more growth factors modulates the expression of CD9, CD63, CD81, or a combination thereof on the surface of the population of exosomes derived from the population of MSCs relative to a non-exposed (native) control. According to some embodiments, the exposure to the one or more growth factors modulates the miRNA cargo of the population of exosomes derived from the population of MSCs to include a signature of upregulated and downregulated miRNAs relative to non-exposed (native) controls. According to some embodiments, the exposure to the one or more growth factors modulates the expression of one or more proteins, one or more miRNAs, or a combination thereof to include proteins, miRNAs, or a combination thereof that are not present in a non-exposed (native) control.

According to some embodiments, miRNA expression in the population of exosomes derived or originated from the population of MSCs is measured by quantifying miRNA using one or more of reverse transcriptase quantitative polymerase chain reaction (RT-qPCR) (i.e., a highly sensitive PCR-based method that uses specialized primers to amplify and quantify specific known miRNA targets using fluorescent dyes or probes), RNA sequencing (RNAseq) (i.e., a next generation sequencing technique that ligates adaptors to all small RNAs, amplifies them, and reads their exact sequences), or microarray analysis (i.e., a hybridization method where labeled miRNA molecules bind to a chip containing complementary DNA probes to measure the relative abundance of thousands of known miRNAs at once).

According to some embodiments, protein expression in the population of exosomes derived or originated from the population of MSCs is measured by quantifying total protein and then detecting specific proteins using one or more of western blotting (e.g., an analytical technique that uses gel electrophoresis to separate proteins by size, followed by transfer to a membrane and detection with specific antibodies, to identify and quantify a particular protein in a sample), enzyme-linked immunosorbent assay (ELISA) (e.g., a plate-based analytical technique that uses antibodies conjugated to enzymes to detect and quantify a specific protein, peptide, antibody, or hormone in a sample), flow cytometry (e.g., an analytical technique that rapidly measures physical and chemical characteristics of individual cells or particles in a fluid stream as they pass through one or more laser beams, enabling simultaneous multi-parameter analysis of cell size, granularity, and surface or intracellular marker expression), or mass spectrometry (i.e., an analytical technique that measures the mass-to-charge ratio of ions to identify and quantify molecules in a sample, typically by ionizing chemical compounds, separating the resulting ions based on their mass-to-charge ratios, and detecting their relative abundances).

According to some embodiments, the cargo of a population of tuned exosomes comprising a protein signature, an miRNA signature, or a combination thereof, is modulated by, in order: (a) exposure of a population of MSCs to an elevated temperature; (b) exposure of the population of MSCs to light; and (c) isolation of the population of exosomes from the population of MSCs exposed to elevated temperatures and light. Control MSCs are non-modulated native MSCs containing nonmodulated exosomes comprising a nonmodulated cargo. According to some embodiments, the exposure to elevated temperatures includes exposure to a temperature of about 41 °C to about 43 °C, inclusive, for a duration of about 10 minutes to about 30 minutes, inclusive. According to some embodiments, the exposure to light includes exposure to red light at a wavelength of about 620 nm to about 750 nm, inclusive, near-infrared light at a wavelength of about 750 nm to about 1440 nm, inclusive, ultraviolet light at a wavelength of about 100 nm to about 380 nm, inclusive, or a combination thereof.

According to some embodiments, the cargo of a population of tuned exosomes, comprising a protein signature, an miRNA signature, or a combination thereof, is modulated by: (a) exposure of a population of MSCs to an elevated temperature; (b) isolation of the tuned population of exosomes from the population of MSCs exposed to elevated temperatures; and (c) exposure of the isolated population of exosomes to light. Control MSCs are non-modulated native MSCs containing nonmodulated exosomes comprising a nonmodulated cargo. According to some embodiments, the exposure to elevated temperatures includes exposure to a temperature of about 41 °C to about 43 °C, inclusive for a duration of about 10 minutes to about 30 minutes, inclusive. According to some embodiments, the exposure to light includes exposure to red light at a wavelength of about 620 nm to about 750 nm, inclusive, near-infrared light at a wavelength of about 750 nm to about 1440 nm, inclusive, ultraviolet light at a wavelength of about 100 nm to about 380 nm, inclusive, or a combination thereof.

According to some embodiments, the exposure to elevated temperatures and light upregulates one or more of miR-29, miR-196a, or miR-9a in the cargo of the population of exosomes. According to some embodiments, the exposure to elevated temperature downregulates one or more of miR-21, miR-145, miR-23, or miR-31 in the cargo of the population of exosomes. In some embodiments, exposure to elevated temperatures and light modulates the miRNA cargo of the population of exosomes originated from the population of MSCs to include a signature of miRNAs that are not present in non-exposed (native) controls.

According to some embodiments, the exposure to elevated temperatures and light modulates the expression of one or more tetraspanins selected from CD9, CD63, CD81, or a combination thereof on the surface of a purified enriched population of exosomes relative to a non-exposed (native) control. According to some embodiments, the exposure to elevated temperatures and light modulates the expression of one or more heat shock proteins selected from HSP40, HSP60, HSP70, HSP90, or a combination thereof relative to a non-exposed (native) control.

According to some embodiments, per isolation process, the composition comprises at least 1×10E12 EVs comprising exosomes of which at least 1×10E8 are exosomes.

According to some embodiments, a therapeutic amount of a purified, enriched population of potent exosomes comprises at least 1×10E8 exosomes.

According to some embodiments, the cargo of the exosomes comprising the therapeutic signature: is configured so as to modulate one or more of the injury, the inflammation, an excess accumulation of extracellular matrix, cell senescence; or is configured so as to modulate a pathway comprising fibrogenic signaling; or is configured so as to slow or reverse progression of the age-related chronic lung disease; or is configured to reprogram a tissue affected by an age-related chronic disease.

According to some embodiments, the age-related chronic disease if left untreated comprises one or more of a progressive injury, progressive inflammation, progressive fibrosis or a combination thereof.

According to some embodiments, the pathway comprises transforming growth factor (TGFβ) signaling. According to some embodiments, the pathway comprising fibrogenic signaling is one or more pathway that upregulates TERT (Telomerase Reverse Transcriptase) transcription including a Smad pathway, a mitogen-activated protein kinase pathway, a phosphoinositide 3-kinase pathway; a canonical Wnt-β catenin pathway; an NF-κB pathway, or a Notch signaling pathway.

According to some embodiments, the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle.

According to some embodiments, the age-related chronic disease comprises a chronic lung disease, a chronic inflammation and immune dysfunction, a mitochondrial dysfunction, organ transplantation dysfunction, organ resuscitation and rejuvenation, a viral infection, neuropathic pain; neurofibrosis, neurodegeneration, connective tissue dysfunction, musculoskeletal repair, dysfunction of the gut microbiome, or an age-related decline in overall health.

### Microbiome Dysfunction

According to some embodiments, a composition of the present disclosure comprising a population of purified enriched potent exosomes with an appropriate therapeutic signature of MSC-derived EVs isolated from a normal healthy subject may modulate microbiome dysfunction.

The human microbiota consists of 10-100 trillion symbiotic microbial cells harbored by each person, primarily bacteria in the gut, while the human microbiome consists of the genes these cells harbor [Ursell, L K et al., “Defining the human microbiome.” Nutr. Rev. (2012) 70 (Suppl. 1) S38-S44]. These microbial cells, and their genetic material, live with humans from birth, and every individual has a unique mix of species. This relationship is important for nutrition, immunity and effects on the brain and behavior, and has been implicated in a number of diseases where the disease is caused by a disturbance in the normal balance of microbes or where the disturbance is another downstream consequence of the disease. The interaction between the human microbiota and the environment is dynamic, meaning that microbial communities are constantly being transferred between surfaces, and that a dynamic interaction exists between environmental microbiota and different human body sites. There is increasing evidence that individuals actually share a core microbiota, with vastly different sets of microbial species that yielded very similar functional molecular interactions, reactions and relation networks for metabolism, genetic information processing, environmental information processing, cellular processes, and organismal systems referred to as KEGG pathways.

There is accumulating evidence that a crosstalk between the gut and lungs exists. The lungs of healthy individuals harbor Fusobacterium, Haemophilus, Prevotella, Streptococcus, and Veillonella as main genera, which are relatively small in size when compared to the enteric microbiota [Ahlawat, S. et al. Virus Res. (2020) 286: 198103, citing He, et al., Y. et al. Crit. Rev. Microbiol. (2017) 43 (1): 81-95]. The emergence and maintenance of lung microbiota is governed by the equilibrium between microbial migration from the upper respiratory tract and microbial removal by the host defense systems, with small contribution from the multiplication of native microbes. Even in small concentrations, the airway microbiome is believed to be crucial to host immunity, such that an imbalance between microbial immigration and removal predisposes its host to the progression and exacerbations of respiratory diseases [Id., citing He, et al., Y. et al. Crit. Rev. Microbiol. (2017) 43 (1): 81-95; Wypych, T P et al. Nat. Immunol. (2019) 20 (10): 1279-90]. Alterations in the lung microbial community including airways also affects the composition of the intestinal microbiota, and respiratory viral infections can alter the intestinal microbiome, where the intestinal microbiome determines the adaptive immune responses against the respiratory pathogens and is necessary for priming the innate immune responses against the pulmonary infection. Indeed, during respiratory viral infections, the level of macrophage response to the respiratory viruses depends on the presence of intestinal microbes [Id., citing Hanada, S. et al. Front. Immunol. (2018) 9: 2640].

Gut microbiota composition and diversity is affected by many factors, including by aging. An apparent age-related gut microbiota imbalance has been described, featuring an altered microbial diversity, a lower abundance of probiotic strains (e.g., Difidobacteria), and a reduced number of species producing butyrate, a short chain fatty acid (SCFA) that plays important metabolic functions and has a major role in maintaining the integrity of intestinal epithelium [Mangiola, F. et al. Eur. Rev. Med. Pharmacol. Sci. (2018) 22: 7404-13].

The integrity of the commensal microbiota can be disturbed by invading viruses, which cause dysbiosis (meaning a general imbalance of gut microbiota) in the host and further influence virus infectivity [Li, N. et al. Frontiers Immunol. (2019) 10: 1551]. It has been shown that RNA viruses, such as poliovirus, benefit from the delivery of various viral genomes into a single target cell, thereby allowing the recombination of multiple virus genomes; this process potentiates those viral progeny with enhanced environmental fitness [Id., citing Aguilera, E R et al. M. Bio. (2017) 8: 16; Chen, Y H et al. Cell (2015) 160: 619-630; Combe, M. et al. Cell Host Microbe (2015) 18: 424-432]. Some viruses (e.g., poliovirus, reovirus) use commensal microbiota components to enhance viral stability and increase infectivity. There is also likely a link between commensal microbiota and the lytic reactivation of viruses. In addition to fostering the generation of immunoregulatory Treg cells and thereby suppress anti-viral immunity, the commensal microbiota also have been reported to directly skew antiviral immunity by suppressing the activation of effector immune cells and by inhibiting the production of various inflammatory cytokines that are pivotal for virus elimination, thus creating a more favorable environment for viral infection [Id., citing Baldridge, M T et al. Science (2015) 347: 266-269].

The commensal microbiota also can prevent viral infection. For example, Enterococcus faecium can prevent infection by influenza viruses upon direct absorptive trapping of these viruses [Id., citing Wang, X Y et al. PLoS One (2013) 8: e53043]. An extracellular matrix binding protein produced by Staphylococcus epidermidis, a Gram-positive bacterium that lives in the human nasal cavity as a commensal, can stably bind to influenza virus and thus block further infection [Id., citing Chen, H W et al. Sci. Rep. (2016) 6: 27870]. The replication of herpes simplex virus-2 (HSV-2) can also be suppressed by commensal microbiota metabolites; lactic acid, a major end product of the carbohydrate fermentation of all Lactobacillus species, can strongly inactivate HSV-2 in the vaginal mucosa by maintaining an acidic pH in the local environment [Id., citing Tuyama, C G et al. J. Infect. Dis. (2006) 194: 795-803]. Commensal microbiota also exert their antiviral activity through cell wall associated bacterial components [Id., citing Mastromarino, P. et al. Anaerobe (2011) 17: 334-336].

Supporting studies have shown that intact healthy commensal microbiota help maintain robust antiviral immunity, while microbiota disruption increases viral infectivity due to the impaired capacity of the immune system to limit viral infection. For example, Clostridium orbiscindens, a human-associated gut microbe produces desaminotyrosine to prime the amplification loop of type I IFN signaling, thereby mediating protection against influenza infection [Id, citing Steed, A L et al. Science (2017) 357: 498-502]. It has been shown that during respiratory influenza virus infection, antibiotic exposure led to a defective generation of virus-specific CD4 and CD8 T cells and antibodies due to an impaired inflammasome-dependent migration of APCs from the lung to the draining lymph nodes [Id., citing Ichinohe, T. et al. Proc. Natl Acad. Sci. USA (2011) 108: 5354-5359].

Studies also suggest an important role of virus infection in inducing microbiota dysbiosis. This is true for HIV/SIV infection, influenza virus infection, HBV or hepatitis C virus infection (HCV) and norovirus infection. For example, microbial diversity in saliva of HIV patients was significantly reduced compared to healthy controls, accompanied by increased abundance of potentially pathogenic Megasphaera, Camplyobacter, Veillonella and Prevotella species, and decreased commensal Veillonella and Streptococcus species [Id., citing Li, Y. et al. J. Clin. Microbiol. (2014) 52: 1400-1411; Dang, A T et al. BMC Microbiol. (2012) 12 (152): 95, 96]. In addition, fungal communities in HIV infected and uninfected individuals differed significantly [Id., citing Mukherjee, P K et al. PLoS Pathog. (2014) 10: e1003996]. In bronchoalveolar lavage fluids, although there were no significant differences among the microbial composition in HIV-infected and uninfected subjects, specific metabolic profiles were associated with bacterial organisms that potentially play a role in the pathogenesis of pneumonia in HIV infected patients [Id., citing Cribbs, S K et al. Microbiome (2016) 4: 3]. Mechanistically, specific immune suppression by HIV is partly responsible for the enrichment of certain potentially pathogenic bacteria.

Several studies have shown that influenza virus infection can result in decreased colonization by healthy bacteria and increased abundance of potentially pathogenic microbiota. There is a consensus that influenza virus infection alters the commensal microbiota of the host, causing corresponding disruptions of the microbiota-host homeostasis, which largely accounts for the mechanisms by which infections are established. Several lines of data have highlighted that the modulation of immune responses by influenza contributes to the dysbiosis of the gut [Id., citing Wang, J. et al. J. Exp. Med. (2014) 211: 2397-2410; Deriu, E. et al. PLoS Pathog. (2016) 12: e1005572].

Although SARS-CoV-2 primarily causes lung infection through binding of ACE2 receptors present on alveolar epithelial cells, SARS-CoV-2 RNA has been found in the feces of infected patients. It is known that intestinal epithelial cells, particularly the enterocytes of the small intestine, also express ACE2 receptors [Dhar, D. and Mohanty, A. Virus Res. (2020) 198018]. Poor outcomes of COVID-19 infection have been observed in elderly patients, particularly those with pre-existing cardiovascular, metabolic and renal disorders, in whom the disease severity and mortality rate are considerably higher [Viana, S D et al. Ageing Research Reviews (2020) 62: 101123, citing Du, R H et al. Eur. Respir. J. (2020) 55; Li, B et al Clin. Res. Cardio. (2020) 109: 531-538; Mehra, M R et al N. Eng. J. Med. (2020) 382 (26): e102; Roncon, L. et al J. Clin. Virol. (2020) 127: 104354; Shi, Q. et al. Diabetes Care (2020) 43 (70: 1382-1391; Siordia, Jr., JA. J. Clin. Virol. (2020) 127: 104357; Team, CC-R. MMWR Morb. Mortal. Wkly Rep. (2020) 69: 343-346; Wang, X. et al. Research (Wash DC) (2020) 2402961; Zhou, F. et al. Lancet (2020) 395: 1054-1062].

ACE2 is the receptor for SARS-CoV2-entry in human cells [Id., citing Hoffmann, M. et al. Cell (2020) 181: 271-280]. There is a constitutive ACE2 expression within the luminal surface of differentiated small intestinal epithelial cells and colonic crypt cells [Id, citing Harmer, D. et al. FEBS Lett. (2002) 532: 107-110], where ACE2 functions as the chaperone for membrane trafficking of the amino acid transporter B°AT1, which mediates the uptake of neutral amino acids into intestinal cells in a sodium dependent manner, even in the absence of collectrin [Id., citing Camargo, S M et al. Gastroenterology (2009) 136: 872-882; Kowalczuk, S. et al. FASEB J. (2008) 22: 2880-2887]. ACE2 shares around 50% homology with collectrin, a type I transmembrane protein that regulates the transporter of neutral amino acids in the kidney (Id., citing Zhang, H. et al. J. Biol. Chem. (2001) 276: 17132-9). It has been suggested that a B°AT1/ACE2 complex in the intestinal epithelium regulates gut microbiota composition and function, with important repercussions on local and systemic immune responses against pathogenic agents, e.g., viruses. A dysbiotic condition (decreased microbial diversity and richness, and impaired Firmicutes to Bacteroides ratio) has been reported in the elderly and in age-related cardiovascular, metabolic, renal and pulmonary conditions. In an ACE2/γ-Akita mouse model, gut barrier disruption and exacerbated diabetes-induced dysbiosis have been described [Id., citing Duan, Y. et al. Cir. Res. (2019) 125: 969-988]. Some patients with COVID-19 have presented with intestinal microbial dysbiosis. It therefore has been hypothesized that pre-existing disorders displaying an altered gut microbiome may worsen SARS-CoV-2 infection due to a loss of ACE2 integrity/functionality in the gut.

In a pilot study of 15 patients with COVID-19, persistent alterations in the fecal microbiome during the time of hospitalization were found compared with controls. Fecal microbiota alterations were associated with fecal levels of levels of SARS-CoV-2 and COVID-19 severity. Over the course of hospitalization, bacterial species which downregulate expression of ACE2 in murine gut correlated inversely with SARS-CoV-2 load in fecal samples [Zuo, T. et al. Gastroenterology (2020) 159: 944-945].

### Mitochondrial Dysfunction

According to some embodiments, a composition of the present disclosure comprising a purified enriched population of potent exosomes comprising an appropriate therapeutic signature may modulate mitochondrial dysfunction. Mitochondrial dysfunction has been defined as changes in gene expression of mitochondrial markers [Montgomery, MK. Biology (Basel) (2019) 8 (2): 33, citing Heilbaronnn, L K et al. J. Clin. Endocrinol. Metab. (2007) 92: 1467-1473; Mootha, V K et al. Nat. Rev. Genet. (2003) 34: 267-273], protein content, or enzymatic activities of mitochondrial proteins [Id., citing Heilbronn, L K et al. J. Clin. Endocrinol. Metab. (2007) 92: 1467-1473; Ritov, VB, Diabetes (2005) 54: 8-14], changes in mitochondrial size and shape [Id., citing Rritov, V B et al. Diabetes (2005) 54: 8-14; Kelly, D E et al. Diabetes (2002) 51: 2944-2950], as well as functional assessment of mitochondrial oxidative capacity [Id., citing Kelley, D E et al. Am. J. Physiol. (1999) 277: E1130-E1141] and ROS generation [Id., citing Anderson, E J et al. J. Clin. Invest. (2009) 119: 573-581]. Mitochondrial defects are present in the heart in individuals with insulin resistance and diabetes [Id., citing Montaigne, D. et al. Circulation (2014) 130: 554-564; Dhalla, N S et al. Heart Fail. Rev. (2014) 19: 87-99; Mackenzie, R M et al. Clin. Sci. (2013) 124: 403-411; Croston, T L et al. Am. J. Physiol. Heart Cir. Physiol. (2014) 307: H54-H65] as well as in rodent models [Id., citing Marciniak, C. et al. Cardiovasc. Diabetol. (2014) 13: 118; Pham, T. et al. Am. J. Physiol. Cell Physiol. (2014) 307: C499-C507; Hicks, S. et al. Am. J. Physiol. Heart Cir. Physiol. (2013) 304: H903-H915; Yan, W. et al. Basic Res. Cardiol (2013) 108: 329; Luo, M. et al. J. Clin. Invest. (2013) 123: 1262-1274; Vazquez, E J et al. Cardiovasc. Res. (2015) 107: 453-465]. Alterations in mitochondrial energy metabolism are a common feature of various forms of heart disease [Id., citing Lopaschuk, G D, et al. Physiol Rev. (2010) 90: 207-258; Fillmore, N. et al. Br. J. Pharmacol. (2014) 171 (8): 2080-2090]. Diabetic cardiomyopathies (ventricular dysfunction in patients with diabetes mellitus) are associated with dysregulated oxidative substrate selection. In humans and rodents, obesity and insulin resistance are associated with increased myocardial fatty acid uptake and fatty acid oxidation [Id., citing Szczepaniak, L S et al. Magn. Reson. Med. (2003) 49: 417-423; Peterson, L. et al. Circulation (2004) 109: 2191-2196], with a simultaneous decrease in glucose oxidation [Id., citing Mazumder, P K et al. Diabetes (2004) 53: 2366-2374]. As fatty acid oxidation produces less ATP (per mol oxygen consumed), this scenario makes the heart less energy efficient [Id., citing Peterson, L. et al. Circulation (2004) 109: 2191-2196]. In addition, increased fatty acid supply to the heart is also associated with oxidative stress [Ansley, D M and Wang, B. J. Pathol. (2013) 229: 232-141] and accumulation of bioactive lipid intermediates that directly interfere with insulin signaling [Id., citing Zhang, L. et al. Cardiovasc. Res. (2011) 89: 148-156], and lead to a greater decline in heart function with age [Id., citing Kuramoto, K. et al. J. Biol. Chem. (2012) 287: 23852-23863]. Exosomes containing increased content of mitochondrial lipids, proteins and nucleic acids were found to be released from adipose tissue of obese diabetic and obese non-diabetic rats [Id., citing Lee, J. et al. Protein J. (2015) 34: 220-235], from pulmonary cells after cigarette smoke injury [Id., citing Szczesny, B. et al. Sci. Rep. (2018) 8: 914], and are found in plasma upon infection with the human T-lymphotropic retrovirus type 1 (HTLV-1) [Id., citing Jeannin, P. et al. Sci. Rep. (2018) 8: 5170] and in circulating exosomes of breast cancer patients [Id., citing Kannan, A. et al. Clin. Cancer Res. (2016) 22: 3348-3360] Very little is known about exosomal transfer of mitochondrial cargo in the presence of mitochondrial dysfunction during the development of insulin resistance and T2D.

### Neurodegeneration:

According to some embodiments, a composition of the present disclosure comprising a purified, enriched population of potent exosomes comprising an appropriate therapeutic signature may improve cognitive outcome after repetitive mild traumatic brain injury (rmTBI), may reduce symptoms of neurologic diseases comprising spread of toxic proteins within the nervous system; or may reduce neurological manifestations of a severe viral infection with a neurotropic virus, e.g., SARS-CoV-2.

miR-124-3p level in microglial exosomes from injured brain has been reported to be significantly altered in the acute, sub-acute, and chronic phases after repetitive mild traumatic brain injury (rmTBI). In in vitro experiments, microglial exosomes with upregulated miR-124-3p alleviated neurodegeneration in repetitive scratch-injured neurons. The effects were exerted by miR-124-3p targeting Rela, an inhibitory transcription factor of ApoE that promotes the β-amyloid proteolytic breakdown, thereby inhibiting β-amyloid abnormalities. In mice with rmTBI, the intravenously injected microglial exosomes were taken up by neurons in injured brain. miR-124-3p in the exosomes also was transferred into hippocampal neurons and alleviated neurodegeneration by targeting the Rela/ApoE signaling pathway [Ge, X. Mol. Ther. (2020) 28 (20): 503-522].

It has been suggested that EVs may be involved in the spread of toxic proteins within the nervous system in a number of neurologic diseases, such as Alzheimer's disease, Parkinson's disease, prion diseases, multiple sclerosis, brain tumor, and schizophrenia [Tsilioni, I. et al. Clinical-Therapeutics (2014) 36 (6): 882-888, citing Vella, L J et al. Eur. Biophys. J. (2008) 37: 323-332; Banigan, M G et al. PLoS One (2013) 8: e48814; Vingtdeux, V. et al. J. Biol. Chem. (2007) 282: 18197-18205; Guest, W C et al. J. Toxicol. Environ. Health. A (2011) 74: 1433-1459; Saenz-Cuesta, M. et al. Front. Cell Neurosci. (2014) 8: 100; Gonda, D D et al. Neurosurgery (2013) 72: 501-510]. In all of these diseases, exosomes are involved in the spread of “toxic” proteins that are mutated or misfolded and serve as templates for the formation of disease-producing oligomers [Id., citing Vella, L J et al. Eur. Biophys. J. (2008) 37: 323-332; Fruhbeis, C. et al. Front. Physiol. (2012) 3: 119]. Without being limited by theory, neurons may try to dispose of these proteins by processing them through the endosomal pathway, which leads wither to degradation from lysosomes or to incorporation into MVBs and release into the extracellular space as exosomes.

### SARS-CoV-2

Neurological investigations and isolation of SARS-CoV-2 from cerebrospinal fluid indicate that SARS-CoV-2 is a neurotropic virus and causes multiple neurological manifestations [Ahmed, M U et al. Frontiers Neurology (2020) 11: 518]. A retrospective case series study involving 214 patients, of which 78 (36.4%) had some neurological symptoms, in Wuhan, China showed that patients with severe COVID-19 develop more neurological symptoms, such as acute cerebrovascular accidents, altered level of consciousness, and skeletal muscle damage compared to those with mild infection [Id., citing Mao, L. et al. JAMA Neurol. (2020) e201127]. ACE2 receptors are expressed on glial tissues, neurons and brain vasculature, which make them a target for the attack by SARS-CoV-2 [Id., citing Turner, A J et al. Trends Pharmacol. Sci. (2004) 25: 291-294]. The presence of the virus in neuronal and vascular endothelial cells in frontal tissues was detected on an autopsy of a confirmed COVID-19 patient [Id., citing Paniz-Mondolfi, A. et al. J. Med. Virol. (2020) doi: 10.1002/jmv.25915].

The presence of the virus in general circulation may enable virus entry into cerebral circulation, where sluggish blood movement in microvessels enables interaction of the viral spike protein with ACE2 receptors of capillary endothelium [Id., citing Baig, A M et al. ACS Chem. Neurosci. (2020) 11: 995-998]. This interaction subsequently leads to viral budding from capillary endothelium; resultant damage to the endothelial lining favors viral entry into the brain, where viral interaction with ACE2 receptors expressed over neurons can result in damage to the neurons without substantial inflammation, which has been described [Id., citing Wrapp, D. et al. Science (2020) 367: 1260-1263]. Endothelial damage in cerebral capillaries with resulting bleeding alone can have fatal consequences in COVID-19 patients. The avid binding of the virus to the ACE2 receptors also may result in their destruction via unknown mechanisms, leading to hemorrhage in the brain. Since ACE2 is a cardio-cerebral vascular protecting factor, its damage may cause a leak of the virus in the CNS [Id., citing Turner, A J et al. Trends Pharmacol. Sci. (2004) 25: 291-294]. Neurotropic viruses also can reach the CNS by anterograde and retrograde transport with the help of motor proteins kinesins and dynein via sensory and motor nerve endings [Id., citing Swanson, PA 2d and McGavern, DB. Curr. Opin. Virol. (2015) 11: 44-54], especially via afferent nerve endings of the vagus nerve from the lungs [Id., citing Li, Y C et al. J. Med. Virol. (2020) 92: 552-555]. SARS-CoV-2 also can cause gastrointestinal tract infection and spread to the CNS via enteric nerve and sympathetic afferents [Id, citing Wong, S H et al. J. Gastroenterol. Hepatol. (2020) 35: 744-748]. In addition, cytokine storms characterized by increased levels of inflammatory cytokines and activities of T lymphocytes, macrophages and endothelial cells can also cause neuronal damage; the release of IL-6 can cause vascular leakage and activation of complement and coagulation cascades [Abdullahi, A. et al. Front. Neurology (2020) 11: 687, citing Mehta, P et al. Lancet (2020) 395: 1033-1034].

The spectrum of neurological manifestations of COVID-19 documented by case reports includes encephalitis, anosmia (loss of the sense of smell)/hyposmia (meaning reduced ability to smell), viral meningitis, post-infectious acute disseminated encephalomyelitis; post-infectious brainstem encephalitis, Guillain Barre syndrome, and acute cerebrovascular disease. The prevalence of neurological and musculoskeletal manifestations of COVID-19 was 35% for smell impairment, 33% for taste impairment, 19% for myalgia, 12% for headache, 10% for back pain, 3% for acute cerebrovascular disease, and 2% for impaired consciousness [Abdullahi, A. et al. Front. Neurology (2020) 11: 687].

### Pain Conditions

According to some embodiments, a composition comprising a purified, enriched population of potent exosomes with a distinct therapeutic signature that addresses specific inflammatory and/or fibrotic pathways and/or cellular senescence may treat pain conditions.

There is some support in the literature for this use. Overexpression of MiR-21-5p supports a pro-inflammatory phenotype of macrophages attracted on the site of damage, while intrathecal delivery of a miR-21-5p antagomir appears to avoid an extension of inflammatory condition and neuropathic hypersensitivity onset [D'Agnelli, S. et al. Mol. Pain (2020) 16: PMC7493250, citing Simeoli, R. Nat. Commun. (2017) 8: 1778]. A proteome characterization of exosomes from mouse spared nerve injury (SNI) model suggested the cargo sorting of vesicular proteins as a crucial step in mediating signaling mechanisms underlying neuropathic pain and evidenced unique patterns of proteins. In particular, significant upregulation of complement component 5a (C5a) and Intercellular Adhesion Molecule 1 are detected in exosomes from SNI model compared to sham control [Id., citing Jean-Toussaint, R. et al. J. Proteomics (2020) 211: 103540]. The involvement of exosomes in neuropathic pain is also underlined by many studies on complex regional pain syndrome (CRPS). It is a chronic neuropathic pain disorder, disabling for sensory, motor, and autonomic dysfunctions as well as of allodynia, hyperalgesia, dystonia, and tremors [Id., citing Bruehl, S. BMJ (2015) 351: h2730]. A different miRNA-exosomal profile between responders and non-responders to treatment in CRPS patients has been identified, suggesting a potential tool to prior identify a subgroup of patients with higher possibility to have benefit by that specific treatment, in this case from plasmapheresis [Id., citing Ramanathan, S. et al. J. Transl. Med. (2019) 17: 81] In a mouse model of CRPS, the mechanism of action of macrophage-derived exosomes and their cargo has been investigated. A decrement of thermal hyperalgesia following a single injection of macrophage-derived exosomes has been found, suggesting a potential immunoprotective role. In the same study, serum-derived exosomes from CRPS patients were analyzed and 127 miRNAs were significantly different comparing CRPS exosomes with control-derived exosomes. Among them, three miRNAs (miR-21-3p, miR-146a, and miR-146b), known to be involved in the control of overactivation of innate immune response, are over expressed in both murine and human model [Id., citing McDonald, M K et al. Pain (2014) 155: 1527-1539].

### Solid Organ Transplantation.

In view of their anti-fibrotic and pro-angiogenic effects, a composition comprising a purified, enriched population of potent exosomes with an appropriate therapeutic signature may improve graft survival by, for example, reducing ischemia reperfusion injury, by improving recovery from acute damage, and by modulating tolerance towards the graft.

According to some embodiments, a pharmaceutical composition comprising a purified, enriched population of potent exosomes with a distinct therapeutic signature may be helpful for preserving donor organs when administered before transplant in the context of hypothermic or normothermic perfusion machines. According to some embodiments, addition of EVs to the perfusion solution may improve donor organ viability and functionality.

There is some support for this notion in the literature. For example, perfusion solution containing EVs has shown some success in organ preconditioning. One report demonstrated that EVs released by MSCs, delivered in the perfusate during organ cold perfusion (4 h), preserved and protected kidney function. Histological and genetic analyses on EV-treated kidneys revealed upregulation of enzymes involved in energy metabolism and reduction of global ischemic damage [Id., citing Gregorini, M. et al. J. Cell Mol. Med. (2017) 21: 3381-3393] Another report demonstrated that EVs isolated from the venal perfusate of rats subjected to remote ischemia preconditioning ameliorated renal function when injected into another animal with ischemia-reperfusion injury (IRI). To explore the underlying mechanism, the authors tested in the same IRI model in vivo the effect of EVs released by human proximal tubular cells cultured in hypoxia, supporting the hypothesis that remote ischemia preconditioning activates a repairing program into tubular cells by the release of pro-regenerative EVs [Id., citing Zhang, G. et al. Biomed. Pharmacother. (2017) 90: 473-478]. The use of EVs released by stem cells to improve the viability of pre-transplant livers was recently assessed in a model of ex vivo rat liver NMP. EVs isolated from human liver stem cells (HLSC-EVs) were added to the perfusate 15 min after the initiation of normothermic machine perfusion (NMP) and administered for 4 h within the perfusate. The results showed that HLSC-EVs limited the progression of ischemic injury, with a significant reduction of the levels of aspartate aminotransferase and alanine aminotransferase and a decrease of histological damage compared with results of NMP alone [Id., citing Rigo, F. et al. Transplantation (2018) 102: e205-e210].

Bone marrow MSC-EVs have been shown to recapitulate the therapeutic effects of the cells against acute GVHD [Id., citing Selmani, Z. et al. Cells (2008) 26: 212-222]. A systemic infusion of MSC-EVs in mice with acute GVHD was associated with the suppression of CD4+ and CD8+ T cells and with the preservation of circulating naive T cells, possibly due to the unique microRNA profiles of MSC-EVs. The analysis on microRNA cargo in MSC-EVs identified that their target genes were involved in regulation of the cell cycle, T-cell receptor signaling, and GVHD.

In testing the effect of EVs in a rat model of IRI after DCD renal transplantation, MSC-EVs derived from Wharton's jelly, intravenously injected after renal transplantation, were shown to mitigate renal damage, and improve survival and function. In particular, MSC-EVs were shown to reduce cell apoptosis and inflammation, to stimulate HGF production, and subsequently to alleviate fibrosis [Id., citing Wu, X. et al. J. Cell Biochem. (2018) 119: 1879-1888].

According to some embodiments, the chronic lung disease is a fibrotic lung disease. According to some embodiments, the chronic lung disease is due to chronic smoking or a severe viral infection. According to some embodiments, the severe lung infection is due to a severe coronavirus infection. According to some embodiments, the age-related chronic lung disease comprises reduced forced vital capacity compared to a normal healthy control. According to some embodiments, the treatment results in stabilization or improvement of forced vital capacity in a subject compared to an untreated control.

### Method for Diagnosis and Treatment

According to another aspect, the present disclosure provides a method for diagnosing a human subject aged over 50 years with an age-related chronic disease characterized by disease related dysfunction and optimally treating the subject, comprising
(a) diagnosing a stage of the age-related chronic disease by: isolating a population of extracellular vesicles (EVs) from mesenchymal stem cells derived from a biological sample obtained from the subject and from a normal healthy control aged 21-40, inclusive; purifying and enriching a purified enriched population of potent exosomes derived from the mesenchymal stem cells from the subject and the normal healthy control; wherein the purified, enriched exosomes comprise an identity signature comprising expression three or more biomarkers selected from CD9, CD63, CD81, or Tsg101+; measuring a level of expression of each of a plurality of miRNAs in the purified enriched exosomes from the subject and from the normal healthy control; determining that expression of the one or more miRNAs in the purified, enriched population of exosomes from the subject is dysregulated compared to the healthy control; and identifying the subject as one that can benefit therapeutically from being treated for the age-related chronic disease; and
(b) treating the age-related chronic disease by administering a composition comprising the population of purified enriched potent exosomes comprising an appropriate therapeutic signature derived from the normal healthy subject, wherein the exosomes comprise an identity signature of three or more biomarkers selected from CD9, CD63, CD81, or Tsg101+; the exosomes comprise total protein of about 1 mg; the exosomes comprise total RNA content greater than 20 μg; the exosomes comprise a cargo comprising a therapeutic signature of one or more, two or more, three or more, four or more, or five or more miRNAs selected from miRNA-29a, miRNA-10a, miRNA-34a, miRNA-125, miRNA-181a, miRNA-181c, miRNA-Let-7a, miRNA-Let-7b, miRNA-Let-7d, miRNA-146a, miRNA-145, miRNA-21, miRNA-101, and miRNA-199; and size of the exosomes is 90-110 nm, inclusive; wherein lot to lot and batch to batch variability relative to prior lots of the composition is below a significance level of ≤0.05; and
(c) managing the age related chronic disease by reducing or slowing the dysfunction.

According to some embodiments, identity of the MSCs from which the purified, enriched potent exosomes are isolated is confirmed by a signature comprising expression of CD29, CD44, and CD105. According to some embodiments, the cargo comprises a potency signature of expression of one or more, two or more, three or more, four or more, or five or more of angiopoietin 2 (Ang-2), fibroblast growth factor (FGF), hepatic growth factor (HGF), interleukin 8 (IL-8), a tissue inhibitor of metalloproteinases (TIMP), vasculoendothelial growth factor (VEGF), platelet derived growth factor (PDGF), or tumor necrosis factor alpha (TNFα). According to some embodiments, the tissue inhibitor of metalloproteinases is TIMP1, TIMP2, or TIMP1 and TIMP2.

According to some embodiments, the composition is a pharmaceutical composition comprising a therapeutic amount of the purified enriched population of exosomes and a pharmaceutically acceptable carrier. According to some embodiments, per isolation process, there are at least 1×10E8 EVs comprising the purified enriched population of exosomes. According to some embodiments, a therapeutic amount of exosomes comprises at least 1×10E8 exosomes. According to some embodiments, the administering is by inhalation or by intravenous administration.

According to some embodiments, the age-related chronic disease if left untreated comprises one or more of a progressive injury, progressive inflammation, progressive fibrosis or a combination thereof.

According to some embodiments, the therapeutic signature of the exosome cargo may modulate one or more of an injury, inflammation, an excess accumulation of extracellular matrix, cell senescence; or a pathway comprising fibrogenic signaling; or may reprograms a tissue affected by the age-related chronic disease. According to some embodiments, the pathway comprises transforming growth factor (TGFβ) signaling. According to some embodiments, the pathway comprising fibrogenic signaling is one or more pathway that upregulates TERT (Telomerase Reverse Transcriptase) transcription including a Smad pathway, a mitogen-activated protein kinase pathway, a phosphoinositide 3-kinase pathway; a canonical Wnt-β catenin pathway, an NF-κB pathway, or a Notch signaling pathway. According to some embodiments, the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle.

According to some embodiments the age-related chronic disease is a progressive chronic lung disease, chronic inflammation and immune dysfunction, mitochondrial dysfunction, organ transplantation dysfunction; fibrotic disposition of a donor organ, rejection of a donor organ; graft failure; ex vivo lung perfusion dysfunction, musculoskeletal disorders, neurodegeneration, gut dysbiosis or microbiome dysfunction, or age-related decline. According to some embodiments the progressive chronic lung disease is a fibrotic lung disease. According to some embodiments, the fibrotic lung disease is pulmonary fibrosis. According to some embodiments, the fibrotic lung disease is idiopathic pulmonary fibrosis. According to some embodiments, the chronic lung disease is due to chronic smoking or a severe viral infection. According to some embodiments the severe lung infection is due to a severe coronavirus infection. According to some embodiments the age-related progressive chronic lung disease comprises reduced forced vital capacity compared to a normal healthy control. According to some embodiments, the treating of the progressive chronic lung disease is effective to stabilize or improve forced vital capacity in the subject compared to an untreated control.

### Microbiome Dysfunction

According to some embodiments, a composition of the present disclosure comprising a population of purified enriched potent exosomes with an appropriate therapeutic signature of MSC-derived EVs isolated from a normal healthy subject may modulate microbiome dysfunction.

The human microbiota consists of 10-100 trillion symbiotic microbial cells harbored by each person, primarily bacteria in the gut, while the human microbiome consists of the genes these cells harbor [Ursell, L K et al., “Defining the human microbiome.” Nutr. Rev. (2012) 70 (Suppl. 1) S38-S44)]. These microbial cells, and their genetic material, live with humans from birth, and every individual has a unique mix of species. This relationship is important for nutrition, immunity and effects on the brain and behavior, and has been implicated in a number of diseases where the disease is caused by a disturbance in the normal balance of microbes or where the disturbance is another downstream consequence of the disease. The interaction between the human microbiota and the environment is dynamic, meaning that microbial communities are constantly being transferred between surfaces, and that a dynamic interaction exists between environmental microbiota and different human body sites. There is increasing evidence that individuals actually share a core microbiota, with vastly different sets of microbial species yielded very similar functional molecular interactions, reactions and relation networks for metabolism, genetic information processing, environmental information processing, cellular processes, and organismal systems referred to as KEGG pathways.

There is accumulating evidence that a crosstalk between the gut and lungs exists. The lungs of healthy individuals harbor Fusobacterium, Haemophilus, Prevotella, Streptococcus, and Veillonella as main genera, which are relatively small in size when compared to the enteric microbiota [Ahlawat, S. et al. Virus Res. (2020) 286: 198103, citing He, et al., Y. et al. Crit. Rev. Microbiol. (2017) 43 (1): 81-95]. The emergence and maintenance of lung microbiota is governed by the equilibrium between microbial migration from the upper respiratory tract and microbial removal by the host defense systems, with small contribution from the multiplication of native microbes. Even in small concentrations, the airway microbiome is believed to be crucial to host immunity, such that an imbalance between microbial immigration and removal predisposes its host to the progression and exacerbations of respiratory diseases [Id., citing He, et al., Y. et al. Crit. Rev. Microbiol. (2017) 43 (1): 81-95; Wypych, T P et al. Nat. Immunol. (2019) 20 (10): 1279-1290]. Alterations in the lung microbial community including airways also affects the composition of the intestinal microbiota, and respiratory viral infections can alter the intestinal microbiome, where the intestinal microbiome determines the adaptive immune responses against the respiratory pathogens and is necessary for priming the innate immune responses against the pulmonary infection. Indeed, during respiratory viral infections, the level of macrophage response to the respiratory viruses depends on the presence of intestinal microbes [Id., citing Hanada, S. et al. Front. Immunol. (2018) 9: 2640].

Gut microbiota composition and diversity is affected by many factors, including by aging. An apparent age-related gut microbiota imbalance has been described, featuring an altered microbial diversity, a lower abundance of probiotic strains (e.g., Difidobacteria), and a reduced number of species producing butyrate, a short chain fatty acid (SCFA) that plays important metabolic functions and has a major role in maintaining the integrity of intestinal epithelium [Mangiola, F. et al. Eur. Rev. Med. Pharmacol. Sci. 2018] 22: 7404-7413].

The integrity of the commensal microbiota can be disturbed by invading viruses, which cause dysbiosis (meaning a general imbalance of gut microbiota) in the host and further influence virus infectivity [Li, N. et al. Frontiers Immunol. (2019) 10: 1551]. It has been shown that RNA viruses, such as poliovirus, benefit from the delivery of various viral genomes into a single target cell, thereby allowing the recombination of multiple virus genomes; this process potentiates those viral progeny with enhanced environmental fitness [Id., citing Aguilera, E R et al. M. Bio. (2017) 8: 16; Chen, Y H et al. Cell (2015) 160: 619-30; Combe, M. et al. Cell Host Microbe (2015) 18: 424-432]. Some viruses (e.g., poliovirus, reovirus) use commensal microbiota components to enhance viral stability and increase infectivity. There is also likely a link between commensal microbiota and the lytic reactivation of viruses. In addition to fostering the generation of immunoregulatory Treg cells and thereby suppress anti-viral immunity, the commensal microbiota also have been reported to directly skew antiviral immunity by suppressing the activation of effector immune cells and by inhibiting the production of various inflammatory cytokines that are pivotal for virus elimination, thus creating a more favorable environment for viral infection [Id., citing Baldridge, M T et al. Science (2015) 347: 266-269].

The commensal microbiota also can prevent viral infection. For example, Enterococcus faecium can prevent infection by influenza viruses upon direct absorptive trapping of these viruses [Id., citing Wang, X Y et al. PLoS One (2013) 8: e53043]. An extracellular matrix binding protein produced by Staphylococcus epidermidis, a Gram-positive bacterium that lives in the human nasal cavity as a commensal, can stably bind to influenza virus and thus block further infection [Id., citing Chen, H W et al. Sci. Rep. (2016) 6: 27870]. The replication of herpes simplex virus-2 (HSV-2) can also be suppressed by commensal microbiota metabolites; lactic acid, a major end product of the carbohydrate fermentation of all Lactobacillus species, can strongly inactivate HSV-2 in the vaginal mucosa by maintaining an acidic pH in the local environment [Id., citing Tuyama, C G et al. J. Infect. Dis. (2006) 194: 795-803]. Commensal microbiota also exert their antiviral activity through cell wall associated bacterial components [Id., citing Mastromarino, P. et al. Anaerobe (2011) 17: 334-336].

Supporting studies have shown that intact healthy commensal microbiota help maintain robust antiviral immunity, while microbiota disruption increases viral infectivity due to the impaired capacity of the immune system to limit viral infection. For example, Clostridium orbiscindens, a human-associated gut microbe produces desaminotyrosine to prime the amplification loop of type I IFN signaling, thereby mediating protection against influenza infection [Id, citing Steed, A L et al. Science (2017) 357: 498-502]. It has been shown that during respiratory influenza virus infection, antibiotic exposure led to a defective generation of virus-specific CD4 and CD8 T cells and antibodies due to an impaired inflammasome-dependent migration of APCs from the lung to the draining lymph nodes [Id., citing Ichinohe, T. et al. Proc. Natl Acad. Sci. USA (2011) 108: 5354-5359].

Studies also suggest an important role of virus infection in inducing microbiota dysbiosis. This is true for HIV/SIV infection, influenza virus infection, HBV or hepatitis C virus infection (HCV) and norovirus infection. For example, microbial diversity in saliva of HIV patients was significantly reduced compared to healthy controls, accompanied by increased abundance of potentially pathogenic Megasphaera, Camplyobacter, Veillonella and Prevotella species, and decreased commensal Veillonella and Streptococcus species [Id., citing Li, Y. et al. J. Clin. Microbiol. (2014) 52: 1400-1411; Dang, A T et al. BMC Microbiol. (2012) 12 (152): 95, 96]. In addition, fungal communities in HIV infected and uninfected individuals differed significantly [Id., citing Mukherjee, P K et al. PLoS Pathog. (2014) 10: e1003996]. In bronchoalveolar lavage fluids, although there were no significant differences among the microbial composition in HIV-infected and uninfected subjects, specific metabolic profiles were associated with bacterial organisms that potentially play a role in the pathogenesis of pneumonia in HIV infected patients [Id., citing Cribbs, S K et al. Microbiome (2016) 4: 3]. Mechanistically, specific immune suppression by HIV is partly responsible for the enrichment of certain potentially pathogenic bacteria.

Several studies have shown that influenza virus infection can result in decreased colonization by healthy bacteria and increased abundance of potentially pathogenic microbiota. There is a consensus that influenza virus infection alters the commensal microbiota of the host, causing corresponding disruptions of the microbiota-host homeostasis, which largely accounts for the mechanisms by which infections are established. Several lines of data have highlighted that the modulation of immune responses by influenza contributes to the dysbiosis of the gut [Id., citing Wang, J. et al. J. Exp. Med. (2014) 211: 2397-2410; Deriu, E. et al. PLoS Pathog. (2016) 12: e1005572].

Although SARS-CoV-2 primarily causes lung infection through binding of ACE2 receptors present on alveolar epithelial cells, SARS-CoV-2 RNA has been found in the feces of infected patients. It is known that intestinal epithelial cells, particularly the enterocytes of the small intestine, also express ACE2 receptors [Dhar, D. and Mohanty, A. Virus Res. (2020) 198018]. Poor outcomes of COVID-19 infection have been observed in elderly patients, particularly those with pre-existing cardiovascular, metabolic and renal disorders, in whom the disease severity and mortality rate are considerably higher [Viana, S D et al. Ageing Research Reviews (2020) 62: 101123, citing Du, R H et al. Eur. Respir. J. (2020) 55; Li, B et al Clin. Res. Cardio. (2020) 109: 531-538; Mehra, M R et al N. Eng. J. Med. (2020) 382 (26): e102; Roncon, L. et al J. Clin. Virol. (2020) 127: 104354; Shi, Q. et al. Diabetes Care (2020) 43 (70: 1382-1391; Siordia, Jr., JA. J. Clin. Virol. (2020) 127: 104357; Team, CC-R. MMWR Morb. Mortal. Wkly Rep. (2020) 69: 343-346; Wang, X. et al. Research (Wash DC) (2020) 2402961; Zhou, F. et al. Lancet (2020) 395: 1054-1062].

ACE2 is the receptor for SARS-CoV2-entry in human cells [Id., citing Hoffmann, M. et al. Cell (2020) 181: 271-280]. There is a constitutive ACE2 expression within the luminal surface of differentiated small intestinal epithelial cells and colonic crypt cells [Id, citing Harmer, D. et al. FEBS Lett. (2002) 532: 107-110], where ACE2 functions as the chaperone for membrane trafficking of the amino acid transporter B°AT1, which mediates the uptake of neutral amino acids into intestinal cells in a sodium dependent manner, even in the absence of collectrin [Id., citing Camargo, S M et al. Gastroenterology (2009) 136: 872-882; Kowalczuk, S. et al. FASEB J. (2008) 22: 2880-2887]. ACE2 shares around 50% homology with collectrin, a type I transmembrane protein that regulates the transporter of neutral amino acids in the kidney (Id., citing Zhang, H. et al. J. Biol. Chem. (2001) 276: 17132-17139). It has been suggested that a B°AT1/ACE2 complex in the intestinal epithelium regulates gut microbiota composition and function, with important repercussions on local and systemic immune responses against pathogenic agents, e.g., viruses. A dysbiotic condition (decreased microbial diversity and richness, and impaired Firmicutes to Bacteroides ratio) has been reported in the elderly and in age-related cardiovascular, metabolic, renal and pulmonary conditions. In an ACE2/γ-Akita mouse model, gut barrier disruption and exacerbated diabetes-induced dysbiosis have been described [Id., citing Duan, Y. et al. Cir. Res. (2019) 125: 969-988]. Some patients with COVID-19 have presented with intestinal microbial dysbiosis. It therefore has been hypothesized that pre-existing disorders displaying an altered gut microbiome may worsen SARS-CoV-2 infection due to a loss of ACE2 integrity/functionality in the gut.

In a pilot study of 15 patients with COVID-19, persistent alterations in the fecal microbiome during the time of hospitalization were found compared with controls. Fecal microbiota alterations were associated with fecal levels of levels of SARS-CoV-2 and COVID-19 severity. Over the course of hospitalization, bacterial species which downregulate expression of ACE2 in murine gut correlated inversely with SARS-CoV-2 load in fecal samples [Zuo, T. et al. Gastroenterology (2020) 159: 944-945].

### Mitochondrial Dysfunction

According to some embodiments, a composition of the present disclosure comprising an appropriate therapeutic signature of a population of purified enriched potent exosomes isolated from a normal healthy subject may modulate mitochondrial dysfunction. Mitochondrial dysfunction has been defined as changes in gene expression of mitochondrial markers [Montgomery, MK. Biology (Basel) (2019) 8 (2): 33, citing Heilbronn, L K et al. J. Clin. Endocrinol. Metab. (2007) 92: 1467-1473; Mootha, V K et al. Nat. Rev. Genet. (2003) 34: 267-14], protein content, or enzymatic activities of mitochondrial proteins [Id., citing Heilbronn, L K et al. J. Clin. Endocrinol. Metab. (2007) 92: 1467-1473; Ritov, VB, Diabetes (2005) 54: 8-14], changes in mitochondrial size and shape [Id., citing Rritov, V B et al. Diabetes (2005) 54: 8-14; Kelly, D E et al. Diabetes (2002) 51: 2944-2950], as well as functional assessment of mitochondrial oxidative capacity [Id., citing Kelley, D E et al. Am. J. Physiol. (1999) 277: E1130-41] and ROS generation [Id., citing Anderson, E J et al. J. Clin. Invest. (2009) 119: 573-581]. Mitochondrial defects are present in the heart in individuals with insulin resistance and diabetes [Id., citing Montaigne, D. et al. Circulation (2014) 130: 554-564; Dhalla, N S et al. Heart Fail. Rev. (2014) 19: 87-99; Mackenzie, R M et al. Clin. Sci. (2013) 124: 403-411; Croston, T L et al. Am. J. Physiol. Heart Cir. Physiol. (2014) 307: H54-H65] as well as in rodent models [Id., citing Marciniak, C. et al. Cardiovasc. Diabetol. (2014) 13: 118; Pham, T. et al. Am. J. Physiol. Cell Physiol. (2014) 307: C499-C507; Hicks, S. et al. Am. J. Physiol. Heart Cir. Physiol. (2013) 304: H903-H915; Yan, W. et al. Basic Res. Cardiol. (2013) 108: 329; Luo, M. et al. J. Clin. Invest. (2013) 123: 1262-1274; Vazquez, E J et al. Cardiovasc. Res. (2015) 107: 453-465]. Alterations in mitochondrial energy metabolism are a common feature of various forms of heart disease [Id., citing Lopaschuk, G D, et al. Physiol Rev. (2010) 90: 207-258; Fillmore, N. et al. Br. J. Pharmacol.]. diabetic cardiomyopathies (ventricular dysfunction in patients with diabetes mellitus) are associated with dysregulated oxidative substrate selection. In humans and rodents, obesity and insulin resistance are associated with increased myocardial fatty acid uptake and fatty acid oxidation [Id., citing Szczepaniak, L S et al. Magn. Reson. Med. (2003) 49: 417-423; Peterson, L. et al. Circulation (2004) 109: 2191-2196], with a simultaneous decrease in glucose oxidation [Id., citing Mazumder, P K et al. Diabetes (2004) 53: 2366-2374]. As fatty acid oxidation produces less ATP (per mol oxygen consumed), this scenario makes the heart less energy efficient [Id., citing Peterson, L. et al. Circulation (2004) 109: 2191-2196]. In addition, increased fatty acid supply to the heart is also associated with oxidative stress [Ansley, D M and Wang, B. J. Pathol. (2013) 229: 232-241] and accumulation of bioactive lipid intermediates that directly interfere with insulin signaling [Id., citing Zhang, L. et al. Cardiovasc. Res. (2011) 89: 148-156], and lead to a greater decline in heart function with age [Id., citing Kuramoto, K. et al. J. Biol. Chem. (2012) 287: 23852-23863]. Exosomes containing increased content of mitochondrial lipids, proteins and nucleic acids were found to be released from adipose tissue of obese diabetic and obese non-diabetic rats [Id., citing Lee, J. et al. Protein J. (2015) 34: 220-235], from pulmonary cells after cigarette smoke injury [Id., citing Szczesny, B. et al. Sci. Rep. (2018) 8: 914], and are found in plasma upon infection with the human T-lymphotropic retrovirus type 1 (HTLV-1) [Id., citing Jeannin, P. et al. Sci. Rep. (2018) 8: 5170] and in circulating exosomes of breast cancer patients [Id., citing Kannan, A. et al. Clin. Cancer Res. (2016) 22: 3348-3360] Very little is known about exosomal transfer of mitochondrial cargo in the presence of mitochondrial dysfunction during the development of insulin resistance and type 2 diabetes (T2D).

### Neurodegeneration:

According to some embodiments, a composition of the present disclosure comprising a population of purified enriched potent exosomes with an appropriate therapeutic signature may improve cognitive outcome after repetitive mild traumatic brain injury (rmTBI), in neurologic diseases comprising spread of toxic proteins within the nervous system; or neurological manifestations of a severe viral infection with a neurotropic virus, e.g., SARS-CoV-2.

miR-124-3p level in microglial exosomes from injured brain has been reported to be significantly altered in the acute, sub-acute, and chronic phases after repetitive mild traumatic brain injury (rmTBI). In in vitro experiments, microglial exosomes with upregulated miR-124-3p alleviated neurodegeneration in repetitive scratch-injured neurons. The effects were exerted by miR-124-3p targeting Rela, an inhibitory transcription factor of ApoE that promotes the β-amyloid proteolytic breakdown, thereby inhibiting β-amyloid abnormalities. In mice with rmTBI, the intravenously injected microglial exosomes were taken up by neurons in injured brain. miR-124-3p in the exosomes also was transferred into hippocampal neurons and alleviated neurodegeneration by targeting the Rela/ApoE signaling pathway [Ge, X. Mol. Ther. (2020) 28 (20): 503-522].

It has been suggested that EVs may be involved in the spread of toxic proteins within the nervous system in a number of neurologic diseases, such as Alzheimer's disease, Parkinson's disease, prion diseases, multiple sclerosis, brain tumor, and schizophrenia [Tsilioni, I. et al. Clinical-Therapeutics (2014) 36 (6): 882-888, citing Vella, L J et al. Eur. Biophys. J. (2008) 37: 323-332; Banigan, M G et al. PLoS One (2013) 8: e48814; Vingtdeux, V. et al. J. Biol. Chem. (2007) 282: 18197-18205; Guest, W C et al. J. Toxicol. Environ. Health. A (2011) 74: 1433-1459; Saenz-Cuesta, M. et al. Front. Cell Neurosci. (2014) 8: 100; Gonda, D D et al. Neurosurgery (2013) 72: 501-510]. In all of these diseases, exosomes are involved in the spread of “toxic” proteins that are mutated or misfolded and serve as templates for the formation of disease-producing oligomers [Id., citing Vella, L J et al. Eur. Biophys. J. (2008) 37: 323-332; Fruhbeis, C. et al. Front. Physiol. (2012) 3: 119]. Without being limited by theory, neurons may try to dispose of these proteins by processing them through the endosomal pathway, which leads wither to degradation from lysosomes or to incorporation into MVBs and release into the extracellular space as exosomes.

### SARS-CoV-2

According to some embodiments, a composition according to the present disclosure comprising a population of purified enriched potent exosomes with a distinct therapeutic signature may address neurological manifestations of infection by SARS-CoV-2.

Neurological investigations and isolation of SARS-CoV-2 from cerebrospinal fluid indicate that SARS-CoV-2 is a neurotropic virus and causes multiple neurological manifestations [Ahmed, M U et al. Frontiers Neurology (2020) 11: 518]. A retrospective case series study involving 214 patients, of which 78 (36.4%) had some neurological symptoms, in Wuhan, China showed that patients with severe COVID-19 develop more neurological symptoms, such as acute cerebrovascular accidents, altered level of consciousness, and skeletal muscle damage compared to those with mild infection [Id., citing Mao, L. et al. JAMA Neurol. (2020) e201127]. ACE2 receptors are expressed on glial tissues, neurons and brain vasculature, which make them a target for the attack by SARS-CoV-2 [Id., citing Turner, A J et al. Trends Pharmacol. Sci. (2004) 25: 291-294]. The presence of the virus in neuronal and vascular endothelial cells in frontal tissues was detected on an autopsy of a confirmed COVID-19 patient [Id., citing Paniz-Mondolfi, A. et al. J. Med. Virol. (2020) doi: 10.1002/jmv.25915].

The presence of the virus in general circulation may enable virus entry into cerebral circulation, where sluggish blood movement in microvessels enables interaction of the viral spike protein with ACE2 receptors of capillary endothelium [Id., citing Baig, A M et al. ACS Chem. Neurosci. (2020) 11: 995-998]. This interaction subsequently leads to viral budding from capillary endothelium; resultant damage to the endothelial lining favors viral entry into the brain, where viral interaction with ACE2 receptors expressed over neurons can result in damage to the neurons without substantial inflammation, which has been described [Id., citing Wrapp, D. et al. Science (2020) 367: 1260-1263]. Endothelial damage in cerebral capillaries with resulting bleeding alone can have fatal consequences in COVID-19 patients. The avid binding of the virus to the ACE2 receptors also may result in their destruction via unknown mechanisms, leading to hemorrhage in the brain. Since ACE2 is a cardio-cerebral vascular protecting factor, its damage may cause a leak of the virus in the CNS [Id., citing Turner, A J et al. Trends Pharmacol. Sci. (2004) 25: 291-294]. Neurotropic viruses also can reach the CNS by anterograde and retrograde transport with the help of motor proteins kinesins and dynein via sensory and motor nerve endings [Id., citing Swanson, PA 2d and McGavern, DB. Curr. Opin. Virol. (2015) 11: 44-54], especially via afferent nerve endings of the vagus nerve from the lungs [Id., citing Li, Y C et al. J. Med. Virol. (2020) 92: 552-555]. SARS-CoV-2 also can cause gastrointestinal tract infection and spread to the CNS via enteric nerve and sympathetic afferents [Id, citing Wong, S H et al. J. Gastroenterol. Hepatol. (2020) 35: 74474-8]. In addition, cytokine storms characterized by increased levels of inflammatory cytokines and activities of T lymphocytes, macrophages and endothelial cell can also cause neuronal damage; the release of IL-6 can cause vascular leakage and activation of complement and coagulation cascades [Abdullahi, A. et al. Front. Neurology (2020) 11: 687, citing Mehta, P et al. Lancet (2020) 395: 1033-1034].

The spectrum of neurological manifestations of COVID-19 documented by case reports includes encephalitis, anosmia (loss of the sense of smell)/hyposmia (meaning reduced ability to smell), viral meningitis, post-infectious acute disseminated encephalomyelitis; post-infectious brainstem encephalitis, Guillain Barre syndrome, and acute cerebrovascular disease. The prevalence of neurological and musculoskeletal manifestations of COVID-19 was 35% for smell impairment, 33% for taste impairment, 19% for myalgia, 12% for headache, 10% for back pain, 3% for acute cerebrovascular disease, and 2% for impaired consciousness [Abdullahi, A. et al. Front. Neurology (2020) 11: 687].

### Pain Conditions

According to some embodiments, a composition of the present disclosure comprising a population of purified enriched potent exosomes with an appropriate therapeutic signature isolated from a normal healthy subject may treat pain conditions.

Overexpression of MiR-21-5p supports a pro-inflammatory phenotype of macrophages attracted on the site of damage, while intrathecal delivery of a miR-21-5p antagomir appears to avoid an extension of inflammatory condition and neuropathic hypersensitivity onset [D'Agnelli, S. et al. Mol. Pain (2020) 16: PMC7493250, citing Simeoli, R. Nat. Commun. (2017) 8: 1778]. A proteome characterization of exosomes from mouse spared nerve injury (SNI) model suggested the cargo sorting of vesicular proteins as a crucial step in mediating signaling mechanisms underlying neuropathic pain and evidenced unique patterns of proteins. In particular, significant upregulation of complement component 5a (C5a) and Intercellular Adhesion Molecule 1 are detected in exosomes from SNI model compared to sham control [Id., citing Jean-Toussaint, R. et al. J. Proteomics (2020) 211: 103540]. The involvement of exosomes in neuropathic pain is also underlined by many studies on complex regional pain syndrome (CRPS). It is a chronic neuropathic pain disorder, disabling for sensory, motor, and autonomic dysfunctions as well as of allodynia, hyperalgesia, dystonia, and tremors [Id., citing Bruehl, S. BMJ (2015) 351: h2730]. A different miRNA-exosomal profile between responders and non-responders to treatment in CRPS patients has been identified, suggesting a potential tool to prior identify a subgroup of patients with higher possibility to have benefit by that specific treatment, in this case from plasmapheresis [Id., citing Ramanathan, S. et al. J. Transl. Med. (2019) 17: 81] In a mouse model of CRPS, the mechanism of action of macrophage-derived exosomes and their cargo has been investigated. A decrement of thermal hyperalgesia following a single injection of macrophage-derived exosomes has been found, suggesting a potential immunoprotective role. In the same study, serum-derived exosomes from CRPS patients were analyzed and 127 miRNAs were significantly different comparing CRPS exosomes with control-derived exosomes. Among them, three miRNAs (miR-21-3p, miR-146a, and miR-146b), known to be involved in the control of overactivation of innate immune response, are over expressed in both murine and human model [Id., citing McDonald, M K et al. Pain (2014) 155: 1527-1539].

### Solid Organ Transplantation.

In view of their anti-fibrotic and pro-angiogenic effects, a composition of the present disclosure comprising a population of purified enriched exosomes with an appropriate therapeutic signature isolated from a normal healthy subject may improve graft survival by, for example, reducing ischemia reperfusion injury, improving recovery from acute damage, and modulate tolerance towards the graft.

According to some embodiments, a composition of the present disclosure comprising a population of purified enriched potent exosomes with an appropriate therapeutic signature derived from EV-derived MSCs of a normal healthy subject may be helpful for preserving donor organs when administered before transplant in the context of hypothermic or normothermic perfusion machines. According to some embodiments, addition of the composition to the perfusion solution may improve donor organ viability and functionality.

Perfusion solutions containing EVs have shown some success in organ preconditioning. One report demonstrated that EVs released by MSCs, delivered in the perfusate during organ cold perfusion (4 h), preserved and protected kidney function. Histological and genetic analyses on EV-treated kidneys revealed upregulation of enzymes involved in energy metabolism and reduction of global ischemic damage [Id., citing Gregorini, M. et al. J. Cell Mol. Med. (2017) 21: 3381-3393]. Another report demonstrated that EVs isolated from the venal perfusate of rats subjected to remote ischemia preconditioning ameliorated renal function when injected into another animal with ischemia-reperfusion injury (IRI). To explore the underlying mechanism, the authors tested in the same IRI model in vivo the effect of EVs released by human proximal tubular cells cultured in hypoxia, supporting the hypothesis that remote ischemia preconditioning activates a repairing program into tubular cells by the release of pro-regenerative EVs [Id., citing Zhang, G. et al. Biomed. Pharmacother. (2017) 90: 473-478]. The use of EVs released by stem cells to improve the viability of pre-transplant livers was recently assessed in a model of ex vivo rat liver NMP. EVs isolated from human liver stem cells (HLSC-EVs) were added to the perfusate 15 min after the initiation of normothermic machine perfusion (NMP) and administered for 4 h within the perfusate. The results showed that HLSC-EVs limited the progression of ischemic injury, with a significant reduction of the levels of aspartate aminotransferase and alanine aminotransferase and a decrease of histological damage compared with results of NMP alone [Id., citing Rigo, F. et al. Transplantation (2018) 102: e205-e210].

Bone marrow MSC-EVs have been shown to recapitulate the therapeutic effects of the cells against acute GVHD [Id., citing Selmani, Z. et al. Cells (2008) 26: 212-222]. A systemic infusion of MSC-EVs in mice with acute GVHD was associated with the suppression of CD4+ and CD8+ T cells and with the preservation of circulating naive T cells, possibly due to the unique microRNA profiles of MSC-EVs. The analysis on microRNA cargo in MSC-EVs identified that their target genes were involved in regulation of the cell cycle, T-cell receptor signaling, and GVHD

In testing the effect of EVs in a rat model of IRI after DCD renal transplantation, MSC-EVs derived from Wharton's jelly, intravenously injected after renal transplantation, were shown to mitigate renal damage, and improve survival and function. In particular, MSC-EVs were shown to reduce cell apoptosis and inflammation, to stimulate HGF production, and subsequently to alleviate fibrosis [Id., citing Wu, X. et al. J. Cell Biochem. (2018) 119: 1879-1888].

### Method for Reprogramming a Donated Organ or Tissue Comprising a Fibrotic Disposition

According to another aspect, the present disclosure provides a method for reprogramming a donated organ or tissue comprising a fibrotic disposition comprising
(a) treating the donated organ or tissue ex vivo with a composition of the present disclosure comprising a population of purified enriched potent exosomes derived from extracellular vesicles (EVs) derived from mesenchymal stem cells of a normal healthy subject comprising purified, enriched potent exosomes with an appropriate therapeutic signature, wherein the EVs comprise an identity signature of expression of three or more biomarkers selected from CD9, CD63, CD81, or Tsg101+; the exosomes comprise total protein of about 1 mg; the exosomes comprise total RNA content greater than 20 μg; the exosomes comprise a cargo comprising a therapeutic signature including attributes of age, gender, estrogen receptor function and status, environmental impact/stressors, donor cell or tissue type, health of the donor organ or tissue, genomics of the donor cell or tissue, size of the exosomes is 50-130 nm, inclusive; wherein lot to lot and batch to batch variability relative to prior lots of the composition is below a significance level of ≤0.05; and
(b) reducing or slowing dysfunction of the organ or tissue.

According to some embodiments, the purified, enriched population of potent exosomes derived from extracellular vesicles derived mesenchymal stem cells (MSCs) of a normal healthy subject is derived from a tissue or a body fluid of a human subject. According to some embodiments, the tissue is placental tissue, adipose tissue, umbilical cord tissue, lung tissue, heart tissue, or dental pulp; or wherein the tissue is bone marrow of normal healthy subjects aged 21-40 years old; or the body fluid is blood, amniotic fluid or urine. According to some embodiments, the MSCs derived from placental tissue are derived from one or more of chorionic membrane (CM), chorionic trophoblast without villi (CT-V), chorionic villi (CV), or decidua (DC). According to some embodiments, the blood is umbilical cord blood or peripheral blood.

According to some embodiments, identity of the MSCs is confirmed by a marker signature comprising CD29, CD44, and CD105. According to some embodiments, the exosome cargo comprises a potency signature comprising expression of one or more, two or more, three or more, four or more, or five or more of angiopoietin 2 (Ang-2), fibroblast growth factor (FGF), hepatic growth factor (HGF), interleukin 8 (IL-8), a tissue inhibitor of metalloproteinases (TIMP), vascular endothelial growth factor (VEGF), platelet derived growth factor (PDGF), or tumor necrosis factor alpha (TNFα). According to some embodiments, the tissue inhibitor of metalloproteinases is TIMP1, TIMP2, or TIMP1 and TIMP2.

According to some embodiments, the composition is a pharmaceutical composition comprising a therapeutic amount of the composition and a pharmaceutically acceptable carrier. According to some embodiments, the composition comprises at least 1×10¹² EVs comprising exosomes per isolation. According to some embodiments, a therapeutic amount of exosomes comprises at least 1×10⁹ exosomes.

According to some embodiments, the therapeutic signature comprises one or more two or more, three or more, four or more, or five or more miRNAs selected from miR-29a, miR-10a, miR-34a, miR-125, miR-181a, miR-181c, miR-Let-7a, miR-Let-7b, miR-Let7d, miR-146a, miR-145, miR-21, miR-101, and miR-199.

According to some embodiments, the organ or tissue if left untreated comprises one or more of a progressive injury, progressive inflammation, progressive fibrosis or a combination thereof. According to some embodiments, the exosome cargo comprising the therapeutic signature modulates one or more of the injury, the inflammation, an excess accumulation of extracellular matrix, cell senescence; or modulates a pathway comprising fibrogenic signaling; or reprograms a tissue affected by the age-related chronic disease. According to some embodiments, the pathway comprises transforming growth factor (TGFβ) signaling. According to some embodiments, the pathway comprising fibrogenic signaling is a TERT (telomerase reverse transcriptase) activation pathway including one or more of a Smad pathway, a mitogen-activated protein kinase pathway, a phosphoinositide 3-kinase pathway; a canonical Wnt-β catenin pathway, a Notch signaling pathway, or a NF-κB pathway. According to some embodiments, the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle tissue.

### Formulations

According to some embodiments, the composition is a pharmaceutical composition comprising a therapeutic amount of the purified enriched exosomes comprising a cargo comprising a therapeutic signature and a pharmaceutically acceptable carrier. The phrase “pharmaceutically acceptable carrier” is art recognized. It is used to mean any substantially non-toxic carrier conventionally useable for administration of pharmaceuticals in which the isolated EVs of the present invention will remain stable and bioavailable. The pharmaceutically acceptable carrier must be of sufficiently high purity and of sufficiently low toxicity to render it suitable for administration to the mammal being treated. It should further maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetracetic acid (EDTA), sorbitol, tartaric acid, phosphoric acid, and the like.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil. According to some embodiments, the pharmaceutically acceptable carrier comprises a pulmonary surfactant. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Specific modes of administration will depend on the indication. The selection of the specific route of administration and the dose regimen is to be adjusted or titrated by the clinician according to methods known to the clinician in order to obtain the optimal clinical response. The amount of active agent to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

The local delivery of therapeutic amounts of a composition for the treatment of a lung injury or fibrotic lung disease can be by a variety of techniques that administer the compound at or near the targeted site. Examples of local delivery techniques are not intended to be limiting but to be illustrative of the techniques available. Examples include local delivery catheters, site specific carriers, implants, direct injection, or direct applications, such as topical application and, for the lungs, administration by inhalation. Local delivery by an implant describes the surgical placement of a matrix that contains the pharmaceutical agent into the affected site. The implanted matrix releases the pharmaceutical agent by diffusion, chemical reaction, or solvent activators.

Pharmaceutical formulations containing the active agents of the described invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the described invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The pharmaceutical compositions of the described invention can be formulated for parenteral administration, for example, by injection, such as by bolus injection or continuous infusion. The pharmaceutical compositions can be administered by continuous infusion subcutaneously over a predetermined period of time. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents.

For oral administration, the pharmaceutical compositions can be formulated readily by combining the active agent(s) with pharmaceutically acceptable carriers well known in the art. Such carriers enable the actives of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragecanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the described invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In addition to the formulations described previously, the compositions of the described invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising any one or plurality of the active agents disclosed herein also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

For parenteral administration, a pharmaceutical composition can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques.

The described invention relates to all routes of administration including intramuscular, subcutaneous, sublingual, intravenous, intraperitoneal, intranasal, intratracheal, topical, intradermal, intramucosal, intracavernous, intrarectal, into a sinus, gastrointestinal, intraductal, intrathecal, intraventricular, intrapulmonary, into an abscess, intraarticular, subpericardial, into an axilla, into the pleural space, intradermal, intrabuccal, transmucosal, transdermal, via inhalation, via nebulizer, and via subcutaneous injection. Alternatively, the pharmaceutical composition may be introduced by various means into cells that are removed from the individual. Such means include, for example, microprojectile bombardment, via liposomes or via other nanoparticle device.

According to some embodiments, the pharmaceutical compositions of the claimed invention comprises one or more therapeutic agent other than the EVs as described. Examples of such additional active therapeutic agents include one or more immunomodulators, analgesics, senolytics, anti-inflammatory agents, anti-fibrotic agents, proton pump inhibitors, or oxygen therapy.

Examples of immunomodulators include corticosteroids, for example, prednisone, azathioprine, mycophenolate, mycophenolate mofetil, colchicine, and interferon-gamma 1b.

Examples of analgesics include capsaisin, codeine, hydrocodone, lidocaine, oxycodone, methadone, resiniferatoxin, hydromorphone, morphine, and fentanyl.

Examples of senolytics include dasatinib, quercetin, fisetin, navitoclax, and piperlongumide.

Examples of anti-inflammatory agents include aspirin, celecoxib, diclofenac, diflunisal, etodolac, ibuprofen, indomethacin, ketoprofen, ketorolac nabumetone, naproxen, nintedanib, oxaprozin, pirfenidone, piroxicam, salsalate, sulindac, and tolmetin.

Examples of anti-fibrotic agents are nintedanib and pirfenidone.

Examples of proton pump inhibitors are omeprazole, lansoprazole, dexlansoprazole, esomeprazole, pantoprazole, rabeprazole, and ilaprazole.

According to the foregoing embodiments, the pharmaceutical composition may be administered once, for a limited period of time or as a maintenance therapy over an extended period of time, for example until the condition is ameliorated, cured or for the life of the subject. A limited period of time may be for 1 week, 2 weeks, 3 weeks, 4 weeks and up to one year, including any period of time between such values, including endpoints. According to some embodiments, the pharmaceutical composition may be administered for about 1 day, for about 3 days, for about 1 week, for about 10 days, for about 2 weeks, for about 18 days, for about 3 weeks, or for any range between any of these values, including endpoints. According to some embodiments, the pharmaceutical composition may be administered for more than one year, for about 2 years, for about 3 years, for about 4 years, or longer.

According to the foregoing embodiments, the composition or pharmaceutical composition may be administered once daily, twice daily, three times daily, four times daily or more.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Age-related lung disease and inflammation. Age-related changes in the lung tissue result in impaired cellular protective mechanisms that predispose to chronic respiratory diseases (3). Age-related changes in innate and adaptive immune responses may further predispose aging lungs to development and progression of chronic lung disease (7). As the percentage of persons over 65 years old increases worldwide (5.87% in 1985, 7.28% in 2005, 8.28% in 2015, and 16.5% in 2019) (https://www.statista.com/statistics/457822/share-of-old-age-population-in-the-total-us-population/), so does the prevalence of lung disease: whereas globally COPD is reported in 200 per 10,000 patients under the age of 45, there are 1,200 cases reported per 10,000 patients over the age of 65. The predominance of cases in the older age group is also reported in patients with IPF. An estimated 200,000 Americans currently suffer from age-associated fibrotic lung disease (8-10). Approximately 50,000 adults aged 60 and over are diagnosed annually with IPF. Overall, there is an almost five-fold increase in incidence of IPF and COPD based solely on age (11).

Inflammation, induced by cigarette smoke, is a common risk factor for patients with IPF and COPD (12). Decreased cellular repair, increased production of pro-inflammatory cytokines, fibroblasts that acquire a senescent phenotype with decreased numbers of alveolar epithelial type 2 cells (AEC2) characterize the histology of COPD lung tissue (3). In interstitial lung diseases like IPF, the activity of a fibro-proliferative phenotype in fibroblasts (myofibroblasts) and ongoing epithelial apoptosis are mediated by factors that promote alveolar damage(2-15). Older lungs are characterized by diminished/dysregulated responses of alveolar macrophages to pathogenic stimuli, and their impaired ability to remove damaged neutrophils and resolve inflammation and tissue damage (16-18). Reprogramming the age-related inflammatory phenotype of the lung and other organs with exosome therapy could have major benefits in the aging population.

Exosomal microRNAs; their age and sex-related dimorphism. Important and incompletely resolved issues center on the age and sex of exosome donors, and it is not clear what characterizes an ideal profile of therapeutically “effective” exosomes. With regard to age, multiple studies, including our published and preliminary data, show that the cumulative effect of age-related cellular damage is responsible for reduced MSC/exosome functionality (39-42). Age-related miRNA profile changes during normal aging (43) could explain reduced functionality of old MSCs/exosomes and most likely compromise their potential therapeutic effects (44). Our proposed comparative studies of adult and old exosomes will be critical in that regard, aiming to identify the “effective” signature of therapeutic exosomes for human age-related lung diseases.

Similarly, sex-related dimorphic effects on the human MSC exosome secretome are not well studied. Sex-related differences arise at the molecular level due to differential gene expression (45, 46), differential alternative transcripts (47) and/or epigenetic modifications (48). Transcriptional differences are a consequence of the genetic sex and are not strictly dependent on gonadal hormones. A recent meta-analysis of microarrays comparing hMSC of male and female donors highlighted chromosomal segments and genes differently expressed by sex (49). There were twenty genes over or under expressed in male vs female samples that regulated inflammation and cell communication pathways.

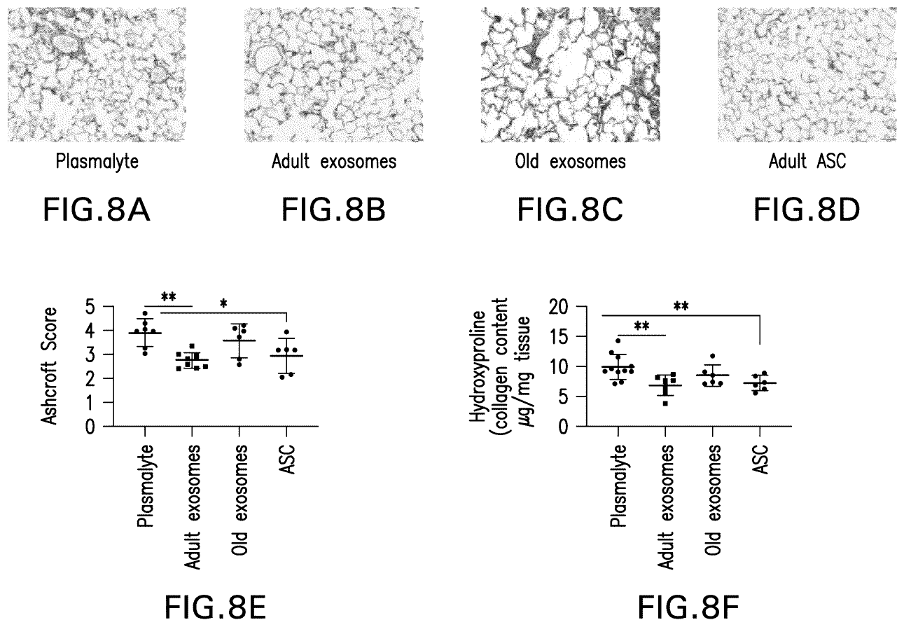

MitomiRs, Immflamma-miRs and Senescence-associated (SA)-miRs. Age-related diseases share a common inflammatory phenotype. It remains incompletely understood whether the inflammatory mediators originate from senescent cells with the altered secretome (senescence-associated secretory phenotype, SASP), from increased barrier permeability (e.g. gut or lung) for microbial products that stimulate inflammation, from accumulated proinflammatory immune cells or a combination of these and other sources. Aged cells can further contribute to the inflammatory/senescent phenotype through production and delivery of SA-miRs, mitomiRs, and inflamma-miRs. A signature group of miRNAs including increased miR-146a, and decreased miR-181a, -181c have been suggested to define aging and belong to all three groups of miRNAs (50). We will specifically test whether these or other miRs mark inefficient vs efficient EV therapy. Our preliminary data (FIG. 8) illustrate feasibility and robustness of exosome therapy in fibrotic lung tissue of old mice, providing us with a clinically relevant readout. Using this readout, we will be able to first correlate miRNA signatures of exosomes to their reparative potential and defining biomarkers for exosome selection in Phase II of our exosome program.

Therapeutic advantage of exosomes over other aging interventions. As of 2020 about 567 distinct chemical substances are noted to increase lifespan in non-disease models (https://genomics.senescence.info/drugs/). Multiple treatments have been investigated in rodent models with some disparity in results most likely due to strain/genetic differences. Some compounds, such as polyphenols, occur naturally and act as senolytics to modulate oxidative damage, inflammation and cell senescence (51, 52). Two natural metabolites, the NAD precursors nicotinamide riboside (NCT02950441) and nicotinamide mononucleotide (53) are also being investigated. Other drugs are being repurposed to test senotherapeutic ability (54, 55), including the TAME study to test the anti-hyperglycemic agent, metformin, on 65- to 80-year-old individuals without diabetes who are at high risk for the development of chronic diseases of aging (NCT02432287). Clinical trials with rapamycin (Sirolimus), an mTOR inhibitor, are also ongoing, however one completed trial (NCT02874924) showed no impact upon immune, cognitive, and functional consequences in healthy aging subjects compared to placebo controls. Overall, there are few clinical trials using compounds that are able to target multiple indications. To this end, exosome therapy offers a major advantage with powerful miRNA and anti-inflammatory cytokine delivery that have multitargeted potential. Indeed, pre-clinical studies suggest that methods of regulating microRNAs altered in aging and age-related diseases could restore a healthy aging phenotype (12, 56).

### Target Product Profile

MSCs and their exosomes may modify multiple pathways simultaneously, lending support for the proposed use of exosomes as potential therapeutic agents that “seek and repair” multiple targets associated with the age-related disease phenotypes in multiple organs. To date, many potential geroscience therapies fall short in this regard. With a defined therapeutic exosome signature, our pleiotropic therapy will seek to treat age-related chronic lung disease by targeting the associated inflammation and immune aging thereby improving lung function through both repair and potential restoration (and, if successful, in follow-up studies, other tissues). Our preliminary data using in vivo mouse models provides evidence for this approach, as it demonstrates age- and sex-related differences in exosomes' functional impact on lung tissue which we believe correlate or will correlate to their differential miRNA cargos. Pre-clinical studies suggest that methods of regulating microRNAs altered in age-related diseases and senescence could restore a healthy aging phenotype (12, 56). To this end, exosome therapy with a defined pleiotropic signature may offer a major advantage.

Exosomes have several advantages over MSCs as potential therapeutic agents. Exosomes easily enter the systemic circulation and are encapsulated. This process protects their cargo from unfavorable conditions, such as changes in pH or degradation in vivo. Moreover, whereas infused MSCs quickly diminish post-infusion, it may be that delivery of MSC-derived exosomes can achieve a higher circulating “dose” that lasts longer. Tumor formation risk should be lower or nonexistent with exosomes, which are not self-replicating. They can be stably stored for several months at −20 °C or −80 °C, without cryo-preservatives, remaining biologically active. This cell-free “cell therapy 2.0” can help to circumvent complicated handling issues of biological therapeutics containing viable cells.

To the extent that exosomes derived from MSCs can be used as an instrument for cell-free regenerative medicine, much will depend on the quality, reproducibility, and potency of their production, in the same manner that these parameters dictate the development of cell-based MSC therapies. As detailed in Phases I & II, we will carefully address all these issues in detail, bearing in mind that the MSC exosome's contents are not static, but rather a product of the MSC tissue of origin, its activities, and the immediate intercellular environment of the MSCs. Careful attention to detail in selection of the MSC donor source, as well as the characterization and production of MSC exosomes is therefore needed to provide a new therapeutic paradigm for cell-free MSC-based therapies with decreased risk. Production and standardization issues are as follows:

A major bottleneck in the advancement of exosome-based therapy into the clinic is the development of high scale, reproducible and efficient production of clinical-grade efficacious exosomes. This requires sterile generation of exosomes with therapeutic payloads, produced in sufficient amounts for clinical testing, without batch-to-batch variation that could compromise efficacy. Development and optimization of production methods, including methods for isolation and storage of exosome formulations, are required for realizing exosome-based therapeutics. In addition, improvement of therapeutic potential and delivery efficiency of exosomes would be highly therapeutically relevant.

A significant challenge for the use of exosomes for scientific and medical applications is the isolation process, as exosomes can be found in various complex biological matrices that are composed of a plethora of nanosized biomaterials that overlap in size and density. Common isolation techniques for the separation of EVs from other fluid components are ultracentrifugation (UC), density gradients (DG), and size exclusion chromatography (SEC). However, these methods can be limited by the input volume, time consumption, and exosome yield.

UC protocols are able to process relatively large volumes, but they result in low recovery rates, have time-consuming centrifugation steps, frequently damage the exosome structure, and cause the coprecipitation of contaminants.

DG and SEC usually result in EV formulations with high purity; however, these protocols are time-consuming, result in poor yields, and require small volumes of starting material for processing.

Clinical translation of the above-mentioned methods is also challenging due to sterility and scale-up requirements for clinical-grade manufacturing.

Improved techniques that enable exosome concentration in a scalable, reproducible, and sterile manner, are necessary for future clinical translation.

Following “the process is the product” principle is useful for translational research during development of complex biological therapeutics in a very early stage of manufacturing and even during early clinical safety and proof-of-concept testing. This strategy helps bridge the initial gaps in knowledge regarding the active substance and mechanism or modes of action (MOA) responsible for a certain therapeutic activity until proof of mechanisms and therapeutic activity are identified. Our data using in vivo mouse models indicates age- and sex-related differences in exosomes' functional impact on lung tissue, accompanied by differential miRNA cargos. Developing a miRNA signature for “effective” exosome donors will be undertaken by comparing outcomes/pathways of “adult” compared to “old” exosomes; we will further compare donor cargo with the high vs. low potential therapeutic efficacy using predefined endpoints. Success will be defined by the determination of differentially enriched miRNA cargos in young vs. old and male vs. female human exosomes. Correlative analysis of miRNA cargos with functional efficacy in protecting lung tissues in a murine inflammation model will validate our target product profile. These studies will lead to the preparation of an exosome candidate suitable for entry into focused preclinical drug development with input from the FDA.

Recognizing the increasing potential for their clinical utilization, the imperative to optimize an exosome isolation method for maximum yield, purity and assay reproducibility has been addressed. We have partnered with Zen-Bio to address the exosome productivity challenges.

As a result, a consistent and validated manufacturing protocol for the production of MSC exosomes has been established, which utilizes a fed-batch process for large-scale expansion of MSCs and a protocol for exosome production in suspension bioreactors that is scalable to 15 L and yields consistent extracellular vesicles.

A cornerstone to today's challenges related to therapeutic exosome development has been industrializing exosome platforms to make them more productive.

cGMP lots of defined signature exosomes prepared with our manufacturing partner will be synthesized and Investigational New Drug (IND)-enabling studies will be conducted in mouse and ex vivo human models to further test the potency of our signature. Purified GMP investigational product, TPP Exosome Therapeutic, will show the typical nanoparticle tracking analysis profile and expression of the main exosome markers (CD9/CD63/CD81/Tsg101). The GMP manufacturing method utilized will guarantee high exosome yield and consistent removal of contaminating proteins. The resulting GMP product, will be tested for safety, purity, identity, and potency in vitro, showing functional immunomodulatory and inflammation resolution activity.

The standardized production method and testing strategy for large-scale manufacturing of GMP TPP Exosome Therapeutic opens new perspectives for reliable human therapeutic applications including age-related chronic respiratory disease as well as targeted age- and organ-specific inflammation.

Target product profile-defined exosomal signature. The present disclosure will exploit the age and sex related decline that occurs in MSC/exosome functionality involving the local and systemic environment of aging tissues/cells, mitochondria! dysfunction, and increased oxidative stress to define our optimal expected exosomal signature summarized in Table 3.

TABLE 3
Expression of exosome derived microRNAs
that might be altered during aging.

| Expected | | | | |
|---|---|---|---|---|
| expression of | | | | |
| proposed | Female | Male | Female | Male |
| “signature” | young | young | aging | aging |
| miRNAs | exosomes | exosomes | exosomes | exosomes |
| miR-10a (cell | ↓ | ↓ | ↑↑ | ↑ |
| senescence MSCs | | | | |
| (57, 58)) | | | | |
| miR-34a | ↓↓ | ↓ | ↑↑ | ↑ |
| (inflammation) | | | | |
| miR-125 | | | ↓ | ↓ |
| (inflamma-miR | | | | |
| (59, 60) | | | | |
| miR-146a (immune | ↑↑ | ↑↑ | ↓ | ↓ |
| response (57) | | | | |
| miR-181a | | | ↓ | ↓ |
| (inflamma-miR | | | | |
| (61) | | | | |
| miR-181c | | | ↓ | ↓ |
| (inflamma-miR(62) | | | | |
| miR-Let-7b(SA- | | | ↓ | ↓ |
| mitomiR)Complex | | | | |
| I, IV and V(50, 59) | | | | |

* indicates known to be regulated by change in estrogen status of post-menopausal women.

The large-scale manufacturing of exosomes will be demonstrated using a GMP-compliant process with well-defined criteria for the release of the product for human use. These criteria include the size distribution of the exosome preparation, ascertained by NanoSight; flow cytometry analyses of defined markers on exosomes; and potency assays. The proposed study will provide the first quantitative POC analysis of safety and efficacy using what appears to be the most therapeutically relevant, most potent and practical MSC-derived product (Exosomes) to have entered the clinic. Success of these studies will provide basis for pivotal trials.

The bone marrow derived exosome product of interest in this study will be manufactured as follows. The TPP Exosome Therapeutic will be isolated from Bone Marrow-Derived Mesenchymal Stem Cells (BM-MSCs) of an adult (21-40 year old) healthy donor who will have met a strict donor acceptance criteria including current and past medical history in addition to passing highly sensitive nucleic acid testing for hepatitis B, hepatitis C, and HIV. The exosomes will be tested for sterility and have been manufactured in FDA-registered facilities that meet Current Good Manufacturing Practices (cGMP). (See Phase II below).

### Manufacturing and Quality Control

Donor and Cell Source—Human Mesenchymal Stem Cells used to generate the TPP Exosome Therapeutic will originate from a single donor bone marrow source of adherent stromal progenitor cells that were expanded under GTP/cGMP compliant conditions and controls to generate a Master Cell Bank for Phase II studies. We will file a Master File at the FDA detailing donor selection, testing, qualification, master cell bank production and cell characterization for identity and purity. These cells will be characterized for expansion potential, cell surface marker expression, and differentiation potential into adipocytes, osteocytes, and chondrocytes (63). In addition, functional analyses for angiogenic cytokine secretion and inducible Indoleamine-pyrrole 2,3-dioxygenase (IDO) activity will be performed and data reported.

Current Good Manufacturing Practice (cGMP) compliant processes and testing will be implemented from cell donor selection to final distribution. Details of the Master Cell Bank and growth media will be filed with the FDA. Inspections and testing will be in place throughout manufacturing process. These will include required monitoring of equipment and environment, monitoring of pH, glucose, and lactate content of cell medium, visual monitoring of cell density, nanoparticle tracking analysis to establish final particle count and size distribution, and USP<71> sterility testing of final product. Product tracking controls and cold supply chain monitoring protocols are in place. We will audit key suppliers, manufacturer, and testing organizations to ensure compliance.

The exosome product will be filtered and purified prior to filling, and all containers will be visually inspected prior to cryopreservation to ensure normal appearance, intact container closure, and the absence of visible extraneous particulate matter. Product will be released for clinical use only if all safety, identity, quality, and purity specifications are met and all applicable GMPs are appropriately followed as determined by Zen-Bio Quality Assurance. These include assurance of a sterile product by in-process and release testing for bacteriology, mycology, mycoplasma, endotoxin, and nonspecific viral adventitious agents. In addition, Quality control tests will be performed for each lot of GMP exosome product. The vesicles will be characterized by their protein concentration, estimated defined RNA signature concentration, particle concentration/and particle size to ensure there is an acceptable concentration, viability, and purity. Stability testing will also be conducted to verify both frozen storage and post-thaw stability. The final cell suspension of TPP Exosome Therapeutic Thawed/Diluted product will be released for administration based on assessment of Exosome dose (0.8-10″), mycoplasma testing (must be negative), Endotoxin (<1.65 EU/ml), FLOW Cytometric assessment of exosome identity surface markers (CD9+/CD63+/CD81+/Tsg 101+), defined mRNA signature and 14-day sterility (must be negative) immediately prior to transfer to the final container (60 ml cryobag). The product will be placed in a validated transport container fitted with the continuous temperature monitoring device.

Final product: TPP Exosome Therapeutic re-suspended in sterile Saline.

Exosome target product properties and outcomes are listed in Table 4.

TABLE 4
Target Product Profile

| Product | Minimum acceptable | Ideal |
|---|---|---|
| Properties | result | Result |
| Primary | Chronic lung disease | Age-sex related chronic |
| indication | | lung diseases |
| Patient | Adults with age-related | Adults and children with |
| population | chronic lung disease | lung disease |
| Treatment | Chronic | Short term leading to |
| duration | | long term improvement |
| Delivery mode | IV | IV, Injectable, inhalation |
| Dosage Form | Exosomes suspended | Suspension or |
| | in sterile saline | lyophilized |
| Regimen | The preclinical studies | One or two doses |
| | suggest that at least | |
| | 1 dose will be needed | |
| Efficacy | Stabilization or | Improvement of FVC |
| | improvement of FVC | to above normal range |
| Risks/Side | Bronchitis-Clinically | None |
| Effects | significant bronchitis | |
| | was not reported in | |
| | AETHER trial | |

Overall, interventions that can modulate (even partially) several of the aging hallmarks need to be studied to provide new insight into druggable targets against age-related diseases. In that context, exosomes fulfill the criteria as promising candidate therapeutics for chronic age-related diseases. The described plan for manufacturing, characterizing, standardizing, quality-controlling and delivering exosomes is intended to fully and thoroughly assess their anti-geronic therapeutic potential.

Preliminary data suggest why adult (21-40 years old) donor exosomes are superior to exosomes derived from older (>65 years old, and likely >55 years old, based on our preliminary data) donors and may provide new ways to functionally modulate/reverse adverse effects of aging. There are three novel conceptual aspects of this work:
(i) We will, for the first time, dissect how sex-related differences in the signature of male vs. female bone-marrow-derived exosomes could lead to enhanced therapeutic options for treatment of age-related diseases.
(ii) We will test the hypothesis that age-induced miRNA changes in donor exosomes may provide a signature to optimize efficacy of donor exosome selection.
(iii) We will dissect and identify some of the components lacking in old donor exosomes that lead to loss of their therapeutic effects, paving the way for potential supplementation of such components in future trials.

Rigor and Reproducibility: Age-related studies will use adult (21-40 years old) donors, compared to older (65 years old and older) donors, unless indicated otherwise. This definition will be used across the proposal. For in vivo experiments, as outlined in Vertebrate Animals, the number of mice in each group will be selected based on power calculations to achieve at least 80% power to detect a 20% difference in outcome at p<0.05. Experiments will be repeated at least twice. All ex vivo (3D lung punches) assays will be performed with a minimum of 3 biological replicates/sex, three technical triplicates, and at least three independent experiments. Data will be analyzed by investigators blinded to the experimental group.

### Characterization of Age- and Sex-Related Differences in Exosome Function and Cargo

Rationale: Our data using in vivo mouse models suggests that important age- and sex-related differences in exosomes has functional impact on lung tissue as well as in their miRNA cargos. Defining an miRNA signature for “effective” exosome donors will be undertaken by comparing outcomes following transfer of adult and older exosomes and correlating these outcomes to donor cargo so as to putatively define exosome preparations with the highest potential therapeutic efficacy.

Success Metrics: Correlative analysis of miRNA cargos with functional efficacy protecting lung tissues in a murine bleomycin lung injury model. These studies will lead to the preparation of an exosome candidate suitable for entry into focused preclinical drug development with input from the FDA.

miRNA Identification and In Vivo Efficacy Milestones Must be Satisfied Prior to Transitioning to Phase II.

### Preliminary Results

An age-related increase in oxidative stress may erode MSC function (64, 65) potentially reducing their therapeutic efficacy and altering their exosome signature.

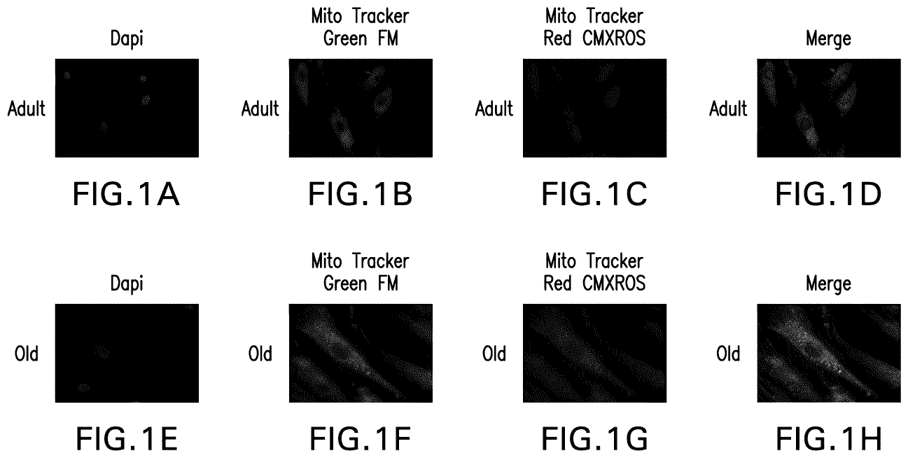

FIG. 1. Adult (39 years) and older (65 years) human male adipose derived stem cells (ASCs) were incubated with Dapi (FIG. 1A, FIG. 1E) or Mito Tracker Green (mitochondria) number FIG. 1B, FIG. 1F) or Red (mitochondria) activity FIG. 1C, FIG. 1G). Localization and activity were merged using confocal microscopy (FIG. 1D, FIG. 1H).

FIG. 1 shows that old donor adipose-derived mesenchymal stem cells (ASCs) exhibit hallmarks of aging that are reversible.

Old human ASCs displayed increased mitochondrial activity as shown by mito tracker Green and Red (FIG. 1F and FIG. 1G) compared to adult ASCs (FIG. 1C). Old mouse and human ASCs also exhibited decreased mRNA expression of nuclear factor (erythroid-derived 2)-like 2 (Nrf2, 6-fold lower in old vs adult ASCs), a transcription factor regulator of multiple antioxidant enzymes (unpublished data). Finally, mRNA for TGF-13, a profibrotic cytokine, was elevated 1.5-fold in old compared with the adult ASCs (not shown).

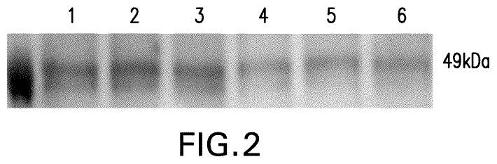

Both catalase protein expression (FIG. 2) and superoxide dismutase (SOD2) and catalase mRNA levels (Table 5), were found to be decreased in old ASCs compared to adult counterparts (FIG. 2, lanes 4-6).

TABLE 5

| Human (female) | SOD2 mRNA | Catalase mRNA |
|---|---|---|
| ASC (age) | expression | expression |
| 29 yrs #1 | 6643 | 3741 |
| 29 yrs #2 | 17460 | 2332 |
| 23 yrs | 4528 | 2035 |
| 80 yrs | 76 | 593 |
| 66 yrs | 6 | 14 |
| 63 yrs | 334 | 427 |
| 59 yrs | 75 | 205 |
| 58 yrs | 363 | 498 |

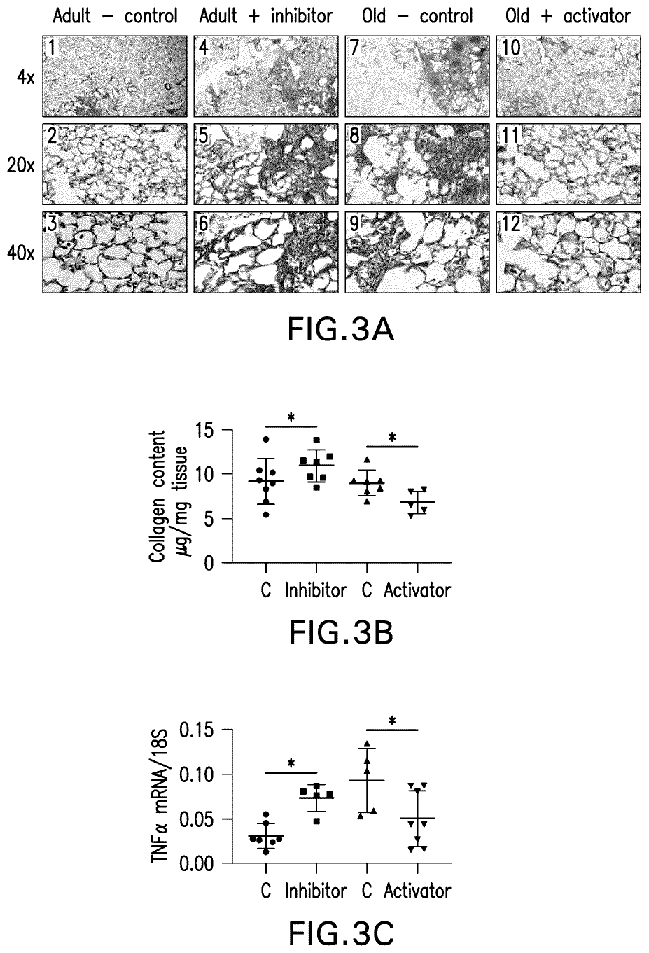

Reversing the aging-related phenotype in old human ASCs (>58 years old) leads to repair of age-related lung fibrosis and improvement in wound healing: Aberrant antioxidant defense may be important in the pathogenesis of age-related lung diseases including pulmonary fibrosis and COPD (66, 67). Our published and preliminary studies show that adult ASCs prevent or repair fibrotic lung tissue in a model of pulmonary fibrosis; in contrast, old human ASCs were unable to do so (39). We investigated whether the age-related differences in antioxidant mRNA and protein expression observed in a separate batch of ASCs (see Table 5 and FIG. 2), could in part explain functional differences in old human ASCs. Using CRISPR plasmids, human adult (<35 years old) and older (>58 years old) ASCs were transfected with catalase inhibitor and activator, respectively. 5×10⁵ cells were injected intravenously into the bleomycin lung injury male mouse model (39). As judged by the amount of red stain (collagen), adult but not older control ASCs corrected fibrosis. Infusion of adult ASC transfected with catalase inhibitor (adult+inhibitor, FIG. 3A, panels 4-6) did not reduce severity of fibrosis in the lung, whereas old ASCs transfected with catalase activator did (old+activator FIG. 3, panels 10-12). Similarly, adult+inhibitor increased lung hydroxyproline (a measure of collagen accumulation, FIG. 3B) and TNFa mRNA expression (FIG. 3C), relevant endpoints in the lung fibrosis model (39). Old cells transfected with catalase activator protected against fibrosis in a manner similar to adult control cells.

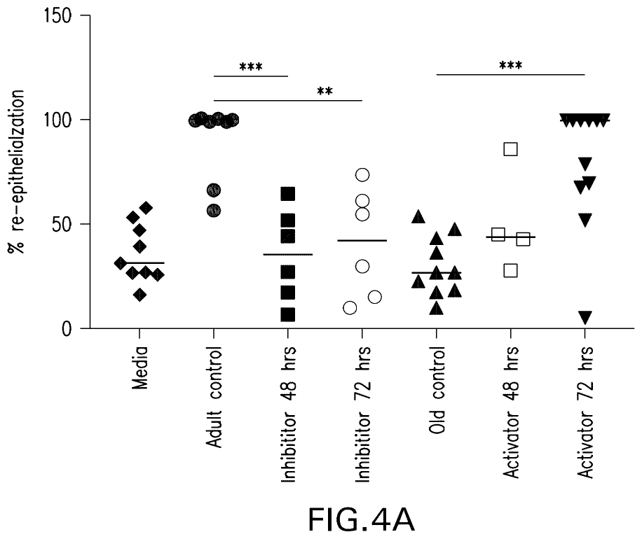

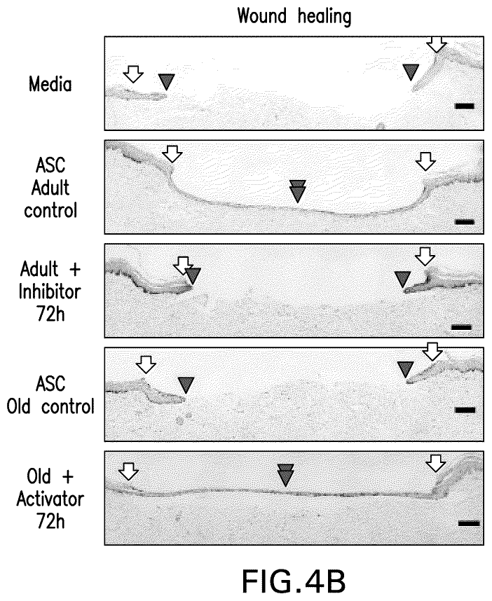

Antioxidant dysregulation also contributed to decreased wound healing ability in old ASCs, as demonstrated by the fact that the catalase knock-in “normalized” wound healing. Young human skin punch wounds were obtained and 5×10⁵ human ASCs were injected radially. After daily media changes on wounds for four days, wounds were embedded and stained with hematoxylin and eosin (FIG. 4B). FIG. 4 shows that wound healing capacity of ASC is age- and catalase-dependent. At baseline adult ASCs promote wound healing and old ASCs inhibit wound healing of ex vivo human wounds. (FIG. 4A is a graph representing quantification of wound healing using histomorphometric analyses 4 days after wounding. Catalase inhibitor reduced the capability of adult ASCs to promote wound closure, while catalase activation rescued ability of old ASCs to stimulate wound closure (n=3 biological replicates from 2-3 experimental ASC isolates/group). Data are graphed as mean±SEM % of vehicle **P<0.01, ***P<0.001 Data were analyzed using one-way Kruskal-Wallace and Mann-Whitney tests. FIG. 4B) shows representative ex vivo wounds stained with hematoxylin and eosin. White arrows indicate wound edges after initial wounding, whereas red arrowheads point at the epithelialized edges of the migrating fronts 4 days after wounding; scale bar=200 μm. Healing (formation of the thick epidermal layer, see arrows) occurred in the presence of either adult−control or adult+inhibitor ASCs prevented normal wound healing.

Taken together, decreasing catalase expression in adult ASCs to comparable levels of antioxidant expression in old ASCs, reduces the effectiveness of the adult ASCs in repair of bleomycin-induced lung injury and ex-vivo wounds.

The above results support our strategy to understand the phenotype and cargo of old ASCs as the first step in determining a therapeutic signature for their exosomes.

Cigarette smoke (CS) exposure induces inflammation in multiple organs. CS exposure increases oxidative stress in the lung, kidney and skin of female mice. Cigarette smoke is the most common factor associated with COPD, a disease that is at least six-fold higher in individuals over the ages of >55, and the third leading cause of death in the U.S. (www.lung.org). Our published data showed that the combination of smoking and estrogen deficiency in old female mice contributed to an emphysematous phenotype in the lung, glomerulosclerosis in the kidney and decreased wound healing in the skin (68-70). In the lung we noted that CS induced a decrease in collagen content, macrophage number, and respiratory chain complex-1 protein. Lung sections revealed patchy areas of subpleuretic fibrosis and increased number of inflammatory cells with perivascular edema and additional hyperplasia of columnar epithelium.

CS also resulted in an increase in matrix metalloproteinase-2 (MMP) activity and total number of apoptotic cells in lung tissue. In the same mouse used to study the lung, CS induced MMP dysregulation of kidney and skin, as well as other dysregulated pathways, suggesting that this model may be useful to test our preferred donor signature across different organs and chronic age-related pathologies. In addition, the CS model may also be useful to determine the importance of our signature for multiple age-related conditions since miRNAs are readily influenced by environmental chemicals found in CS. Thus, the lungs of rodents exposed to CS demonstrate down-regulation of miRNAs (71, 72). Schembri et al. found that most of the differentially expressed miRNAs in the human bronchial airway epithelium were downregulated in smokers and were inversely correlated with their predicted targets(73). Graff et al. noted that a decrease in global miRNA expression was more pronounced in heavy smokers, suggesting that the magnitude of miRNA repression is related to the extent of smoking history (74). These data raise the possibility that CS may modulate the aging phenotype in part though changes in mesenchymal stem cells/exosomes. We will utilize this model in our studies (Phase II) to obtain a generalizable proof-of principle of anti-geronic therapeutic effect of exosomes.

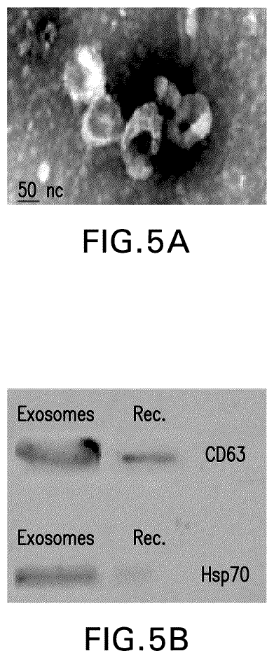

Exosomes as an off-the-shelf therapy. It is generally accepted that membrane-bound and soluble factors play a major role in the therapeutic function of MSCs, and that membrane-bound vesicles (exosomes) are the main vectors for MSC paracrine effects. Therefore, in preparation for Phase I and the entire project, we isolated and characterized ASC-derived exosomes (isolation performed by Zen-Bio Inc., our manufacturing partner). Exosome size was approximately 30-150 μm. Transmission electron microscopy (TEM) was performed on isolated exosomes (FIG. 5A. Transmission electron microscopy of isolated exosomes. Image magnification. Scale bar=50 nm). Expression of exosome markers CD63 and Hsp70 by the isolated exosomes were confirmed by Western analysis (FIG. 5B). Phase I of this proposal will utilize exosomes isolated from human male and female bone marrow mesenchymal stem cells.

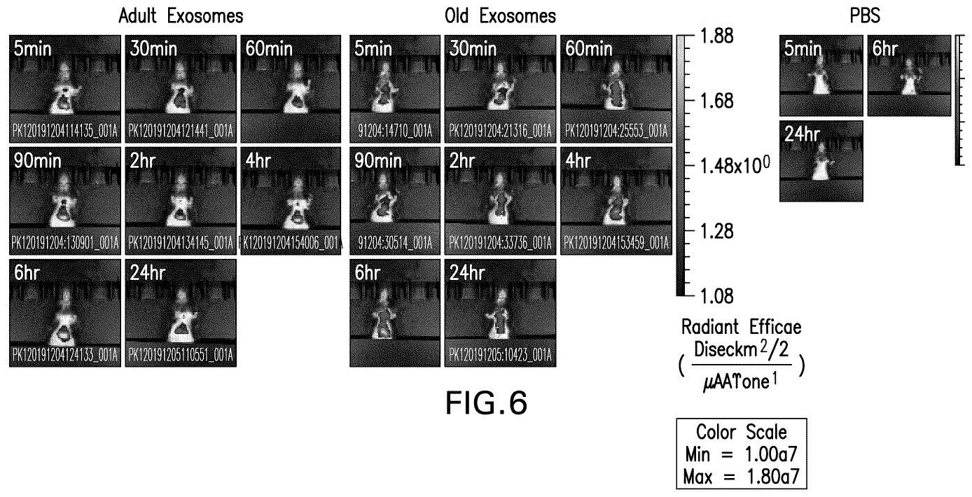

Biodistribution of ASC-derived exosomes: We assessed the time course of biodistribution of young and old human ASC exosomes injected into 16-month-old male C57/BL/6 mice using an in vivo bioluminescent imaging system. Exosomes were located in the lung within 5 minutes post-tail vein injection. Images were taken at 5 min, 30 min, 60 min, 90 min and 2, 4, 6 and 24 hours (FIG. 6). Shown are representative in vivo bioluminescence images to study the biodistribution of ExoGlow™ labeled adult and old exosomes in 16-month-old C57BL/6 mice. Time points are 5 min, 30 min, 60 min, 90 min, 2, 4, 6 and 24 hours. Inset shows PBS injected mice at 5 mins, 6 and 24 hours. Intensity of luminescence seen in bar from lowest (red) to highest (yellow). The signal was abolished by 24 hours except for the liver where exosomes were identified by additional postmortem organ examination. There were no adverse events noted in the mice that were followed for 20 days.

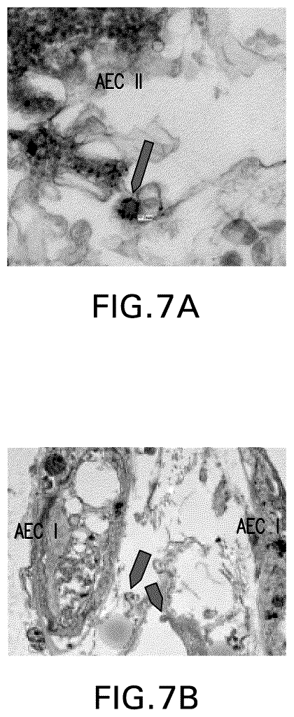

Exosomes derived from ASCs enter alveolar epithelial cells (AEC). To visualize whether exosomes were present after injection into 3D lung punches, we utilized TEM. TEM revealed exosomes labeled with gold nanoparticles in alveolar (AEC) type I and AEC type II cells (red arrows, FIG. 7A and FIG. 7B) as noted by a pulmonary pathologist (Dr. Shahzeidi, experienced pulmonary pathologist on research team, personal communication).

Similar to our previous studies (39), adult human ASC exosome (35 year-old male) infusion prevented bleomycin-induced fibrosis while old human ASC exosomes (65 year-old male) did not (FIG. 8). We performed dose and time course experiments of exosomes in our pre-clinical protection model of bleomycin lung injury using 10 μg (10⁶ particles), 20 μg (10¹⁰ particles) and 40 μg (5×10¹⁶ particles) of exosomes collected from approximately 100×10⁶ MSCs. This dosing regimen was based on results from the AETHER trial in which 100×10⁶ MSCs delivered by intravenous infusion to patients with IPF was safe; secondary endpoints suggested potential efficacy of this dose (75). We found that 5×10¹⁶ particles/100 μI (40 μg) of exosomes was the most effective dose using this for all further experiments. We performed tail vein injections in 18 month old male C57BL/6 mice at day 10, a time point that represents the end of the inflammatory phase and the initial stages of the fibrotic phase of lung injury (76, 77). Adult exosomes rescued the effects of bleomycin-induced fibrosis and weight loss. Mice receiving bleomycin had a mean Ashcroft score of 3.88±0.22 compared to those receiving BLM+adult exosomes 2.7±0.11 or BLM+ASCs (2. 9±0.29). Mice treated with old exosomes had a score of 3.55±0.28.

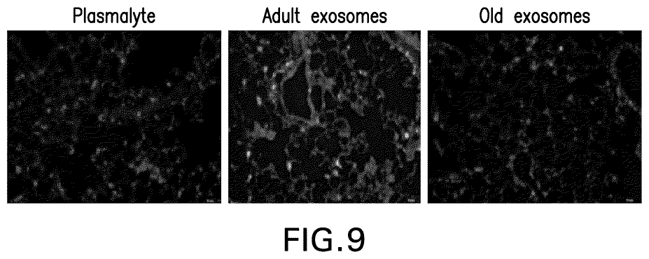

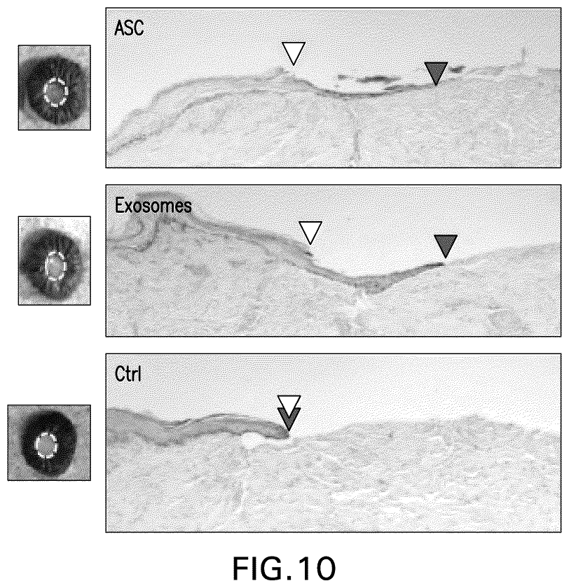

Immunofluorescent staining with SP1, a marker of alveolar type 2 cells, revealed an increase in positive cells (yellow color) compared to plasmalyte control or old exosomes (FIG. 9). Since Type 2 alveolar epithelial cells (AEC2) are regarded as the progenitor population of the alveolus responsible for injury repair and homeostatic maintenance, these data suggest that adult exosomes are stimulating a repair mechanism. We also found that adult ASC exosomes promote wound healing in human skin as effectively as whole cell ASCs. As shown in FIG. 10, gross photos show visual signs of wound closure and correspond to histology assessments. White dashed line indicates initial wound edge; white arrowhead indicates initial wounding edge in the H&E stained sections; red arrowhead is pointing to epithelial tongue location at day 4 post-wounding.

Exosomes isolated from old male human ASCs downregulate microRNAs found at high levels in adult ASCs. We hypothesized that analysis of differentially expressed miRNAs and their targets in human old donor ASCs could elucidate pathways relevant to loss of therapeutic potential. miRNA was extracted from adult and old human ASC exosomes using the ExoQuick and miRNAeasy kits. The results of the human miRNA Exiqon array indicated 25 miRNAs were either up or downregulated with a greater than five-fold difference. Target genes were derived from the miRWalk database (78). We found that senescent (SA) related miR-146a and -181a were upregulated in old exosomes, while another SAmiR-10a, as well as mitomiRs-125 and let-7b were decreased. These data were confirmed by qPCR and suggest that aging exosomes may carry cargo mirroring or promoting the aging phenotype. Based on these data we expect that adult exosome cargo potentially contribute to the repair process in age-related diseases.

Is exosome cargo different in males and females? The postmenopausal estrogen deficient state sustains the development of inflamma-aging and regulates multiple pathways differently than in aging males (79). 118 miRNAs are located on the human X chromosome but only 4 are present on the Y chromosome. In addition, 15% of the miRNA encoded by the X chromosome are able to escape X-inactivation (17, 80). These data suggest that the cargo delivered by the exosomes may exhibit sexual dimorphism.

Experimental design: Based on our preliminary and published data, we will isolate exosomes in collaboration with our partners at Zen-Bio. We will use 5 male and female adult MSCs (21-40 years of age) to isolate exosomes from bone marrow derived MSCs (available from Rooster, AlICells, and Lonza). The old MSCs (preferably >65 years of age, n=3/sex) will be isolated from healthy bone marrow donors (see letter from Dr. Ulrickson). We also wanted to avoid any postmenopausal effects in 45-60 year-old females. The identification of miRNA expression profiles will be performed by next-generation sequencing with sequencing-by-synthesis technology; this will allow a dynamic range of detection (from very low to highly abundant RNAs) and measurement of relatively limited differences in expression between samples. Using RNA derived from human exosomes (200 ng), miRNA-seq libraries will be prepared using the Illumina TruSeq Small RNA Sample Kit and sequenced with Illumina platform MiSeq (MiSeq Reagent Kit v3). Bowtie (version 0.12.5) will be used to perform a stepwise alignment of fastq files to Illumina databases. Differentially expressed miRNAs will be identified using Bioconductor's limma package. Data will also be validated by qPCR experiments. Primary data will be analyzed using bioinformatics pipeline software (NIH) [genboree.org/theCommons/projects/exrnatools may2014/wiki/Small % 2ORNAseq %20Pipeline].

Once we have compiled a list of validated miRNAs, we will confirm the relevance of this “miR signature set”. We will transfect exosomes with a mimic or inhibitor of relevant miRNAs (81) and inject into-22 month-old C57BL/6 male and female mice (equivalent to −61 to 65-year-old males and females) and determine inflammatory and fibrosis endpoints. We will choose one donor (out of 5) with the desired signature to test in duplicate and compare the outcomes of mice receiving “desired” signature exosomes with outcomes from mice receiving old exosomes. Due to the short timeline of phase I we will only test our research exosomes in the old bleomycin lung injury model (Table 6). This model has a well-established inflammatory phase at day 7 (82) and replicates several of the specific pathogenic molecular changes associated with IPF (76). BLM (2.0 Ukg/BW in 50p1 saline) or 50p1 of sterile saline (controls) will be administered by direct intratracheal instillation via intubation as in our preliminary experiments above. 1010 particles of either male or female exosomes will be injected in the tail vein at day 7 post-BLM, a time that injury has occurred in both young and old mice (39, 82). At sacrifice, (day 21 post-BLM) lungs will be inflated and perfused.

TABLE 6

| Human | Male | Male | Female | Female | Male | Male | Female | Female | |
|---|---|---|---|---|---|---|---|---|---|
| exosome | + | + | + | + | + | + | + | + | |
| isolate | Exo | vehicle (V) | Exo | V | Exo | V | Exo | V | Total |
| Mouse | BLM | BLM | BLM | BLM | Saline | Saline | Saline | Saline | |
| treatment | | | | | | | | | |
| 21-40 yr | 8 | 8 | 8 | 8 | 8 | 8 | 8 | 8 | 64 |
| female | | | | | | | | | |
| exo | | | | | | | | | |
| >50 yr | 8 | 8 | 8 | 8 | 8 | 8 | 8 | 8 | 64 |
| female | | | | | | | | | |
| exo | | | | | | | | | |
| 21-40 yr | 8 | 8 | 8 | 8 | 8 | 8 | 8 | 8 | 64 |
| male exo | | | | | | | | | |
| >50 yr | 8 | 8 | 8 | 8 | 8 | 8 | 8 | 8 | 64 |
| male exo | | | | | | | | | |

Assessments: At sacrifice, lungs will be inflated, perfused, and studied as follows (39): 1) Histology (assessed by a pulmonary pathologist blinded to the groups) and immunohistochemistry of the lung (epithelial lung markers; SPC and AQ5, aSMC-actin and endothelial lung marker) performed; 2) Bio-Plex Pro Mouse Cytokine 23-plex Assay that includes IL-113, IL-6, IL-10 and TNF, as well as anti-inflammatory markers. Using this as a screening tool we will also measure sTNFR1 and 2 and CRP (83), because together with IL-6 these markers provide the most accurate measures of inflammation that correlate to all-cause morbidity and mortality. Sensitivity of IL-6 measurement in a multiplex could also be insufficient and in that case, we will use a high-sensitivity IL-6 ELISA kit (LEGEND MAX™ Mouse IL-6 ELISA, BioLegend®, Inc. Kit) for all measurements. We will also assess quantitative measures of fibrotic disease (Ashcroft, collagen types I and III expression and hydroxyproline) (39), as well as associated molecular markers and 3) downstream pathways of inflammation and aging (e.g. avintegrin, matrix metalloproteinases (MMP), AKT phosphorylation). We will measure Nrf-2 activation which has been shown to protect against the oxidative stress seen in cigarette smoke-induced emphysema. In addition, pertinent to age-related diseases, the NAD+-dependent deacetylase and anti-inflammatory factor SIRT1 is suppressed in aging (84). SIRT1 deacetylates Foxo3a, which enhances stress resistance through several antioxidants including MnSOD, and catalase (85). Furthermore, experiments in which SIRT1 is either reduced or overexpressed show that it modulates senescence of both BM-derived MSCs and ASCs (86). Therefore, we will analyze lungs to assess SIRT1 expression as well as antioxidant expression (Table 7).

Statistics. All murine studies will employ an n=8 animals per cohort with independent male and female cohorts for all studies, time points and strains. Data will be evaluated by ANOVA for comparisons between two or more groups followed by post-hoc Tukey's multiple comparison tests. Results will be reported as the mean±standard deviation or standard error while significance will be defined by p values <0.05. Power for the primary outcome—reduction of fibrosis—is >80% to detect a 20% or larger difference at p<0.05. Data will be analyzed by investigators blinded to experimental group.

Success metrics/Alternative outcomes/Potential difficulties: Success metrics and go/no-go decisions are summarized in Table 7.

TABLE 7
Readouts of lung and milestones

| Aims | Milestones | Month |
|---|---|---|
| Compare | 1. Produce exosomes— | 1-3 (go/no go) |
| Adult and | project is a go if enough | |
| Old Male and | exosomes produced can treat | |
| Female | ≈. 100 mice (5 × 10¹²) | |
| exosomes | 2. Exosomes biophysical | 2-3 (go/no go) |
| | characterization—project is a | |
| | go if >90% of EVs are 50- | |
| | 150 um in size and have | |
| | positive CD63, CD90 | |
| | 3. Exosomes biophysical | 4-6 (descriptive |
| | characterization—project is | milestone, will be |
| | ago if >90% of EVs are 50- | go/no-go together |
| | 150 um in size and have | with milestones |
| | positive CD63, CD90; | # 2, 4 & 5 |
| Test exosomes | 4. Therapeutic effects of | 5-10 (this milestone, |
| in the old | adult and old exosomes from | like milestones 3 |
| bleomycin lung | male and female donors. To | and 5, are all |
| injury model | be considered efficacious, | playing into a go/ |
| | exosome will have to rescue | no-go decision to |
| | 90% of mice from | identify optimal |
| | bleomycin-induced death by | signature and guide |
| | fibrosis, and to reduce | selection of donors) |
| | fibrosis by 80% as judged by | |
| | (Ashcroft score, collagen | |
| | content and related fibrotic | |
| | inducing pathways) | |
| | 5. Using siRNA or knock-in | 8-12 (data from 3&4 |
| | transfections, identify | will help guide these |
| | exosome signatures and | experiments, that will |
| | molecules that are | themselves be go/no- |
| | therapeutic. Criteria from #4 | go decision makers) |
| | for therapeutic efficacy will | |
| | apply. project is a go if we | |
| | have identified at least 3 | |
| | molecules from adult | |
| | exosomes that provide | |
| | superior efficacy to old | |
| | exosomes if up- or down- | |
| | regulated, and can be used | |
| | for exosome donor | |
| | selection″ | |

First, because our preliminary data primarily used exosomes from ASCs and we propose to generate exosomes from bone marrow MSCs, we will ascertain that our exosome preparations have reproducible effects, and that their aging also recapitulates our prior results. The production of exosomes from an individual cell line is not static, therefore we will test at least two exosome preparations from the same donor. We expect to find a signature similar to Table 3 as noted in the TPP section above. Exosomes from estrogen replete (adult) females may have a different profile than old female exosomes since normal estrogens/estrogen receptor function protects against excess cellular oxidative stress, regulates SIRT1, mitochondria! function and microRNAs (87, 88). Although the adult female signature may be different than the adult male signature based on our preliminary data, we anticipate that adult (young) exosomes, regardless of sex, will have anti-inflammatory and antifibrotic effects. Our experiments will ascertain whether the extent of these changes are sex dependent. If not sex dependent, then the number of available donors for MSC/exosome production would increase since historically, most clinical trials have utilized adult male donors predominately. We realize that obtaining bone marrow from healthy donors over 65 may be a challenge therefore we may have to lower our donor age to 55-60 and document the length of time that the female donors have been in menopause (89). We recognize that the proposed “therapeutic” signature may not be exhaustive, but our sequencing and bioinformatics should be informative. In either case, the signature will guide and better define criteria for donor selection.

We do not expect significant obstacles to our approach to optimize the beneficial exosome signature by exosome transfections using knock-in or knock-down miRNAs. Our manufacturing partner, Zen-Bio, Inc. will take the lead with these experiments since they have extensive experience in this field. It is possible that we may be unable to completely alter the signature, however, as we have shown in our prior publication, inhibiting or activating a single miRNA has a dramatic effect on targeted protein expression and relevant downstream pathways, including an ultimate therapeutic effect (81).

We do not expect any off-target effects since our biodistribution data suggest that exosomes are quickly cleared in mouse organs roughly 24 hours following administration, except for the liver. However, after 20 day follow-up of the mice, no adverse events were noted.

### Example 2. Phase II AIM 2

A. Perform cGMP isolation of exosomes and B. conduct IND-enabling studies. We will pursue two complementary sets of approaches utilizing cGMP exosomes: in vivo animal studies and ex vivo human lung punches from male and female donors >65 years old.

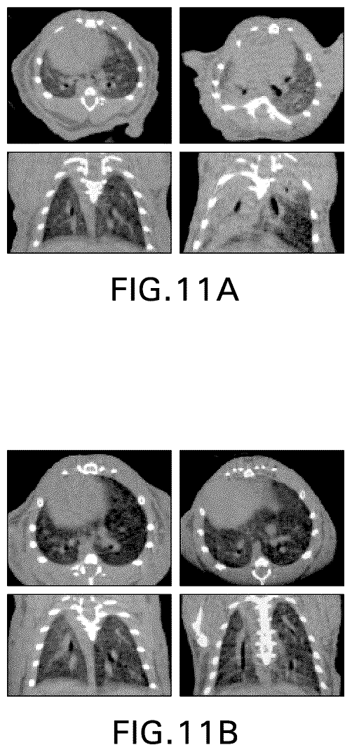

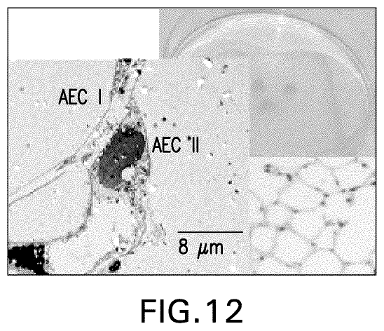

1. Preliminary Data and Tools for Phase II: CT scans verify lung injury. Baseline chest pCT prior to bleomycin administration demonstrated well-aerated lungs without evidence of pulmonary edema or increased tissue density (FIG. 11). By day 7 post-BLM, chest pCT demonstrated increased signal attenuation over the lung fields and loss of aeration indicating lung injury in mice given intra-tracheal instillation of bleomycin. Control mice treated with intra-tracheal instillation of saline showed no significant changes or evidence of lung injury compared to their baseline chest pCT (FIG. 11). We will perform CT scans on our mice post-cigarette smoking (CS) and post SARS-CoV-2 infection to ensure that lung injury has occurred prior to delivery of exosomes. Our preliminary data show that the 3D lung punch model (90) recapitulates cellular and tissue interplay in the lung (91), recognizing that they lack immune recruitment and systemic perfusion (hypoxic condition). Lungs from patients with lung fibrosis and/or smoking history (obtained from surgical biopsies/lung transplant-UA will be instilled with warm agarose at the pressure of 25 cm H2O. The trachea will be ligated just caudal to the larynx and the contents of thorax removed as one unit to maintain airways and alveolar integrity. Lung segments will be cooled on ice for 30 min to allow solidification of the agarose. Using a biopsy punch device, punches (4 mm) will be transferred to an air-liquid interface. Punches will be injected with 5×10¹⁰ particle number of exosomes radially around the punch followed by collection four days later. Punches will be embedded, cut and stained with trichrome as shown in FIG. 12, where we validated preserved normal architecture of a 16-month-old mouse as assessed by EM examination by a pathologist (FIG. 12).

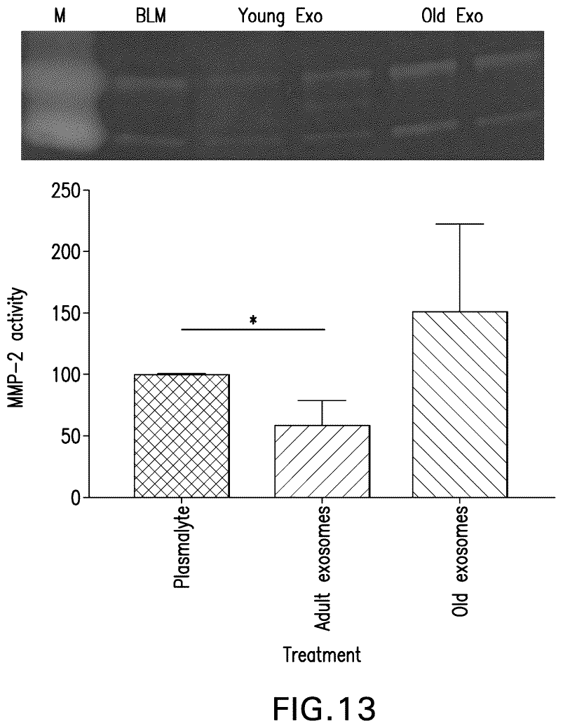

In preliminary experiments, we have injected adult and old ASC-derived exosomes into lung punches. These punches were obtained from C57BL/6 mice 10 days post-bleomycin treatment, at the peak of inflammation. Adult exosomes were effective in interrupting bleomycin-induced fibrotic pathways while old exosomes were not. Bleomycin-installation increased baseline lung matrix metalloproteinase-2 (MMP-2) activity in the punches (FIG. 12). Since MMPs are important for ECM accumulation and airway remodeling in COPD, CKD and other diseases associated with inflammation (92-94), the decrease noted in the punches after treatment with young exosomes (FIG. 13) suggests a potential therapeutic benefit. We will conduct IND-enabling studies in mice and ex vivo human models to further test the efficacy of our signature.

### 2. Experimental Design and Methods:

We will produce cGMP lots of defined exosomes prepared with our manufacturing partner and conduct Investigational New Drug (IND)-enabling studies in mouse and ex vivo human models to further test the potency of our signature. To date, a bottleneck in the advancement of exosome therapy into the clinic has been the development of high quality, reproducible, and efficient production of clinical grade exosomes. Our collaborator Zen-Bio, Inc. has now developed such a pipeline, as shown in section 2a.

a. Manufacturing: Zen-Bio routinely isolates EVs containing exosomes from mesenchymal stem cells using a hollow-fiber cartridge containing bioreactor as previously described (95). The bioreactor is amenable to culturing any cell type, although adherent cells are preferred. To prepare the bioreactor, a total of 1×10⁹ cells are seeded onto the cartridge. After seeding, the separate cell cartridge is filled with 40 mL of serum-free medium for cell maintenance and EV production, and growth medium is continually circulated through the bioreactor, with medium exchanges every 3-5 days. Cell metabolism is monitored by measuring the glucose level in the circulating medium; when levels drop 50% the circulating medium is changed. Bioreactor cultures have been maintained in this manner for up to three months while the MSCs retain their International Society for Cellular Therapy (ISCT)-mandated MSC characteristics (63).

EVs are collected every 3-5 days after the bioreactor culture is established. The conditioned serum-free medium will be removed from the cell cartridge and subjected to sterile filtration through a 0.22 μm filter, followed by tangential flow filtration (TFF) to concentrate the EVs and reduce the overall volume to 1 mL. The concentrated EVs are then subjected to size exclusion chromatography (SEC) over a 70 nm porous resin particle column. Each column is standardized, quality assured and certified to ISO 13485 standard (Medical Devices) making it perfect for clinical testing. Fractions are collected from the column and are analyzed for EVs using Nanoparticle Tracking Analysis (NTA). Peak fractions are identified and pooled for use and further analysis.

A typical yield from 40 mL of conditioned medium has been 3.6±0.6×10¹¹ particles, and this yield can generate up to 1×10¹³ EVs per cartridge over the life of the bioreactor run.

b. EV physical characterization and surface marker expression: We will characterize the EV preparations for average particle size and concentration using Zeta View® BASIC NTA—Nanoparticle Tracking Video Microscope PMX-120. Protein concentration, DNA concentration, and RNA concentration will be determined by a Nanodrop ND-1000 spectrophotometer. EVs are then characterized for CD9, 63, and 81 expression to confirm the presence of exosomes. Exosome-specific protein markers will be determined by high throughput flow cytometry analysis using the MACSPIex Exosome Kit and MACSQuant analyzer to show the necessary presence of CD9, CD63 and CD81. Human foreskin fibroblast derived exosomes will be used as a control since fibroblasts and MSCs have a similar morphology and surface marker expression (96).

Success metrics and alternative approaches for EV production: Success criteria are based on Zen-Bio's historical read-outs of EV properties and results as follows: particle yield should be at least 1×10⁹ per isolation; particle diameter should be consistent, to include exosomes (30-150 nm); total protein should be ≈1 mg; total RNA content should be >20 pg; and exosome and specific biomarkers (CD9, 63, and 81) must be present by 90%. Should any of the lots fail to produce exosomes that pass our quality control metrics, the lots will be stored for future analysis but not subjected to cell-based testing. In addition. as outlined in Phase_milestones (Table 7), we will utilize exosome preparations that meet our desired donor signature.

c. EV Transcriptome/miRNAome: In parallel with EV surface protein expression described above, we will analyze the miRNA using the MacsPlex miRNA Kit (Miltenyi). We will monitor the relative abundance of 39 miRNAs in a high throughput manner. This will allow us to determine consistency within EV batches, to assess population composition, and to assess batch-to-batch and lot-to-lot consistency. Each sample is tested in triplicate using independent experiments on different days to ensure data quality. Lot-to-lot and batch-to-batch consistency is determined using ANOVA and/or t-tests depending on the comparison and differences between measures are considered significant with a p value≤0.05.

Using this methodology, we have analyzed multiple lots of EVs and detected elevated levels of several miRNAs related to our desired therapeutic properties and exosomal signature.

TABLE 8
SET1000 PRODUCT RELEASE TESTS AND SPECIFICATIONS

| Test/Reference | Specification | Control |
|---|---|---|
| EV Quality | 80-100 Billion EVs | Particle yield should be at |
| | | least 1 × 10⁹ per isolation |
| EV Size | 50-130 nm | Mean of 165 ± 50 nm and |
| | | mode of 120 ± 20 nm; |
| | | p value ≤0.05 |
| MSC Identity | CD28, CD44, CD105 | p value ≤0.05 |
| Exosome Identity | CD9, CD63, | p value ≤0.05 |
| | CD81, Tsg101+ | |
| Exosome Potency | Ang-2, FGF, HGF, | Total protein should be |
| | IL-8, TIMP-1, TIMP-2, | about 1 mg; p value ≤0.05 |
| | VEGF, PDGF, TNF-α | |
| Therapeutic | miRNA: | Total RNA content |
| Signature | 29a/10a/34a/125/181a/ | should be >20 ug; |
| | 181c/Let-7b/146a/ | p value ≤0.05 |
| | 199/153/101/199 | |
| Sterility USP <71> | Sterile, no evidence of | |
| CFR 610.12 | microbial growth | |
| Mycoplasma | Negative | |

### 3. In Vivo Models:

To translate our preclinical findings from Phase 1 into the clinic, we will test cGMP-grade compliant young donor bone marrow-derived MSC exosomes. We will evaluate the exosome signature from 3 adult donors and choose one donor/group (male and female based on the data from Phase 1) for subsequent experiments based on an anti-aging signature. We will test duplicate exosome preps from the same donor to assure that this signature is reproducible. Controls will utilize an old exosome preparation that does not have a “therapeutic” signature. We will utilize a variety of models and deliver 5×10¹⁰ exosomes in 100μ1 of PBS by tail vein injection in mice. The control mice will receive tail vein injection of 100μ1 of PBS.

To expand our in vivo models and test generalized anti-geronic activity of exosomes, we elected the cigarette smoke (CS) model and the pneumonia model since the large majority of patients with IPF and COPD have a past history of CS exposure (97, 98), and because older adults are highly vulnerable to pneumonia. Although CS exposure confers long-term risk of many diseases even decades after cessation, these effects are not well understood (99) CS exposed lung tissue removed from rodents and humans have subsets of dysregulated miRNAs (71, 74, 99). We propose to expose mice to 6 months of CS and determine the ability of exosomes to repair the lung though delivery of miRNA that may be dysregulated in the lung post CS. As a model of viral pneumonia, we will include the SARS-CoV-2 infection model as an important lung injury model fundamentally different from both bleomycin and CS. Specifically, in the case of COVID-19 infection, autopsy data supports adequate expansion of the lungs despite an inability to oxygenate hemoglobin in contrast to the lungs of patients with COPD and IPF. Mechanisms of COVID-19 lung injury remain incompletely understood as the prevalence of patients with chronic lung disease (“Ionghaulers”) increases. Therefore, addressing whether exosomes can protect against this type of lung injury has the potential to fundamentally broaden the antigeronic spectrum of exosome therapeutic indications.

### 2. Experimental Design and Methods:

We will produce cGMP lots of defined exosomes prepared with our manufacturing partner and conduct Investigational New Drug (IND)-enabling studies in mouse and ex vivo human models to further test the potency of our signature. To date, a bottleneck in the advancement of exosome therapy into the clinic has been the development of high quality, reproducible, and efficient production of clinical grade exosomes. Our collaborator Zen-Bio, Inc. has now developed such a pipeline, as shown in section 2a.

Manufacturing: Zen-Bio routinely isolates EVs containing exosomes from mesenchymal stem cells using a hollow-fiber cartridge containing bioreactor as previously described (95). The bioreactor is amenable to culturing any cell type, although adherent cells are preferred. To prepare the bioreactor, a total of 1×10⁹ cells are seeded onto the cartridge. After seeding, the separate cell cartridge is filled with 40 mL of serum-free medium for cell maintenance and EV production, and growth medium is continually circulated through the bioreactor, with medium exchanges every 3-5 days. Cell metabolism is monitored by measuring the glucose level in the circulating medium; when levels drop 50% the circulating medium is changed. Bioreactor cultures have been maintained in this manner for up to three months while the MSCs retain their International Society for Cellular Therapy (ISCT)-mandated MSC characteristics (63).

EVs are collected every 3-5 days after the bioreactor culture is established. The conditioned serum-free medium will be removed from the cell cartridge and subjected to sterile filtration through a 0.22 μm filter, followed by tangential flow filtration (TFF) to concentrate the EVs and reduce the overall volume to 1 mL. The concentrated EVs are then subjected to size exclusion chromatography (SEC) over a 70 nm porous resin particle column. Each column is standardized, quality assured and certified to ISO 13485 standard (Medical Devices) making it perfect for clinical testing. Fractions are collected from the column and are analyzed for EVs using Nanoparticle Tracking Analysis (NTA). Peak fractions are identified and pooled for use and further analysis.

A typical yield from 40 mL of conditioned medium has been 3.6±0.6×10¹¹ particles, and this yield can generate up to 1×10¹³ EVs per cartridge over the life of the bioreactor run.

EV physical characterization and surface marker expression: We will characterize the EV preparations for average particle size and concentration using Zeta View® BASIC NTA-Nanoparticle Tracking Video Microscope PMX-120. Protein concentration, DNA concentration, and RNA concentration will be determined by a Nanodrop ND-1000 spectrophotometer. EVs are then characterized for CD9, 63, and 81 expression to confirm the presence of exosomes. Exosome-specific protein markers will be determined by high throughput flow cytometry analysis using the MACSPIex Exosome Kit and MACSQuant analyzer to show the necessary presence of CD9, CD63 and CD81. Human foreskin fibroblast derived exosomes will be used as a control since fibroblasts and MSCs have a similar morphology and surface marker expression (96).

Success metrics and alternative approaches for EV production: Success criteria are based on Zen-Bio's historical read-outs of EV properties and results as follows: particle yield should be at least 1×109 per isolation; particle diameter should be consistent, to include exosomes (30-150 nm); total protein should be ~1 mg; total RNA content should be >20 pg; and exosome and specific biomarkers (CD9, 63, and 81) must be present by 90%. Should any of the lots fail to produce exosomes that pass our quality control metrics, the lots will be stored for future analysis but not subjected to cell-based testing. In addition. as outlined in Phase 1 milestones (Table 7), utilize exosome preparations that meet our desired donor signature.

EV Transcriptome/miRNAome: In parallel with EV surface protein expression described above, we will analyze the miRNA using the MacsPlex miRNA Kit (Miltenyi). We will monitor the relative abundance of 26 miRNAs in a high through put manner. This will allow us to determine consistency within EV batches, to assess population composition, and to assess batch-to-batch and lot-to-lot consistency.

### 3. In Vivo Models:

To translate our preclinical findings from Phase 1 into the clinic, we will test cGMP-grade compliant young donor bone marrow-derived MSC exosomes. We will evaluate the exosome signature from 3 adult donors and choose one donor/group (male and female based on the data from Phase 1) for subsequent experiments based on an anti-aging signature. We will test duplicate exosome preps from the same donor to assure that this signature is reproducible. Controls will utilize an old exosome preparation that does not have a “therapeutic” signature. We will utilize a variety of models and deliver 5×10¹⁰ exosomes in 100μ1 of PBS by tail vein injection in mice. The control mice will receive tail vein injection of 100μ1 of PBS.

To expand our in vivo models and test generalized anti-geronic activity of exosomes, we elected the cigarette smoke (CS) model and the pneumonia model since the large majority of patients with IPF and COPD have a past history of CS exposure (97, 98), and because older adults are highly vulnerable to pneumonia. Although CS exposure confers long-term risk of many diseases even decades after cessation, these effects are not well understood (99) CS exposed lung tissue removed from rodents and humans have subsets of dysregulated miRNAs (71, 74, 99). We propose to expose mice to 6 months of CS and determine the ability of exosomes to repair the lung though delivery of miRNA that may be dysregulated in the lung post CS. As a model of viral pneumonia, we will include the SARS-CoV-2 infection model as an important lung injury model fundamentally different from both bleomycin and CS. Specifically, in the case of COVID-19 infection, autopsy data supports adequate expansion of the lungs despite an inability to oxygenate hemoglobin in contrast to the lungs of patients with COPD and IPF. Mechanisms of COVID-19 lung injury remain incompletely understood as the prevalence of patients with chronic lung disease (“Ionghaulers”) increases. Therefore, addressing whether exosomes can protect against this type of lung injury has the potential to fundamentally broaden the antigeronic spectrum of exosome therapeutic indications.

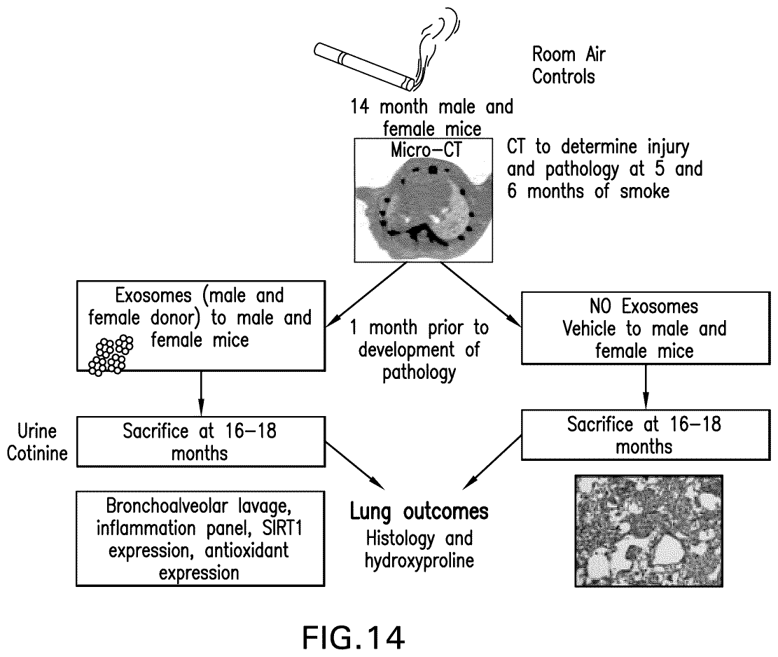

Smoking protocol: Mice will be exposed to 6 months of mainstream and sidestream cigarette smoke (CS) following the protocol previously published by our group (Teague smoke machine, 3 hours a day, 5 days a week) (68, 70). A similar protocol by Matulionis revealed the presence of fibrosis in male C57BL/6 mice that were exposed to CS, starting at 8-10 months of age (100). Initially, we will smoke 14-month-old mice and perform a time course with CT and sacrifice at 5 and 6 months post-CS to determine lung injury at these time points. Based on these data we will inject exosomes one month prior to sacrifice (FIG. 14). We will test male and female exosomes in male and female mice since the age-related hormone changes could result in differing responses to the exosomes. In order to investigate the effects of smoke exposure on airway and lung inflammation, we will assess; the airway and alveolar lumen (by bronchoalveolar lavage), and the lung parenchyma (by histology and immunohistochemistry on lung tissue sections), and other assessments outlined above for the bleomycin mouse model in SA 1. on cell suspensions of lung digests. The experiments will be performed on 10 mice/group. The reference cigarettes (University of Kentucky, 1R6F), manufactured by Tobacco and Health Research Institute, will be conditioned in a humidifier for 48 hours before use at 23 °C, 60% relative humidity. Water intake and weight will be monitored weekly. To ensure that all mice received adequate CS exposure, urine levels of cotinine, a metabolite of nicotine, will be measured by ELISA (OriGene Technologies, Inc). Our published data show CS-induction of kidney and skin fibrosis in aging mice therefore we will collect these organs and the liver for further histologic and pathway assessment (69, 70).

Success metrics: Since CS induces high levels of inflammation, we anticipate a reduction in inflammatory markers, at least in BAL, after exosome infusion by 20% or more. Not surprisingly, changes in antioxidant and SIRT1 expression have been reported in the lungs of CS exposed mice. Based on our preliminary data we expect that these and other proteins will be increased after treatment with exosomes. It is possible that we will not realize these metrics and will require additional dosing regimens which we are prepared to initiate.

TABLE 9

| | | | Male and | | |
|---|---|---|---|---|---|
| | | | female | Vehicle | |
| | | | exosome | IV | |
| | | | IV | injection | 5 or 6 |
| | CS | | injection | one | months post |
| | and Air | | one month | month | CS or room |
| | Male and | 5 and 6 | prior to | prior to | air sacrifice |
| | Female 14 | month | develop- | develop- | (either |
| | month old | post | ment | ment | depending on |
| | C57BL/6 | CS or air | of inflam- | of inflam- | preliminary |
| Smoking | mice | analysis | mation | mation | findings) |
| | | CT scans | CS Males | CS Males | Collect BAL; |
| | | 4 months | and | and | determine |
| | | with sac | Females | Females | inflammation |
| | | (10 mice) | 8/group + | 8/group + | markers, lung |
| | | | exosomes | exosomes | histology |
| | | CT scans | Room air | Room air | |
| | | 6 months | Males and | Males and | |
| | | with sac | Females | Females | |
| | | (10 mice) | 8/group + | 8/group + | |
| | | | exosomes | exosomes | |
| | | Assess | | | |
| | | injury for | | | |
| | | timing of | | | |
| | | exosome | | | |
| | | injections | | | |
| SARS- | | | | | D30 |
| CoV-2 | D0 | D4 | D7 | D14 | survival/ |
| infection | infection | analysis | analysis | analysis | analysis |
| | 24 adult & | 4 mice/ | 4 mice/ | 4 mice/ | 12 mice/group |
| | 24 old | group | group lung | group lung | survival; lung |
| | hACE- | lung | pathology, | pathology, | pathology, |
| | 20 Tg | pathology, | cytokines, | cytokines, | cytokines, |
| | mice + | cytokines, | viral titer, | viral titer, | viral titer, |
| | exosomes | viral titer | immune | immune | immune |
| | | | response | response | response |
| | 24 adult | 4 mice/ | 4 mice/ | 4 mice/ | 12 mice/group |
| | and 24 old | group | group lung | group lung | survival; lung |
| | hACE2-Tg | lung | pathology, | pathology, | pathology, |
| | mice + | pathology, | cytokines, | cytokines, | cytokines, |
| | PBS | cytokines, | viral titer, | viral titer, | viral titer, |
| | | viral titer | immune | immune | immune |
| | | | response | response | response |
| | 12 | 2 mice/ | 2 mice/ | 2 mice/ | 6 mice/group |
| | uninfected | group | group lung | group lung | survival; lung |
| | adult and | lung | pathology, | pathology, | pathology, |
| | old hACE- | pathology, | cytokines, | cytokines, | cytokines, |
| | 2 Tg | cytokines, | viral titer, | viral titer, | viral titer, |
| | controls | viral titer | immune | immune | immune |
| | | | response | response | response |

b. SARS-CoV-2 Protocol:

For the coronavirus infection, we will use adult (3-month old) and old (>18 month old) C57BL/6 male mice transgenically expressing the human ACE2 molecule under the control of the epithelial keratin 18 promoter (B6.Cg-Tg(K18-ACE2)2Prlmn/J, Jax #034860). This is necessary because mice are not susceptible to SARS-COV2 unless the human ACE2 molecule is present in their cells via transgenesis or transduction. The K18-hACE2 Tg mice have been breeding in the Nikolich lab since June 2020 and are also available from Jackson Laboratories and we do not foresee any problem with generating the numbers outlined below; old mice of this strain will be aged in the Nikolich lab mouse colony and will be used in the ABSL/3 suite of the Keating Building at UArizona. Female mice will be used for confirmation and sex-related difference studies once all parameters are established in male mice.

Mice will be infected with 104 plaque-forming units of SARS-CoV2 strain WA-1/2020, which in the Nikolich lab produces 100% mortality within one week (see Table 10), with breathing difficulties and tachypnea observable from day 4 onwards (severe lethal COVID-19). 3 month old K18-hACE2 mice on a C57BL/6 background were infected intranasally with the indicated doses of the SARS-COV2 strain WA-1/2020; two days later 2 mice/group were sacrificed and virus titer in lung homogenates determined by plaque assay. Mortality was scored until day 21 and is shown as number of dead animals out of total, with the day of last death indicated. In Exp. #1, mice were also evaluated on d5 for breathing frequency and were found to have accelerated breathing.

TABLE 10
SARSCoV-2 infection in K18-hACE2 mice

| | | K18- | D2 lung | | Mortality |
|---|---|---|---|---|---|
| | Sars2 | hACE2 | titers (PFU, | Accelerated | (died/total; |
| | Dose | (mice | n = 2/ | breathing | day of last |
| Experiment | (PFU) | (#) | group) | (Y/N) | death) |
| #1 | 10⁴ | 6 | 10⁷-10⁸ | Y | 4/4 (d8) |
| #2 | 10¹ | 6 | 10⁵ | NT | 0/4 |
| | 10² | 6 | ND | NT | 1/4 (d6) |
| | 10³ | 5 | 10⁵ | NT | 1/3 (d6) |
| | 10⁴ | 2 | 10⁷ | NT | NA |

24 and 72 h later, 5×10¹⁰ exosome particles will be injected and the animals assessed as follows: 1) We will determine survival over 30 days, weight, temperature and activity of treated and control animals; 2) We will measure lung virus titers (by plaque assays) in the lungs on d4, 7, and 14; 3) we will determine induction of type I interferon (via CXCL10 production) and of other cytokines and chemokines as described in Phase I (see Bleomycin model Assessment), with all additional considerations mentioned therein) (101, 102). Final selection of cytokines and chemokines will be guided by the observed findings in pilot experiments with mice, as well as those elevated in severe COVID-19 patients (101). Histology and immunohistochemistry of the lung will be assessed (surfactant protein C, and aquaporin 5, a-smooth muscle cell actin and CD31 as endothelial marker). Design of groups and numbers of mice are depicted in Table 9, lower half.

Success metrics: Extension of survival by at least 20% over the control group to 14 days and beyond, will be considered our smallest acceptable level of efficacy. A secondary endpoint will be reduction of virus titers by an order of magnitude or more. Virus in these mice spreads to other organs as well, and by the time of these experiments, we will map precisely how much the virus spreads in old vs. adult mice; to that effect, we may be able to also analyze whether exosomes can reduce viral replication and/or damage in/to other organs, most notably the brain, heart and kidney (plaque assays and gross pathology). The analysis of immune responses in these animals (antibody response as described in our published work (103); T cell responses after stimulation with combined peptide pools as in (104), and in our prior work (105-110) we will test whether injection of exosomes, due to its anti-inflammatory properties, may interfere with the immune responses in adult or old animals, an issue of obvious clinical importance in therapeutics.

Pitfalls and alternatives: Lethal infection may prove a very high bar and we may need to reduce the virus does to 103 or below, although in that case we will have to perform additional pilot experiments to ascertain whether the extent of lung injury may be sufficient (as judged by expression of the same lung injury markers, surfactant protein C, and aquaporin 5, a-smooth muscle cell actin and CD31 as endothelial marker). At this dose, some mice die (see Table 10), which could be advantageous as we could aim for partial lethality improvement. Moreover, due to broad whole-body expression of hACE2 in the above transgenic mice, there could be virus pathology in other organs that kills the animal. As our alternative model, we will use infection of old and adult mice with the adeno-associated virus (AAV) encoding human ACE2 (AAV-hACE2), as described (104, 111). Fourteen days later, mice will be infected with SARS-COV2 and treated with exosomes and examined as above. It is possible that are dose concentration may have to be modified, however based on our preliminary data using the bleomycin mouse model we do not anticipate any problems. If for any reason we encounter problems with the extent of injury or reproducibility in the SARS-CoV-2 infection model, we will use the well-established S. pneumoniae model, with the influenza virus A PR/34 as the second backup model.

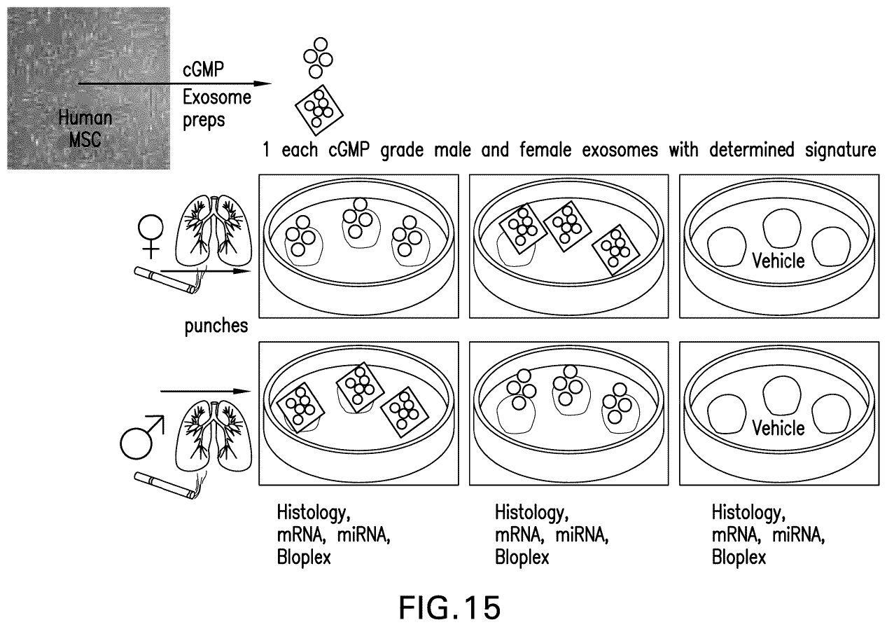

c. Ex vivo human tissue: To further test our signature in human lung tissue, we will perform 3-D punch experiments using male and female lung derived from patients who smoked and deemed not suitable for transplant at our hospital, Banner—University Medical Center Phoenix AZ, (see accompanying letter). We will cannulate and inject a bronchial branch with 2% agarose and perform punch experiments as described above. A series of punches will be collected that will receive vehicle injection. Four days after injection punches will be fixed in 10% formalin (Sigma-Aldrich), processed for paraffin embedding and stained with trichrome to assess collagen content. Parallel punches will be utilized to measure mRNA, miRNA expression, the inflammation panel and other relevant cytokines as outlined above (FIG. 15).

Success metrics: Based on our ongoing experiments, exosomes alter relevant for inflammation and fibrosis pathways in human lung punches. We would expect that lung punches treated with exosomes should have at least a 50% reduction over baseline in the inflammation panel. Lung punches obtained from subjects that smoked will express dysregulated inflamma-Mirs and others discussed in Significance section 5. Exosomes should normalize these miRNAs somewhat.

Success metrics/Alternative outcomes/Potential difficulties: The environment can influence the release of cargo once exosomes are circulating. This will be addressed by repeating the experiments in Phase ll with biological duplicates from the same donor and technical triplicates. We acknowledge the incomplete nature of the punch assays, omitting important components of the immune system, including macrophages and neutrophils. We also recognize that the conditions used in these experiments may be hypoxic. If we find that to be the case, we will utilize an incubator to regulate oxygen amounts during the four days of the experiment and compare outcomes. However, these studies are a complement to our pre-clinical mouse studies prior to submission to the FDA.

### Example 3. IND Enabling Studies

There is a well-established pathway for pharmaceutical small molecule drug development and review by the FDA. However, developers of biological products, such as EVs, have to modify certain parts of this established pathway, specifically that which is related to ADME-Toxicity testing (96, 112). Based on experience (95, 113, 114), the guidance provided by a 2018 position paper on the minimal information for studies of extracellular vesicles (115), and current open-enrollment information for therapies using EVs and exosomes, our team will perform the IND-enabling studies as outlined in the timeline. These studies are critical to define the product and as such will all be carried out.
1) EV Morphology-Size: A consistent particle size distribution between lots and batches is required as part of our release criteria and is necessary for further clinical development. Exosomes have been described as being 30-150 nm in diameter (116, 117). Microvesicles are larger than exosomes and are often described as being 100-300 nm in diameter. The degree of overlap in the sizes for these classes of EVs varies depending upon the technology used to make the measurement. Since we are measuring particle quantity and size, we can assign an exosome and microsome population percentage to each EV preparation, further defining our EV preparations into exosome and microsome composition, with a goal towards setting parameters for product release and purity. If EV size fractionation is suggested by the FDA prior to IND submission, these data will provide the necessary information to design effective EV size exclusion/recovery approaches. EV sizing and concentration characterizations will be performed on each sample in triplicate using each pore size membrane. Additionally, three independent analyses will be performed on different days. We will compare differences between the two sizing technologies using Student's t-tests. Success metrics: Particle yield should be at least 1×10⁹ per isolation; particle diameter should be consistent with a mean of 165±50 nm and a mode of 120±20 nm to include exosomes (30-150 nm). Significant variation from the above parameters at p≤0.05 will be considered unacceptable and would signal a no-go decision for a particular batch of exosomes.
2) EV Phenotype: We will continue using the MacsPlex Exosome Kit (Miltenyi) for analysis of 37 different surface proteins, including CD9, CD63, and CD81, which are exosome markers present in our EV preparations. We will monitor the relative abundance of these exosome markers in a high throughput manner across batches to assess population composition and assess batch-to-batch consistency for exosomes. Since these markers are detected using the capture antibody technology, the data generated for the additional surface proteins will reflect only those present on (captured) exosomes. We will analyze each EV lot across batches to determine the consistency of marker expression as part of our release criteria. Each analysis will be performed in triplicate through independent experiments on different days. Statistical differences between lots and batches will be determined using ANOVA and/or t-tests depending on the comparison. Success metrics: Exosome (CD9, CD63, CD81) and MSC-specific (CD29, CD44, CD105) biomarkers must be present in all lots, and variability between lots must remain below the significance level of p≤0.05.
3) EV miRNA content: Using the MacsPlex miRNA Kit (Miltenyi) we have analyzed 39 different micro-RNAs (miRNAs) that may be present in EV cargo. In parallel with EV surface protein expression described above, we will monitor the relative abundance of these miRNAs in a high through put manner. Each sample will be tested in triplicate using independent experiments on different days to ensure data quality. Lot-to-lot and batch-to-batch consistency will be determined using ANOVA and/or t-tests depending on the comparison and differences between measures will be considered significant with a p value≤0.05. We expect consistent lot-to-lot and batch-to-batch miRNA content to include members of the let7, miR200, miR335 and miR145 families. Success metrics: For a lot to be considered consistent with production criteria, variability relative to prior lots must remain below the significance level of p≤0.05.
4) Affinity: Information on how well the EVs bind to cell membranes is needed for subsequent pharmacokinetic studies, in addition to determining a dosage regimen for toxicity analysis. We will use our previously established lipophilic dye transfer assay in a time course of binding study (118). An aliquot of EVs will be labeled with Vybrant Dil cell labeling solution for 20 minutes at 37 °C and excess dye removed. Recipient lung cells will be seeded in triplicate at 70% confluence in 48 well plates and allowed to attach overnight. Growth medium will be removed and 25 million dye-loaded EVs added to recipient cells. Dye transfer from the labeled EVs to cultured recipient cells will be measured by flow cytometry after 1, 2, 4, 8, 12, 16, and 24 hours of incubation at 37 °C. Controls will include dye-labeled EVs incubated with recipient cells at 4 °C. (Negative control) and recipient cells alone (Background). Once the optimal incubation time is identified, an ascending dose of EVs (1-100 million) will be tested to determine the saturating dose of EVs and a potential EC50 value for competitive assays. An in vitro competitive assay will also be performed to investigate equilibrium binding at a fixed concentration of dye-labeled EVs (EC75) in the presence of increasing concentrations of unlabeled EV competitor. All assays will be performed in triplicate using at least two donor recipient cell lots of MSCs. Additionally, three independent experiments will be performed on different days to ensure the reproducibility of the response. Success metrics: Statistical differences between EVs derived from different MSC treatments will be analyzed by ANOVA and/or t-tests depending on the comparison and significance will be considered acceptable if p value is >0.05. Alternatively, these studies can also be extended to include EV miRNA labeling studies (ExoGlow™-RNA EV Labeling Kit, SBI) to assess EV miRNA cargo delivery to recipient cells.
5) Immune Response-in vitro: Previous in vitro and in vivo studies suggest that human MSCs do not induce an immune response, and in some studies higher passage human MSCs actually were immunosuppressive in mixed lymphocyte reactions (MLRs) (119, 120). In the typical MLR, an inactivated stimulator cell population is used to stimulate a naïve T-cell population from a different donor. These naïve T-cells then recognize the foreign cells and are activated and begin to proliferate (121). A one-way MLR using EVs as the stimulator population will be performed as previously described (122). Briefly, splenocytes from BALB/c mice will be isolated and seeded in a volume of 100 pL at a density of 1×10⁵ cells per well in 96-well round-bottom plates, in triplicate. An ascending dose of 30, 100, and 500 million EV particles from C57BL/6 mice will be added as a stimulator to obtain a 100 pL final volume. After 3 days of incubation, 1 pCi/well [³H] thymidine will be added overnight and thymidine incorporation measured using a [3-scintillation counter. Values will be compared to positive (phytohemagglutinin) and negative (vehicle alone) controls. Although we feel this is unlikely, an increase in [³H] thymidine counts for the experimental compared to the negative control will indicate a positive immune response. In addition to the murine MLR assay, we will test EVs for a similar response using human CD3+ T-cells freshly isolated at Zen-Bio (121). The one-way MLR will be performed using responder T-cells with the following stimulator cell populations that have been inactivated by treatment with mitomycin C: autologous PBMCs (from the T-cell donor, negative control) and allogeneic PBMCs (positive control). T-cells alone will also serve as a background control. Responder cells will be seeded in triplicate in a volume of 100 pL at a density of 1×10⁵ cells per well in 96-well round-bottom plates. Ascending doses of mitomycin-treated stimulator cells (1,000-20,000) or EVs (30, 100, and 500 million particles) will be added to a final volume of 200 pl. Proliferation will be determined after 72 hours using a chemiluminescent cell proliferation assay for BrDu incorporation (Roche) to assess active DNA synthesis and Cell Titer Blue for correlation with live cell numbers. All assays will be performed in triplicate using at least 5 different donor responder cell lots, and three independent experiments performed on different days to ensure the reproducibility of the response. Success metrics: Statistical analysis will be performed using student's t-test for comparing treatment groups with a p value≤0.05 being considered as significant. Only the batches that are not significantly different in immunostimulatory capacity from negative control will be considered acceptable.
6) Immune Response-in vivo: We anticipate that the in vitro MLR will be negative. As such, we will proceed with the in vivo rodent T-cell-dependent antibody response (TDAR) assay to complete the immune-response analysis. The TDAR assay is commonly used to assess new drug potential for immunotoxicity and immunosuppression, and is dependent on antigen uptake and presentation, B-cell activation with T-cell help, and antibody production. We will conduct these studies at Synchrony Labs using C57BL/6 mice according to previously described detailed pharmaceutical guidance to provide enough power to identify significant differences (123). Four groups of 20 mice (10 female and 10 male) will be injected subcutaneously with saline, 300 pg keyhole limpet hemocyanin, 1×10⁸ untreated MSC-derived EVs or 1×10⁸ optimally produced EVs from Aims 1 and 2. Injections of saline and EVs will be performed daily for 28 days as suggested by EPA guidelines for unknown entities; KLH will be injected at day 0 and again on day 14. Blood samples will be taken for analysis on Days 0, 5, 7, 10, 14, 19, 21, 24 and 28. We will assess the effect of EVs on the immune system by ELISA for total IgM, IgG subclass, IgA, and IgE in responses to EV challenge (124). Values will be compared to positive (KLH) and negative (saline) controls to determine the positive or negative immune response in vivo. Success metrics: Statistical analysis will be performed using GraphPad Prism software and analyzed by ANOVA with multiple and pair-wise comparisons and/or t-tests depending on the comparison, and significance will be considered as a p value ≤0.05. Only the batches that are not significantly different in immunostimulatory capacity from negative control will be considered acceptable
7) Toxicity: To generate an initial assessment of EV toxicity, we will perform repeated dose 28-day and 90-day toxicity studies in mice with slight modifications to the OECD guidelines (125, 126). Male and female C57BL/6 mice will be used for these studies. Two groups of 30, 6-7 week old mice (15 male and 15 female) will be treated daily with a 100 μL subcutaneous injection of saline or 1×10⁸ optimal EVs determined from Aims 1 and 2. Animals will be observed daily for any overt signs of general toxicity including, changes in skin, fur, eyes, mucous membranes, unusual respiratory pattern, changes in response to handling, and repetitive or bizarre behaviors. Weekly measures of body weight, and food and water consumption will be recorded. On day 28, 10 animals from each group (5 male and 5 female) will have blood collected and be euthanized for post-mortem analyses. The remaining animals will continue to be treated until day 90 whereupon they will have blood collected and be euthanized for post-mortem analysis. Endpoint analyses for these studies consist of an evaluation of clinical observations (body weight, food and water consumption), blood cell analysis (red and white cell counts), blood chemistry (glucose, BUN, creatinine, total protein, ALP, AST), whole body gross necropsy, and histopathologic examination of heart, liver, lungs, spleen, kidneys, and brain. Pathology analytical services will be provided by Infinium Pathology Consultants. Success metrics: Toxicity endpoints will include increased mortality and unfavorable physiological and biochemical changes in the EV treated group compared to the control saline group. Statistical analysis will be performed using Student's T-test for comparing treatment groups with a p value≤0.05 being considered as significant, and samples with significant toxicity will be considered unacceptable.
8) Tumorigenicity: Unlike many regenerative medicine therapeutic products which contain live cells as the active component, EVs are non-replicating, thereby eliminating the possibility that EVs themselves will become malignantly transformed. As such, we will test whether optimized EVs facilitate tumor formation in immunocompromised mice as an initial assessment of tumorigenicity. Due to the strict requirements of handling and housing immunocompromised mice, we will contract out this study to Charles River Labs using their in-house protocols. Twenty athymic nude mice will be randomly sorted into two groups for 200 pL injections of saline or 1×10⁸ optimal EVs from Aims 1 and 2 into the subcutaneous dorsal flank. The mice will be monitored for survival and tumor growth 2-3 times a week for up to 8 weeks, measuring the length and width of the tumor using a slide caliper (127). Should tumors in these immunocompromised rodents grow to a diameter of 150-200 mm³, they will be removed from euthanized animals, weighed and measured for tumor size and volume, and preserved appropriately for histological investigation. As an alternative, we will consider transplanting EVs into the kidney capsule of mice to determine if EVs can generate a tumor in specific organ tissue (128). Success metrics: Statistical analysis will be performed using Student's t-test for comparing EV treated animals to the control group with a p value≤0.05 being considered as significant; any thus defined significant tumorigenicity will be considered unacceptable.
9) EV preservation and shelf-life: We have previously lyophilized EVs and stored them at room temperature for several weeks before reconstituting them for size analyses and in vivo studies. Throughout this process we did not observe any differences in particle size or in vivo efficacy compared to freshly isolated EVs. In order to provide a more thorough analysis of EV preservation, shelf-life and integrity, we will perform a time course study over 12 months and include endpoint assays of in vitro morphology, phenotype, miRNA content, affinity, and efficacy analysis in human primary cells (proliferation, migration and angiogenesis). Nine 1×10E8 aliquots of all EV lots prepared will be subjected to lyophilization and stored in the dark at room temperature in an air-tight desiccator. Parallel aliquot samples will include EVs suspended in PBS immediately after isolation and stored at −80, −20, and +4 degrees Celsius. Triplicate samples will be thawed or suspended in PBS at 3, 6 and 12 months and subjected to the battery of in vitro assays listed above. Statistical analysis will be performed using GraphPad Prism software and analyzed by ANOVA with multiple and pair-wise comparisons and/or t-tests depending on the comparison, and significance will be considered as a p value≤0.05. Success metrics: Based on our previous results, we expect that the 4 °C stored EVs will lose efficacy and show degradation prior to all other storage conditions. Additionally, we expect that the −80 °C stored EVs will maintain their physical and biochemical properties for 12 months with less than 15% reduction in these characteristics. Batches that diminish in potency by more than 15% will be considered unacceptable. We expect that these in vitro assays will serve as in-process controls during product manufacturing and be useful in determining preservation conditions for optimal product shelf-life.
10) Sterility and Bioburden: All of our human tissue from which we isolated MSCs is tested by a third-party using PCR to identify the presence of common viral pathogens (HIV I & II and hepatitis B & C) and tested internally for bacterial and fungal contamination. Any tissues found to be positive are discarded as well as any cells derived from those tissues. We also perform sterility testing during cell expansion prior to seeding the bioreactor, during bioreactor medium collection and post-EV isolation. As part of our pre-clinical development, we will subject our EV products to bioburden and sterility testing using Eurofins Scientific. EV products will be tested for sterility, mycoplasma, endotoxin, and bioburden according to their methodologies accepted by regulatory agencies. Success metrics: Any positive bioburden samples will be analyzed to identify contaminating organism(s). Any EV batch which is not sterile will be discarded. To date, we have not produced any EV lots that have tested positive for mycoplasma or endotoxin, but we will continue to monitor for these contaminants as we proceed through development. Triplicate blinded samples containing vehicle or optimal EVs from Aims 1 and 2 from at least 3 batches will be sent to Eurofins for analysis.

Timeline: Phase II is expected to take 24 months to complete. Our timeline allows for additional exosomes to be prepared as necessary.

### REFERENCES FOR EXAMPLES 1-3

2. W. Merkt, M. Bueno, A. L. Mora, D. Lagares, Senotherapeutics: Targeting senescence in idiopathic pulmonary fibrosis. Semin Cell Dev Biol 101, 104-110 (2020).
3. K. Ascher, S. J. Elliot, G. A. Rubio, M. K. Glassberg, Lung Diseases of the Elderly: Cellular Mechanisms. Clin Geriatr Med 33, 473-490 (2017).
4. D. M. E. Bowdish, The Aging Lung: Is Lung Health Good Health for Older Adults? Chest 155, 391-400 (2019).
5. D. Furman et al., Chronic inflammation in the etiology of disease across the life span. Nature Medicine 25, 1822-1832 (2019).
6. B. K. Kennedy et al., Geroscience: linking aging to chronic disease. Cell 159, 709-713 (2014).
7. S. Navarro, B. Driscoll, Regeneration of the Aging Lung: A Mini-Review. Gerontology 63, 270-280 (2017).
8. J. F. Cordier, V. Cottin, Neglected evidence in idiopathic pulmonary fibrosis: from history to earlier diagnosis. Eur Respir J 42, 916-923 (2013).
9. D. J. Lederer, F. J. Martinez, Idiopathic Pulmonary Fibrosis. N Engl J Med 379, 797-798 (2018).
10. L. Richeldi, H. R. Collard, M. G. Jones, Idiopathic pulmonary fibrosis. Lancet 389, 1941-1952 (2017).
11. S. Meiners, O. Eickelberg, M. Konigshoff, Hallmarks of the ageing lung. Eur Respir J 45, 807-827 (2015).
12. P. J. Barnes, Oxidative stress-based therapeutics in COPD. Redox Biology 33, 101544 (2020).
13. P. J. Barnes, J. Baker, L. E. Donnelly, Cellular Senescence as a Mechanism and Target in Chronic Lung Diseases. American journal of respiratory and critical care medicine 200, 556-564 (2019).
14. P. J. Barnes, Small airway fibrosis in COPD. The international journal of biochemistry & cell biology 116, 105598 (2019).
15. P. J. Barnes, Pulmonary Diseases and Ageing. Subcell Biochem 91, 45-74 (2019).
16. S. J. Cho, H. W. Stout-Delgado, Aging and Lung Disease. Annu Rev Physiol 82, 433-459 (2020).
17. U. Kulkarni et al., Excessive neutrophil levels in the lung underlie the age-associated increase in influenza mortality. Mucosal Immunol 12, 545-554 (2019).
18. C. K. Wong et al., Aging Impairs Alveolar Macrophage Phagocytosis and Increases Influenza-Induced Mortality in Mice. J Immunol 199, 1060-1068 (2017).
39. J. Tashiro et al., Therapeutic benefits of young, but not old, adipose-derived mesenchymal stem cells in a chronic mouse model of bleomycin-induced pulmonary fibrosis. Transl Res 166, 554-567 (2015).
40. J. Fafián-Labora et al., Influence of mesenchymal stem cell-derived extracellular vesicles in vitro and their role in ageing. Stem cell research & therapy 11, 13 (2020).
41. D. Gnani et al., An early-senescence state in aged mesenchymal stromal cells contributes to hematopoietic stem and progenitor cell clonogenic impairment through the activation of a pro-inflammatory program. Aging cell, e12933 (2019).
42. R. Vono, E. Jover Garcia, G. Spinetti, P. Madeddu, Oxidative Stress in Mesenchymal Stem Cell Senescence: Regulation by Coding and Noncoding RNAs. Antioxidants & redox signaling 29, 864-879 (2018).
43. F. Olivieri et al., Circulating miRNAs and miRNA shuttles as biomarkers: Perspective trajectories of healthy and unhealthy aging. Mechanisms of Ageing and Development 165, 162-170 (2017).
44. J. Boulestreau, M. Maumus, P. Rozier, C. Jorgensen, D. Noel, Mesenchymal Stem Cell Derived Extracellular Vesicles in Aging. Front Cell Dev Biol 8, 107 (2020).
45. J. L. Rinn, M. Snyder, Sexual dimorphism in mammalian gene expression. Trends in Genetics 21, 298-305 (2005).
46. T. Connallon, L. L. Knowles, Intergenomic conflict revealed by patterns of sex-biased gene expression. Trends in Genetics 21, 495-499 (2005).
47. L. M. McIntyre et al., Sex-specific expression of alternative transcripts in Drosophila. Genome Biology 7, R79 (2006).
48. A. Vigé, C. Gallou-Kabani, C. Junien, Sexual Dimorphism in Non-Mendelian Inheritance. Pediatric Research 63, 340-347 (2008).
49. E. Bianconi et al., Sex-Specific Transcriptome Differences in Human Adipose Mesenchymal Stem Cells. Genes (Basel) 11, (2020).
50. A. Giuliani et al., Mitochondrial (Dys) Function in Inflammaging: Do MitomiRs Influence the Energetic, Oxidative, and Inflammatory Status of Senescent Cells? Mediators Inflamm 2017, U.S. Pat. No. 2,309,034 (2017).
51. G. L. Russo et al., Mechanisms of aging and potential role of selected polyphenols in extending healthspan. Biochem Pharmacol 173, 113719 (2020).
52. F. Gurau et al., Anti-senescence compounds: A potential nutraceutical approach to healthy aging. Ageing Res Rev 46, 14-31 (2018).
53. K. Tsubota, The first human clinical study for NMN has started in Japan. NPJ Aging Mech Dis 2, 16021 (2016).
54. J. Campisi et al., From discoveries in ageing research to therapeutics for healthy ageing. Nature 571, 183-192 (2019).
55. M. Fuentealba et al., Using the drug-protein interactome to identify anti-ageing compounds for humans. PLOS Computational Biology 15, e1006639 (2019).
56. D. Zhang et al., Exosome-Mediated Small RNA Delivery: A Novel Therapeutic Approach for Inflammatory Lung Responses. Molecular Therapy 26, 2119-2130 (2018).
57. R. Kangas et al., Aging and serum exomiR content in women-effects of estrogenic hormone replacement therapy. Sci Rep 7, 42702 (2017).
58. J. Li et al., miR-10a restores human mesenchymal stem cell differentiation by repressing KLF4. Journal of cellular physiology 228, 2324-2336 (2013).
59. L. S. Huang et al., The Mitochondrial Cardiolipin Remodeling Enzyme Lysocardiolipin Acyltransferase (LYCAT) is a Novel Target in Pulmonary Fibrosis. American journal of respiratory and critical care medicine, (2014).
60. J. M. Yu et al., Age-related changes in mesenchymal stem cells derived from rhesus macaque bone marrow. Aging cell 10, 66-79 (2011).
61. L. Liu et al., MicroRNA-181a Regulates Local Immune Balance by Inhibiting Proliferation and Immunosuppressive Properties of Mesenchymal Stem Cells. STEM CELLS 30, 1756-1770 (2012).
62. K. Watanabe et al., Functional similarities of microRNAs across different types of tissue stem cells in aging. Inflammation and Regeneration 38, 9 (2018).
63. T. Squillaro, G. Peluso, U. Galderisi, Clinical Trials With Mesenchymal Stem Cells: An Update. Cell Transplant 25, 829-848 (2016).
64. A. Stolzing, E. Jones, D. McGonagle, A. Scutt, Age-related changes in human bone marrow-derived mesenchymal stem cells: consequences for cell therapies. Mech Ageing Dev 129, 163-173 (2008).
65. S. Bork et al., DNA methylation pattern changes upon long-term culture and aging of human mesenchymal stromal cells. Aging cell 9, 54-63 (2010).
66. C. R. Kliment, T. D. Oury, Oxidative stress, extracellular matrix targets, and idiopathic pulmonary fibrosis. Free Radical Biology and Medicine 49, 707-717 (2010).
67. A. van der Vliet, Y. M. W. Janssen-Heininger, V. Anathy, Oxidative stress in chronic lung disease: From mitochondrial dysfunction to dysregulated redox signaling. Mol Aspects Med 63, 59-69 (2018).
68. M. K. Glassberg et al., Estrogen deficiency promotes cigarette smoke-induced changes in the extracellular matrix in the lungs of aging female mice. Transl Res 178, 107-117 (2016).
69. N. Kassira et al., Estrogen deficiency and tobacco smoke exposure promote matrix metalloproteinase-13 activation in skin of aging B6 mice. Ann Plast Surg 63, 318-322 (2009).
70. S. J. Elliot et al., Smoking induces glomerulosclerosis in aging estrogen-deficient mice through cross-talk between TGF-beta1 and IGF-I signaling pathways. Journal of the American Society of Nephrology: JASN 17, 3315-3324 (2006).
71. A. Izzotti et al., Downregulation of microRNA expression in the lungs of rats exposed to cigarette smoke. FASEB J 23, 806-812 (2009).
72. A. Izzotti, G. A. Calin, V. E. Steele, C. M. Croce, S. De Flora, Relationships of microRNA expression in mouse lung with age and exposure to cigarette smoke and light. FASEB journal: official publication of the Federation of American Societies for Experimental Biology 23, 3243-3250 (2009).
73. F. Schembri et al., MicroRNAs as modulators of smoking-induced gene expression changes in human airway epithelium. Proc Natl Acad Sci USA 106, 2319-2324 (2009).
74. J. W. Graff et al., Cigarette smoking decreases global microRNA expression in human alveolar macrophages. PloS one 7, e44066 (2012).
75. M. K. Glassberg et al., Allogeneic Human Mesenchymal Stem Cells in Patients With Idiopathic Pulmonary Fibrosis via Intravenous Delivery (AETHER): A Phase I Safety Clinical Trial. Chest 151, 971-981 (2017).
76. R. Peng et al., Bleomycin induces molecular changes directly relevant to idiopathic pulmonary fibrosis: a model for “active” disease. PloS one 8, e59348 (2013).
77. N. Srour, B. Thebaud, Mesenchymal Stromal Cells in Animal Bleomycin Pulmonary Fibrosis Models: A Systematic Review. Stem cells translational medicine, (2015).
78. H. Dweep, C. Sticht, P. Pandey, N. Gretz, miRWalk—database: prediction of possible miRNA binding sites by “walking” the genes of three genomes. Journal of biomedical informatics 44, 839-847 (2011).
79. A. Jusic et al., Approaching Sex Differences in Cardiovascular Non-Coding RNA Research. International journal of molecular sciences 21, 4890 (2020).
80. R. Song et al., Many X-linked microRNAs escape meiotic sex chromosome inactivation. Nat Genet 41, 488-493 (2009).
81. S. Elliot et al., MicroRNA let-7 Downregulates Ligand-Independent Estrogen Receptor-mediated Male-Predominant Pulmonary Fibrosis. American journal of respiratory and critical care medicine 200, 1246-1257 (2019).
82. G. Izbicki, M. J. Segel, T. G. Christensen, M. W. Conner, R. Breuer, Time course of bleomycin-induced lung fibrosis. Int J Exp Pathol 83, 111-119 (2002).
83. L. Ferrucci et al., The origins of age-related proinflammatory state. Blood 105, 2294-2299 (2005).
84. S.-i. Imai, L. Guarente, NAD+ and sirtuins in aging and disease. Trends in Cell Biology 24, 464-471 (2014).
85. A. Brunet et al., Stress-dependent regulation of FOXO transcription factors by the SIRT1 deacetylase. Science 303, 2011-2015 (2004).
86. H. F. Yuan et al., SIRT1 is required for long-term growth of human mesenchymal stem cells. J Mol Med (Berl) 90, 389-400 (2012).
89. LEFT BLANK
90. K. Zscheppang et al., Human Pulmonary 3D Models For Translational Research. Biotechnol J 13, (2018).
91. H. N. Alsafadi et al., Applications and Approaches for Three-Dimensional Precision-Cut Lung Slices. Disease Modeling and Drug Discovery. Am J Respir Cell Mol Biol 62, 681-691 (2020).
92. G. B. Fields, The Rebirth of Matrix Metalloproteinase Inhibitors: Moving Beyond the Dogma. Cells 8, (2019).
93. K. Ostridge et al., Relationship between pulmonary matrix metalloproteinases and quantitative CT markers of small airways disease and emphysema in COPD. Thorax 71, 126 (2016).
94. M. Provenzano et al., The Association of Matrix Metalloproteinases with Chronic Kidney Disease and Peripheral Vascular Disease: A Light at the End of the Tunnel? Biomolecules 10, (2020).
95. W. G. L. Whitford, J. W.; Cadwell, J. J. S., Continuous production of exosomes. Genetic Engineering and Biotechnology News 35, 34 (2015).
96. M. Mendt et al., Generation and testing of clinical-grade exosomes for pancreatic cancer. JCI Insight 3, (2018).
97. K. B. Baumgartner, J. M. Samet, C. A. Stidley, T. V. Colby, J. A. Waldron, Cigarette smoking: a risk factor for idiopathic pulmonary fibrosis. American journal of respiratory and critical care medicine 155, 242-248 (1997).
98. W. I. Choi et al., Risk factors for interstitial lung disease: a 9-year Nationwide population-based study. BMC Pulm Med 18, 96 (2018).
99. R. Joehanes et al., Epigenetic Signatures of Cigarette Smoking. Circulation: Cardiovascular Genetics 9, 436-447 (2016).
100. D. H. Matulionis, Chronic cigarette smoke inhalation and aging in mice: 1. Morphologic and functional lung abnormalities. Exp Lung Res 7, 237-256 (1984).
101. T. Takahashi et al., Sex differences in immune responses that underlie COVID-19 disease outcomes. Nature, (2020).
102. Y. Zhao et al., Longitudinal COVID-19 profiling associates IL-IRA and IL-10 with disease severity and RANTES with mild disease. JCI Insight 5, (2020).
103. T. J. Ripperger et al., Orthogonal SARS-COV-2 Serological Assays Enable Surveillance of Low-Prevalence Communities and Reveal Durable Humoral Immunity. Immunity, (2020).
104. M. Hassert et al., mRNA induced expression of human angiotensin-converting enzyme 2 in mice for the study of the adaptive immune response to severe acute respiratory syndrome coronavirus 2. bioRxiv, (2020).
105. J. L. Uhrlaub et al., Dysregulated TGF-beta Production Underlies the Age-Related Vulnerability to Chikungunya Virus. PLoS Pathog 12, e1005891 (2016).
106. J. L. Uhrlaub, M. J. Smithey, J. Nikolich-Zugich, Cutting Edge: The Aging Immune System Reveals the Biological Impact of Direct Antigen Presentation on CD8 T Cell Responses. J Immunol 199, 403-407 (2017).
107. J. D. Brien, J. L. Uhrlaub, A. Hirsch, C. A. Wiley, J. Nikolich-Zugich, Key role of T cell defects in age-related vulnerability to West Nile virus. J Exp Med 206, 2735-2745 (2009).
108. L. Cicin-Sain et al., Cytomegalovirus infection impairs immune responses and accentuates T-cell pool changes observed in mice with aging. PLoS Pathog 8, e1002849 (2012).
109. G. Li, M. J. Smithey, B. D. Rudd, J. Nikolich-Zugich, Age-associated alterations in CD8alpha+ dendritic cells impair CD8 T-cell expansion in response to an intracellular bacterium. Aging cell 11, 968-977 (2012).
110. M. J. Smithey, K. R. Renkema, B. D. Rudd, J. Nikolich-Zugich, Increased apoptosis, curtailed expansion and incomplete differentiation of CD8+ T cells combine to decrease clearance of L. monocytogenes in old mice. Eur J Immunol 41, 1352-1364 (2011).
111. B. Israelow et al., Mouse model of SARS-CoV-2 reveals inflammatory role of type I interferon signaling. J Exp Med 217, (2020).
112. G. R. Willis, S. Kourembanas, S. A. Mitsialis, Toward Exosome-Based Therapeutics: Isolation, Heterogeneity, and Fit-for-Purpose Potency. Front Cardiovasc Med 4, 63 (2017).
113. J. Basu, J. W. Ludlow, Cell-based therapeutic products: potency assay development and application. Regen Med 9, 497-512 (2014).
114. J. Basu, J. W. Ludlow, Exosomes for repair, regeneration and rejuvenation. Expert Opin Biol Ther 16, 489-506 (2016).
115. C. Thery et al., Minimal information for studies of extracellular vesicles 2018 (MISEV2018): a position statement of the International Society for Extracellular Vesicles and update of the MISEV2014 guidelines. J Extracell Vesicles 7, 1535750 (2018).
116. R. A. Dragovic et al., Sizing and phenotyping of cellular vesicles using Nanoparticle Tracking Analysis. Nanomedicine 7, 780-788 (2011).
117. E. van der Pol et al., Optical and non-optical methods for detection and characterization of microparticles and exosomes. J Thromb Haemost 8, 2596-2607 (2010).
118. M. C. Deregibus et al., Endothelial progenitor cell derived microvesicles activate an angiogenic program in endothelial cells by a horizontal transfer of mRNA. Blood 110, 2440-2448 (2007).
119. K. McIntosh et al., The immunogenicity of human adipose-derived cells: temporal changes in vitro. Stem Cells 24, 1246-1253 (2006).
120. K. R. McIntosh et al., Immunogenicity of allogeneic adipose-derived stem cells in a rat spinal fusion model. Tissue Eng Part A 15, 2677-2686 (2009).
121. L. M. Muul et al., Measurement of proliferative responses of cultured lymphocytes. Curr Protoc Immunol Chapter 7, Unit7 10 (2011).
122. F. Djouad et al., Immunosuppressive effect of mesenchymal stem cells favors tumor growth in allogeneic animals. Blood 102, 3837-3844 (2003).
123. H. Lebrec et al., The T-cell-dependent antibody response assay in nonclinical studies of pharmaceuticals and chemicals: study design, data analysis, interpretation. Regul Toxicol Pharmacol 69, 7-21 (2014).
124. K. Hata et al., Differential regulation of T-cell dependent and T-cell independent antibody responses through arginine methyltransferase PRMT1 in vivo. FEBS Lett 590, 1200-1210 (2016).
125. OECD, Test No. 407: Repeated Dose 28-day Oral Toxicity Study in Rodents. (2008).
126. OECD. (2018).
127. M. M. Tomayko, C. P. Reynolds, Determination of subcutaneous tumor size in athymic (nude) mice. Cancer Chemother Pharmacol 24, 148-154 (1989).

### Example 4. Sex Disparity in COVID-19 Viral Infection

### Specific Aims:

In most countries, men are disproportionately dying of COVID infections (globalhealth5050.org/covid19; accessed Jun. 13, 2020), while the incidence and prevalence of infection is equal between men and women (1, 2). This is particularly evident in cohorts over 50 (globalhealth5050.org/covid19). Therefore biological (age, hormonal state, chromosome contribution, immune function, higher rate of comorbid conditions) and gender-related (lifestyle and socioeconomic status) factors in patients with COVID-19 need to be assessed to determine why men may have increased mortality. For example, levels of ACE2, the mechanism for viral infection, are generally higher in men in a study examining patients with cardiovascular disease. Initial investigations into sex differences have begun to dissect disparate responses to SARS-CoV2, with males showing higher plasma levels of certain chemokines (CCL5) and cytokines (IL-8&18), as well as increased induction of non-classical monocytes; by contrast, females have exhibited a more robust cell activation. Understanding how and why one or the other type of reactivity provides advantage or disadvantage in COVID-19 remains a critical task. This proposal has high impact since sex chromosome genes and/or sex hormone receptor activation could potentiate protective factors in females or harmful factors in males. Discovery of these mechanisms may guide new therapies that prevent or ameliorate COVID-19 lung disease in both sexes.

COVID-19 infection induces a lung inflammatory response that can progress to inflammation with cytokine storm. This can lead to acute lung injury (ALI), acute respiratory distress syndrome (ARDS), organ failure, and death. Therefore, in this proposal we will focus on the lung, although we recognize that other organs including the heart, kidney, gastrointestinal tract, liver, pancreas, nervous system and skin are also viral targets. Sex-specific disease outcomes following virus infections are attributed to sex-dependent production of steroid hormones, different copy numbers of immune response X-linked genes, and the presence of disease susceptibility genes in males and females (3, 4). An ongoing clinical trial is testing whether transdermal estrogen (NCT04359329) may have a protective effect in males and females with COVID-19. Therefore, understanding the hormonal and chromosomal underpinnings of the documented sex disparities in COVID-19 is critical to the efforts to treat, promote earlier detection, and evaluate drugs for this formidable disease.

This disclosure hypothesizes that activation of gonadal hormone receptors and sex chromosome complement in alveolar cells (epithelial and endothelial) of aging lungs are integral components driving the pronounced sex disparity. To test this hypothesis, we propose to isolate exosomes from the serum or plasma from age-matched and disease severity matched male and female patients with COVID-19 that had been collected and banked by at the University of Arizona.

We will utilize exosomes since cells employ exosomes to transfer regulatory factors that modulate the response of local and distant cells and systemic responses. In cells with active viral or bacterial infections, the exosome machinery can package pathogen-derived factors that alter the phenotype of the recipient cells (5, 6). We will isolate exosomes from adult and older male and female patients to investigate biological differences that may be related not only to sex but age. We will inject exosomes into lung punches. Our preliminary data show that naïve lung punches contain alveolar cell types important in COVID-19 infection. We also show that the normal lung phenotype can be converted to a diseased phenotype through the introduction of fibrotic cell-derived exosomes into the punch. This is mediated in part by exosome delivery of microRNAs which may differ between males and females. We will test the following specific aims

Aim 1: Determine the contribution of hormone receptor expression and subsequent downstream signaling in sex disparity of COVID-19-associated lung disease. Determine if sex, age or disease severity of COVID-19-derived exosomes influence sex hormone receptor expression, signaling or cytokine and chemokine response to SARS-COV2 differently in male and female control or GDX mouse lung punches.

Aim 2: Determine the contribution of sex chromosomes to differences in sex disparity in Covid-19 lung disease using the four core genotypes (FCG) mouse model. Compare lung punches derived from FCG mice that have the same type of gonads to determine if sex differences in COVID-19 are caused in part by genes encoded by the sex chromosomes which are inherently differently expressed in XX and XY lungs. Test whether these mice themselves are showing sex-determined differences in immune responses and in susceptibility to SARS-CoV2 using lung-restricted transduction of hACE2, followed by SARS-COV2 infection. Investigate, in the same model, whether sex and age interact to pronounce the susceptibility.

These studies provide critical initial insights into the fundamental basis of sex-related differences in susceptibility to COVID-19 and will pave the way towards detailed mechanistic studies of this key topic.

Significance: The convergence of aging and gonadal hormones. Gonadal hormone production/activation declines during reproductive aging and has been linked to multiple age-associated diseases including cardiovascular disease (7, 8), diabetic kidney disease (9-12), prostate cancer (13), lung cancer (14), and other lung diseases (15, 16). Underlying differences between males and females may become more apparent with age-associated changes of gonadal hormones and their signaling due to either loss of protection and/or gain of harmful effects, or by the emergence of sex chromosome effects that may be suppressed by gonadal hormones. Our prior studies support a protective effect of estrogen in the lungs of aged female mice; E2 replacement partially restored the destruction of inter-alveolar septa in the lungs of the aged mice (15, 16). To date and to our knowledge there are no comparable studies in aged male mice. Recent population-based studies continue to suggest a protective effect of estrogens as menopause is associated with accelerated lung function decline (17). However, changes of gonadal hormones in aged men are not as predictable as in women making comparable population studies in males more challenging (18).

Sex chromosomes and aging. Although gonadal sex is a major difference between men and women, it is not the only difference and may not account for all differences in age-associated lung disease. Since the number of X chromosomes is different between the sexes, the presence or absence of the Y chromosome independently or in concert with gonadal hormones may promote or protect against age-associated lung disease. Approximately 15% of human X-linked genes escape silencing and are expressed from both X chromosomes in females, resulting in the higher expression in XX females as compared to XY males (19). Because the ratio of estrogens to androgens becomes more similar in males and females with age, over time the influence of gonadal hormones may be less important than the modulation of disease phenotypes by sex chromosomes (18, 20). This will be addressed in specific Aim (SA) 2.

Sex differences, viral infections, immunity and aging. As discussed, both above and below, issues of sex differences in response to viral infections, particularly in older adults and animals, have not been thoroughly dissected. Our collaboration between experts in sex genetics and hormone biology and its impact on lung physiology and leaders in antiviral immunity in older subjects is an ideal vehicle to address such gaps in knowledge

STRENGTHS/WEAKNESSES OF CITED STUDIES: Gonadal hormones/receptors and the human lung.

The human lung is a gonadal hormone target tissue (21). Gonadal hormones regulate normal lung development, physiology, and are implicated in several lung diseases including asthma, pulmonary fibrosis, and pulmonary hypertension in males and females (21, 22). AR and ER are present in the lung; however, their signaling remains poorly understood. Our published data support the idea that steroid hormone receptors participate in signaling pathways relevant to fibrosis. In SA1 the experiments will lead us to characterize mechanisms that target viral pathways stimulated by sex steroid hormone receptors (23)

Hormones and coronavirus infection: Both the Middle East respiratory syndrome coronavirus (MERS-CoV) and the severe respiratory syndrome coronavirus 1 (SARS-CoV1) were found to infect and clinically affect more men than women (24). Similar data was derived from pre-clinical studies of SARS-CoV1 infection. Male mice were more susceptible to infection than female mice (25). The enhanced susceptibility of male mice to SARS-CoV1 correlated with a moderate increase in virus titer and extensive alveolar macrophages and neutrophil accumulation in the lungs (25). Gonadectomy had no effect on disease outcome in male mice, while oophorectomy or treating female mice with an estrogen receptor (ER) antagonist resulted in increased mortality to SARS-COV infection (25). These findings suggest that estrogen signaling protects female mice from a lethal infection (25).

Sex chromosomes and viral infection: Sex chromosome genes and sex hormones, including estrogens, progesterone and androgens, contribute to the differential regulation of immune responses between the sexes. Women are functional mosaics for X-lined genes, and the X-chromosome contains a high density of immune-related genes; therefore, women generally mount stronger innate and adaptive immune responses than men (26). This results in faster clearance of pathogens and greater vaccine efficacy in females than in males but also contributes to their increased susceptibility to inflammatory and autoimmune diseases. With regard to SARS-COV2, a recent study showed sex-related dichotomies between innate and adaptive responses in the blood and correlated them to clinical outcomes (27). Male subjects exhibited increased chemokine (CCL5) and cytokine (IL-8, IL-18) levels, along with increased activation of non-classical monocytes; females exhibited increased and sustained T cell activation, and poor T cell activation correlated to poor disease outcomes in males (27). The contribution of immunological response mounted in women as compared to men, particularly in the respiratory system, and its association with decreased mortality requires investigation.

Aging and viral infection: Immune responses to viruses are compromised in older adults at several levels, including both innate and adaptive immune responses (28, 29). Perhaps not surprisingly, that has held true in a few studies with coronaviruses, most notably with the mouse-adapted strains of SARS-COV1 (30, 31). Our group has been at the forefront of characterization and mechanistic dissection of such defects (32-37) and we will apply that expertise to the studies herein. Of interest, relatively few studies have examined sex differences in antiviral responses and vulnerability to viruses in older humans and animals, and we will fill this gap in SA2 of this proposal.

Exosomes: Extracellular vesicles (EVs) are a heterogeneous group of bilayer membrane structures that, according to their size, shape, biogenesis, and composition, are classified into two major categories known as exosomes and micro-vesicles (MVs) (38, 39). Exosomes are particles of endosomal origin with sizes ranging from 30 to 150 nm in diameter and are generated from the internal budding of multivesicular bodies and released via exocytosis (40). MVs are exosome-like vesicles with a size ranging from 50 to 1000 nm in diameter that are released by the budding of the cell membrane (41). EVs are secreted by a variety of cell types such as mesenchymal stem cells (MSCs), T cells, B cells, and dendritic cells, and can be isolated from all biological body fluids including serum, blood, breast milk, urine, and semen (41-43). Due to their size, EVs are transported through blood and biological body fluids where they interact with target cells. EVs contain proteins, soluble factors, and microRNAs based on their cellular origin and exert biological action in target cells via both an endocrine effect on distant cells and a paracrine effect on adjacent cells (44).

EVs facilitate viral and bacterial pathogenesis through their cargo of microRNA: Viral and bacterial pathogens can subvert exosome functions to promote pathogen replication, survival, or pathology. Cells employ EVs to transfer regulatory factors that modulate the response of local and distant cells and systemic responses. In cells with active viral or bacterial infections, the exosome machinery can package pathogen-derived factors that alter the phenotype of the recipient cells (5, 6). The major functions of viral miRNAs (carried by EVs) across divergent virus families have been broadly attributed to immune evasion, autoregulation of the viral life cycle and tumorigenesis (45). Dysregulation of miRNAs, including increased miR-155 expression, enhanced host innate immunity and promoted T cell differentiation in human T-cell lymphotropic virus type 1(HTLV-1) infection (46). miRNAs dysregulated by influenza virus infection also exerted an effect on virus replication and host immune responses.

INNOVATION: To our knowledge, this is the first study to focus and determine mechanisms regarding sex disparity in an age- and sex-associated viral lung disease, COVID-19.

There are two novel aspects to this proposal:

Preliminary data in this proposal (Aim 1) suggest that disease phenotype can be conferred by exosomes. We also show that this is easily assessed in lung punches. We will utilize this combination to dissect the role of gonadal hormones and their respective receptors in discovery of sex disparities in COVID-19 mortality.

This proposal will utilize the FCG mouse model to investigate the contribution of sex chromosomes to viral lung disease. Results from the FCG mice experiments will lead to further studies to find the X or Y genes responsible for the sex chromosome effect, and to study how those gene(s) interact with sex steroid-sensitive pathways to affect disease. Thus, we are breaking down sex into its component parts, to be studied one by one, to build pathways of regulation of disease. We believe that results found in mice will be most easily translated to humans when specific X or Y genes, which are common to human and mouse sex chromosomes, are discovered in mice to generate hypotheses for their action in human. Examining sex effects in this way and in Aim 2 is innovative for the viral field and a rigorous next step toward understanding the increased male mortality in COVID-19.

### Preliminary Data:

We have previously shown that exosomes can be isolated from diverse biofluids: Serum-derived and cell derived exosome isolation was performed by Zen Bio Inc (Research Triangle, NC). Exosome size from blood and cells was approximately 30-150 μm. From 10 ml of serum, we isolate between 10¹⁰-10¹² particles of exosomes. This provides enough material from the same patient for the experiments outlined in SA1 and 2. EM was performed on isolated exosomes (FIG. 5A). CD63, an exosome marker, was confirmed by Western analysis (FIG. 5B).

All aims in this proposal will utilize exosomes isolated from male and female patient serum. Patient samples will be matched for age and disease severity as outlined in Table 11, below and are available in the UA Biobank.

Exosomes derived from lungs of patients with and without lung disease enter alveolar epithelial cells. To visualize whether exosomes were present after injection into lung punches, we utilized Transmission electron microscopy (TEM). TEM revealed exosomes labeled with gold nanoparticles in alveolar (AEC) type I and type II cells (red arrows, FIG. 7) as noted by a pulmonary pathologist (Dr. Shahzeidi, personal communication). We also noted increased collagen bands (panels B and C) in those punches injected with exosomes from patients with lung disease which were not seen in punches injected with control exosomes (panel A).

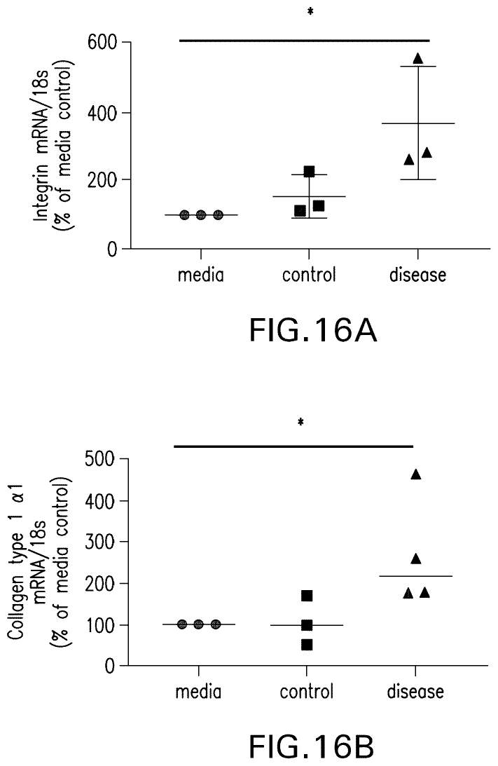

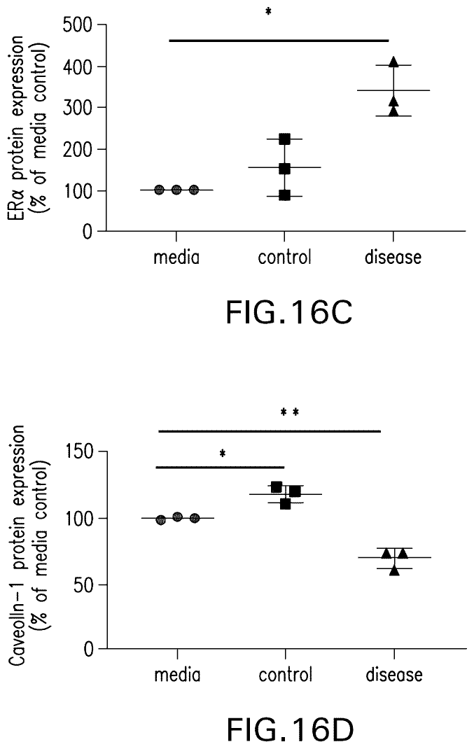

We have also previously shown that exosomes confer disease to lung punches via miRNAs. Lung punches from a healthy 15 month old male mouse were treated with exosomes derived from lung cells with and without fibrotic lung disease. As shown in FIG. 16, the diseased exosomes transferred the diseased phenotype characterized by increased integrin (FIG. 16A), collagen type I mRNA (FIG. 16B), increased ERa (FIG. 16C) and decreased Cav-1 (FIG. 16D) protein.

Statistical analysis for in vitro and in vivo studies will be as follows: data will be expressed as mean±SE. For single time point studies, unpaired Student's t test will be used for comparison between two groups, while 1-way ANOVA followed by Tukey post-hoc test will be used for multiple comparisons. For all studies statistical significance is defined as p<0.05. For in vivo experiments, as outlined in Vertebrate Animals, the number of mice in each group will be selected based on power calculations. All assays will be performed with a minimum of 3 biological replicates/sex, three technical triplicates, and at least three independent experiments. Data will be analyzed by investigators blinded to experimental group.

### Research Strategy

Sex as a biological variable: We include equal numbers of each sex in every experiment with subset analyses. Age as a biological variable: Since the mortality of COVID-19 is higher in patients >50, we will compare exosomes from adult (21-40 years old) and older (>50) patients to evaluate age-related biological differences in males and females.

### Aim 1: Determine the Contribution of Hormone Receptor Expression and Subsequent Downstream Signaling in Sex Disparity of COVID-19 Associated Lung Disease.

Rationale: Males are disproportionately dying of COVID-19 infections (http://globalhealth5050.org/covid19; accessed Jun. 13, 2020), while the incidence and prevalence of infection is equal between males and females (1, 2). This is particularly evident in cohorts over the age of 50. Gonadal hormones regulate normal lung development, physiology, and are implicated in several lung diseases including asthma, pulmonary fibrosis, and pulmonary hypertension in males and females (21, 22). In addition, since the lung is primarily affected by SARS-CoV2, in this aim we will investigate the effect of steroid hormone receptor signaling that may be stimulated differently in the male and female lung by exosomes derived from male and female patients with COVID-19 that will confer disease to the punch.

Experimental design: We will use lung punches from young adult (3-month-old C57BL6 male and female mice transgenic for human ACE2 molecule (B6.Cg-Tg(K18-ACE2)2Prlmn/J, Jax #034860). This is necessary because mice are not susceptible to SARS-COV2 unless the human ACE2 molecule is present in their cells via transgenesis or transduction. The K18-hACE2 Tg mice are breeding in the Nikolich lab since June 2020 and we do not foresee any problem with generating the numbers outlined below. We will isolate exosomes from n=3 male and n=3 female patients diagnosed with COVID-19 from each of the following groups: 21-40 years old with severe (hospitalized to Intensive Care Unit—ICU) COVID-19—adult severe (AS); >50 years of age with severe COVID-19—older severe (OS); 21-40 years old with moderate/mild (not hospitalized) COVID-19—adult moderate (AM); >50 years of age with moderate/mild COVID-19—older moderate (OM), for a total of 24 serum samples, isolated within the first two weeks of infection. Exosomes will be extracted from each serum sample, and samples paired so that one pairwise comparison is made between one female and one male exosome sample within the same group (e.g. one male and one female AS (or OS, or AM or OM) donor will form the AS comparison set 1, (FIG. 15) etc. Each exosome “set” will be incubated with three technical replicate punch biopsies from the same mouse; and with three biological punch replicates (each from a different mouse, Table 11 below.

TABLE 9
Experiment matrix. Note that each exosome group contained a total
of 81 mice.

| | Male | Female | | | M + GD | | M + GDX + | F + GDX | |
|---|---|---|---|---|---|---|---|---|---|
| | (M) | (F) | M + GD | F + GD | X + | F + GD | DHT + | + | Placebo |
| Exosome type | intact) | intact | X | X | DHT | X + E2 | Flutamide | E2 + ICI | control |
| 21-40 years | 9 | 9 | 9 | 9 | 9 | 9 | 9 | 9 | 9 |
| (Adult) | | | | | | | | | |
| Severe (AS) | | | | | | | | | |
| Male and | | | | | | | | | |
| Female | | | | | | | | | |
| >50 years | 9 | 9 | 9 | 9 | 9 | 9 | 9 | 9 | 9 |
| (Older, O); | | | | | | | | | |
| Severe (OS), | | | | | | | | | |
| Male and | | | | | | | | | |
| Female | | | | | | | | | |
| 21-40 years | 9 | 9 | 9 | 9 | 9 | 9 | 9 | 9 | 9 |
| (adult) | | | | | | | | | |
| Moderate/mild; | | | | | | | | | |
| Female | | | | | | | | | |
| >50 years | 9 | 9 | 9 | 9 | 9 | 9 | 9 | 9 | 9 |
| (older, O); | | | | | | | | | |
| Moderate/mild; | | | | | | | | | |
| Male and | | | | | | | | | |
| Female | | | | | | | | | |

Because one mouse can give us approximately nine equivalent lung punches from each mouse, we will get both biological and technical replicates in each group (3 punches will be vehicle control). Groups of mice vs exosome sets are shown in Table 11. Our preliminary data show that 3D lung punch model (47) recapitulates cellular and tissue interplay in the lung (47, 48), recognizing that they lack immune recruitment and systemic perfusion. Lungs from 3-month-old mice will be instilled with warm agarose at the pressure of 25 cm H2O. The trachea will be ligated just caudal to the larynx and the contents of thorax removed as one unit to maintain airways and alveolar integrity. Lung segments will be cooled on ice for 30 min to allow solidification of the agarose. Using a biopsy punch device, punches (4 mm) (FIG. 12) will be transferred to an air-liquid interface. Punches will be infected with 200 plaque-forming units of SARS-COV2 strain WA-1/2020. Six hours later, 10E10 exosomes will be injected around the punch followed by collection four days later. Punches will be embedded, cut and stained with trichrome as shown in FIG. 5., where we validated preserved normal architecture of a 15 mo old mouse as assessed by EM examination by a pathologist (FIG. 12). Assessments: We will leverage this method to determine the effect of the patient exosomes on the lung. 1) We will measure ACE2, ER subtype and androgen receptor (AR) mRNA and protein expression in the punch 2) In parallel we will measure metrics of viral infection: virus titers and 3) induction of type I interferon (via CXCL10 production) and of other cytokines and chemokines (TNFα; IFNg; IL-1b, IL-IRA; IL-2, IL-4; IL-6, IL-8, IL-10, IL-12, 13, GM-CSF, G-CSF, CCL2, 3, 5, CXCL5 and others by Bioplex (BioRad, Hercules CA) (27, 49). Final selection of cytokines and chemokines will be guided by the observed sex differences in COVID-19 patients (27). Finally, we will assess associated molecular markers and downstream pathways of ER and AR induced relevant pathways of infection (e.g. TIMPRSS). Histology (assessed by a pulmonary pathologist blinded to the groups) and immunohistochemistry of the lung will be assessed (epithelial lung markers; SPC and AQ5, αSMC-actin and endothelial lung marker).

Expected results/Alternative outcomes/Potential difficulties: We expect that exosomes from severely sick older males may modulate innate immune defenses of lung epithelia adversely, and that they will be either quantitatively or qualitatively worse for the host lower chemokine induction, increased inflammation (increased IL-6 and 8) compared to older females, younger males, and in particularly, young adult females. Clearly, divergent results will lead us to modify our hypotheses but will nonetheless begin to establish the basis of sex differences in susceptibility to SARS-COV2 across ages. We recognize the difficulty we may have in “matching” male and female patient exosome preps since we do not have viral load of patients at the time of collection. However, because we will be studying the impact of exosomes on uniform exposure to viral infection of the punch (at a fixed virus infection in vitro), that will be of lesser concern. Although we determined the number of particles necessary to confer disease in vivo and in lung punches, it is possible we may have to perform a dose response with one set of exosome preps. To ensure we have enough exosomes for both SA1 and 2, we will isolate exosomes from at least 2 samples of serum/patient. We have also elected early times post infection (first 10 days) for exosome harvest, expecting that early changes set up the sex-related pathogenesis. Depending on initial results, this timeline may have to be adjusted subsequently. We have preliminary data that hormone receptors in lung punches are regulated by exosome treatment (FIG. 16), therefore we expect that punch sex steroid hormone receptor expression will be regulated by COVID exosomes in a sex dependent manner. Once we have determined “background hormone receptor expression”, we will perform GDX on mice so that females lack estrogens and males lack androgens (Table 11). After 2 weeks we will perform lung punches and treat them with exosomes. If these experiments result in assessment changes, we will stimulate GDX mice with E2 or DHT pellets. We will also block these treatments with the complete ER antagonist ICI 182, 780 (0.1 mg/pellet) or AR antagonist (Flutamide, 25 mg/pellet) prior to using the mice for punches, to target the respective receptor and demonstrate specificity of receptor binding. Lung punches will then be performed and infection and treatment with exosomes will ensue. The activation of AR, as well as ERs, is in part dependent on the recruitment of transcriptional modulating factors (coactivator and corepressor proteins) which interact with ligand bound receptor dimers and are critical mediators of processes necessary for gene expression such as chromatin remodeling (50). Therefore, altered expression of these positive and negative co-regulators (approximately 200) can result in significant changes in steroid receptor-mediated cell processes and may not be associated with viral pathways. These studies are beyond the aegis of this disclosure.

### Aim 2: Determine the Contribution of Sex Chromosomes to Differences in Sex Disparity in COVID-19 Lung Disease Using the Four Core Genotypes (FCG) Mouse Model.

We will compare XX and XY mice, that have the same type of gonads, to determine if sex differences after exposure to COVID-19 derived exosomes are caused in part by genes encoded by the sex chromosomes, which are inherently differently expressed in XX and XY lungs.

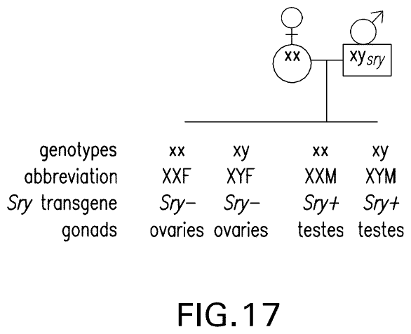

Rationale: Because females appear to be less likely to die of COVID-19, relative to males, we ask what inherently sex-biased factors might protect females or harm males. Although most sex differences have been attributed to different effects of gonadal hormones, sex differences are increasingly found to be caused also by sex-biased effects of X or Y genes (51-53). Y genes affect only males, and numerous X genes are expressed inherently higher or lower in XX than XY because they escape X inactivation or are parentally imprinted (54). This may help explain sex differences in COVID-19 mortality as the X chromosome contains a high density of immune-related genes; therefore, women generally mount stronger innate and adaptive immune responses than men (26). The ACE2 gene is located on the X chromosome, which suggests that women might have higher ACE2 levels and thus be protected against more severe disease compared to men (55). We will test our hypothesis using the FCG mice. Because the sex of the gonads is no longer based on sex chromosome complement (XX vs. XY), The FCG model produces XX and XY gonadal males (XXM, XYM), and XX and XY gonadal females (XXF, XYF) (FIG. 17). We hypothesize that XX and XY mice (both gonadal males and gonadal females) will differ in their lung response to male or female COVID exosomes. However, the model also tests for the effects of gonadal hormones, in further support of SA1, by comparing gonadal males and females XX, or XY.

Experimental Design: We have an active breeder colony of FCG mice (Table 12).

TABLE 12
Description of mouse for lung punches

| Description of mouse for lung punches | Total mouse number |
|---|---|
| XX Male | 9 |
| XY Male | 9 |
| XXY Female | 9 |
| XY Female | 9 |
| XX Male GDX | 9 |
| XY Male GDX | 9 |
| XX Female GDX | 9 |
| XY Female GDX | 9 |

Because the FCG mice would not be susceptible to SARS-COV2, before taking the biopsies, we will infect them with the adeno-associated virus (AAV) encoding human ACE2 (AAV-hACE2), as described in (27, 34). Four days later, punch biopsies would occur, infected with SARS-CoV2, and examined as in SA1. To confirm whether or not there is hormonal involvement, 3 month old mice will be GDX and lung punches performed. We will inject a set of male and female human COVID exosomes per mouse and test each set in punches from 3 mice. We will perform all assessments on the punches as described in SA 1. Time and resources-permitting, we will next use the exosomes that produce the greatest sex difference and test them in a full in vivo SARS-COV2 infection model. Exosomes will be injected IV into XX and XY male and female mice day 4 after induction of hACE2 in their lungs (AAV-hACE2 virus). Animals will be infected with SARS-CoV2 6 h later, and their ability to clear the virus from the lungs (viral titers), weight, temperature, activity and 30-d mortality will be followed. This same group will be bled every 7 days to assess antibodies against spike RBD, S2 and neutralizing Ab, using our newly developed serological assays described in (56). A cross-sectional cohort of 6 mice/group will be sacrificed on d10 to evaluate T cell responses as in (56) and in our prior work (32-37)-we will use a 30-color spectral flow cytometry and stimulation with SARS-COV2 overlapping peptide pools to detect and compare CD4 and CD8 T cell responses. These experiments will begin to address whether exosomes directly modulate SARS-COV2 pathogenicity in mice in a sex-dependent manner, and whether that modulation may involve modulation of immunity. Histology and other assessments will be performed as described in SA1.

Expected outcomes and future studies: We may find no difference among groups that differ in sex chromosome complement with the same type of gonads (XXF=XYF; XXM=XYM), but that gonadal males have more severe outcome after male-derived exosomes than gonadal females (XXM>XXF; XYM>XYF). That outcome would cause us to focus exclusively on understanding the protective or harmful effects of gonadal hormones. Alternatively, we may find that XX mice have worse or better outcomes than XY independent of the gonadal sex (XXF< or >XYF; XXM< or >XYM), from which we would conclude that X or Y genes contribute to sex differences in viral outcomes. Because of the male predominance of in COVID-19 mortality, we might expect that XY mice fare worse than XX. However, in other disease models such as obesity and ischemic cardiovascular disease (51, 52, 57), the effects of gonadal hormones counteract the effects of sex chromosome complement. For example, gonadal males weigh more than gonadal females, but XX weigh more than XY. Thus, sex differences in hormones and chromosomes can reduce the effects of each other. We believe that results found in mice will be most easily translated to humans when specific X or Y genes, which are common to human and mouse sex chromosomes, are discovered in mice to formulate hypotheses for their action in human. These genes and their downstream pathways may become targets for novel therapies.

Timeline: SA1 experiments will begin immediately and will proceed for 18-20 months. In vitro part of SA2 will also start immediately and proceed for two years; as the in vivo part of SA2 builds on initial results from SA1 as well as the in vitro results from SA2, it will start as soon as the first data from these two experimental sets become available, most likely 4-6 months from the start of the project, and will continue to the end of Y2.

Long-term goals and perspectives: Our unique and transdisciplinary collaboration on the above experimental plan will yield new and important insights to begin to unravel the impact of sex hormones and genes on epithelial lung physiology, innate cellular defenses in alveolar epithelium and, if possible, whole animal immune defense and antiviral resistance. Results from these experiments will allow us to generate refined, specific hypotheses and will pave the way to in-depth, mechanistic and whole animal studies of sex, aging, hormonal signaling and immunity in response to the NIAID PAR-20-178 or similar initiatives.

### REFERENCES CITED

1. Gebhard C, Regitz-Zagrosek V, Neuhauser H K, Morgan R, Klein S L. Impact of sex and gender on COVID-19 outcomes in Europe. Biol Sex Differ 2020; 11: 29.
2. Guan W J, Ni Z Y, Hu Y, Liang W H, Ou C Q, He J X, Liu L, Shan H, Lei C L, Hui D S C, Du B, Li L J, Zeng G, Yuen K Y, Chen R C, Tang C L, Wang T, Chen P Y, Xiang J, Li S Y, Wang J L, Liang Z J, Peng Y X, Wei L, Liu Y, Hu Y H, Peng P, Wang J M, Liu J Y, Chen Z, Li G, Zheng Z J, Qiu S Q, Luo J, Ye C J, Zhu S Y, Zhong N S, China Medical Treatment Expert Group for C. Clinical Characteristics of Coronavirus Disease 2019 in China. N Engl J Med 2020; 382: 1708-1720.
3. Klein S L, Flanagan K L. Sex differences in immune responses. Nat Rev Immunol 2016; 16: 626-638.
4. Robinson D P, Huber S A, Moussawi M, Roberts B, Teuscher C, Watkins R, Arnold A P, Klein S L. Sex chromosome complement contributes to sex differences in coxsackievirus B3 but not influenza A virus pathogenesis. Biol Sex Differ 2011; 2: 8.
5. Anderson M R, Kashanchi F, Jacobson S. Exosomes in Viral Disease. Neurotherapeutics 2016; 13: 535-546.
6. Schorey J S, Harding C V. Extracellular vesicles and infectious diseases: new complexity to an old story. J Clin Invest 2016; 126: 1181-1189.
7. Umetani M, Domoto H, Gormley A K, Yuhanna I S, Cummins C L, Javitt N B, Korach K S, Shaul P W, Mangelsdorf D J. 27-Hydroxycholesterol is an endogenous SERM that inhibits the cardiovascular effects of estrogen. Nat Med 2007; 13: 1185-1192.
8. Umetani M, Ghosh P, Ishikawa T, Umetani J, Ahmed M, Mineo C, Shaul P W. The cholesterol metabolite 27-hydroxycholesterol promotes atherosclerosis via proinflammatory processes mediated by estrogen receptor alpha. Cell Metab 2014; 20: 172-182.
9. Doublier S, Lupia E, Catanuto P, Elliot S J. Estrogens and progression of diabetic kidney damage. Curr Diabetes Rev 2011; 7: 28-34.
10. Elliot S J, Karl M, Berho M, Potier M, Zheng F, Leclercq B, Striker G E, Striker L J. Estrogen deficiency accelerates progression of glomerulosclerosis in susceptible mice. Am J Pathol 2003; 162: 1441-1448.
11. Elliot S J, Karl M, Berho M, Xia X, Pereria-Simon S, Espinosa-Heidmann D, Striker G E. Smoking induces glomerulosclerosis in aging estrogen-deficient mice through cross-talk between TGF-beta1 and IGF-I signaling pathways. J Am Soc Nephrol 2006; 17: 3315-3324.
12. Karl M, Berho M, Pignac-Kobinger J, Striker G E, Elliot S J. Differential effects of continuous and intermittent 17beta-estradiol replacement and tamoxifen therapy on the prevention of glomerulosclerosis: modulation of the mesangial cell phenotype in vivo. Am J Pathol 2006; 169: 351-361.
13. Nelson A W, Tilley W D, Neal D E, Carroll J S. Estrogen receptor beta in prostate cancer: friend or foe? Endocr Relat Cancer 2014; 21: T219-234.
14. Siegfried J M, Stabile L P. Estrongenic steroid hormones in lung cancer. Semin Oncol 2014; 41: 5-16.
15. Glassberg M K, Catanuto P, Shahzeidi S, Aliniazee M, Lilo S, Rubio G A, Elliot S J. Estrogen deficiency promotes cigarette smoke-induced changes in the extracellular matrix in the lungs of aging female mice. Transl Res 2016; 178: 107-117.
16. Glassberg M K, Choi R, Manzoli V, Shahzeidi S, Rauschkolb P, Voswinckel R, Aliniazee M, Xia X, Elliot S J. 17beta-estradiol replacement reverses age-related lung disease in estrogen-deficient C57BL/6J mice. Endocrinology 2014; 155: 441-448.
17. Triebner K, Matulonga B, Johannessen A, Suske S, Benediktsdottir B, Demoly P, Dharmage S C, Franklin K A, Garcia-Aymerich J, Gullon Blanco J A, Heinrich J, Holm M, Jarvis D, Jogi R, Lindberg E, Moratalla Rovira J M, Muniozguren Agirre N, Pin I, Probst-Hensch N, Puggini L, Raherison C, Sanchez-Ramos J L, Schlunssen V, Sunyer J, Svanes C, Hustad S, Leynaert B, Gomez Real F. Menopause Is Associated with Accelerated Lung Function Decline. Am J Respir Crit Care Med 2017; 195: 1058-1065.
18. Vermeulen A, Kaufman J M, Goemaere S, van Pottelberg I. Estradiol in elderly men. Aging Male 2002; 5: 98-102.
19. Libert C, Dejager L, Pinheiro I. The X chromosome in immune functions: when a chromosome makes the difference. Nat Rev Immunol 2010; 10: 594-604.
20. Zheng H Y, Li Y, Dai W, Wei C D, Sun K S, Tong Y Q. Imbalance of testosterone/estradiol promotes male CHD development. Biomed Mater Eng 2012; 22: 179-185.
21. Sathish V, Martin Y N, Prakash Y S. Sex steroid signaling: implications for lung diseases. Pharmacol Ther 2015; 150: 94-108.
22. Sathish V; Prakash Y. Sex differences in pulmonary anatomy and physiology: Implications for health and disease. Sex differences in physiology; 2016. p. 89-106.
23. Elliot S, Periera-Simon S, Xia X, Catanuto P, Rubio G, Shahzeidi S, El Salem F, Shapiro J, Briegel K, Korach K S, Glassberg M K. MicroRNA let-7 Downregulates Ligand-Independent Estrogen Receptor-mediated Male-Predominant Pulmonary Fibrosis. Am J Respir Crit Care Med 2019; 200: 1246-1257.
24. Alghamdi I G, Hussain, II, Almalki S S, Alghamdi M S, Alghamdi M M, El-Sheemy M A. The pattern of Middle East respiratory syndrome coronavirus in Saudi Arabia: a descriptive epidemiological analysis of data from the Saudi Ministry of Health. Int J Gen Med 2014; 7: 417-423.
25. Channappanavar R, Fett C, Mack M, Ten Eyck P P, Meyerholz D K, Perlman S. Sex-Based Differences in Susceptibility to Severe Acute Respiratory Syndrome Coronavirus Infection. J Immunol 2017; 198: 4046-4053.
26. Schurz H, Salie M, Tromp G, Hoal E G, Kinnear C J, Moller M. The X chromosome and sex-specific effects in infectious disease susceptibility. Hum Genomics 2019; 13: 2.
27. Takahashi T, Wong P, Ellingson M, Lucas C, Klein J, Israelow B, Silva J, Oh J, Mao T, Tokuyama M, Lu P, Venkataraman A, Park A, Liu F, Meir A, Sun J, Wang E, Wyllie A L, Vogels C B F, Earnest R, Lapidus S, Ott I, Moore A, Casanovas A, Dela Cruz C, Fournier J, Odio C, Farhadian S, Grubaugh N, Schulz W, Ko A, Ring A, Omer S, Iwasaki A. Sex differences in immune responses to SARS-CoV-2 that underlie disease outcomes. medRxiv 2020: 2020. 2006.2006.20123414.
28. Montgomery R R, Shaw A C. Paradoxical changes in innate immunity in aging: recent progress and new directions. J Leukoc Biol 2015; 98: 937-943.
29. Nikolich-Zugich J. The twilight of immunity: emerging concepts in aging of the immune system. Nat Immunol 2018; 19: 10-19.
30. Rockx B, Baas T, Zornetzer G A, Haagmans B, Sheahan T, Frieman M, Dyer M D, Teal T H, Proll S, van den Brand J, Baric R, Katze M G. Early upregulation of acute respiratory distress syndrome-associated cytokines promotes lethal disease in an aged-mouse model of severe acute respiratory syndrome coronavirus infection. J Virol 2009; 83: 7062-7074.
31. Sheahan T, Whitmore A, Long K, Ferris M, Rockx B, Funkhouser W, Donaldson E, Gralinski L, Collier M, Heise M, Davis N, Johnston R, Baric R S. Successful vaccination strategies that protect aged mice from lethal challenge from influenza virus and heterologous severe acute respiratory syndrome coronavirus. J Virol 2011; 85: 217-230.
32. Brien J D, Uhrlaub J L, Hirsch A, Wiley C A, Nikolich-Zugich J. Key role of T cell defects in age-related vulnerability to West Nile virus. J Exp Med 2009; 206: 2735-2745.
33. Cicin-Sain L, Brien J D, Uhrlaub J L, Drabig A, Marandu T F, Nikolich-Zugich J. Cytomegalovirus infection impairs immune responses and accentuates T-cell pool changes observed in mice with aging. PLoS Pathog 2012; 8: e1002849.
34. Li G, Smithey M J, Rudd B D, Nikolich-Zugich J. Age-associated alterations in CD8alpha+dendritic cells impair CD8 T-cell expansion in response to an intracellular bacterium. Aging Cell 2012; 11: 968-977.
35. Smithey M J, Renkema K R, Rudd B D, Nikolich-Zugich J. Increased apoptosis, curtailed expansion and incomplete differentiation of CD8+ T cells combine to decrease clearance of L. monocytogenes in old mice. Eur J Immunol 2011; 41: 1352-1364.
36. Uhrlaub J L, Pulko V, DeFilippis V R, Broeckel R, Streblow D N, Coleman G D, Park B S, Lindo J F, Vickers I, Anzinger J J, Nikolich-Zugich J. Dysregulated TGF-beta Production Underlies the Age-Related Vulnerability to Chikungunya Virus. PLoS Pathog 2016; 12: e1005891.
37. Uhrlaub J L, Smithey M J, Nikolich-Zugich J. Cutting Edge: The Aging Immune System Reveals the Biological Impact of Direct Antigen Presentation on CD8 T Cell Responses. J Immunol 2017; 199: 403-407.
38. Batsali A K, Georgopoulou A, Mavroudi I, Matheakakis A, Pontikoglou C G, Papadaki H A. The Role of Bone Marrow Mesenchymal Stem Cell Derived Extracellular Vesicles (MSC-EVs) in Normal and Abnormal Hematopoiesis and Their Therapeutic Potential. J Clin Med 2020; 9.
39. Dilsiz N. Role of exosomes and exosomal microRNAs in cancer. Future Sci OA 2020; 6: FSO465.
40. Rani S, Ryan A E, Griffin M D, Ritter T. Mesenchymal Stem Cell-derived Extracellular Vesicles: Toward Cell-free Therapeutic Applications. Mol Ther 2015; 23: 812-823.
41. Giebel B, Kordelas L, Börger V. Clinical potential of mesenchymal stem/stromal cell-derived extracellular vesicles. Stem Cell Investig 2017; 4: 84-84.
42. Colombo M, Raposo G, Théry C. Biogenesis, Secretion, and Intercellular Interactions of Exosomes and Other Extracellular Vesicles. Annual Review of Cell and Developmental Biology 2014; 30: 255-289.
43. Keller S, Ridinger J, Rupp A-K, Janssen J W G, Altevogt P. Body fluid derived exosomes as a novel template for clinical diagnostics. J Transl Med 2011; 9: 86-86.
44. Lou G, Chen Z, Zheng M, Liu Y. Mesenchymal stem cell-derived exosomes as a new therapeutic strategy for liver diseases. Experimental & Molecular Medicine 2017; 49: e346-e346.
45. Mishra R, Kumar A, Ingle H, Kumar H. The Interplay Between Viral-Derived miRNAs and Host Immunity During Infection. Frontiers in Immunology 2020; 10.
46. Bellon M, Lepelletier Y, Hermine O, Nicot C. Deregulation of microRNA involved in hematopoiesis and the immune response in HTLV-I adult T-cell leukemia. Blood 2009; 113: 4914-4917.
47. Zscheppang K, Berg J, Hedtrich S, Verheyen L, Wagner D E, Suttorp N, Hippenstiel S, Hocke A C. Human Pulmonary 3D Models For Translational Research. Biotechnol J 2018; 13.
48. Alsafadi H N, Uhl F E, Pineda R H, Bailey K E, Rojas M, Wagner D E, Königshoff M. Applications and Approaches for Three-Dimensional Precision-Cut Lung Slices. Disease Modeling and Drug Discovery. Am J Respir Cell Mol Biol 2020; 62: 681-691.
49. Zhao Y, Qin L, Zhang P, Li K, Liang L, Sun J, Xu B, Dai Y, Li X, Zhang C, Peng Y, Feng Y, Li A, Hu Z, Xiang H, Ogg G, Ho L-P, McMichael A, Jin R, Knight J C, Dong T, Zhang Y. Longitudinal COVID-19 profiling associates IL-1RA and IL-10 with disease severity and RANTES with mild disease. JCI Insight 2020; 5: e139834.
50. Bulynko Y A, O'Malley B W. Nuclear receptor coactivators: structural and functional biochemistry. Biochemistry 2011; 50: 313-328.
51. Arnold A P, Cassis L A, Eghbali M, Reue K, Sandberg K. Sex Hormones and Sex Chromosomes Cause Sex Differences in the Development of Cardiovascular Diseases. Arterioscler Thromb Vasc Biol 2017; 37: 746-756.
52. Chen X, McClusky R, Chen J, Beaven S W, Tontonoz P, Arnold A P, Reue K. The number of x chromosomes causes sex differences in adiposity in mice. PLoS Genet 2012; 8: e1002709.
53. Smith-Bouvier D L, Divekar A A, Sasidhar M, Du S, Tiwari-Woodruff S K, King J K, Arnold A P, Singh R R, Voskuhl R R. A role for sex chromosome complement in the female bias in autoimmune disease. J Exp Med 2008; 205: 1099-1108.
54. Arnold A P. A general theory of sexual differentiation. J Neurosci Res 2017; 95: 291-300.
55. Bhatia K, Zimmerman M A, Sullivan J C. Sex Differences in Angiotensin-Converting Enzyme Modulation of Ang (1-7) Levels in Normotensive W K Y Rats. American Journal of Hypertension 2013; 26: 591-598.
56. Rippinger T W, M.; Wong, R.; Uhrlaub, J. L.; Castaneda, Y.; Pizzato, H.; Thompson, M. R.; Bradshaw, C.; Erickson, H. L.; Weinkauf, C. C.; Bime, C.; Know, K.; Bixby, B.; Dake, M. D.; Parthasarthy, S.; Edwards, T.; Kaplan, M. E.; Scott, S. J.; Sprissler, R.; Nikolich-Zuglich, J.′; Bhattacharya, D. Detection, prevalence and duration of humoral responses to SARS-CoV2 under conditions of limited population exposure (In press).
57. Li J, Chen X, McClusky R, Ruiz-Sundstrom M, Itoh Y, Umar S, Arnold A P, Eghbali M. The number of X chromosomes influences protection from cardiac ischaemia/reperfusion injury in mice: one X is better than two. Cardiovasc Res 2014; 102: 375-384.

### Example 5. Production of Exosomes in a Large-Scale Bioreactor Platform

Process development runs in which exosome products have been successfully produced and tested using a large scale, bioreactor method have been completed. In short, MSC products are expanded in the 3D Bioreactor until 80% confluence. Expansion media is then washed with PBS and replaced with serum-free DMEM. Conditioned media is collected from the waste outlet of the bioreactor and subjected to ultracentrifugation for exosome precipitation. This collection procedure is performed daily for up to 96 hours. Nanosight nanoparticle tracking analysis reveals the product to have a mean particle size of 101.2±7.5 nm, which is the expected size of exosomes. The protocol consistently produces high concentrations and total particle yields suitable for multiple clinical doses.

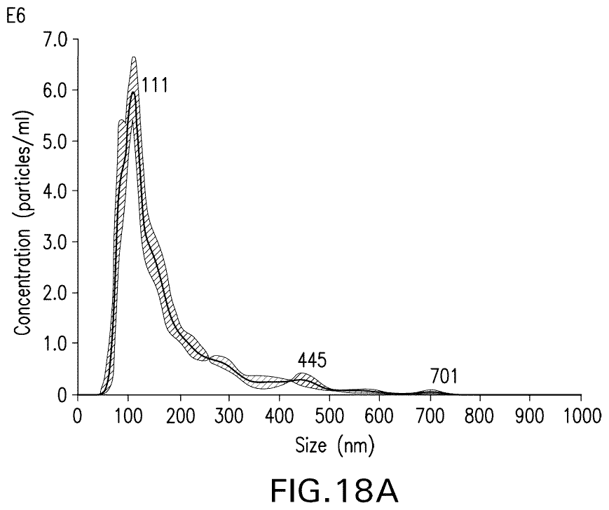

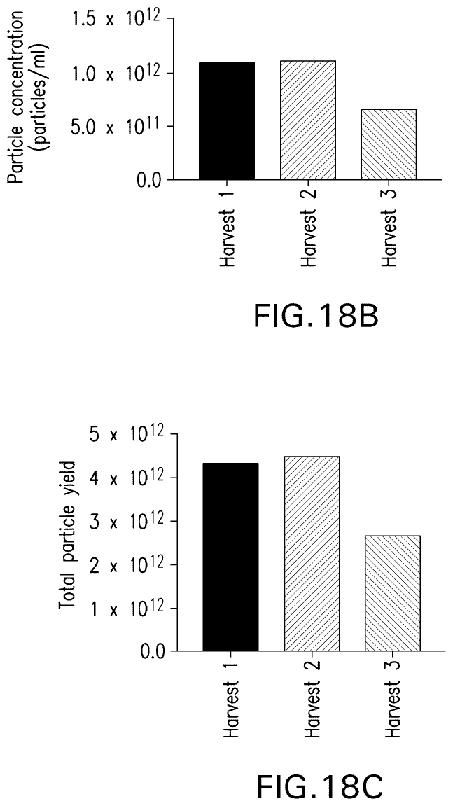

FIG. 18 shows exosome products processed from three daily conditioned media harvests that were subjected to the nanosight nanoparticle analysis to record the mean size (FIG. 18A) and the sample's particle number concentration (FIG. 18B). The particle number concentration was then multiplied by the total volume of the sample to calculate the total particle yield (FIG. 18C). Collected flow cytometry data of three independent batches shows a consistent population of CD63+, CD9+, and CD105− particles.

TABLE 13
Flow cytometry analysis of three independent batches

| | CD63 | CD9 | CD105 |
|---|---|---|---|
| Batch #1 | 92.2% | 75.5% | 0.37% |
| Batch #2 | 94.4% | 89.7% | 1.33% |
| Batch #3 | 90.9% | 61.4% | 1.50% |

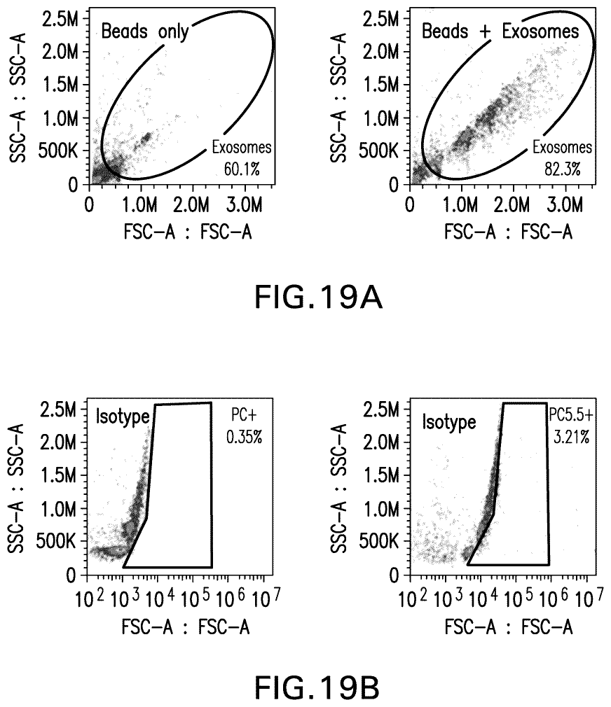

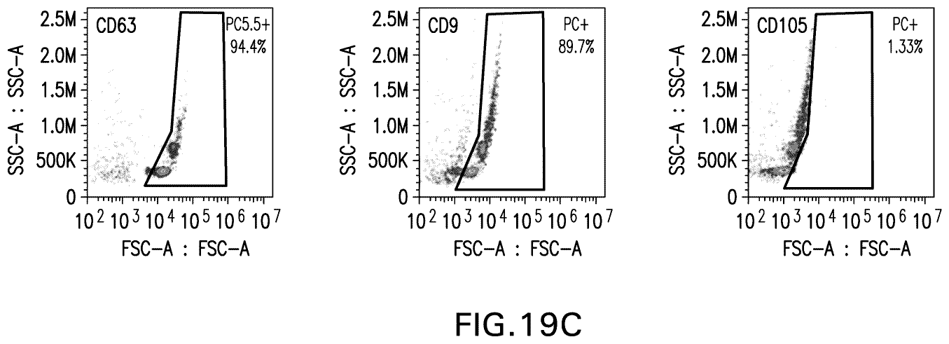

FIG. 19 shows flow cytometry characterization of exosome products. FIG. 19A shows that CD63 magnetic selection beads enabled the flow cytometry detection of exosomes from a representative product. FIG. 19B shows gating, which was based on the PE and PC5.5 isotype controls. FIG. 19C sample analysis reveals positive expression of CD63, CD9 and negative expression of CD105. Thus, initial side-by-side dot plot comparison of the beads-only negative control and the beads+exosome unstained control revealed the positive presence of CD63+ exosomes. Using isotype set gating, the exosome products were found to have high positivity of CD63 and CD9, while being negative for the MSC surface marker CD105.

### Example 6: Production and Characterization of Tuned Extracellular Vesicles Derived from Adipose-Derived Stem Cells Using a Hollow Fiber Bioreactor System

A hollow fiber bioreactor-based production system has been developed for manufacturing large quantities of extracellular vesicles (EVs) containing exosomes from adult human adipose-derived stem cells (ASCs) as described in Ludlow, J. W., Buehrer, B. M. (2024). Analyses and Utilization of Selectively Tuned Human Adipose-Derived Stromal/Stem Cell Exosomes. In: Gimble, J., et al., Adipose-Derived Stem Cells. Methods in Molecular Biology, vol 2783.

By manipulating the cellular bioreactor environment, it has been found that ASC EV production, secretion, and surface protein composition can be altered. The methodology described herein includes culturing and tuning of adipose ASCs in a bioreactor as well as collecting and isolating populations of EVs containing exosomes demonstrating increased HSP70 content from the cultured and tuned ASCs (Ludlow, J. W., Buehrer, B. M. (2024). Analyses and Utilization of Selectively Tuned Human Adipose-Derived Stromal/Stem Cell Exosomes. In: Gimble, J., et al., Adipose-Derived Stem Cells. Methods in Molecular Biology, vol 2783).

Several studies support the hypothesis that EVs containing exosomes can mediate the pro-healing effects of stem cells in different wound-healing models [4-10]. Stem cell-derived EVs are a particularly compelling therapeutic candidate because their protein and miRNA cargos are delivered to cells directly through membrane fusion with no requirement for receptor-mediated signal transduction.

Commercial-scale stromal/stem cell-derived exosomes can be generated using a closed-system hollow fiber bioreactor capable of continuous EV production [11]. In so doing, the large quantities of EVs required for therapeutic product development can be attained in a cost-effective manner. Verification of particle size, particle concentration, and ability to deliver exosomes to cells in culture provides the basis for further therapeutic utility. In addition, manipulation of the bioreactor cell culture environment, e.g., by heat-shocking [12] results in tuning of the exosomes to express increased HSP70 protein levels compared to non-heat-shocked controls.

The steps to expand ASCs in culture to generate sufficient quantities to seed a preconditioned, hollow fiber bioreactor cartridge are described below. Continued cell growth and maintenance in the bioreactor are monitored, and the methodology to heat-shock the ASC culture and the collection of EV-containing conditioned medium for isolation of ASC exosomes through ultracentrifugation is described. Once collected, the EVs are characterized for size, concentration, and their ability to transfer their cargo to human primary fibroblasts. Additional verification of exosome surface markers by western blot analysis is further described.

### Materials

### Adult Human Primary Cells

1. Cryopreserved human primary adipose-derived stem cells (ASCs) isolated from abdominoplasty, where solid tissue is recovered. In contrast, cells isolated from liposuction involves recovery of liquefied material.
2. Human primary dermal fibroblasts (DFs) (e.g., isolated from skin explant and cryopreserved).

### Culture Plasticware

1. Sterile tissue culture flasks, 225 cm².
2. 96-well tissue culture plates.
3. Cell factories (or equivalent multitray cell culture systems) in 2-, 4-, 10-decks (meaning the number of vertical growth plates stacked) comprising a port designed to facilitate pouring and aseptic filling techniques.
4. Pipettes, 5, 10, and 25 mL volumes.
5. Centrifuge tubes, 15 and 50 mL volumes.
6. 60 mL and 3 mL Luer-Lok syringes.

### Equipment

1. Class II biosafety cabinet.
2. Hemocytometer with cover slips.
3. Centrifuge with swinging-bucket rotor.
4. Microcentrifuge.
5. Ultracentrifuge with fixed angle rotor.
6, Ultracentrifuge tubes.
7. Inverted microscope for cell culture analysis.
8. −80 °C freezer.
9. Cell culture incubator-humidified, 37 °C, and 5% CO₂/95% air.
10. Adjustable temperature water bath.
11. Zeta View® or comparable Nanoparticle Tracking Analysis (NTA) technology.
12. ArrayScan™ XTI or comparable high-content fluorescence microscope.
13. XCell SureLock™ Mini-Cell Electrophoresis System or
equivalent.
14. XCell II™ Blot Module or equivalent.
15. Power sources for electrophoresis and western blotting.
16. ChemiDoc™ XRS+ Imaging System (Bio-Rad®) or equivalent.

### Reagents

1. Vybrant™ DiD (a far-red fluorescent carbocyanine dye) cell labeling solution or equivalent.
2. NuPAGE™ 4 to 12%, Bis-Tris, 1.0 mm, Mini Protein Gels or equivalent.
3. Spectra™ Multicolor Broad Range Protein Ladder or equivalent.
4. Nitrocellulose or polyvinylidene difluoride (PVDF) membrane.
5. NuPAGE™ MES SDS Buffer Kit (for Bis-Tris Gels) or equivalent.
6. 25× Tris-Glycine Transfer Buffer.
7. XCell SureLock™ Mini-Cell Electrophoresis System or equivalent.
8. XCell II™ Blot Module or equivalent.
9 Blocking buffer: 5% bovine serum albumin (BSA) in Tris-buffered saline (TBS), 0.02% sodium azide.
10. 10×TBS, with 1% Tween 20.
11. 20×TBS Solution, pH 7.4, Molecular Biology Grade, Ultrapure.
12. Gel Loading Buffer II (Denaturing PAGE) or equivalent.
13. Western blotting filter paper, extra thick, 20 cm×20 cm.
   Hollow Fiber Bioreactor (FiberCell®) (fibercellsystems.com)
1. FiberCell® Systems Duet Pump.
2. FiberCell® Hollow Fiber Cartridge.

### Culture Media Components and Supplements

Storage conditions, shelf life, and expiration dates for all media, components, and supplements are provided by the manufacturer. All manipulations take place in a biosafety cabinet to reduce the risk of microbial contamination.
1. Dulbecco's phosphate-buffered saline (DPBS).
2. 0.25% trypsin EDTA, 1×.
3. 0.4% trypan blue solution in DPBS.
4. ASC growth medium: DMEM/F-12, 10% fetal bovine serum (FBS), 0.1% antibiotic/antimycotic. Store at 4 °C, shelf life of 1 month.
5. Serum-free ASC growth medium: DMEM/F-12, 0.1% antibiotic/antimycotic. Store at 4 °C, shelf life of 1 month.
6. EV collection medium: Phenol red-free DMEM, 0.1% antibiotic/antimycotic. Store at 4 °C, shelf life of 1 month.
7. DPBS flush medium: DPBS, 0.1% antibiotic/antimycotic. Store at 4 °C, shelf life of 1 month.
8 Dermal fibroblast growth medium: DMEM HG, 10% FBS, 0.1% antibiotic/antimycotic. Store at 4 °C, shelf life of 1 month.

### Immunoreagents

1. Anti-HSP70 antibody [5A5].
2. Anti-Actin antibody [AC-40].
3. Goat Anti-Mouse IgG H&L conjugated to alkaline phosphatase (AP).
4. Novex™ AP Mouse Chemiluminescent Detection Kit or equivalent.

### Methods

All manipulations take place in a biosafety cabinet to reduce the risk of microbial contamination.

### Adipose ASC Culture

1. Seed the culture vessels at 2000-4000 cells/cm².
2. Remove the growth medium every 2-3 days and replace with fresh medium.
3. Once the culture is 80-90% confluent, trypsinize the cells to remove from the culture vessel.
4. Stain the cells with 0.4% trypan blue solutions and count the cells using a hemocytometer and an inverted microscope.
5. Passage the cells into a new culture vessel at 2000-4000 cells/cm².
6. Repeat steps 1-5 until 0.5-1×10E+09 total live cells have been collected.

### Conditioning the Bioreactor

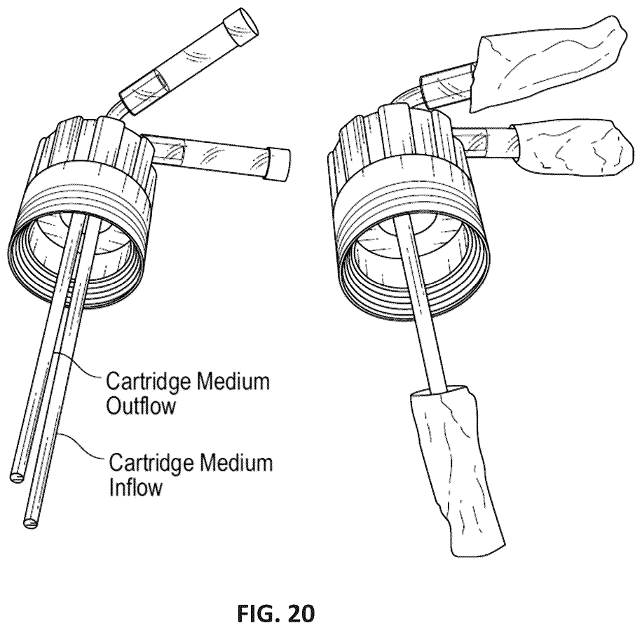

FIG. 20 shows bioreactor reservoir medium bottle tops sterilized prior to use, with foil loosely covering the inflow and outflow tube ends to maintain sterility during handling and assembly of the bioreactor.

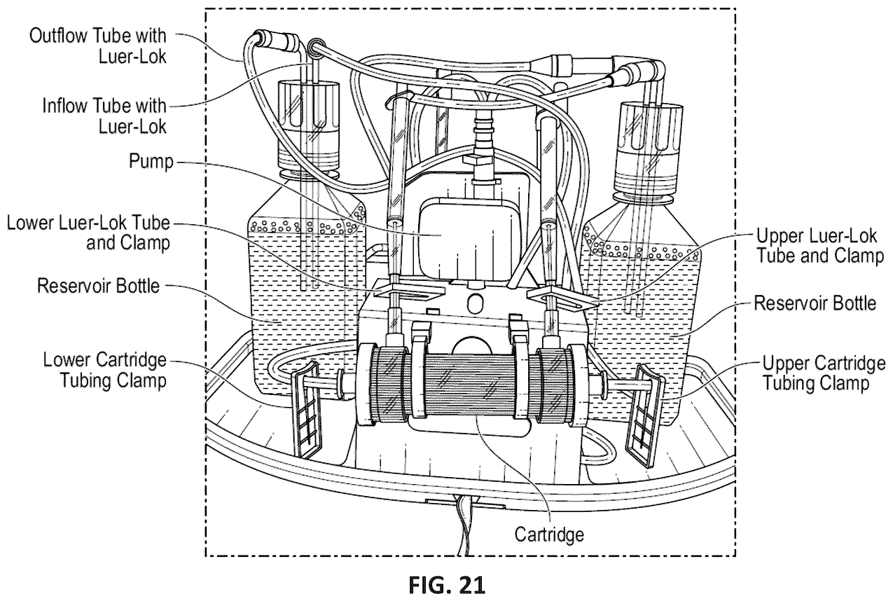

FIG. 21 shows a fully assembled bioreactor in a two-cartridge configuration, wherein the second cartridge is obscured from view by the circulating pump.
1. Clamp off both Luer-Lok-fitted tubes on top of the cartridge.
2. Attach a 1 L reservoir bottle containing DPBS flush medium (warmed to 37 °C), making sure that the cartridge medium inflow (longer of the two metal tubes) is connected to the lower cartridge tubing, and the cartridge medium outflow (shorter of the two metal tubes) is connected to the upper cartridge tubing.
3. Turn on the circulating pump (flow rate of 20-25 mL/min).
4. Allow the pump to circulate medium until air bubbles are no longer observed coming from the cartridge medium out-flow in the reservoir bottle.
5. Turn off the circulating pump.
6. Clamp off the tubing on the lower and upper ends of the cartridge.
7. Unclamp both Luer-Lok-fitted tubes on top of the cartridge.
8. Attach a 60 mL syringe containing 40 mL of DPBS flush medium (warmed to 37 °C) to the lower Luer-Lok-fitted tube on top of the cartridge.
9. Attach an empty 60 mL syringe to the upper Luer-Lok-fitted tube on top of the cartridge.
10. Slowly add 40 mL of DPBS flush medium to the cartridge by depressing the syringe plunger (air and any DPBS flush medium entering the cartridge when the circulating pump was on will be displaced into the empty 60 mL syringe attached to the upper Luer-Lok-fitted tube on top of the cartridge).
11. Gently flush the cartridge by manually pushing the DPBS flush medium back and forth between the syringes to remove any air trapped inside the cartridge.
12. Clamp both Luer-Lok-fitted tubes on top of the cartridge.
13. Optionally remove both 60 mL syringes and replace with empty 3 mL Luer-Lok syringes.
14. Turn on the circulating pump (flow rate of 20-25 mL/min).
15. Continuously flush the hollow fiber cartridge with the DPBS flush medium for 24 h in a cell culture incubator (humidified, 37 °C, 5% CO_{2/95}% air).
16. The next day, repeat steps 1-14 with 1 L of serum-free growth medium (warmed to 37 °C) for another 24 h in a cell culture incubator.
17. On day 3, repeat steps 1-14 with 1 L of growth medium warmed to 37 °C) for 24 h in a cell culture incubator.
   Seeding the Bioreactor Cartridge with Human Adipose ASCs
1. On day 4, resuspend the 0.5-1×10E+09 human ASCs in 40 mL of growth medium warmed to 37 °C.
2. Turn off circulating pump.
3. Clamp off tubing on both ends of the cartridge.
4. Unclamp both Luer-Lok-fitted tubes on top of the cartridge.
5. Attach a 60 ml syringe containing 40 mL cell suspension to the lower Luer-Lok-fitted tube on top of the cartridge.
6. Attach an empty 60 mL syringe to the upper Luer-Lok-fitted tube on top of the cartridge.
7. Slowly add 40 mL of the cell suspension by depressing the syringe plunger (air and any growth medium in the cartridge will be displaced into the empty 60 mL syringe attached to the upper Luer-Lok-fitted tube on top of the cartridge) (Since some cells may be flushed through and found in the 60 mL syringe attached to the upper Luer-Lok fitted tube on top of the cartridge, count the cells recovered during this step to calculate the total number of cells actually seeding the cartridge.
8. Clamp both Luer-Lok-fitted tubes on top of the cartridge.
9. Remove both 60 mL syringes and replace with empty 3 mL Luer-Lok syringes.
10. Replace the reservoir medium bottles with fresh medium, warmed to 37 °C.
11. Return the bioreactor to the cell culture incubator for 3 h without circulating the growth medium to allow the cells to sufficiently attach to the hollow fibers in the cartridge.
12. At the end of the 3 h incubation, start the circulating pump (flow rate of 20-25 mL/min).
13. Allow the pump to circulate for 24 h.

### Conditioned Medium Collection

1. The next day after seeding the cartridge, remove the bioreactor from the incubator, turn off the circulating pump, and clamp off the tubing on both ends of the cartridge.
2. Unclamp both Luer-Lok-fitted tubes on top of the cartridge.
3. Attach a 60 ml syringe containing 40 mL EV collection medium to the lower Luer-Lok-fitted tube on top of the cartridge.
4. Attach an empty 60 mL syringe to the upper Luer-Lok-fitted tube on top of the cartridge.
5. Heat-shocking step. Slowly add 40 mL of EV collection medium (warmed to 42 °C) by depressing the syringe plunger (air and any growth medium in the cartridge will be displaced into the empty 60 mL syringe attached to the upper Luer-Lok-fitted tube on top of the cartridge). Count the cells recovered during this step, which should be low. If heat-shocking is not desired, warm the media to 37 °C.
6. Clamp both Luer-Lok-fitted tubes on top of the cartridge.
7. Unclamp the tubing from both ends of the cartridge.
8. Remove both 60 mL syringes and replace with empty 3 mL Luer-Lok syringes.
9. Replace the reservoir medium bottles with fresh medium, warmed to 42 °C. If heat-shocking is not desired, warm the media to 37 °C.
10. Return the bioreactor to the cell culture incubator and start the circulating pump (flow rate of 20-25 mL/min).
11. On days 3-4 after adding the EV collection medium to the cartridge, turn off the circulating pump, and clamp off the tubing on both ends of the cartridge.
12. Unclamp both Luer-Lok-fitted tubes on top of the cartridge.
13. Attach a 60 mL syringe containing 40 mL of EV collection medium to the lower Luer-Lok-fitted tube on top of the cartridge.
14. Attach an empty 60 mL syringe to the upper Luer-Lok-fitted tube on top of the cartridge.
15. Slowly add fresh 40 mL of EV collection medium, warmed to 42 °C. (If heat-shocking is not desired, warm the media to 37 °C) by depressing the syringe plunger (air and EV collection medium in the cartridge will be displaced into the empty 60 ml syringe attached to the upper Luer-Lok-fitted tube on top of the cartridge).
16. Clamp both Luer-Lok-fitted tubes on top of the cartridge.
17. Remove both of the 60 mL syringes and replace with empty 3 mL Luer-Lok syringes.
18. Replace the reservoir medium bottles with fresh medium, warmed to 42 °C. (If heat-shocking is not desired, warm the media to 37 °C).
19. Unclamp the tubing on both ends of the cartridge.
20. Return the bioreactor to the cell culture incubator and start the circulating pump (flow rate of 20-25 mL/min).
21. Retain 40 mL of conditioned EV collection medium in the 50 mL syringe and transfer to a 50 mL centrifuge tube.
22. Centrifuge the conditioned EV collection medium at 300× g for 5 min to pellet unattached cells (Count cells recovered from this step, which should be low).
23. Centrifuge the clarified supernatant at 3000×g for 20 min to remove additional cellular debris and large EVs.
24. Store the recovered supernatant at −20 °C until exosome isolation.
25. Repeat steps 1-24 every 3-4 days for 6 weeks (In our experience, the exosome yield begins to decline between 6 and 8 weeks).
   Exosome Isolation from Conditioned EV Collection Medium
1. Thaw the stored conditioned medium (Although samples may be pooled for exosome isolation, monitoring exosomes isolated from each individual sample will enable determination of when the exosome production slows down).
2. Sterile filter the conditioned medium through a 0.22 μm membrane.
3. Load the necessary number of ultracentrifuge tubes.
4. Centrifuge the tubes at 100,000×g for 2 h at 4 °C.
6. Remove the supernatant and suspend the exosome pellet in about 1 mL of DPBS (Smaller volumes may be used to keep the number of exosomes/unit volume higher).
7. Collect size and quantity data using the Zeta View® nanoparticle analyzer.
8 Aliquot and store the exosomes. Storage at 4 °C is adequate for up to 1 month. For storage longer than 1 month, store the exosomes at −80 °C.

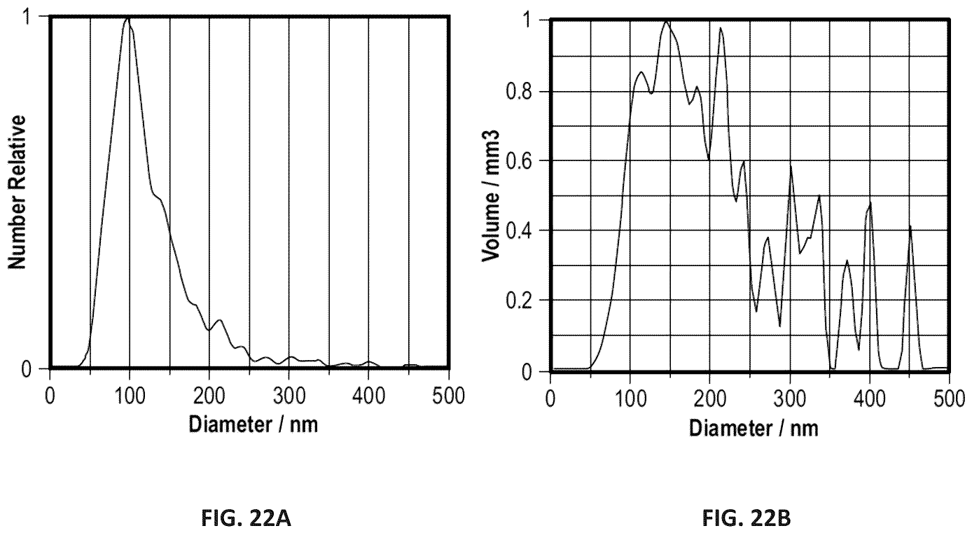

FIG. 22 and Table 14 below provide for an exemplary analysis result). Nanoparticle Tracking Analysis (NTA) is an analytical technique for measuring the size distribution and concentration of nanoparticles between 10 nm and 1500 nm in liquid suspension simultaneously. It utilizes lasers to illuminate particles, allowing a microscope-mounted camera to track their Brownian motion in real time. FIG. 22A and FIG. 22B shows Zeta View® Nanoparticle Tracking Analysis (NTA) of exosomes derived from adipose-derived stem cells (ASCs) indicating a particle diameter of 121.9 nm and a mode diameter of 97.6 nm, with a concentration of 1.3×10E+11 particles/mL. FIG. 22A shows the particle size distribution based on the relative number of particles detected at each size. FIG. 22B shows the particle size distribution weighted by volume.

TABLE 14
ZetaView ® Nanoparticle Tracking Analysis of ASC-Derived
Exosomes

| | | Full Width at Half | |
|---|---|---|---|
| | | Maximum | |
| Diameter/nm | Number Absolute | (FWHM)/nm | Percentage |
| 96.6 | 7.1E+1 | 59.2 | 92.2 |
| 209.4 | 9.2E+0 | 23.3 | 7.8 |

X values (all sizes given in nm)

| | Number | Concentration | Volume |
|---|---|---|---|
| X10 | 69.6 | 69.6 | 103.7 |
| X50 | 106.2 | 106.2 | 192.7 |
| X90 | 183.1 | 183.1 | 393.7 |
| Span | 1.1 | 1.1 | 1.5 |
| Mean | 121.9 | 121.9 | 223.2 |
| StdDev | 52.6 | 52.5 | 111.6 |

### Exosome Transfer to Dermal Fibroblasts in Culture

1. Seed human primary dermal fibroblasts at a density of 10,000 cells/cm² in each well of a 96-well plate using the dermal fibroblast growth medium.
2. Culture the cells until they are 80-90% confluent, exchanging medium every 2-3 days as necessary.
3. Stain the exosomes with the DiD labeling solution using 4 μL for every 100 μL of exosome solution and incubate at 37 °C for 30 min. DiD (1,1′-dioctadecyl-3,3,3′,3′-tetramethylindodicarbocyanine) is a lipophilic, far-red fluorescent carbocyanine dye used for cell membrane labeling, tracking, and imaging.
4. Collect the stained exosomes by ultracentrifugation at 100,000× g for 2 h. Remove the supernatant and suspend the pellet in PBS. Confirm the exosome concentration and size via Zeta View® particle analysis.
5. Add 1×10E+8 DiD-labeled exosomes to the wells containing dermal fibroblasts and 100 μL medium. It is preferable to use phenol red-free medium during treatments and image capture to reduce background). Place in the incubator for 24 h.
6. To stain the cellular nuclei, add 10 μL of 10 μg/mL Hoechst stain to each well for 1 μg/mL final concentration.
7. Live image the cells for DiD expression using an ArrayScan XTI High Content Analyzer. Use the Spot Detector BioApplication for DiD expression. Establish background detection of the vehicle control of the medium only. Capture at least 12 images (for more than 200 images per well) for each well based on random sampling. Objectives: 20× LD Plan Neofluar objective with 0.4 NA.

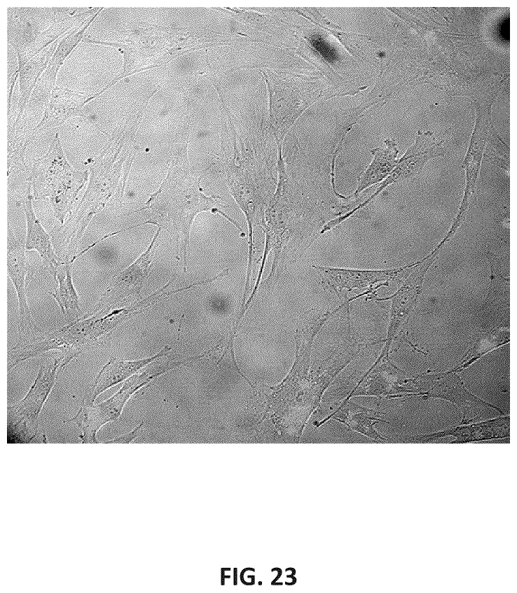

FIG. 23 is a representative microscopy image showing the transfer of DiD-stained ASC-derived exosomes to human primary dermal fibroblasts (DFs) in culture. The exosomes stained with DiD (a far-red fluorescent carbocyanine dye) were incubated with DFs for 24 hours at 37 °C, followed by nuclear staining with Hoechst to enumerate the cells. The image was collected on an ArrayScan® XTI high-content analysis system.

### SDS-PAGE and Western Blotting

1. Determine the protein concentration of the exosomal preparations using a BCA assay or comparable assay. A BCA (bicinchoninic acid) assay is a laboratory technique for determining total concentration of protein in a solution. The assay measures protein levels through a two-step colorimetric reaction. Peptide bonds in the protein reduce cupric ions (Cu²⁺) to cuprous ions (Cu⁺) in an alkaline environment. The amount of Cu⁺ produced is directly proportional to the amount of protein present. Two molecules of BCA chelate with each Cu⁺ ion, forming a highly intense, purple colored complex. The purple complex absorbs light at a wavelength of 562 nm. The amount of protein in the sample can be measured using the light absorbance compared to a standard curve of known protein concentrations.

Dilute exosome preparations 3- to 10-fold in DPBS to generate a dilution series of samples. Prepare the BCA standards as described in the assay kit (1-200 μg/mL) and add the exosome and BCA dilutions to a 96 well microplate. Add the BCA Working Reagent and incubate for 2 h at 37 °C before reading the absorbance at 562 nm. Use the BCA standard curve and exosome dilution absorbance values to back-calculate the concentration of the exosomal preparations.
2. Aliquot 30-50 μg of protein for each exosome sample to Eppendorf® tubes and add the same volume of Gel Loading Buffer, mix, and heat at 95 °C for 5 min. Cool and briefly spin down the tube contents.
3. Load the denatured samples into 4-12% precast gel wells; include the molecular weight markers on each gel.
4. Add the running buffer and separate proteins using a 200 V constant voltage.
5. Remove the gel from the plastic casing and set up for a Western transfer using a PVDF membrane and a western blotting filter paper saturated with the Transfer buffer. Place in the XCell™ Blot Module and transfer the proteins to the membrane using a constant voltage.
6. Block the transferred membrane with the BSA blocking buffer (or 3-5% nonfat dry milk) for at least 1 h at room temperature. One exchange of the buffer helps to remove any remaining transfer buffer.
7. Dilute the primary antibodies (1:1000-1:5000) into the BSA blocking buffer and add to the membrane for incubation overnight at 4 °C.
8 Wash the membranes at least five times with 1×TBST (TBS+Tween®) to remove traces of the primary antibody.
9. Dilute the secondary antibody (1:5000-1:10,000) in the BSA blocking buffer and add to the washed membrane. Incubate for 1 h at room temperature.
10. Wash the membranes again at least five times with 1×TBST to remove traces of the secondary antibody.
11. Prepare the chemiluminescent detection reagents according to the manufacturer's instructions and add to the membrane. For example, the Immun-Star™ chemiluminescent detection system (Bio-Rad®) uses the powerful CDP-Star chemiluminescent substrate and enhancer, which is activated by an alkaline phosphatase enzyme conjugate. Recall that the secondary antibody reagent (goat Anti-Mouse IgG, which binds to both the heavy (H) and light (L) chains of the mouse antibody is conjugated to alkaline phosphatase. The membrane is then treated with the chemiluminescent substrate alone for PVDF membranes. The concentrated enhancer is added to the 1×substrate solution and does not add any extra incubation or wash steps. The blot is then imaged by an imager capable of detecting chemiluminescent signals, such as the Bio-Rad® VersaDoc™ or Cheemioc™ system.
12. Quantify the results using an appropriate density analysis software.

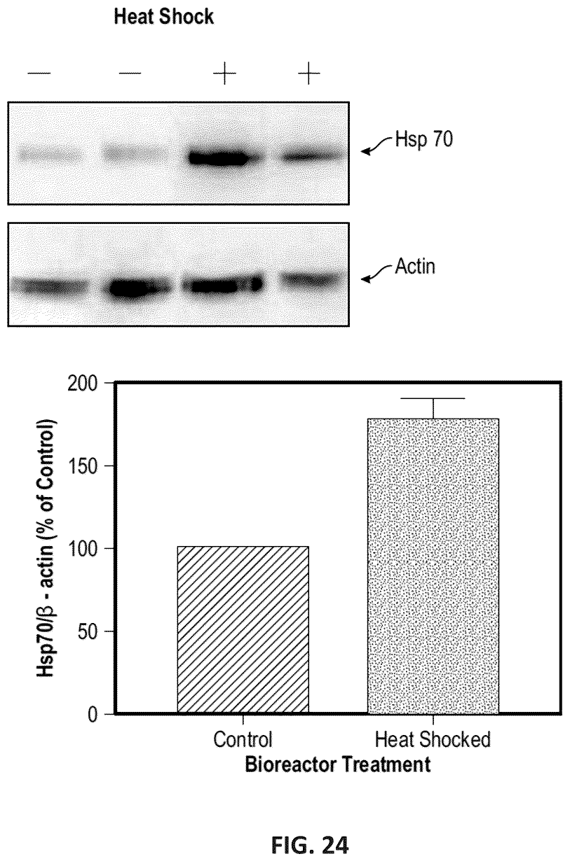

FIG. 24 is an image of a western blot and quantified western blot data demonstrating increased HSP70 protein content in exosomes isolated from a heat-shocked (42 °C) bioreactor compared to a control that was maintained at 37 °C. Actin was used as an internal control. Band intensities were measured using a ChemiDoc™ XRS+ System.

### REFERENCES

4. Basu J, Ludlow J W (2016) Exosomes for repair, regeneration and rejuvenation. Expert Opin Biol Ther 16(4):489-506.
5. Geiger A, Walker A, Nissen E (2015) Human fibrocyte-derived exosomes accelerate wound healing in genetically diabetic mice. Biochem Biophys Res Commun 467(2):303-309.
6. Guo S-C, Tao S-C, Yin W-J et al (2017) Exo-somes derived from platelet-rich plasma promote the re-epithelization of chronic cutaneous wounds via activation of YAP in a diabetic rat model. Theranostics 7(1): 81-96. PMID: 28042318.
7. Hu L, Wang J, Zhou X et al (2016) Exosomes derived from human adipose mesenchymal stem cells accelerates cutaneous wound healing via optimizing the characteristics of fibroblasts. Sci Rep 6:32993.
8. Shabbir A, Cox A, Rodriguez-Menocal L et al (2015) Mesenchymal stem cell exosomes induce proliferation and migration of normal and chronic wound fibroblasts, and enhance angiogenesis in vitro. Stem Cells Dev 24(14): 1635-1647.
9. Zhang J, Guan J, Niu X et al (2015) Exosomes released from human induced pluripotent stem cells-derived MSCs facilitate cutaneous wound healing by promoting collagen synthesis and angiogenesis. J Transl Med 13(1):49.
10. Zhou Y, Xu H, Xu W et al (2013) Exosomes released by human umbilical cord mesenchymal stem cells protect against cisplatin-induced renal oxidative stress and apoptosis in vivo and in vitro. Stem Cell Res Ther 4(2):34.
11. Whitford W, Ludlow J W, Cadwell J J S (2015) Continuous production of exosomes. Gen Eng Biotech News 35(16):34.
12. Ludlow J W, Buehrer B M (2022) Manipulating the cellular environment generates differential extracellular vesicle packaging and biological activity. In: Advances in exosome research, Biocompare ebooks, 12-17 Mar. 2022.

### Example 7: Production of Extracellular Vesicles Derived from Mesenchymal Stem Cells Using a Bioreactor

### Bioreactor Conditioning

Initially, the bioreactor (capillary cartridge) was conditioned with 500 mL of DPBS for 48 hours at room temperature. Next, the bioreactor was conditioned with 500 mL of complete culture medium (DMEM/F12 containing 10% fetal bovine serum) for 24 hours at 37 °C in a humidified, 5% CO₂ containing atmosphere.

### Cell Seeding and Growth

The bioreactor was seeded with 300×10E+6 MSCs (placental or adipose origin) and maintained in a 37 °C, humidified, 5% CO₂ containing atmosphere. Glucose levels in the medium were monitored and circulating medium was replaced with fresh medium when glucose levels were equal to or less than 150 mg/dL. Cells were allowed to grow for 2 weeks before beginning exosome harvest.

### Exosome Harvest Preparation

The bioreactor was washed 5 times with serum-free DMEM/F12 to remove exosomes, which are contained in the cell culture medium, from the bioreactor. Then, serum-free DMEM/F12 was added to the bioreactor and the circulating culture medium reservoir (DMEM/F12 containing 10% fetal bovine serum) was replaced with fresh medium. Then, the serum-free DMEM/F12 medium was recovered from the bioreactor after 48 hours and replaced with fresh medium.

This is repeated for the life of the bioreactor (approximately 3 months).

### Heat Shock Tuning

For tuning exosomes to contain increased heat shock proteins (i.e., HSP70), serum-free DMEM/F12 warmed to 41 °C was added to the bioreactor and the circulating culture medium reservoir (DMEM/F12 containing 10% fetal bovine serum) was replaced with fresh medium warmed to 41 °C. The bioreactor was then placed in a 41 °C, humidified, 5% CO₂ containing atmosphere for 1 hour.

After 1 hour of heat shocking at 41 °C, serum-free DMEM/F12 warmed to 37 °C was added to the bioreactor and the circulating culture medium reservoir (DMEM/F12 containing 10% fetal bovine serum) was replaced with fresh medium warmed to 37 °C. The bioreactor was then placed in a 37 °C, humidified, 5% CO₂ containing atmosphere. The serum free DMEM/F12 was recovered from the bioreactor after 48 hours.

This is repeated for the life of the bioreactor (approximately 3 months).

### Example 8: Optimizing the Production of Selectively-Tuned Human ASC Extracellular Vesicles for Chronic Wound Healing

A combination of heat shock (HS) and a pharmaceutical grade, lab-produced recombinant human platelet-derived growth factor-BB (PDGF-BB) (sourced at for example, Sigma-Aldrich®, R&D Systems®, ThermoFisher®) tested at two concentrations (20 ng/mL and 200 ng/mL) was found to improve the healing capacity of hASC-derived EVs in vitro and in vivo.

Heat shocking the cells by warming reservoir media in a bioreactor to 45 °C and exchanging the media in the bioreactor cartridge itself with serum free media warmed to 42 °C, resulted in exosome yields (10E+12) which far exceeded expected quantities. Two lots of EVs were produced from the bioreactor using a combination of heat shocking and 200 ng/mL of PDGF-BB. A Particle Metrix® ZetaView® for nanoparticle tracking analysis (NTA) was acquired to assess particle diameter and concentration. This device was used instead of a tunable resistive pulse sensing (TRPS) device since NTA is more sensitive.

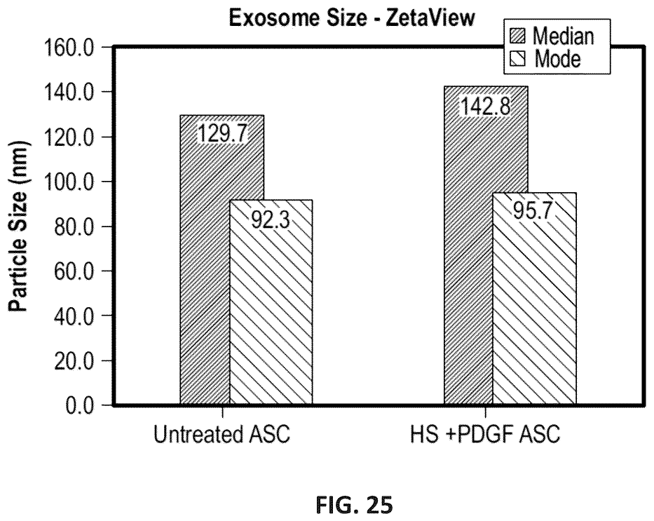

As shown in FIG. 25, EV samples from untreated and HS+200 ng/mL PDGF-treated ASCs were analyzed. The size was found to be slightly smaller than that observed using TRPS (data not shown) within the operational size defining EVs as exosomes (mean 163.6±25.4 nm and mode 116.9±11.5 nm). The difference in values between the two different technologies is attributable to NTA being an indirect measurement made by light scattering and TRPS being a direct measurement of EVs passing through a physical barrier (membrane pore).

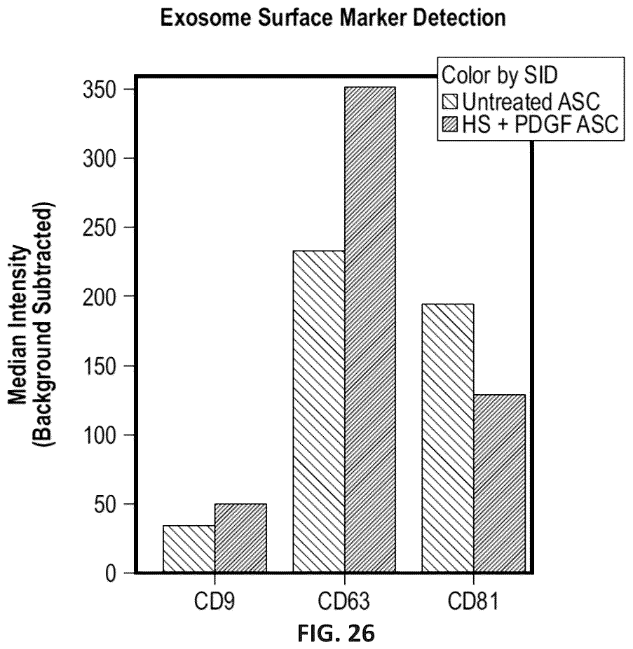

As shown in FIG. 26, tetraspanin distribution on the untreated vs. HS+200 ng/mL PDGF treated exosomes was variable. Specifically, elevation in CD9 and CD63 abundance was observed in EVs isolated from HS+200 ng/mL PDGF-BB treated cells, whereas a reduction in CD81 was observed in EVs isolated from HS+200 ng/mL PDGF-BB treated cells compared to untreated control cells (see, FIG. 26).

In a separate experiment, the effect of alternative growth factors known to affect wound healing were also tested. Adipose-derived MSCs isolated from subcutaneous adipose tissue were treated with DMEM/F12, 5% human platelet lysate (HPL), heat shock treatment, 10 μM β-lapachone, 50 ng/mL TNF-α, or 100 ng/mL epidermal growth factor (EGF). Following EV isolation, analysis was performed using MACSPlex™ Human Exosome Kit (Miltenyi Biotec) for surface antigen expression.

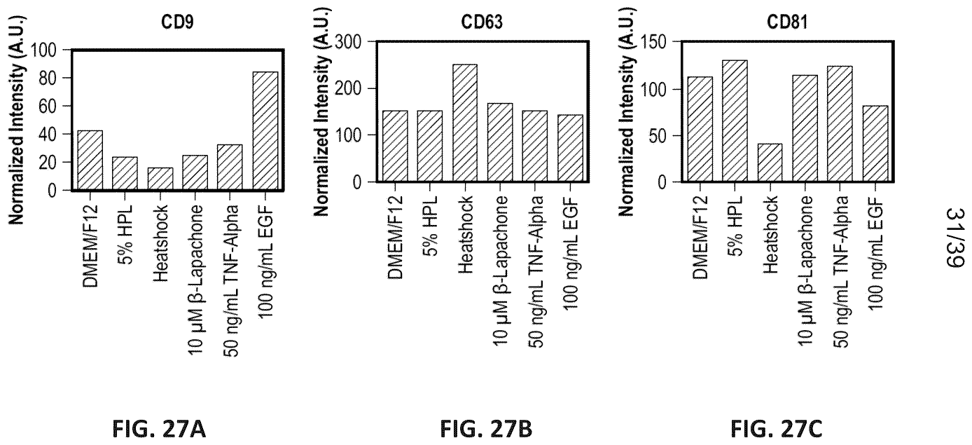

Expression of CD9, CD63, and CD81 (tetraspanins) was differentially modulated (see, FIG. 27A, FIG. 27B, and FIG. 27C). Elevation of CD83 occurred in EVs from heat shocked cells, which is an integral part of the EV manufacturing process.

### Example 9: Adipose-Derived Stem Cell Extracellular Vesicle Compositions for Treating Skin Inflammation

### Background

Inflammatory skin conditions include both chronic diseases and acute sunburn, affecting over 35 million Americans who annually spend over $2 billion to treat their symptoms. Both pro- and anti-inflammatory cytokines are involved in the inflammation process. Sustained elevation of pro-inflammatory cytokines during chronic skin diseases is believed to be responsible for the extensive damage to the skin. These diseases are triggered and maintained by aberrant responses of the cells of the skin immune system. According to the CDC, more than one-third of adults and nearly 70% of children admit to getting sunburned within the past year.

A single acute blistering sunburn during childhood or adolescence more than doubles the chance of developing melanoma in the future, according to the Skin Cancer Foundation. A person's risk for melanoma also doubles if he or she has had five or more acute sunburns at any age. Inflammation resulting from sunburn. The body's attempt at self-protection, occurs to remove harmful stimuli, including cells sustaining DNA damage from UVB rays. While reducing this acute inflammation as quickly as possible is important for a person's immediate well-being, doing so at the expense of not removing DNA damaged cells can have more severe consequences later on in life, such as premature aging and carcinogenesis.

It has been discovered that preparations of extracellular vesicles (EVs) containing exosomes, isolated from the media of cultured adipose-derived adult stem cells, can reduce production of pro-inflammatory cytokines in epidermal skin keratinocytes in response to UVB exposure, while also protecting from harmful DNA damage. Epidermal keratinocytes are highly active immunological cells; they exert a major control over the acute (sunburn) and the chronic (skin diseases) phase of skin inflammation by means of cytokine production (1). Downregulating the expression of pro-inflammatory cytokines is expected to alleviate inflammatory damage.

This example provides an assessment of the use of EV preparations containing exosomes as a topically applied agent for reducing skin inflammation and potential DNA damage.

While the mechanism responsible for acute inflammation of the skin in response to UVB is not well understood, one of the first causal links involving microRNAs (miRNAs) was established by Bernard et. al. (3). miRNAs comprise a family of small non-translated RNAs (approx. 19-24 nucleotides in length) that are expressed in animals, plants and viruses (4, 5). Whole transcriptome sequencing revealed that UVB irradiation of keratinocytes induces alterations in the double-stranded domains of some of these noncoding miRNAs. UVB damage of miRNAs is found to be sufficient to stimulate production of the inflammatory cytokines tumor necrosis factor α (TNFα) and interleukin 6 (IL-6) in non-irradiated cells.

The observation that topical EV application to UVB exposed keratinocytes results in a downregulation of TNF- and IL-8 expression, and that EVs can inhibit STAT3 phosphorylation in response to IL-6, raises the intriguing possibility that EVs may deliver relevant stem cell-derived functional miRNA sufficient to counteract the UVB damaging effect on keratinocyte cellular miRNA. The data generated by an acute inflammation model may also be applied to better understanding and treating chronic inflammatory skin diseases.

Preliminary data, described herein support the continued investigation of stem cell derived EVs containing exosomes as a potential biologic for treating and managing UVB-induced skin inflammation.

The data presented below demonstrates: 1) capabilities in generating reproducible quantities of EVs containing exosomes from the bioreactor system; 2) keratinocyte cell responses to stem cell-derived EVs relevant to UVB-induced inflammation; 3) high-content image analysis that can measure ROS induction and quantitate DNA damage and apoptosis.

Isolating and Characterizing Human Stem Cell-Derived EVs from a Bioreactor

EVs were isolated from human adipose-derived stem cells using ultracentrifugation procedures (7-9). Ultracentrifugation results in optimum EV particle yield compared to precipitation, immunoaffinity chromatography, and ultrafiltration.

For EV production, adult human adipose-derived stem cells (1×10E9 cells) were seeded into FiberCell® Systems Inc. hollow-fiber bioreactor C2011 cartridge (10). Serum-containing medium flows from the reservoir bottle by means of a pulsatile perfusion pump through the oxygenation circuit where it is oxygenated and saturated with CO₂. Serum-free medium (40 mL) was added to the cell-containing bioreactor cartridge.

This scale-up production method has been previously reported (11) and these EVs are currently made commercially available to researchers, one group of which developed a nanoparticle detection technology using these particles (12).

EVs were isolated by removing the serum-free cartridge medium at 3- or 4-day intervals and replacing with fresh medium. The conditioned medium was then subjected to centrifugation in two steps: 1) 3000×g, 20 minutes to remove cell debris; 2) 100,000×g, 2 hours to pellet EVs. EVs were resuspended in DPBS and stored at −80 °C until use. Yields obtained for the bioreactor, compared to T225 flasks, are summarized in Table 15.

TABLE 15
Yields Obtained from Bioreactor Compared to T225 Flasks

| | Medium Volume | Total Number of | Mean Particle Size |
|---|---|---|---|
| | (mL) | EVs (10E11) | (nm) |
| Bioreactor Run # 1 | 40 | 4.1 | 150.6 |
| Bioreactor Run # 2 | 40 | 2.2 | 147.5 |
| Bioreactor Run # 3 | 40 | 4.8 | 149.4 |
| 130 Flasks (T225) | 4000 | 0.16 | 141.0 |

Fluorescent activated cell sorting (FACS) analysis for the stem cell surface marker profiles, as defined by the International Society for Cellular Therapy (ISCT) (13), comprising CD105⁺, CD73⁺, CD90⁺, CD45⁻, CD34⁻, CD14⁻, CD19⁻, HLA-ABC⁺, and HLA-DR⁻, showed that these markers remained constant for cells within the bioreactor over an 8-10 week time frame of medium harvests.

### EV Characterization

To determine the size distribution and concentration of the EVs, samples were analyzed by tunable resistive pulse sensing (TRPS; (qNano®, Izon Science®, Ltd) using a NP150 nanopore membrane at a 47 mm stretch. The concentration of particles was standardized using multi-pressure calibration with 114 nm carboxylated polystyrene beads at a concentration of 1.0×10E13 particles/mL.

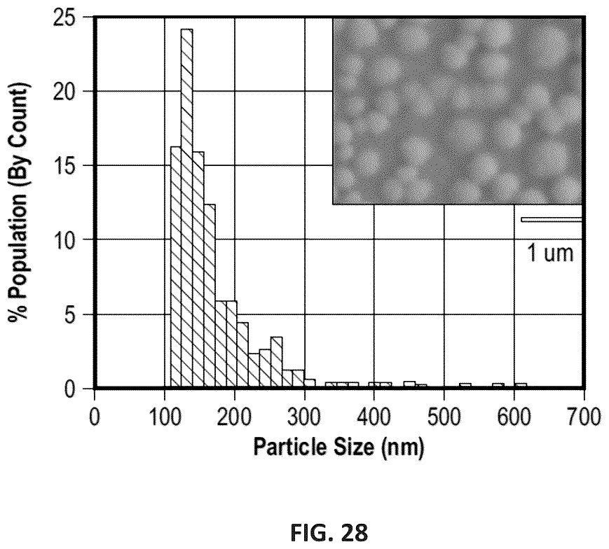

FIG. 28 shows tunable resistive pulse sensing (TRPS) analysis of the size distribution of exosomes isolated from human ASCs in a representative sample using a NP150 nanopore membrane at a 47 mm stretch, demonstrating a mean particle diameter of 144.4 nm, with a large population of particles between 100 and 120 nm. The inset is a scanning electron microscopy (SEM) image demonstrating that the exosome preparations contain discrete particles.

Additional EV samples were analyzed by scanning electron microscopy (SEM) to determine their relative size and shape. EVs were dried on the mounting studs, coated with platinum and visualized as shown in FIG. 28 (inset). The results showed that the EV preparation contained discrete particles of a larger average particle size by SEM than did the preparation shown in FIG. 28; however, this could be due to SEM preparation and drying artifacts.

As part of the characterization process, EV preparations also were analyzed for the presence of exosome marker proteins (14-16) using Miltenyi's MACSPlex® Exosome Kit and flow cytometry. Since the kit uses the exosome markers CD81 (a tetraspanin; cell-surface proteins that mediate signal transduction events) CD63 (postulated role in growth regulation) and CD9 (also a tetraspanin, involved in cell adhesion), to select particles for analysis, it is confirmed that there are exosomes within the EV preparations (data not shown).

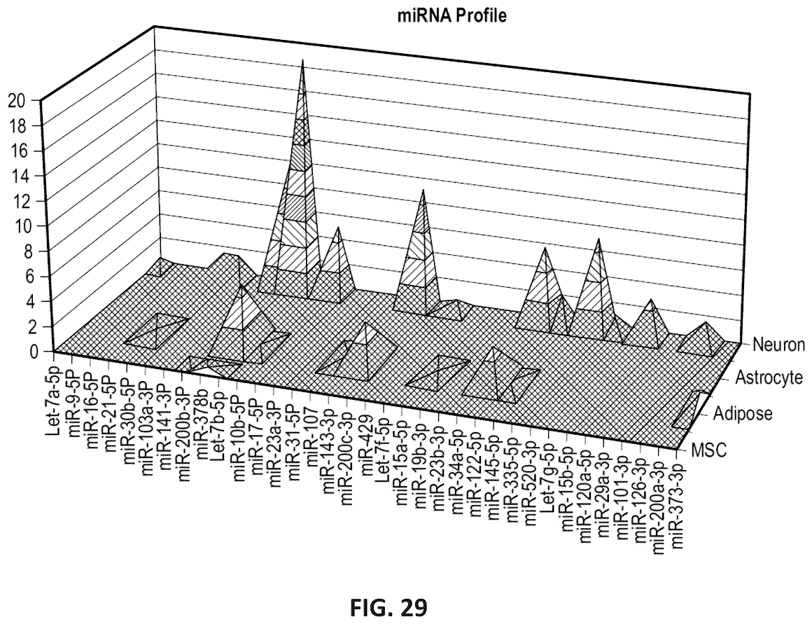

FIG. 29 shows a three-dimensional graph depicting the miRNA profile of exosomes isolated from human ASCs, as analyzed using the MACSPlex® miRNA kit, across four different cell types: (1) MSCs; (2) adipose cells; (3) astrocytes; and (4) neurons, revealing differential miRNA expression among the cell types. that the data show that some of the miRNA enriched in these different cell types include those involved in relieving repression of immune-modulatory cytokines IL-6 and IL-10 (17), in promoting differentiation of keratinocytes (18), in suppressing cell proliferation (10), and in functioning as a possible tumor suppressor (20).

### UVB-Treatment of Keratinocytes

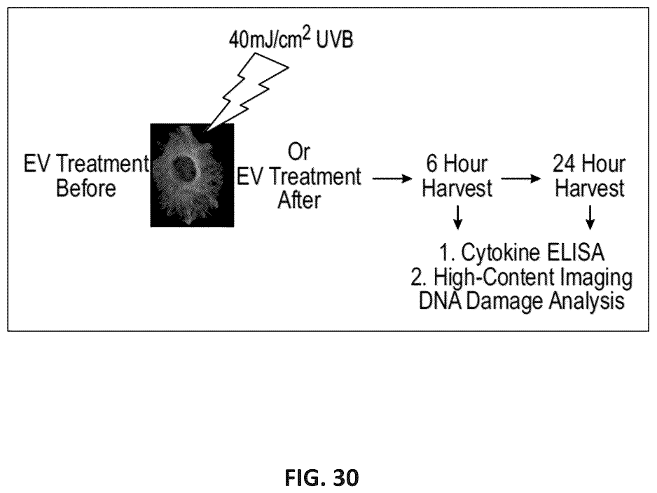

The model system used for these experiments is schematically represented in FIG. 30. Briefly, sub-confluent cultures of keratinocytes were treated for 1 hour with EVs and then exposed to UVB to induce pro-inflammatory cytokines. The choice of 40 mJ/cm² UVB is based on the minimum energy/area/1 hr time in which the reported effects are observed without incurring appreciable cell death. Control cells either were treated with EVs but not exposed to UVB or exposed to UVB only with no EV treatment.

Media was harvested at either 6 hours or 24 hours post-UVB exposure and assayed for IL-8 and TNF-α cytokines by ELISA.

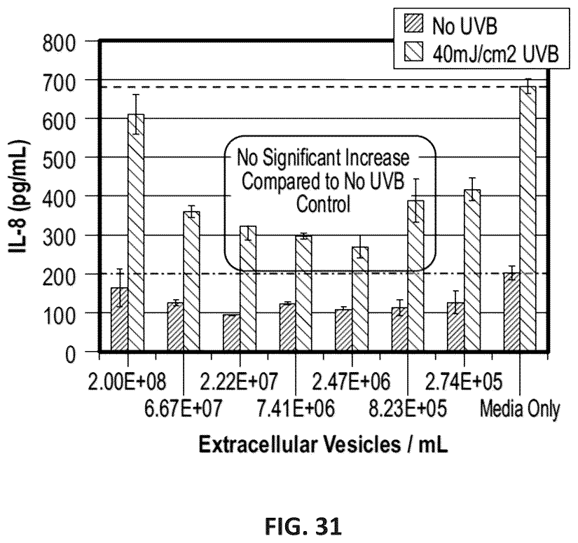

FIG. 31 is a bar graph demonstrating interleukin-8 (IL-8) concentration in culture media harvested 6 hours after keratinocyte exposure to UVB and treatment with EVs isolated from human ASCs at the indicated concentrations.

Significant inhibition of IL-8 production was observed at 6 hours (FIG. 31). This indicates that treatment of keratinocytes with EVs at these concentrations reduces IL-8 to near basal levels. Additional control experiments showed that the EV treatment concentrations used were not toxic and did not affect keratinocyte proliferation (data not shown).

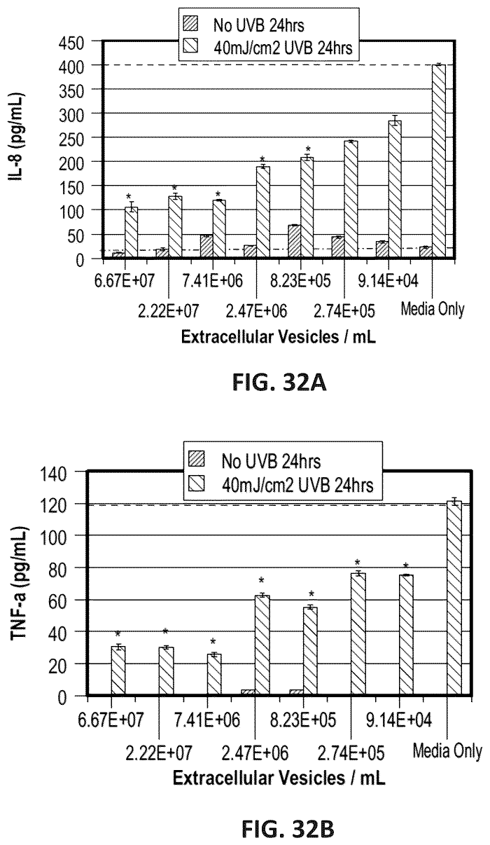

FIG. 32A and FIG. 32B are bar graphs demonstrating interleukin-8 (IL-8) and tumor necrosis factor-alpha (TNF-α) concentrations in culture media harvested 24 hours after keratinocyte exposure to UVB and treatment with EVs isolated from human ASCs at the indicated concentrations. FIG. 32A is a bar graph demonstrating IL-8 concentration in culture media harvested 24 hours after keratinocyte exposure to UVB and treatment with EVs at the indicated concentrations. FIG. 32B is a bar graph demonstrating TNF-α concentration in culture media harvested 24 hours after keratinocyte exposure to UVB and treatment with EVs at the indicated concentrations.

At 24 hours, EV treatment significantly inhibited the production of both IL-8 and TNF-α.

While these data support a protective role for the EVs, a potential restorative role was also tested. In these experiments, keratinocytes were first exposed to UVB for 1 hour, EVs then were added to the culture medium, and ELISA assays performed on the culture medium at 24 hours post-EV treatment.

EV treatment after UVB exposure was found to significantly inhibit the production of IL-8 and TNF-α at the 24 hour time point, supporting a restorative effect of the treatment (FIG. 32A and FIG. 32B).

### DNA Damage

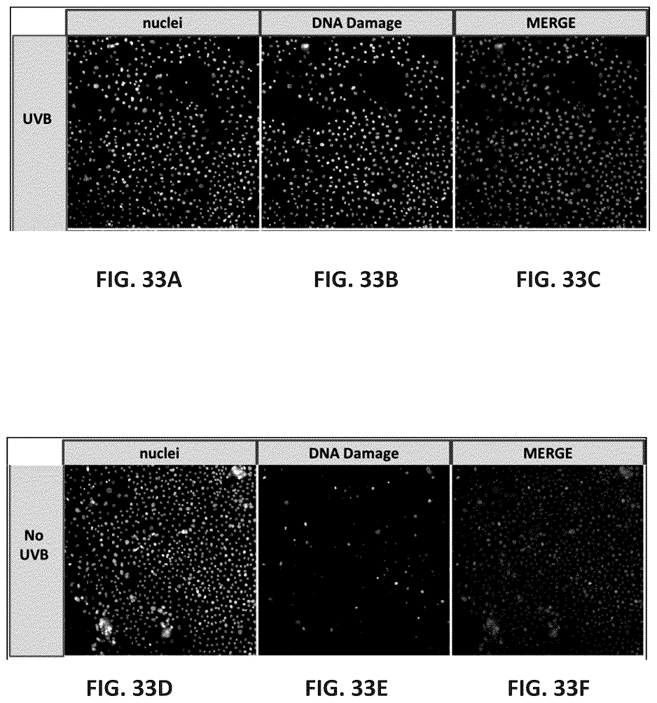

Histone H2AX phosphorylation on a serine four residues from the carboxyl terminus (producing γ-H2AX) is a sensitive marker for DNA double-stranded breaks in keratinocytes following UVB exposure (21-23). This damage is measured using an anti-gamma H2A.X (phospho S139) antibody ([9F3]; Abcam®) and visualized using high-content imaging (FIGS. 33A-33F).

FIG. 33A, FIG. 33B, FIG. 33C, FIG. 33D, FIG. 33E, and FIG. 33F show detection of DNA damage in human keratinocytes following UVB irradiation. Human keratinocytes were cultured to confluence and exposed to 40 mJ/cm² UVB, with unexposed cells serving as a control. Following exposure, cells were fixed and immunostained for DNA double-strand breaks using a phospho-γ-H2AX antibody and Hoechst nuclear stain. FIG. 33A shows the nuclei of cells exposed to UVB. FIG. 33B shows the DNA damage of cells exposed to UVB. FIG. 33C shows the merged image of nuclei (blue) and DNA damage (green) of cells exposed to UVB. FIG. 33D shows the nuclei of unexposed control cells. FIG. 33E shows the DNA damage of unexposed control cells. FIG. 33F shows the merged image of nuclei (blue) and DNA damage (green) of unexposed control cells, demonstrating markedly fewer cells exhibiting DNA damage compared to UVB-exposed cells.

Image quantitation in a separate experiment revealed a 50% decrease in DNA double stranded breaks following EV treatment with 7.41×10E6 particles 1 hour before UVB exposure (data not shown).

### Reactive Oxygen Species (ROS) Induction

UVB radiation induces DNA damage, oxidative stress, and inflammatory processes in human keratinocytes resulting in skin inflammation, photoaging, and photocarcinogenesis (24). Preventing ROS induction improves skin protection against UVB-induced sunburn and photocarcinogenesis (25).

To provide a more accurate and sensitive measure of ROS induction in cultured cells, a platform using High Content Image Analysis (HCA) has been developed for assessing chemical toxin response variability in human endothelial colony cells. This platform has been adapted to test ROS induction in human keratinocytes using UVB as the inducer.

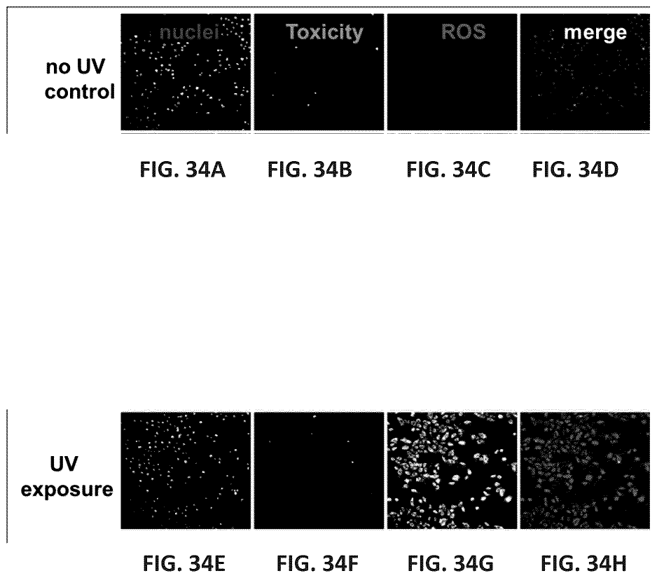

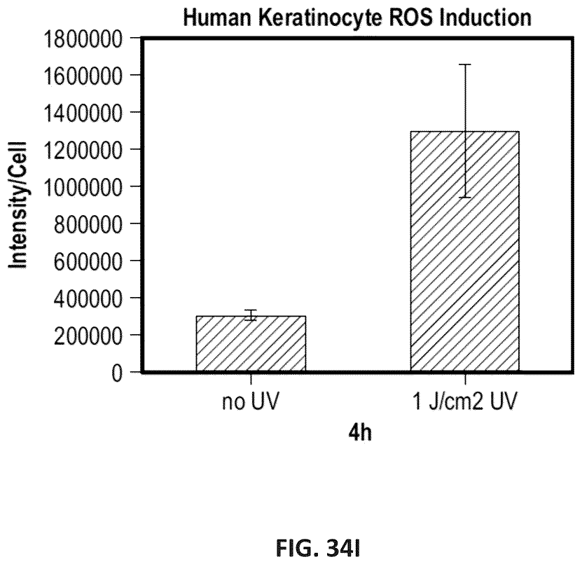

FIG. 34A, FIG. 34B, FIG. 34C, FIG. 34D, FIG. 34E, FIG. 34F, FIG. 34G, FIG. 34H, and FIG. 34I show immunofluorescent imaging analysis and quantification of human keratinocytes dosed with 1 J/cm² UV light. Cells were monitored for toxicity and ROS induction after 4 hours of recovery using high content image analysis (HCA). FIG. 34A shows nuclei labeled with Hoechst stain in keratinocytes that were not exposed to UV irradiation. FIG. 34B shows compromised membrane integrity labeled with ImageIT™ Dead Stain in keratinocytes that were not exposed to UV irradiation. FIG. 34C shows ROS activity labeled with CellROX™ dye in keratinocytes that were not exposed to UV irradiation. FIG. 34D shows the merged image of nuclei (blue), compromised membrane integrity (green), and ROS activity (red) in keratinocytes that were not exposed to UV irradiation. FIG. 34E shows nuclei labeled with Hoechst stain in keratinocytes that were exposed to UV irradiation. FIG. 34F shows compromised membrane integrity labeled with ImageIT™ Dead Stain in keratinocytes that were exposed to UV irradiation. FIG. 34G shows ROS activity labeled with CellROX™ dye in keratinocytes that were exposed to UV irradiation. FIG. 34H shows the merged image of nuclei (blue), compromised membrane integrity (green), and ROS activity (red) in keratinocytes that were exposed to UV irradiation.

FIG. 34I is a bar graph demonstrating the quantification of the immunofluorescent images shown in FIG. 34A, FIG. 34B, FIG. 34C, FIG. 34D, FIG. 34E, FIG. 34F, FIG. 34G and FIG. 34H, indicating robust induction of ROS activity after UV exposure. 25 images per condition were analyzed and total intensity per cell was quantified.

### Exosome Function—Mechanism of Action Study

Signal transducer and activator of transcription (STATs) are inactive transcription factors localized in the cell cytoplasm. In mammals, the STAT family is composed of seven structurally and functionally analogous proteins including, STAT1, STAT2, STAT3, STAT4, STAT5A, STAT5B and STAT6, which have been found to regulate genes expression related to cell proliferation, survival, differentiation, angiogenesis and immune cells function. Binding of many cytokines, hormones and growth factors to their corresponding receptors leads to the activation of receptor-associated Janus activating kinases (JAKs), which can phosphorylate tyrosine residues on the cytoplasmic domain of receptor, providing docking sites for SRC homology 2 (SH2) domain of STAT proteins. Subsequently, these STAT proteins are activated by phosphorylation of their tyrosine residues at their C-terminus by JAKs, leading to their homo- or hetero-dimerization and activation. These STAT dimers translocate to the nucleus and bind to specific sequences in the promoters of target genes and induce their transcription [Gharibi, T. et al. European J. Pharmacology (2020) 878: article 173107].

STAT3 is the key regulator of cell proliferation, survival and apoptosis and is constitutively activated in most human cancers, indicating that it can be a potential therapeutic target for cancer treatment. STAT3 also has important roles in lymphocyte biology, regulation of immune responses and autoimmunity [Id.].

The ability of EVs isolated from human adipose-derived stem cells to downregulate STAT3 phosphorylation in response to IL-6 stimulation of cultured human pulmonary arterial epithelial cells was tested.

Human lung epithelial cells (Invitrogen™) were grown in serum-containing medium to attain confluency. They then were washed on the plate and incubated for 8 hours in serum-free medium (sourced at for example, Miltenyi Biotec®, Sigma-Aldrich®, MilliporeSigma®, Gibco®, Bio-Techne®, Lonza Bioscience®, Corning®, Applied Biological Materials®, ThermoFisher®). EVs (5 or 20 μg) were then added directly to the serum-free medium for an additional 16 hours. IL-6 (5 or 10 ng/mL) (sourced at for example, R&D Systems®, Abcam®, ThermoFisher®, STEMCELL Technologies®, ACROBiosystems®, Proteintech®) was then added and incubated for 10 min (acute stimulation). Cells were then harvested. An ELISA assay to detect pSTAT3 was then run.

As shown in Table 16, EVs (+Exo) effectively blocked the ability of IL-6 to stimulate phosphorylation of STAT3 in this model, thus effectively neutralizing STAT3's transcriptional activity in cytokine-driven signaling pathways (26-28).

TABLE 16
IL-6 and EV (Exo) effects on phosphorylation of STAT3.

| | | | | Fold |
|---|---|---|---|---|
| | P-STAT3 | T-STAT3 | P/T | Induction* |
| Control | 0.11 | 0.24 | 0.46 | 1.00 |
| + IL-6 (5 ng//mL) | 0.44 | 0.24 | 1.80 | 3.94 |
| + IL-6 (20 ng/mL) | 0.39 | 0.24 | 1.59 | 3.46 |
| + Exo (5 μg/mL) | 0.10 | 0.24 | 0.44 | 0.95 |
| + Exo (5 μg/mL) + IL-6 (5 | 0.15 | 0.23 | 0.62 | 1.36 |
| ng/mL) | | | | |
| + Exo (5 μg/mL) + IL-6 (20 | 0.17 | 0.24 | 0.70 | 1.53 |
| ng/mL) | | | | |
| + Exo (20 μg/mL) + IL-6 (5 | 0.12 | 0.24 | 0.50 | 1.10 |
| ng/mL) | | | | |
| + Exo (20 μg/mL) + IL-6 | 0.17 | 0.26 | 0.68 | 1.48 |
| (20 ng/mL) | | | | |

*the ratio (P/T) of phosphorylated (p-STAT3) over total (T-STAT3) compared to the control results in the fold induction of p-STAT3.

### Research Design and Methods

Aim 1. To Examine the Effect of Topically-Applied EVs, Both Pre- and Post-UVB Exposure, on Anti-Inflammatory Cytokine Expression, DNA Damage, Apoptosis, ROS Induction, and Differential miRNA Expression.

Unless otherwise indicated, test samples include EV treatment both before and after 40 mJ/cm² UVB exposure, with sample harvesting at both 6 hours and 24 hours post-UVB exposure. EV test concentrations will contain 2.0×10E8 to 9.14×10E4 particles/mL, inclusive. Testing both pre- and post-UVB exposure is needed to determine whether the EVs are protective (pre), repair/regenerative (post), or both in their effectiveness.

Anti-inflammatory cytokine expression: The Human Inflammation Array ELISA kit (Q3) from RayBiotech™ is used to assess up to 40 different cytokines. This kit includes IL-8 and TNF-α (pro-inflammatory) which were analyzed previously. IL-6, another pro-inflammatory cytokine associated with UVB exposure to skin cells (6) is also included, as well as the anti-inflammatory cytokines IL-10, IL-11, and IL-13. The additional data on other cytokines tested by this kit enables further characterization of the effect of EVs on UVB-exposed keratinocytes, although the focus of this aim is on the aforementioned anti-inflammatory cytokines. RayBiotech™'s Human TGF-1 ELISA kit (ELH-TGF1-1) will also be used, since this cytokine is not included in the Q3 array.

DNA Damage: The preliminary studies will be extended to cover a wider range of particle concentrations (2.0×10E8 to 9.14×10E4 particles/mL, inclusive) and include a time course pre- and post-UVB exposure (T=1, 2, 4, 8, 12, 16, 24 hr) to determine the optimal time for EV treatment to observe the greatest protection against DNA damage (pre-UVB exposure) or elicit repair (post-UVB exposure).

Apoptosis: Two commonly analyzed apoptotic features in high content screening will be employed: nuclear fragmentation and caspase-3 activation upon apoptotic stimulus. Fragmentation of cellular DNA is a hallmark of apoptosis. The TUNEL assay is the most widely used method to detect fragmented DNA in apoptotic cells in tissue samples.

The Click-iT® TUNEL Alexa Fluor® 594 Imaging Assay (ThermoFisher®) utilizes a dUTP modified with an alkyne, a small, bio-orthogonal functional group that enables the nucleotide to be more readily incorporated by TdT than other modified nucleotides, including BrdUTP, biotin-dUTP or fluorescein-dUTP. Compared to other assays using one of the other modified nucleotides, the Click-iT® TUNEL assay is reported to detect a higher percentage of apoptotic cells under identical conditions (29). The assay also allows multiplexing with surface and intracellular biomarker detection, such as caspase-3 (a distinctive feature of early stage apoptosis) and annexin V staining (used to identify intermediate apoptosis stages), which are also assayed. Given that both DNA damage and ROS induction have been previously observed in response to UVB exposure, both of which are associated with apoptosis, it is expected that apoptosis will be observed in this model system, at least for the UVB controls (no EV treatment).

ROS Induction: Keratinocytes are treated with a range of EVs before and after exposing to: a) 1 J/cm² UVB; or b) 40 mJ/cm² UVB. The reason for the two different exposures is that keratinocytes have a high basal level of ROS, and the 40 mJ/cm² UVB, which has been used in other experiments, may not be sufficient to discern ROS induction from basal. Cells are monitored for toxicity and ROS induction after 4 hours post-UVB exposure using high content image analysis. Nuclei are labeled with Hoechst stain in the blue channel. Cells with compromised membrane integrity fluoresce in the green channel using ImageIT® Dead stain (ThermoFisher®), while cells with induced ROS show bright signal in the red channel using CellROX™ dye. A time course post-UVB exposure (T=1, 2, 3, 4) is also performed to determine the optimal time for EV treatment to observe the greatest potential protection against ROS induction.

miRNA analysis of keratinocyte total RNA: miRNA 20b has been shown to be downregulated in keratinocytes following UVB exposure (28); lending more support for the hypothesis that EVs may be delivering relevant stem cell miRNA to counteract the damaging UVB effect on keratinocyte miRNA. The MACPlex® miRNA kit offers a system for semiquantitative analysis of differential miRNA expression, It has been developed for the simultaneous flow cytometric detection of 39 miRNAs, including mir20b. Total RNA samples from the cells are analyzed pre- and post-UVB exposure, as well as +/−EV treatment. This information is used to further address the effects of UVB and potential miRNA restorative potential of EVs.

Considerations: It is possible that an effect on the anti-inflammatory cytokines being targeted may not be observed. Nonetheless, additional cytokine data from the Q3 kit may reveal others which may be affected. For apoptosis, should nuclear DNA fragmentation, caspase-3, and annexin V staining fail to reveal any positive results, mitochondrial membrane potential and other mitochondrial assays may then be tried, as an alternative mechanism of UVB-inducted apoptosis, since mitochondrial changes can occur early in apoptosis. It is also appreciated that UVB exposures between 40 mJ/cm² UVB and 1 J/cm² UVB may need to be titrated to optimize the ROS assay.

Aim 2. To Test the Hypothesis that EV Treatment Downregulate UVB-Induced STAT3 Phosphorylation.

It has previously been shown that EV treatment of cultured human epithelial cells downregulates IL-6-stimulated STAT3 phosphorylation, thus effectively neutralizing STAT3's transcriptional activity in cytokine-driven signaling pathways (26). Since UVB induces phosphorylation of STAT3 in keratinocytes (27), most likely through ROS and DNA damage (30), one question is whether EV treatment can downregulate UVB-induced STAT3 phosphorylation.

To test this, the cells are cultured to confluency. The plate is washed to remove any serum proteins and then incubated for 8 hrs in serum-free medium. EVs (5 or 20 μg) are then added directly to this serum-free medium for an additional 16 hrs. IL-6 (5 or 10 ng/ml) (sourced at for example, R&D Systems®, Abcam®, ThermoFisher®, STEMCELL Technologies®, ACROBiosystems®, Proteintech®) is then added and incubated for 10 min (acute stimulation). Cells are then harvested and ELISA assay run for pSTAT3. p-STAT3 fold induction is then calculated.

Considerations: It is possible that the keratinocyte medium supplement components, EV concentration, IL-6 dosage and incubation time may need to be adjusted to optimize the response.

Summary: Aim 1 is designed to further investigate the cellular responses to UVB exposure with EV treatment. If anti-inflammatory cytokines and ROS induction are not found to be affected by EV treatment, the focus will be on testing for pro-inflammatory cytokine downregulation and DNA damage protection, for which preliminary data currently exists. Likewise, miRNA analysis of keratinocyte total RNA is used to further address the effects of UVB and potential miRNA restorative potential of EVs. Aim 2, testing of the hypothesis that EV treatment downregulates UVB-induced STAT3 phosphorylation is a valid pursuit independent of the results obtained for Aim 1.

### REFERENCES

(1) Albanesi C, Pastore S. Pathobiology of chronic inflammatory skin diseases: interplay between keratinocytes and immune cells as a target for anti-inflammatory drugs. Curr Drug Metab. (March 2010); 11(3): 210-27.
(2) Raposo G, Stoorvogel W. Extracellular vesicles: Exosomes, microvesicles, and friends. J Cell Biol. (Feb. 18, 2013); 200(4): 373-383. doi: 10.1083/jcb.201211138 PMCID: PMC3575529.
(3) Bernard J J, Cowing-Zitron C, Nakatsuji T, Muehleisen B, Muto J, Borkowski A W, Martinez L, Greidinger E L, Yu B D, Gallo R L. Ultraviolet radiation damages self noncoding RNA and is detected by TLR3. Nat Med. (August 2012); 18(8): 1286-90. doi: 10.1038/nm.2861.
(4) Bartel D P. MicroRNAs: genomics, biogenesis, mechanism, and function. Cell. (2004); 116: 281-297.
(5) Grimson A, Srivastava M, Fahey B, Woodcroft B J, Chiang H R. Early origins and evolution of microRNAs and Piwi-interacting RNAs in animals. Nature. (2008); 455: 1193-1197.
(6) Moldovan L, Batte K, Wang Y, Wisler J, Piper M. Analyzing the Circulating MicroRNAs in Exosomes/Extracellular Vesicles from Serum or Plasma by qRT-PCR. Methods Mol Biol. (2013); 1024: 129-145. doi: 10.1007/978-1-62703-453-1 10.
(7) Alvarez M L, Khosroheidari M, Kanchi Ravi R, DiStefano J K. Comparison of protein, microRNA, and mRNA yields using different methods of urinary exosome isolation for the discovery of kidney disease biomarkers. Kidney International. (2012); 82 (9): 1024-32.
(8) Taylor D D, Zacharias W, Gercel-Taylor C. Exosome isolation for proteomic analyses and RNA profiling. Methods in Molecular Biology. (2011); 728: 235-46.
(9) Welton J L, Khanna S, Giles P J, Brennan P, Brewis I A, Staffurth J, Mason M D, Clayton A. Proteomics analysis of bladder cancer exosomes. Molecular & Cellular Proteomics: MCP. (2010); 9 (6): 1324-38.
(10) Cadwell J S. Culture of Placental Derived Cells in a Hollow Fiber Bioreactor Cartridge. http://fibercellsystems.com/documents/FibercellSystemsPlacental % 20Stem %20Cell %20Cultu re.pdf
(11) Whitford W, Ludlow J W, Cadwell J J S. Continuous production of exosomes. Gen Engin & Biotechnol News. (2015); 35(16): 34.
(12) Wunsch B H, Smith J T, Gifford S M, Wang C, Brink M, Bruce R L, Austin R H, Stolovitzky G, Astier Y. Nanoscale lateral displacement arrays for the separation of exosomes and colloids down to 20 nm. Nat Nanotechnol. (Aug. 1, 2016). doi: 10.1038/nnano.2016.134 [Epub ahead of print].
(13) Dominici M, LeBlanc K, Mueller I, Slaper-Cortenbach I, Marini F, Krause D, Deans R, Keating A, Prockop D J, Horwitz E. Minimal criteria for defining multipotent mesenchymal stromal cells. The International Society for Cellular Therapy position statement. Cytotherapy. (2006); 8: 315.
(14) Lancaster G, Febbraio M. Exosome-dependent trafficking of HSP70: a novel secretory pathway for cellular stress proteins. J Biol Chem. (Epub: Apr. 12, 2005); 280(24): 23349-55.
(15) Jakobsen K R, Paulsen B S, Bæk R, Varming K, Sorensen B S, Jørgensen MM. Exosomal proteins as potential diagnostic markers in advanced non-small cell lung carcinoma. J Extracell Vesicles. (Mar. 2, 2015); 4: 26659. doi: 10.3402/jev.v4.26659 eCollection 2015.
(16) Street J M, Barran P E, Mackay C L, Weidt S, Balmforth C, Walsh T S, Chalmers R T, Webb D J, Dear J W. Identification and proteomic profiling of exosomes in human cerebrospinal fluid. Transl Med. (Jan. 5, 2012); 10: 5. doi: 10.1186/1479-5876-10-5.
(17) Schulte L N, Eulalio A, Mollenkopf H J, Reinhardt R, Vogel J. Analysis of the host microRNA response to Salmonella uncovers the control of major cytokines by the let-7 family. EMBO J. (May 18, 2011); 30(10): 1977-89. doi: 10.1038/emboj.2011.94 Epub 2011 Apr. 5.
(18) Wang X L, Zhang T, Wang J, Zhang D B, Zhao F, Lin X W, Wang Z, Shi P, Pang X N. MiR-378b Promotes Differentiation of Keratinocytes through NKX3.1. PLoS One. (Aug. 27, 2015); 10(8): e0136049. doi: 10.1371/journal.pone.0136049.
(19) Qiu M, Liang Z, Chen L, Tan G, Wang K, Liu L, Liu J, Chen H. MicroRNA-429 suppresses cell proliferation, epithelial-mesenchymal transition, and metastasis by direct targeting of BMI1 and E2F3 in renal cell carcinoma. Urol Oncol. (July 2015); 33(7): 332.e9-18. doi: 10.1016/j.urolonc.2015.03.016 Epub 2015 May 4.
(20) Han T, Yi X P, Liu B, Ke M J, Li Y X. MicroRNA-145 suppresses cell proliferation, invasion and migration in pancreatic cancer cells by targeting NEDD9. Mol Med Rep. (June 2015); 11(6): 4115-20. doi: 10.3892/mmr.2015.3294 Epub 2015 Feb. 3.
(21) Bonner W M, Redon C E, Dickey J S, Nakamura A J, Sedelnikova O A, Solier S, Pommier Y. GammaH2AX and cancer. Nat Rev Cancer. (December 2008); 8(12): 957-67. doi: 10.1038/nrc2523 Epub 2008 Nov. 13.
(22) Halicka H D, Huang X, Traganos F, King M A, Dai W, Darzynkiewicz Z. Histone H2AX phosphorylation after cell irradiation with UV-B: relationship to cell cycle phase and induction of apoptosis. Cell Cycle. (February 2005); 4(2): 339-45 Epub 2005 Feb. 21.
(23) Barnes L, Dumas M, Juan M, Noblesse E, Tesniere A, Schnebert S, Guillot B, Molès JP. GammaH2AX, an accurate marker that analyzes UV genotoxic effects on human keratinocytes and on human skin. Photochem Photobiol. (July-August 2010); 86(4): 933-41. doi: 10.1111/j.1751-1097.2010.00744.x Epub 2010 May 13.
(24) Wölfle U, Esser P R, Simon-Haarhaus B, Martin S F, Lademann J, Schempp C M. UVB-induced DNA damage, generation of reactive oxygen species, and inflammation are effectively attenuated by the flavonoid luteolin in vitro and in vivo. Free Radic Biol Med. (May 1, 2011); 50(9): 1081-93. doi: 10.1016/j.freeradbiomed.2011.01.027 Epub 2011 Jan. 31.
(25) Russo P A, Halliday G M. Inhibition of nitric oxide and reactive oxygen species production improves the ability of a sunscreen to protect from sunburn, immunosuppression and photocarcinogenesis. Br J Dermatol. (August 2006); 155(2): 408-15.
(26) Bromberg J, Darnell J E Jr. The role of STATs in transcriptional control and their impact on cellular function. Oncogene. (2000); 19: 2468-2473.
(27) Syed D N, Afaq F, Mukhtar H. Differential Activation of Signaling Pathways by UVA and UVB Radiation in Normal Human Epidermal Keratinocytes. Photochem Photobiol. (2012).
(28) Kraemer A, Chen I P, Henning S, Faust A, Volkmer B, Atkinson M J, Moertl S, Greinert R. UVA and UVB irradiation differentially regulate microRNA expression in human primary keratinocytes. PLoS One. (Dec. 31, 2013); 8(12): e83392. doi: 10.1371/journal.pone.0083392 eCollection 2013.
(29) Huerta S, Goulet E J, Huerta-Yepez S, Livingston E H. Screening and detection of apoptosis. J Surg Res. (May 1, 2007); 139(1): 143-56 Epub 2007 Jan. 25.
(30) Bito T, Sumita N, Masaki T, Shirakawa T, Ueda M, Yoshiki R, Tokura Y, Nishigori C. Ultraviolet light induces Stat3 activation in human keratinocytes and fibroblasts through reactive oxygen species and DNA damage. Exp Dermatol. (Jul. 1, 2010); 19(7): 654-60. doi: 10.1111/j.1600-0625.2010.01084.x Epub 2010 Apr. 20.

### Example 10: Heat and Light-Modulated miRNA Signature in Exosomes Derived from Human Mesenchymal Stem Cells

### Background

Photobiomodulation (PBM) is a safe phototherapy technique that uses primarily visible red light (620-700 nm, inclusive) and near-infrared (700-1440 nm) to modulate cellular functions in skin [Hernandez-Bule, et al., Int. J. Mol. Sci. (2024) 25, 4483]. PBM uses various light sources, notably low-level lasers and light-emitting diodes (LEDs), and has been applied both in clinics and at home. Its appeal in dermatology stems from its non-invasive nature, minimal side effects, and versatility across conditions including acne, wound healing, skin rejuvenation, and scar reduction [Hernandez-Bule, et al., Int. J. Mol. Sci. (2024) 25, 4483].

The core mechanism involves absorption of photons by endogenous chromophores and activation of intracellular signaling pathways. In skin, principal chromophores include melanin in the epidermis, hemoglobin in the dermis, and water across layers, with longer wavelengths penetrating more deeply. Accordingly, red light largely targets melanin and hemoglobin, whereas near-infrared light (about 750 nm to 1,400 nm, inclusive), which is just beyond the visible spectrum and is not visible to the human eye, engages water in deeper layers to elicit modulatory effects [Hernandez-Bule, et al., Int. J. Mol. Sci. (2024) 25, 4483].

Red light photons penetrate skin more effectively than blue (400-500 nm) or green (495-570 nm) photons because they are less scattered and less absorbed by endogenous photosensitizers (EPs), such as melanin and lipofuscin) and therefore produce fewer reactive oxidants while favoring signaling responses. Therefore, less absorption of red light photons by the EPs allow the skin to experience more of the stimulatory effects induced by the light-skin interactions [Herrera, et al., Front. Photonics (2024) 5:1460722].

The skin operates within an optical wavelength window of roughly 600-1300 nm where absorption coefficients of melanin, hemoglobin, and water is relatively low. The reactions favoring the formation of new reactive oxidants decrease with the increase in wavelength, since few EPs are present in human skin in this optical window [Herrera, et al. Front. Photonics (2024) 5:1460722]. PBM affects the skin consistent with the hormesis dose response concept, which is a biphasic dose response phenomenon characterized by low dose stimulation and high dose inhibition. Therefore, Accordingly, at lower light fluencies, there are stimulating effects, while at higher fluencies, there are inhibitory effects [Herrera, et al. Front. Photonics (2024), 5:1460722].

At the mitochondrial level, red light can activate respiratory chain processes attributed in part to cytochrome c oxidase, increasing ATP production and cellular metabolic activity [Couturaud et al., Skin Res Technol. (2023) 29: e13391]. However, multiple photoreceptors likely contribute, and cytochrome c oxidase is not the sole acceptor implicated in red-light responses [Herrera, et al. Front. Photonics (2024), 5:1460722]. Red light exposure also elevates nitric oxide bioactivity, which can promote vasodilation, inhibit lipid peroxidation reactions, stimulate insulin secretion by releasing calcium from mitochondria, and modulate ion channels relevant to skin physiology [Herrera, et al. Front. Photonics (2024), 5:1460722].

PBM has been shown to modulate gene expression in dermal fibroblasts, downregulating metalloproteinases while upregulating collagen and matrix regulators, thereby supporting extracellular matrix renewal [Herrera, et al., Front. Photonics (2024) 5:1460722]. Moreover, in vitro and ex vivo studies have shown that red and near-infrared exposure increases expression of collagen and elastin and reduces MMPs, improving dermal matrix quality [Hernandez-Bule, et al Int. J. Mol. Sci. (2024) 25, 4483]. At 633 nm, fibroblast cultures demonstrate increased procollagen synthesis and growth factor signaling, aligning with clinical observations [Russell et al., Journal of Cosmetic and Laser Therapy (2005) 7: 196-200].

PBM exerts anti-inflammatory effects by reducing pro-inflammatory cytokines such as IL-6, IL-8 and TNF-α, and by modulating keratinocyte and Langerhans cell responses via Nrf2-linked pathways [Hernandez-Bule, et al. Int. J. Mol. Sci. (2024) 25, 4483, Herrera, et al., Front. Photonics (2024) 5:1460722]. Through coordinated effects on wound-healing phases and extracellular matrix remodeling, PBM supports re-epithelialization and tissue repair [Hernandez-Bule, et al., Int. J. Mol. Sci. (2024) 25, 4483].

Light also influences dermal microcirculation, with PBM activating VEGF-linked pathways in endothelial cells and enhancing cell migration associated with angiogenesis and wound healing. These vascular effects complement its metabolic and immunomodulatory actions to restore tissue homeostasis [Herrera, et al., (2024) Front. Photonics., 5:1460722].

Clinical photo-rejuvenation data show that combined red and near-infrared LED treatments improved profilometry measures and patient-reported periorbital wrinkles after nine sessions over 12 weeks [Russell et al. Journal of Cosmetic and Laser Therapy (2005) 7: 196-200]. A home-use red-LED facial mask with a wavelength of 630±10 nm delivering 15.6 J/cm² for 12 minutes twice weekly over three months produced progressive and significant improvements in wrinkles, firmness, dermal density, roughness, pore diameter, sebum, and complexion homogeneity [Couturaud et al. Skin Res Technol [2023], 29: e13391]. These outcomes support PBM's capacity to reverse visible signs of skin aging with good tolerance and durability after treatment cessation [Couturaud et al. Skin Res Technol. (2023) 29: e13391).

Beyond rejuvenation, PBM has demonstrated benefits across dermatologic indications, including reducing inflammatory acne lesions and sebaceous activity, improving wound and scar healing, and assisting in psoriasis and atopic dermatitis management via anti-inflammatory and immunomodulatory mechanisms [Hernandez-Bule, et al. Int. J. Mol. Sci. (2024) 25, 4483].

Red and near-infrared PBM is generally regarded as safe in human skin, with studies noting no DNA damage even at high fluences and only transient mild reactions [Hernandez-Bule, et al. Int. J. Mol. Sci. (2024), 25, 4483]. Appropriate dosing is essential due to biphasic dose-responses and variations in visible-light sensitivity across skin phototypes [Couturaud et al. Skin Res Technol (2023)., 29: e13391].

### Methods

Six independent human mesenchymal stem cell (MSC) cultures were prepared. Each culture was exposed to a 20 minute heat shock at 42 °C as described in Example 6, immediately followed by 1 hour of light exposure at 630 nm (red), 850 nm (near-infrared), or 940 nm (near-infrared).

The infrared apparatus included 180 LEDs with three chips per LED (540 chips total), delivering a uniform irradiance with 170 mW/cm² of red light.

For direct light exposure, vials containing approximately 1×10E10 exosome particles were placed directly on the LEDs and exposed for 60 minutes at 630 nm (red), 850 nm (near-infrared), or 940 nm (near-infrared).

### Results

Following red-light exposure, the exosomal miRNA cargo exhibited a defined signature (Table 17) of upregulated and downregulated miRNAs relative to non-irradiated controls.

TABLE 17
Tuned miRNA Signature Cargo of Exosomes Exposed to Red
Light

| | | | | SEQ |
|---|---|---|---|---|
| | | | | ID |
| miRNA | Expression | Function | Sequence | NO: |
| miR-29 | Upregulated | miR-29 is known to inhibit | UAGCACCAUCUG | 1 |
| | | the expression of collagen | AAAUCGGUUA | |
| | | and other extracellular matrix | | |
| | | proteins, making it | | |
| | | significant in conditions like | | |
| | | fibrosis and tissue scarring. | | |
| | | Numerous studies have | | |
| | | highlighted its role in | | |
| | | regulating genes involved in | | |
| | | cell cycle and apoptosis, | | |
| | | indicating its importance in | | |
| | | maintaining cellular | | |
| | | homeostasis. | | |
| miR-196a | Upregulated | miR-196a regulates gene | UAGGUAGUUUC | 2 |
| | | expression by binding to | AUGUUGUUGGG | |
| | | complementary sequences in | | |
| | | target mRNAs, leading to | | |
| | | their degradation or | | |
| | | inhibition of translation. It is | | |
| | | involved in various biological | | |
| | | processes, including | | |
| | | development, differentiation, | | |
| | | and disease progression | | |
| miR-9a | Upregulated | miR-9a binds to target | UCUUUGGUUAUC | 3 |
| | | mRNAs, leading to their | UAGCUGUAUGA | |
| | | degradation or inhibition of | | |
| | | translation. | | |
| | | This regulation affects | | |
| | | pathways involved in cell | | |
| | | cycle, apoptosis, and | | |
| | | migration. | | |
| miR-21 | Downregulated | Involved in various cellular | UAGCUUAUCAGA | 4 |
| | | processes, including cell | CUGAUGUUGA | |
| | | proliferation, apoptosis, and | | |
| | | inflammation, which can | | |
| | | impact skin aging and repair. | | |
| miR-145 | Downregulated | Involved in various cellular | GUCCAGUUUUCC | 5 |
| | | processes that can affect skin | CAGGAAUCCCU | |
| | | conditions and overall | | |
| | | appearance. | | |
| miR-23 | Downregulated | Involved in various | GGGGUUCCUGGG | 6 |
| | | biological processes, | GAUGGGAUUU | |
| | | including cell proliferation, | | |
| | | differentiation, and | | |
| | | apoptosis. | | |
| miR-31 | Downregulated | Plays a significant role in | AGGCAAGAUGCU | 7 |
| | | skin care and health. This | GGCAUAGCU | |
| | | microRNA is involved in | | |
| | | various skin processes, | | |
| | | including wound healing, | | |
| | | inflammation, and skin | | |
| | | aging. | | |

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A composition comprising a purified population of potent exosomes derived from extracellular vesicles derived from mesenchymal stem cells (MSCs), wherein
a. the population of exosomes comprise an identity signature comprising expression of biomarkers CD9, CD63, and CD81;
b. the population of exosomes comprise total protein of about 1 mg;
c. the population of exosomes comprise total RNA content greater than 20 μg; and
d. the population of exosomes comprise a cargo comprising one or more, miRNAs;
wherein the miRNA cargo is tunable by exposure to heat, light, or both, and
wherein lot to lot and batch to batch variability relative to prior lots of the composition is below a significance level of ≤0.05.

2. The composition of claim 1, wherein the MSCs originate from a tissue or a body fluid of a mammalian subject.

3. The composition of claim 2, wherein the mammalian subject is human.

4. The composition of claim 2, wherein
(a) the MSCs originate from placental tissue, adipose tissue, umbilical cord tissue, lung tissue, heart tissue, or dental pulp; or
(b) the mesenchymal stem cells originate from bone marrow of normal healthy subjects aged 21-40 years old; or
(c) the body fluid is blood, amniotic fluid or urine.

5. The composition of claim 2, wherein
a. the exosomes originate from mesenchymal cells obtained from adipose tissue (ASCs); or
b, wherein the exosomes derived from MSCs obtained from placental tissue originate from one or more of chorionic membrane (CM), chorionic trophoblast without villi (CT-V), chorionic villi (CV), or decidua (DC); or
c. the blood is umbilical cord blood or peripheral blood.

6. The composition of claim 1, wherein the miRNA cargo of the population of exosomes is tunable by exposure of the MSCs first to a heat shock followed by exposure to red light, near infrared light, UV light or a combination thereof or by exposure of the MSCs first to a heat shock, isolation of the population of exosomes from the heat-shocked MSCs, followed by exposure of the isolated heat-shocked exosomes to red light, near infrared light, UV light, or a combination thereof.

7. The composition of claim 5, wherein
a. the heat shock comprises exposure to a temperature of from about 41 °C to about 43 °C, inclusive;
b. the red light is light of a wavelength from about 620 nanometers (nm) to about 750 nanometers, (nm), inclusive;
c. the near infrared light is light of a wavelength from about 700 nm to about 1400 nm, inclusive; and
d. the ultraviolet light is light of a wavelength from about 10 nm to about 400 nm, inclusive.

8. The composition of claim 6, wherein
the red light exposure is at a wavelength of 630 nm,
the near infrared light exposure is at a wavelength of 850 nm,
the near infrared light exposure is at a wavelength of 940 nm,
or exposure is to a combination thereof.

9. The composition of claim 7, wherein the population of exosomes derived from heat-shocked MSCs after exposure to red light, when compared to a non-heat-shocked and non-irradiated (native) control:
a. contain unique miRNA species not found in native exosomes;
b. comprise one or more downregulated miRNA species selected from miR-21, miR-145, miR-23, and miR-31 compared to the miRNA species in native exosomes;
c. comprise one or more upregulated miRNA species selected from miR-29, miR-196a, and miR-9a; or
d. a combination thereof.

10. The composition of claim 8, wherein an improvement in wound healing by the population of exosomes comprising the tuned miRNAs is by at least one of improved collagen production; improved cell proliferation; improved immune regulation; or improved angiogenesis, compared to a native control.

11. The composition of claim 8, wherein the composition is a pharmaceutical composition comprising a therapeutic amount of the isolated tuned potent exosomes and a pharmaceutically acceptable carrier.

12. The composition of claim 8, wherein the population of exosomes is derived from at least 1×10¹² EVs comprising exosomes per isolation.

13. The composition of claim 8, wherein the pharmaceutical composition is formulated for administration by inhalation, topical administration, or intravenous administration.

14. The composition of claim 10, wherein an effective amount of the purified, tuned potent exosomes comprises at least 1×10⁸ exosomes.

15. The composition of claim 8, wherein the population of exosomes is tuned to treat an age-related chronic disease, which if left untreated, comprises at least one of a progressive injury, progressive inflammation, progressive fibrosis of a tissue or a combination thereof.

16. The composition of claim 14, wherein the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle.

17. The composition of claim 14, wherein the age-related chronic disease is a chronic lung disease, chronic inflammation and immune dysfunction, mitochondrial dysfunction, organ transplantation dysfunction, organ resuscitation and rejuvenation, a viral infection, neuropathic pain; neurofibrosis, neurodegeneration, connective tissue dysfunction, musculoskeletal repair, dysfunction of the gut microbiome, or age-related decline.

18. The composition of claim 16, wherein the chronic lung disease is a fibrotic lung disease.

19. The composition of claim 16, wherein the chronic lung disease is due to chronic smoking or a severe viral infection.

20. The composition of claim 16, wherein the severe lung infection is due to a severe coronavirus infection.

21. The composition of claim 16, wherein the age-related chronic lung disease comprises reduced forced vital capacity compared to a normal healthy control.

22. The composition of claim 1, wherein the subject is a mammal.

23. A method for treating a wound comprising, in order:
a) tuning an miRNA cargo derived from extracellular vesicles (EVs) that originate from a population of mesenchymal stem cells (MSCs) that originate from a tissue or a body fluid of a mammalian subject by:
(i) isolating the population of MSCs from the tissue or the body fluid of the human subject;
(ii) adapting the isolated population of MSCs in vitro to culture conditions at 37 °C to form a cultured population of MSCs at 37 °C;
(iii) heat shocking the cultured population of MSCs at 37 °C by exposing the cultured population of MSCs to a temperature of about 41 °C to about 43 °C, inclusive, to form a heat-shocked population of MSCs comprising a population of exosomes comprising a heat-shocked miRNA cargo; and
(iv) irradiating the heat-shocked population of MSCs of (iii); or
isolating from the heat-shocked population of MSCs the population of exosomes comprising the heat shocked miRNA cargo and irradiating the isolated population of heat-shocked exosomes;
wherein the irradiating comprises exposing the heat-shocked population of MSCs or the isolated population of heat-shocked exosomes to red light, near infrared light, UV light or a combination thereof; and
b) administering by inhalation, topically or parenterally the pharmaceutical composition comprising the population of heat-shocked and irradiated exosomes comprising a tuned cargo of miRNAs.

24. The method according to claim 23, wherein the mammalian subject is a human subject.

25. The method according to claim 23, wherein the tissue comprises:
(a) placental tissue, adipose tissue, umbilical cord tissue, lung tissue, heart tissue, or dental pulp; or
(b) bone marrow of normal healthy subjects aged 21-40 years old.

26. The method according to claim 23, wherein the body fluid is blood, amniotic fluid or urine.

27. The method according to claim 23, wherein
(i) the tissue is adipose tissue,
(ii) the population of MSCs is a population of adipose-derived mesenchymal stem cells (ASCs); and
(iii) the population of exosomes comprising the tuned cargo of miRNAs comprise one or more unique miRNA species compared to the miRNA species in native exosomes.

28. The method according to claim 27, wherein the tuned population of heat-shocked and red light irradiated exosomes
comprises one or more upregulated miRNA species selected from miR-29, miR-196a, and miR-9a compared to the miRNA species in native exosomes; and
comprises one or more downregulated miRNA species selected from miR-21, miR-145, miR-23, and miR-31 compared to the miRNA species in native exosomes.

29. The method according to claim 27, wherein the population of exosomes comprising the tuned cargo of miRNAs treats an age-related chronic disease, which if left untreated, comprises at least one of a progressive injury, progressive inflammation, progressive fibrosis of a tissue or a combination thereof.

30. The method according to claim 27, wherein the tissue is lung tissue, cardiac tissue, renal tissue, hepatic tissue, skin, pancreatic tissue, eye tissue, joint tissue, bone marrow, brain tissue, intestinal tissue, peritoneal tissue, retroperitoneal tissue, nerve tissue, spinal tissue, or skeletal muscle.

31. The method according to claim 27, wherein the age-related chronic disease is a chronic lung disease, chronic inflammation and immune dysfunction, mitochondrial dysfunction, organ transplantation dysfunction, organ resuscitation and rejuvenation, a viral infection, neuropathic pain; neurofibrosis, neurodegeneration, connective tissue dysfunction, musculoskeletal repair, dysfunction of the gut microbiome, or age-related decline.

32. The method according to claim 31, wherein the chronic lung disease is a fibrotic lung disease.

33. The method according to claim 31, wherein the chronic lung disease is due to chronic smoking or a severe viral infection.

34. The method according to claim 31, wherein the chronic lung disease comprises reduced forced vital capacity compared to a normal healthy control.


SEQUENCE LISTING (7 sequences)

SEQ ID NO: 1 - RNA, 22 nt, Homo sapiens
  source 1..22: mol_type = other RNA
tagcaccatctgaaatcggtta

SEQ ID NO: 2 - RNA, 22 nt, Homo sapiens
  source 1..22: mol_type = other RNA
taggtagtttcatgttgttggg

SEQ ID NO: 3 - RNA, 23 nt, Homo sapiens
  source 1..23: mol_type = other RNA
tctttggttatctagctgtatga

SEQ ID NO: 4 - RNA, 22 nt, Homo sapiens
  source 1..22: mol_type = other RNA
tagcttatcagactgatgttga

SEQ ID NO: 5 - RNA, 23 nt, Homo sapiens
  source 1..23: mol_type = other RNA
gtccagttttcccaggaatccct

SEQ ID NO: 6 - RNA, 22 nt, Homo sapiens
  source 1..22: mol_type = other RNA
ggggttcctggggatgggattt

SEQ ID NO: 7 - RNA, 21 nt, Homo sapiens
  source 1..21: mol_type = other RNA
aggcaagatgctggcatagct